(19) United States
(12) Patent Application Publication
(10) Pub. No.: US 2024/0016810 A1
(43) Pub. Date: Jan. 18, 2024

(54) COMPOSITIONS AND METHODS FOR THE TREATMENT AND PREVENTION OF NEUROLOGICAL DISORDERS

(71) Applicant: Kineta, Inc., Seattle, WA (US)

(72) Inventors: Daniel TARDIFF, Arlington, MA (US); Robert SCANNEVIN, Hopkinton, MA (US); Kenneth RHODES, Belmont, MA (US)

(21) Appl. No.: 18/007,788

(22) PCT Filed: Jun. 11, 2021
(86) PCT No.: PCT/US2021/037008

§ 371 Date: Dec. 2, 2022

Related U.S. Application Data
(60) Provisional application No. 63/038,056, filed on Jun. 11, 2020.

(51) Int. Cl. A61K 31/5377; A61K 31/5386; A61K 31/519; A61P 25/28

(52) U.S. Cl. CPC A61K 31/5377; A61K 31/5386; A61K 31/519; A61P 25/28

(57) ABSTRACT

The invention provides compositions and methods for treating neurological disorders, such as amyotrophic lateral sclerosis, frontotemporal degeneration, and Alzheimer's disease, among others. Using the compositions and methods described herein, a patient having a neurological disorder, such as a neurological disorder associated with TAR-DNA binding protein (TDP)-43 aggregation, may be administered an inhibitor of FYVE-type zinc finger containing phosphoinositide kinase (PIKfyve) so as to treat an underlying etiology of the disorder and/or to alleviate one or more symptoms of the disease. The inhibitor of PIKfyve may be a small molecule, an anti-PIKfyve antibody or antigen-binding fragment thereof, or a compound, such as an interfering RNA molecule, that attenuates PIKfyve expression. Patients that may be treated using the compositions and methods described herein include those that express are susceptible to developing TDP-43-mediated aggregation and toxicity.

### FIELD OF THE INVENTION

The invention relates to the field of therapeutic treatment of neurological disorders in patients, such as human patients.

### BACKGROUND OF THE INVENTION

Amyotrophic lateral sclerosis (ALS), also known as Lou Gehrig's disease, is an aggressive, debilitating neurological disorder in which affected patients succumb within 2 to 5 years after diagnosis. ALS presents with heterogeneous clinical features but has a common underlying pathology of motor neuron loss that limits the central nervous system's ability to effectively regulate voluntary and involuntary muscle activity. Additionally, without neuronal trophic support muscles being to atrophy, further exacerbating motor deterioration. Cellular and tissue degeneration results in motor impairment such as fasciculations and weakening in the arms, legs and neck, difficulty swallowing, slurred speech and ultimately failure of the diaphragm muscles that control breathing. There remains a need for improved treatments for ALS, as well as other neurological disorders.

### SUMMARY OF THE INVENTION

The present disclosure relates to compositions and methods for treating neurological disorders, such as amyotrophic lateral sclerosis, among others, including neuromuscular disorders and various other neurological conditions. Using the compositions and methods described herein, a patient having a neurological disorder, such as amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, or hereditary inclusion body myopathy, may be administered an inhibitor of FYVE-type zinc finger containing phosphoinositide kinase (PIKfyve) so as to treat an underlying etiology of the disorder and/or to alleviate one or more symptoms of the disease.

The inhibitor of PIKfyve may be, for example, a small molecule, such as a small molecule describe herein. In some embodiments, the PIKfyve inhibitor is an anti-PIKfyve antibody or antigen-binding fragment thereof, or a compound, such as an interfering RNA molecule, that attenuates PIKfyve expression.

Patients that may be treated using the compositions and methods described herein include those that exhibit, and/or that are prone to develop, aggregation of TAR-DNA binding protein (TDP)-43. Example of patients that may exhibit or may be prone to exhibit TDP-43 aggregation are those that express a mutant TDP-43 isoform containing a mutation that renders this protein susceptible to aggregation. For example, patients that may be treated using the compositions and methods described herein include those expressing a TDP-43 isoform having a mutation selected from A315T, Q331 K, M337V, D169G, G294A, G294V, Q343R, G295S, N345K, R361S, N390D, A382T, and G376D, among others that are associated with TDP-43 aggregation and toxicity in vivo.

In a first aspect, the disclosure features a method of treating a neurological disorder in a patient, such as a human patient, by providing to the patient a therapeutically effective amount of a PIKfyve inhibitor. In some embodiments, the patient is one that does not have a mutation that gives rise to an expanded hexanucleotide repeat in a c9orf72 gene. In some embodiments, the patient has a mutation in one or more of genes SETX, ATXN2, SOD1, VABP, ALS2, ANG, SQSTM1, C21ORF2, MATR3, EWSR1, TAF15, HNRPA1, HNRNPA2B1, OPTN, TUBA4A, TARDBP, DCTN1, TUBA4A, TBK1, CHCHD10, CCNF, FUS, UBQLN2, SIGMAR1, TIA1, CHMP2B, VCP, GRN, MAPT, and TMEM106B.

In another aspect, the disclosure features a method of treating a neurological disorder in a patient, such as a human patient, identified as likely to benefit from treatment with a PIKfyve inhibitor on the basis of TDP-43 aggregation. In this aspect, the method may include (i) determining that the patient exhibits, or is prone to develop, TDP-43 aggregation, and (ii) providing to the patient a therapeutically effective amount of a PIKfyve inhibitor. In some embodiments, the patient has previously been determined to exhibit, or to be prone to developing, TDP-43 aggregation, and the method includes providing to the patient a therapeutically effective amount of a PIKfyve inhibitor. The susceptibility of the patient to developing TDP-43 aggregation may be determined, e.g., by analyzing the morphology and gene expression patterns of neuronal cells obtained by differentiation of induced pluripotent stem cells (iPSCs) derived from the patient. For example, to assess the patient's propensity of developing TDP-43 aggregation, a sample of somatic cells may be isolated from the patient and reprogrammed into iPSCs.

The somatic cells may be, for example, hematopoietic cells. The isolated somatic cells may reprogrammed into iPSCs by contacting the cells with one or more agents that increase expression and/or activity of Oct4, Sox2, cMyc, and/or Klf4. Upon reprograming the somatic cells into iPSCs, the iPSCs may then be differentiated into motor neurons. Methods for differentiating iPSCs into motor neurons are described, for example, in Fujimori et al., Nature Medicine 24:1579-1589 (2018); Fujimori et al., Mol. Brain 9:88 (2016); Fujimori et al., Stem Cell Reports 9:1675-1691 (2017); and Matsumoto et al., Stem Cell Reports 6:422-435 (2016), the disclosures of each of which are incorporated herein by reference. Once the iPSCs are differentiated into motor neurons, the motor neurons may be monitored for changes in morphology and gene expression that are consistent with TDP-43 aggregation and the onset of neurological disorders.

For example, (e.g., in embodiments in which the neurological disorder is amyotrophic lateral sclerosis), the patient's propensity to develop TDP-43 aggregation can be assessed by analyzing the time-dependent neurite outgrowth patterns of motor neurons obtained by differentiation of iPSCs reprogrammed from mature hematopoietic cells isolated from the patient. In some embodiments, TDP-43 aggregation is signaled by a finding that neurites on such motor neurons exhibit a decrease in size and/or a decrease in their rate of growth after a period of time following differentiation in vitro. For example, TDP-43 aggregation may be signaled by a finding that neurites on such motor neurons exhibit a decrease in size and/or a decrease in their rate of growth after from about 10 days to 100 days following differentiation (e.g., after from about 20 days to about 60 days following differentiation, after from about 21 days to about 59 days following differentiation, after from about 22 days to about 58 days following differentiation, after from about 23 days to about 57 days following differentiation, after from about 24 days to about 56 days following differentiation, after from about 25 days to about 55 days following differentiation, after from about 26 days to about 54 days following differentiation, after from about 27 days to about 53 days following differentiation, after from about 28 days to about 52 days following differentiation, after from about 29 days to about 51 days following differentiation, after from about 30 days to about 50 days following differentiation, after from about 31 days to about 49 days following differentiation, after from about 32 days to about 48 days following differentiation, after from about 33 days to about 47 days following differentiation, after from about 34 days to about 46 days following differentiation, after from about 35 days to about 45 days following differentiation, after from about 36 days to about 44 days following differentiation, after from about 37 days to about 43 days following differentiation, after from about 38 days to about 42 days following differentiation, after from about 39 days to about 41 days following differentiation, after from about 35 days to about 40 days following differentiation, or after from about 40 days to about 45 days following differentiation).

In some embodiments (e.g., when the neurological disorder is amyotrophic lateral sclerosis), TDP-43 aggregation is signaled by a finding that motor neurons obtained by differentiation from iPSCs reprogrammed from somatic cells (e.g., hematopoietic cells) isolated from the patient begin to undergo apoptosis after a period of time following differentiation in vitro. Apoptosis of such motor neurons may be assessed, for example, by monitoring the presence of leaked lactate dehydrogenase (LDH) and/or cleaved caspase-3 (CC3) in a sample of the motor neurons. In this context, a finding that the concentration of leaked LDH has increased and/or that the relative quantity of CC3+ neurons has increased is indicative of apoptosis. TDP-43 aggregation may be signaled by a finding that such motor neurons exhibit an increase in leaked LDH concentration and/or an increase in CC3 expression after from about 10 days to 100 days following differentiation (e.g., after from about 20 days to about 60 days following differentiation, after from about 21 days to about 59 days following differentiation, after from about 22 days to about 58 days following differentiation, after from about 23 days to about 57 days following differentiation, after from about 24 days to about 56 days following differentiation, after from about 25 days to about 55 days following differentiation, after from about 26 days to about 54 days following differentiation, after from about 27 days to about 53 days following differentiation, after from about 28 days to about 52 days following differentiation, after from about 29 days to about 51 days following differentiation, after from about 30 days to about 50 days following differentiation, after from about 31 days to about 49 days following differentiation, after from about 32 days to about 48 days following differentiation, after from about 33 days to about 47 days following differentiation, after from about 34 days to about 46 days following differentiation, after from about 35 days to about 45 days following differentiation, after from about 36 days to about 44 days following differentiation, after from about 37 days to about 43 days following differentiation, after from about 38 days to about 42 days following differentiation, after from about 39 days to about 41 days following differentiation, after from about 35 days to about 40 days following differentiation, or after from about 40 days to about 45 days following differentiation).

In an additional aspect, the disclosure features a method of treating a neurological disorder in a patient, such as a human patient, identified as likely to benefit from treatment with a PIKfyve inhibitor on the basis of TDP-43 expression. In this aspect, the method includes (i) determining that the patient expresses a mutant form of TDP-43 having a mutation associated with TDP-43 aggregation, and (ii) providing to the patient a therapeutically effective amount of a PIKfyve inhibitor. The mutation in TDP-43 may be, for example, one or more of A315T, Q331K, M337V, D169G, G294A, G294V, Q343R, G295S, N345K, R361S, N390D, A382T, and G376D. In some embodiments, the patient has previously been determined to express a mutant form of TDP-43 having a mutation associated with TDP-43 aggregation, such as an A315T, Q331K, M337V, D169G, G294A, G294V, Q343R, G295S, N345K, R361S, N390D, A382T, or G376D mutation, and the method includes providing to the patient a therapeutically effective amount of a PIKfyve inhibitor.

In some embodiments of any of the above aspects, the PIKfyve inhibitor is provided to the patient by direct administration of the PIKfyve inhibitor to the patient. In some embodiments, the PIKfyve inhibitor is provided to the patient by administration of a prodrug that is converted in vivo to the PIKfyve inhibitor upon administration of the prodrug to the subject. Exemplary prodrugs useful in conjunction with the compositions and methods of the disclosure are esters, phosphates, and other chemical functionalities susceptible to hydrolysis upon administration to a subject. Prodrugs include those known in the art, such as those described, for instance, in Vig et al., Adv. Drug Deliv. Rev. 65:1370-1385 (2013), and Huttunen et al., Pharmacol. Rev. 63:750-771 (2011), the disclosures of each of which are incorporated herein by reference in their entirety.

In another aspect, the disclosure features a method of determining whether a patient (e.g., a human patient) having a neurological disorder is likely to benefit from treatment with a PIKfyve inhibitor by (i) determining whether the patient exhibits, or is prone to develop, TDP-43 aggregation and (ii) identifying the patient as likely to benefit from treatment with a PIKfyve inhibitor if the patient exhibits, or is prone to develop, TDP-43 aggregation. In some embodiments, the method further includes the step of (iii) informing the patient whether he or she is likely to benefit from treatment with a PIKfyve inhibitor. The susceptibility of the patient to developing TDP-43 aggregation may be determined, e.g., by monitoring the morphology and gene expression patterns of motor neurons obtained by differentiation of iPSCs reprogrammed from somatic cells (e.g., hematopoietic cells) isolated from the patient. For example, the patient's propensity to develop TDP-43 aggregation may be signaled by a finding that neurites on such motor neurons exhibit a decrease in size and/or a decrease in their rate of growth after from about 10 days to 100 days following differentiation (e.g., after from about 20 days to about 60 days following differentiation, after from about 21 days to about 59 days following differentiation, after from about 22 days to about 58 days following differentiation, after from about 23 days to about 57 days following differentiation, after from about 24 days to about 56 days following differentiation, after from about 25 days to about 55 days following differentiation, after from about 26 days to about 54 days following differentiation, after from about 27 days to about 53 days following differentiation, after from about 28 days to about 52 days following differentiation, after from about 29 days to about 51 days following differentiation, after from about 30 days to about 50 days following differentiation, after from about 31 days to about 49 days following differentiation, after from about 32 days to about 48 days following differentiation, after from about 33 days to about 47 days following differentiation, after from about 34 days to about 46 days following differentiation, after from about 35 days to about 45 days following differentiation, after from about 36 days to about 44 days following differentiation, after from about 37 days to about 43 days following differentiation, after from about 38 days to about 42 days following differentiation, after from about 39 days to about 41 days following differentiation, after from about 35 days to about 40 days following differentiation, or after from about 40 days to about 45 days following differentiation). In some embodiments, TDP-43 aggregation is signaled by a finding that such motor neurons exhibit an increase in leaked LDH concentration and/or an increase in CC3 expression after from about 10 days to 100 days following differentiation (e.g., after from about 20 days to about 60 days following differentiation, after from about 21 days to about 59 days following differentiation, after from about 22 days to about 58 days following differentiation, after from about 23 days to about 57 days following differentiation, after from about 24 days to about 56 days following differentiation, after from about 25 days to about 55 days following differentiation, after from about 26 days to about 54 days following differentiation, after from about 27 days to about 53 days following differentiation, after from about 28 days to about 52 days following differentiation, after from about 29 days to about 51 days following differentiation, after from about 30 days to about 50 days following differentiation, after from about 31 days to about 49 days following differentiation, after from about 32 days to about 48 days following differentiation, after from about 33 days to about 47 days following differentiation, after from about 34 days to about 46 days following differentiation, after from about 35 days to about 45 days following differentiation, after from about 36 days to about 44 days following differentiation, after from about 37 days to about 43 days following differentiation, after from about 38 days to about 42 days following differentiation, after from about 39 days to about 41 days following differentiation, after from about 35 days to about 40 days following differentiation, or after from about 40 days to about 45 days following differentiation).

In another aspect, the disclosure features a method of determining whether a patient (e.g., a human patient) having a neurological disorder is likely to benefit from treatment with a PIKfyve inhibitor by (i) determining whether the patient expresses a TDP-43 mutant having a mutation associated with TDP-43 aggregation (e.g., a mutation selected from A315T, Q331K, M337V, D169G, G294A, G294V, Q343R, G295S, N345K, R361S, N390D, A382T, and G376D) and (ii) identifying the patient as likely to benefit from treatment with a PIKfyve inhibitor if the patient expresses a TDP-43 mutant. In some embodiments, the method further includes the step of (iii) informing the patient whether he or she is likely to benefit from treatment with a PIKfyve inhibitor. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the PIKfyve inhibitor is provided to the patient by administration of the PIKfyve inhibitor to the patient. In some embodiments, the PIKfyve inhibitor is provided to the patient by administration of a prodrug that is converted in vivo to the PIKfyve inhibitor.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments of any of the above aspects, the patient does not have a mutation that gives rise to an expanded repeat region in a c9orf72 gene.

In some embodiments of any of the above aspects, the PIKfyve inhibitor is a small molecule antagonist of PIKfyve activity. For example, the PIKfyve inhibitor may be a compound of formula (I):

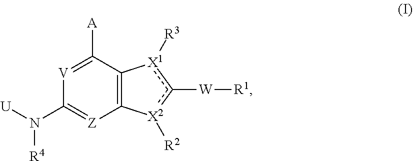

or a pharmaceutically acceptable salt or solvate thereof, wherein
R¹ is hydrogen, halo, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
each of X¹ and X² is independently selected from O, S, N, and C; W is a bond; O; S; (CH₂)n; S(O); SO₂; NRₐ; C(O); C(O)NRₐ; NRₐC(O); SO₂NRₐ; NRₐSO₂; CRₐ═CR_{b}; C═NRₐ; or NRₐ═CR_{b}, wherein n is 1-5 and each of Rₐ and R_{b} is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of R² and R³ is optionally present depending on the valence of the atom to which each is attached, and if present, each of R² and R³ is independently hydrogen, halo, hydroxyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
R⁴ is hydrogen, optionally substituted C₁₋₆ alkyl, C₃₋₈ cycloalkyl, optionally substituted C₆₋₁₀ aryl, optionally substituted C₁₋₉ heteroaryl, and optionally substituted C₁₋₉ heterocyclyl;
U is hydrogen,

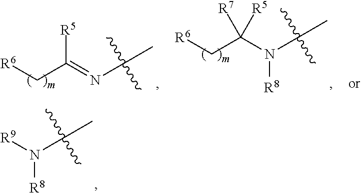

wherein m is 0-3, and each of R⁵, R⁶, R⁷, R⁸, and R^{g} is independently hydrogen, optionally substituted C₁₋₉ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl; or
R₃ and U, together with the nitrogen atom to which they are attached, form 4- to 6-membered heterocyclyl or heteroaryl optionally substituted by one or more substituents selected from hydrogen, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
one of the two [symbol] is a single bond, and the other [symbol] is a double bond; or each of the two [symbol] are aromatic bonds;
each of V and Z is independently N or OH; and
A is optionally substituted C₃₋₈ carbocyclyl, optionally substituted C₁₋₉ heterocyclyl, and optionally substituted C₁₋₉ heteroaryl.

Exemplary compounds of formula (I) are those in Table 1, below, and pharmaceutically acceptable salts thereof.

TABLE 1

| | |
|---|---|
| 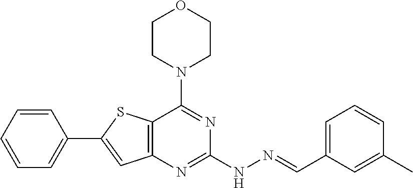 [SMILES: Cc1cccc(/C=N/Nc2nc(N3CCOCC3)c3sc(-c4ccccc4)cc3n2)c1] | (1.1) |
| 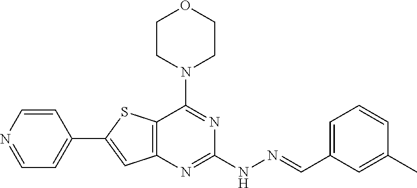 [SMILES: Cc1cccc(/C=N/Nc2nc(N3CCOCC3)c3sc(-c4ccncc4)cc3n2)c1] | (1.2) |
| 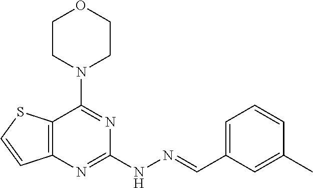 [SMILES: Cc1cccc(/C=N/Nc2nc(N3CCOCC3)c3sccc3n2)c1] | (1.3) |
| 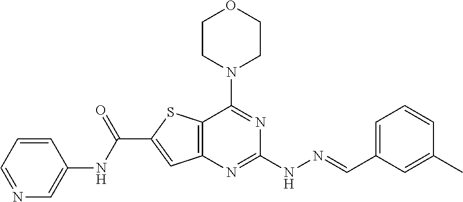 [SMILES: Cc1cccc(/C=N/Nc2nc(N3CCOCC3)c3sc(C(=O)Nc4cccnc4)cc3n2)c1] | (1.4) |
| 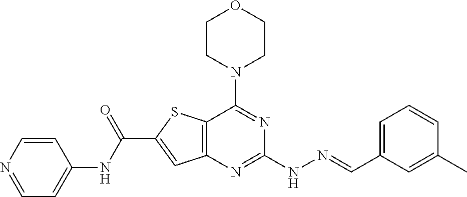 [SMILES: Cc1cccc(/C=N/Nc2nc(N3CCOCC3)c3sc(C(=O)Nc4ccncc4)cc3n2)c1] | (1.5) |
| 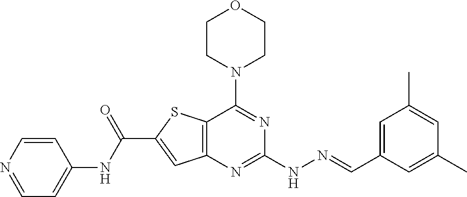 [SMILES: Cc1cc(C)cc(/C=N/Nc2nc(N3CCOCC3)c3sc(C(=O)Nc4ccncc4)cc3n2)c1] | (1.6) |
| 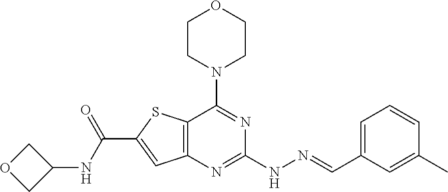 [SMILES: Cc1cccc(/C=N/Nc2nc(N3CCOCC3)c3sc(C(=O)NC4COC4)cc3n2)c1] | (1.7) |
| 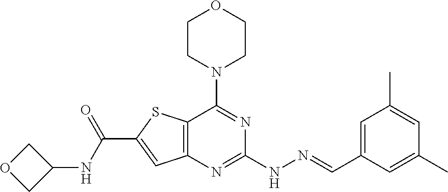 [SMILES: Cc1cc(C)cc(/C=N/Nc2nc(N3CCOCC3)c3sc(C(=O)NC4COC4)cc3n2)c1] | (1.8) |
| 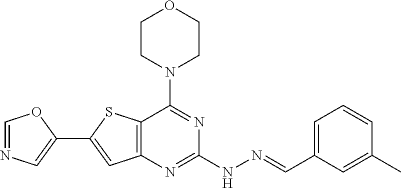 [SMILES: Cc1cccc(/C=N/Nc2nc(N3CCOCC3)c3sc(-c4cnco4)cc3n2)c1] | (1.9) |
| 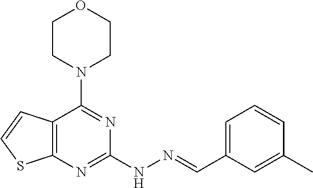 [SMILES: Cc1cccc(/C=N/Nc2nc(N3CCOCC3)c3ccsc3n2)c1] | (1.10) |
| 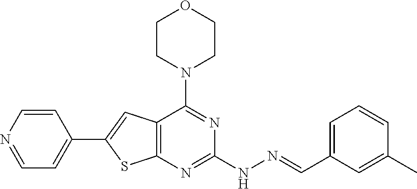 [SMILES: Cc1cccc(/C=N/Nc2nc(N3CCOCC3)c3cc(-c4ccncc4)sc3n2)c1] | (1.11) |
| 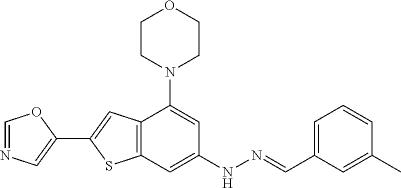 [SMILES: Cc1cccc(/C=N/Nc2cc(N3CCOCC3)c3cc(-c4cnco4)sc3c2)c1] | (1.12) |
| 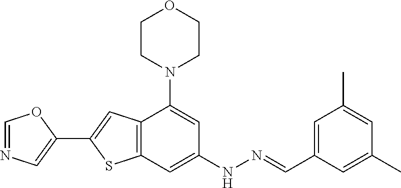 [SMILES: Cc1cc(C)cc(/C=N/Nc2cc(N3CCOCC3)c3cc(-c4cnco4)sc3c2)c1] | (1.13) |
| 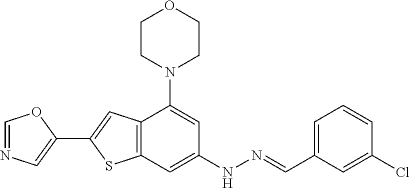 [SMILES: Clc1cccc(/C=N/Nc2cc(N3CCOCC3)c3cc(-c4cnco4)sc3c2)c1] | (1.14) |
| 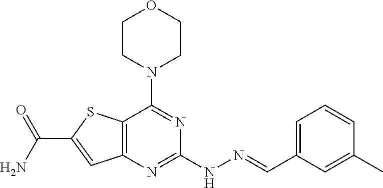 [SMILES: Cc1cccc(/C=N/Nc2nc(N3CCOCC3)c3sc(C(N)=O)cc3n2)c1] | (1.15) |
| 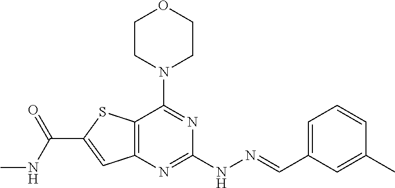 | (1.16) |
| 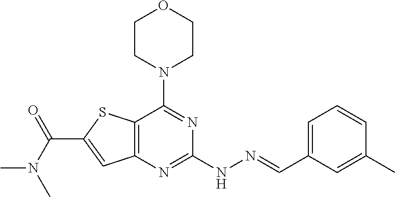 | (1.17) |
| 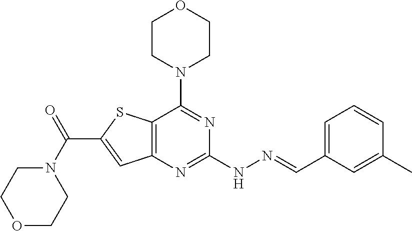 [SMILES: Cc1cccc(/C=N/Nc2nc(N3CCOCC3)c3sc(C(=O)N4CCOCC4)cc3n2)c1] | (1.18) |
| 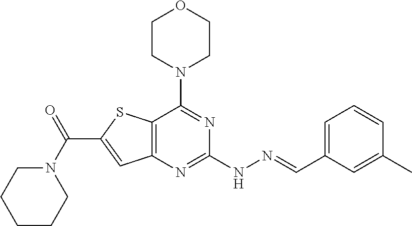 [SMILES: Cc1cccc(/C=N/Nc2nc(N3CCOCC3)c3sc(C(=O)N4CCCCC4)cc3n2)c1] | (1.19) |
| 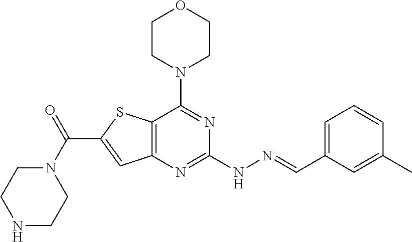 [SMILES: Cc1cccc(/C=N/Nc2nc(N3CCOCC3)c3sc(C(=O)N4CCNCC4)cc3n2)c1] | (1.20) |
| 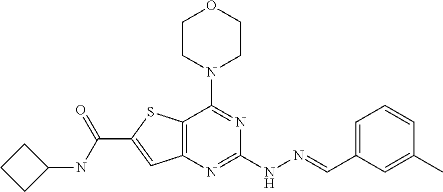 [SMILES: Cc1cccc(/C=N/Nc2nc(N3CCOCC3)c3sc(C(=O)NC4CCC4)cc3n2)c1] | (1.21) |
| 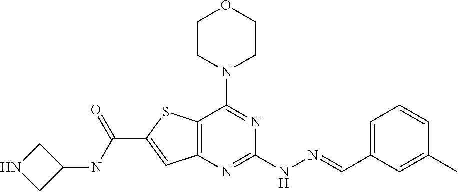 [SMILES: Cc1cccc(/C=N/Nc2nc(N3CCOCC3)c3sc(C(=O)NC4CNC4)cc3n2)c1] | (1.22) |
| 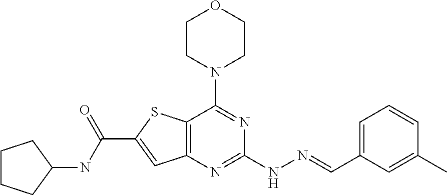 [SMILES: Cc1cccc(/C=N/Nc2nc(N3CCOCC3)c3sc(C(=O)NC4CCCC4)cc3n2)c1] | (1.23) |
| 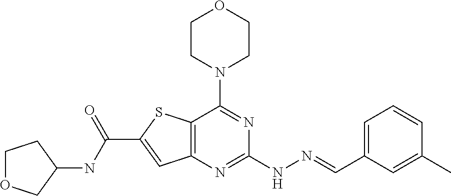 [SMILES: Cc1cccc(/C=N/Nc2nc(N3CCOCC3)c3sc(C(=O)NC4CCOC4)cc3n2)c1] | (1.24) |
| 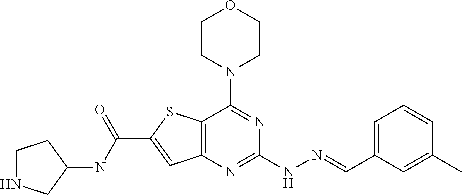 [SMILES: Cc1cccc(/C=N/Nc2nc(N3CCOCC3)c3sc(C(=O)NC4CCNC4)cc3n2)c1] | (1.25) |
| 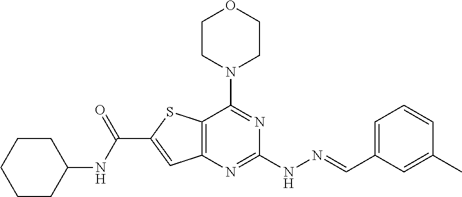 [SMILES: Cc1cccc(/C=N/Nc2nc(N3CCOCC3)c3sc(C(=O)NC4CCCCC4)cc3n2)c1] | (1.26) |
| 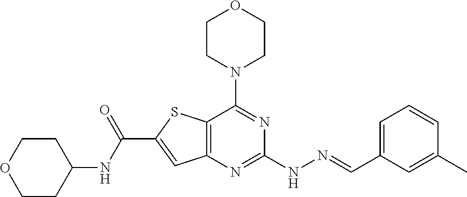 [SMILES: Cc1cccc(/C=N/Nc2nc(N3CCOCC3)c3sc(C(=O)NC4CCOCC4)cc3n2)c1] | (1.27) |
| 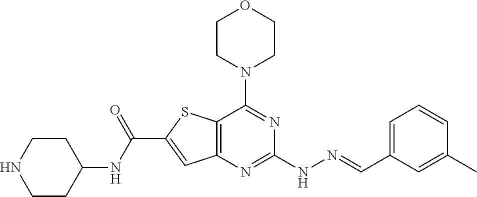 [SMILES: Cc1cccc(/C=N/Nc2nc(N3CCOCC3)c3sc(C(=O)NC4CCNCC4)cc3n2)c1] | (1.28) |
| 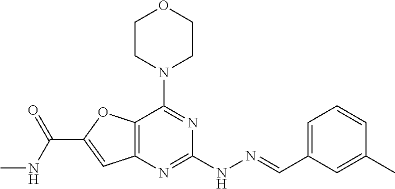 | (1.29) |
| 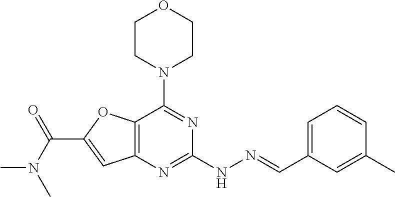 | (1.30) |
| 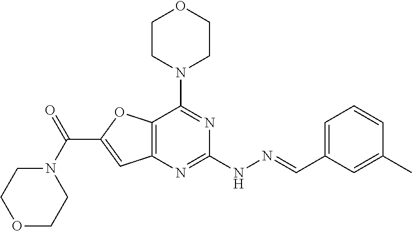 [SMILES: Cc1cccc(/C=N/Nc2nc(N3CCOCC3)c3oc(C(=O)N4CCOCC4)cc3n2)c1] | (1.31) |
| 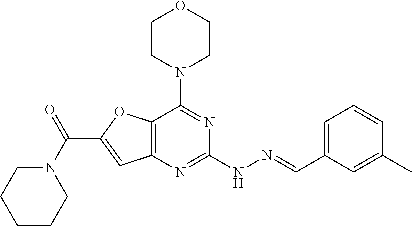 [SMILES: Cc1cccc(/C=N/Nc2nc(N3CCOCC3)c3oc(C(=O)N4CCCCC4)cc3n2)c1] | (1.32) |
| 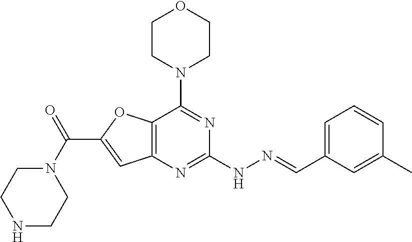 [SMILES: Cc1cccc(/C=N/Nc2nc(N3CCOCC3)c3oc(C(=O)N4CCNCC4)cc3n2)c1] | (1.33) |
| 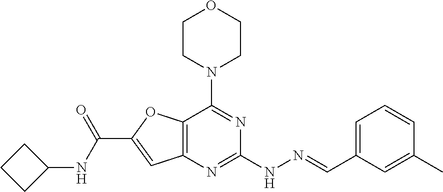 [SMILES: Cc1cccc(/C=N/Nc2nc(N3CCOCC3)c3oc(C(=O)NC4CCC4)cc3n2)c1] | (1.34) |
| 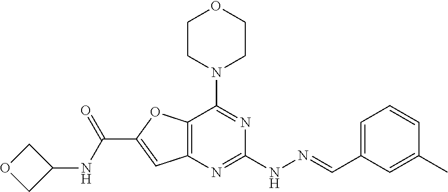 [SMILES: Cc1cccc(/C=N/Nc2nc(N3CCOCC3)c3oc(C(=O)NC4COC4)cc3n2)c1] | (1.35) |
| 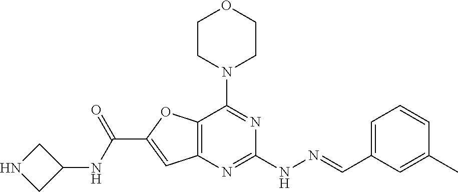 [SMILES: Cc1cccc(/C=N/Nc2nc(N3CCOCC3)c3oc(C(=O)NC4CNC4)cc3n2)c1] | (1.36) |
| 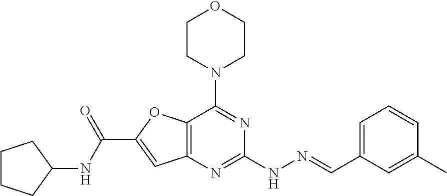 [SMILES: Cc1cccc(/C=N/Nc2nc(N3CCOCC3)c3oc(C(=O)NC4CCCC4)cc3n2)c1] | (1.37) |
| 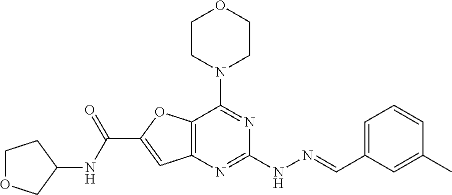 [SMILES: Cc1cccc(/C=N/Nc2nc(N3CCOCC3)c3oc(C(=O)NC4CCOC4)cc3n2)c1] | (1.38) |
| 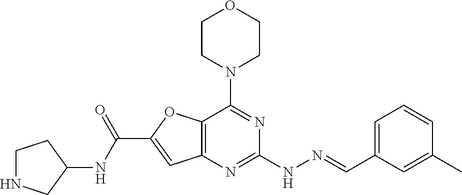 [SMILES: Cc1cccc(/C=N/Nc2nc(N3CCOCC3)c3oc(C(=O)NC4CCNC4)cc3n2)c1] | (1.39) |
| 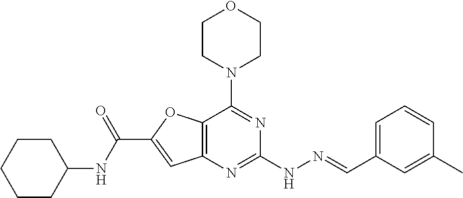 [SMILES: Cc1cccc(/C=N/Nc2nc(N3CCOCC3)c3oc(C(=O)NC4CCCCC4)cc3n2)c1] | (1.40) |
| 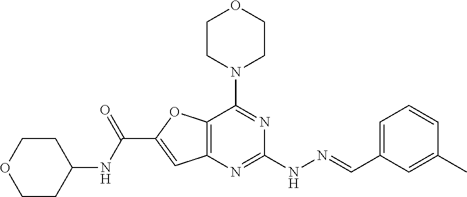 [SMILES: Cc1cccc(/C=N/Nc2nc(N3CCOCC3)c3oc(C(=O)NC4CCOCC4)cc3n2)c1] | (1.41) |
| 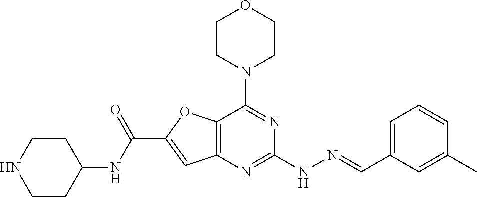 [SMILES: Cc1cccc(/C=N/Nc2nc(N3CCOCC3)c3oc(C(=O)NC4CCNCC4)cc3n2)c1] | (1.42) |
| 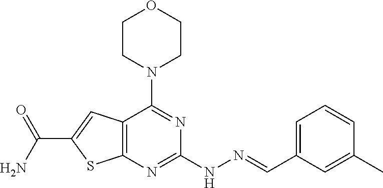 [SMILES: Cc1cccc(/C=N/Nc2nc(N3CCOCC3)c3cc(C(N)=O)sc3n2)c1] | (1.43) |
| 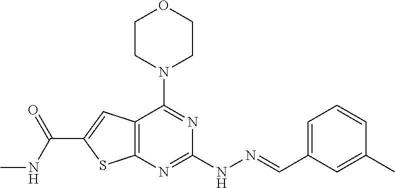 | (1.44) |
| 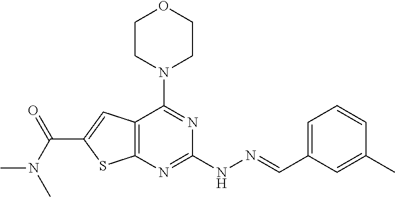 | (1.45) |
| 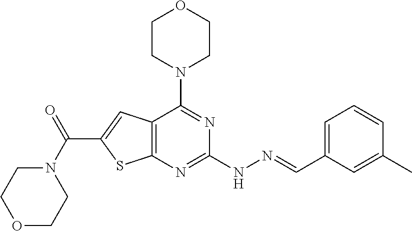 [SMILES: Cc1cccc(/C=N/Nc2nc(N3CCOCC3)c3cc(C(=O)N4CCOCC4)sc3n2)c1] | (1.46) |
| 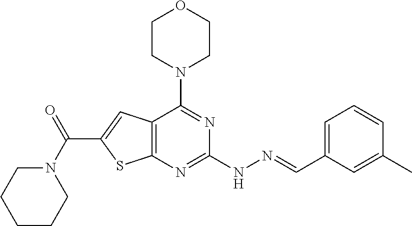 [SMILES: Cc1cccc(/C=N/Nc2nc(N3CCOCC3)c3cc(C(=O)N4CCCCC4)sc3n2)c1] | (1.47) |
| 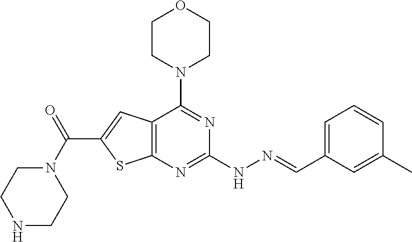 [SMILES: Cc1cccc(/C=N/Nc2nc(N3CCOCC3)c3cc(C(=O)N4CCNCC4)sc3n2)c1] | (1.48) |
| 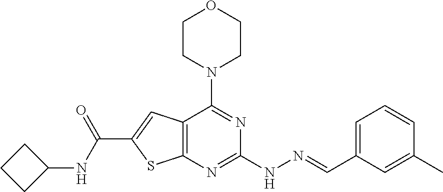 [SMILES: Cc1cccc(/C=N/Nc2nc(N3CCOCC3)c3cc(C(=O)NC4CCC4)sc3n2)c1] | (1.49) |
| 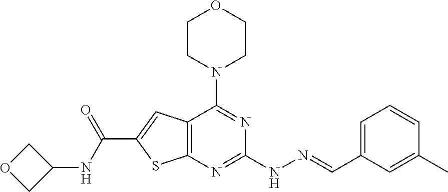 [SMILES: Cc1cccc(/C=N/Nc2nc(N3CCOCC3)c3cc(C(=O)NC4COC4)sc3n2)c1] | (1.50) |
| 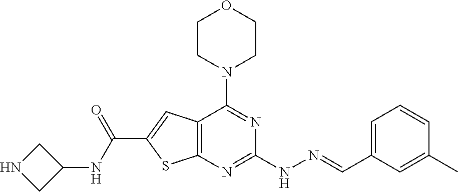 [SMILES: Cc1cccc(/C=N/Nc2nc(N3CCOCC3)c3cc(C(=O)NC4CNC4)sc3n2)c1] | (1.51) |
| 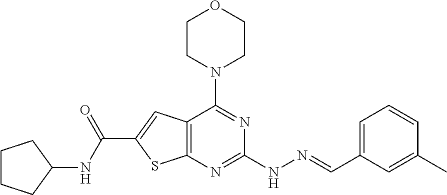 [SMILES: Cc1cccc(/C=N/Nc2nc(N3CCOCC3)c3cc(C(=O)NC4CCCC4)sc3n2)c1] | (1.52) |
| 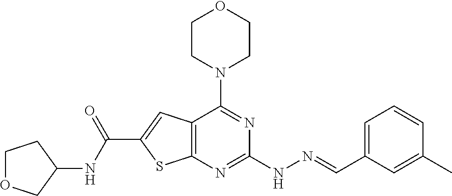 [SMILES: Cc1cccc(/C=N/Nc2nc(N3CCOCC3)c3cc(C(=O)NC4CCOC4)sc3n2)c1] | (1.53) |
| 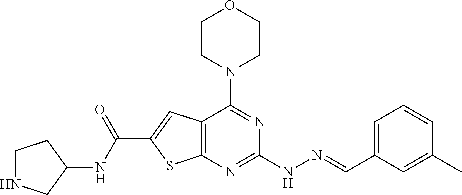 [SMILES: Cc1cccc(/C=N/Nc2nc(N3CCOCC3)c3cc(C(=O)NC4CCNC4)sc3n2)c1] | (1.54) |
| 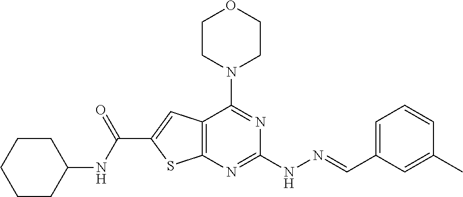 [SMILES: Cc1cccc(/C=N/Nc2nc(N3CCOCC3)c3cc(C(=O)NC4CCCCC4)sc3n2)c1] | (1.55) |
| 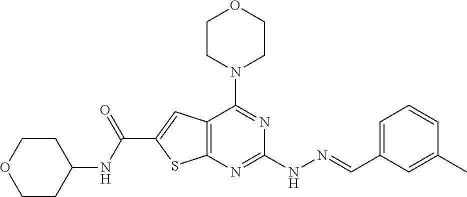 [SMILES: Cc1cccc(/C=N/Nc2nc(N3CCOCC3)c3cc(C(=O)NC4CCOCC4)sc3n2)c1] | (1.56) |
| 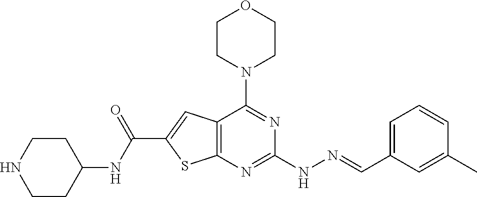 [SMILES: Cc1cccc(/C=N/Nc2nc(N3CCOCC3)c3cc(C(=O)NC4CCNCC4)sc3n2)c1] | (1.57) |
| 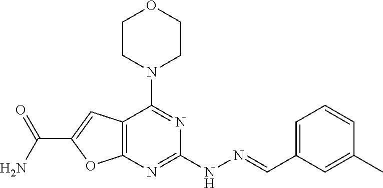 [SMILES: Cc1cccc(/C=N/Nc2nc(N3CCOCC3)c3cc(C(N)=O)oc3n2)c1] | (1.58) |
| 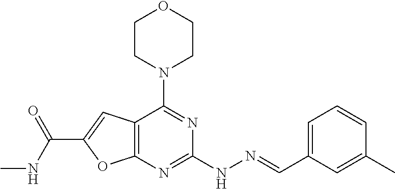 | (1.59) |
| 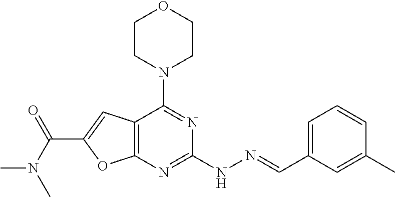 | (1.60) |
| 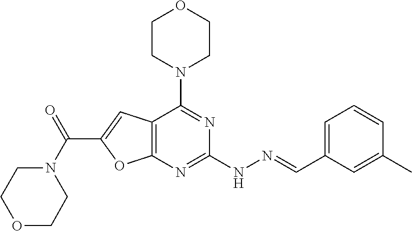 [SMILES: Cc1cccc(/C=N/Nc2nc(N3CCOCC3)c3cc(C(=O)N4CCOCC4)oc3n2)c1] | (1.61) |
| 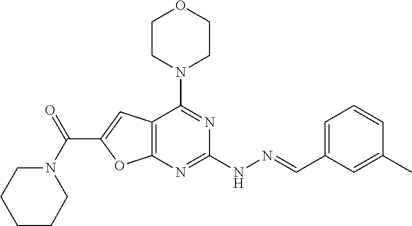 [SMILES: Cc1cccc(/C=N/Nc2nc(N3CCOCC3)c3cc(C(=O)N4CCCCC4)oc3n2)c1] | (1.62) |
| 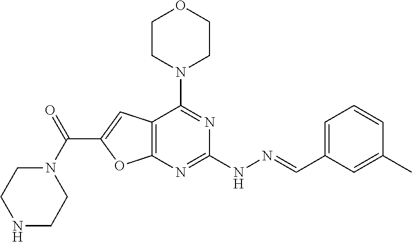 [SMILES: Cc1cccc(/C=N/Nc2nc(N3CCOCC3)c3cc(C(=O)N4CCNCC4)oc3n2)c1] | (1.63) |
| 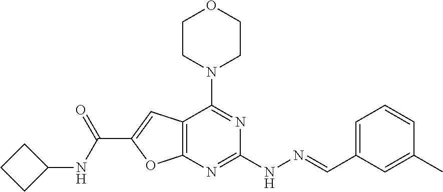 [SMILES: Cc1cccc(/C=N/Nc2nc(N3CCOCC3)c3cc(C(=O)NC4CCC4)oc3n2)c1] | (1.64) |
| 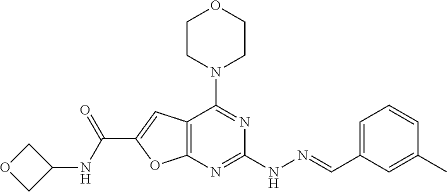 [SMILES: Cc1cccc(/C=N/Nc2nc(N3CCOCC3)c3cc(C(=O)NC4COC4)oc3n2)c1] | (1.65) |
| 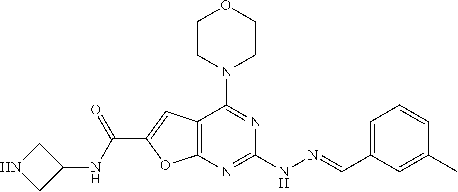 [SMILES: Cc1cccc(/C=N/Nc2nc(N3CCOCC3)c3cc(C(=O)NC4CNC4)oc3n2)c1] | (1.66) |
| 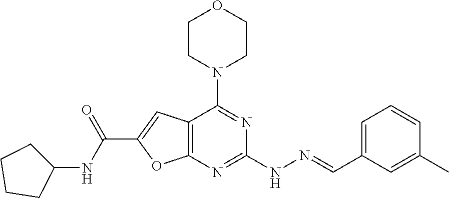 [SMILES: Cc1cccc(/C=N/Nc2nc(N3CCOCC3)c3cc(C(=O)NC4CCCC4)oc3n2)c1] | (1.67) |
| 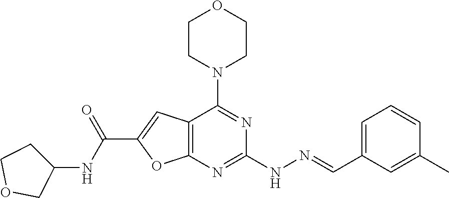 [SMILES: Cc1cccc(/C=N/Nc2nc(N3CCOCC3)c3cc(C(=O)NC4CCOC4)oc3n2)c1] | (1.68) |
| 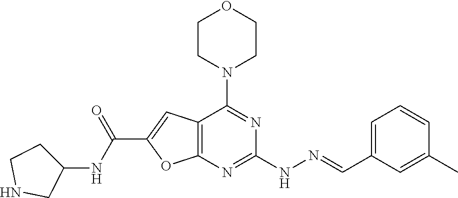 [SMILES: Cc1cccc(/C=N/Nc2nc(N3CCOCC3)c3cc(C(=O)NC4CCNC4)oc3n2)c1] | (1.69) |
| 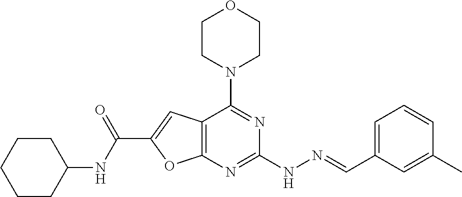 [SMILES: Cc1cccc(/C=N/Nc2nc(N3CCOCC3)c3cc(C(=O)NC4CCCCC4)oc3n2)c1] | (1.70) |
| 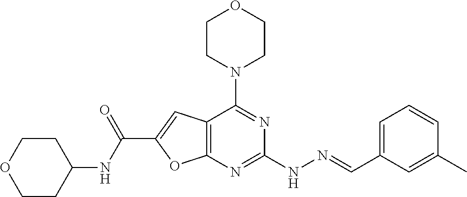 [SMILES: Cc1cccc(/C=N/Nc2nc(N3CCOCC3)c3cc(C(=O)NC4CCOCC4)oc3n2)c1] | (1.71) |
| 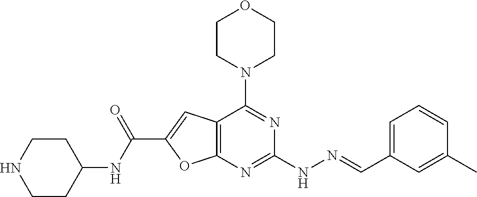 [SMILES: Cc1cccc(/C=N/Nc2nc(N3CCOCC3)c3cc(C(=O)NC4CCNCC4)oc3n2)c1] | (1.72) |
| 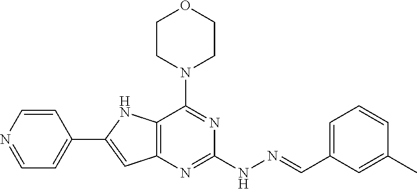 [SMILES: Cc1cccc(/C=N/Nc2nc(N3CCOCC3)c3[nH]c(-c4ccncc4)cc3n2)c1] | (1.73) |
| 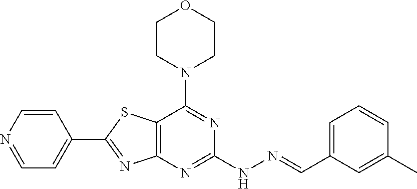 [SMILES: Cc1cccc(/C=N/Nc2nc(N3CCOCC3)c3sc(-c4ccncc4)nc3n2)c1] | (1.74) |
| 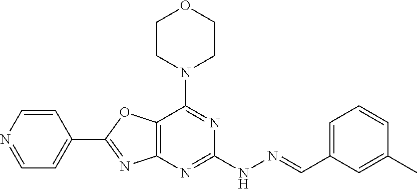 [SMILES: Cc1cccc(/C=N/Nc2nc(N3CCOCC3)c3oc(-c4ccncc4)nc3n2)c1] | (1.75) |
| 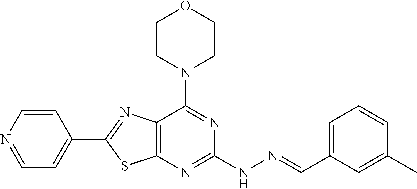 [SMILES: Cc1cccc(/C=N/Nc2nc(N3CCOCC3)c3nc(-c4ccncc4)sc3n2)c1] | (1.76) |
| 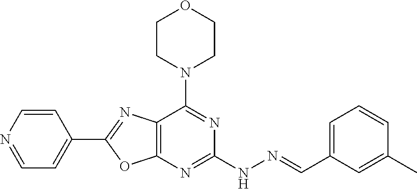 [SMILES: Cc1cccc(/C=N/Nc2nc(N3CCOCC3)c3nc(-c4ccncc4)oc3n2)c1] | (1.77) |
| 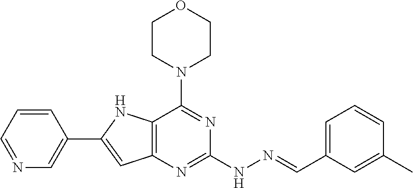 [SMILES: Cc1cccc(/C=N/Nc2nc(N3CCOCC3)c3[nH]c(-c4cccnc4)cc3n2)c1] | (1.78) |
| 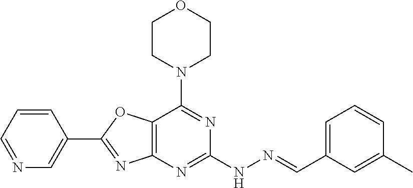 [SMILES: Cc1cccc(/C=N/Nc2nc(N3CCOCC3)c3oc(-c4cccnc4)nc3n2)c1] | (1.79) |
| 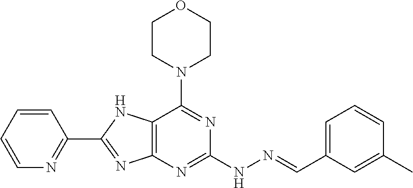 [SMILES: Cc1cccc(/C=N/Nc2nc(N3CCOCC3)c3[nH]c(-c4ccccn4)nc3n2)c1] | (1.80) |
| 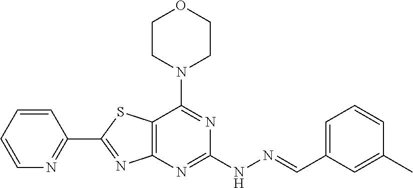 [SMILES: Cc1cccc(/C=N/Nc2nc(N3CCOCC3)c3sc(-c4ccccn4)nc3n2)c1] | (1.81) |
| 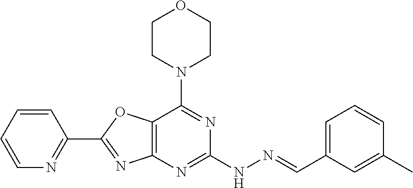 [SMILES: Cc1cccc(/C=N/Nc2nc(N3CCOCC3)c3oc(-c4ccccn4)nc3n2)c1] | (1.82) |
| 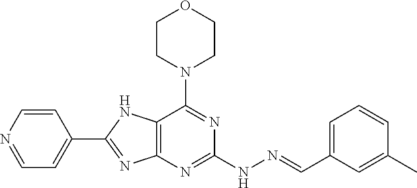 [SMILES: Cc1cccc(/C=N/Nc2nc(N3CCOCC3)c3[nH]c(-c4ccncc4)nc3n2)c1] | (1.83) |
| 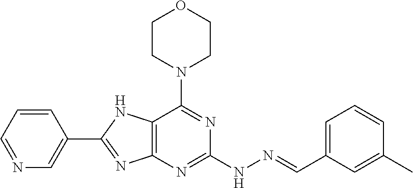 [SMILES: Cc1cccc(/C=N/Nc2nc(N3CCOCC3)c3[nH]c(-c4cccnc4)nc3n2)c1] | (1.84) |
| 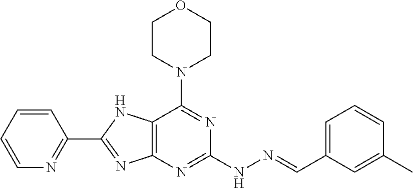 [SMILES: Cc1cccc(/C=N/Nc2nc(N3CCOCC3)c3[nH]c(-c4ccccn4)nc3n2)c1] | (1.85) |
| 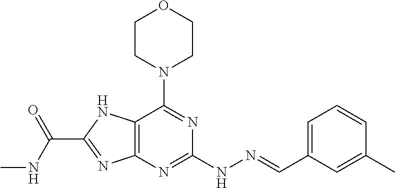 | (1.86) |
| 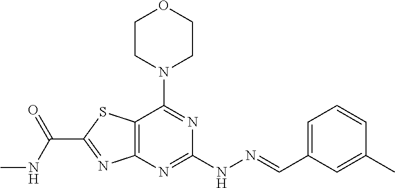 | (1.87) |
| 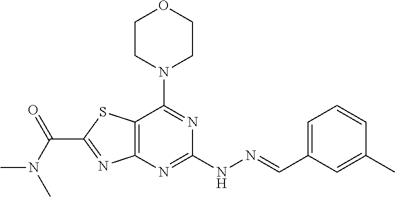 | (1.88) |
| 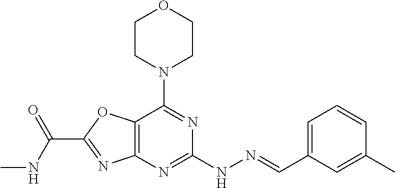 | (1.89) |
| 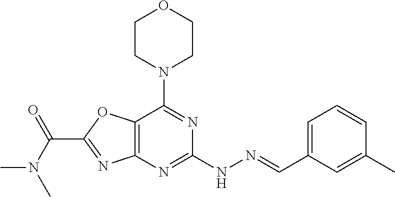 | (1.90) |
| 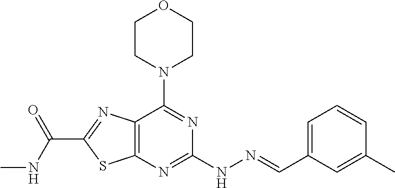 | (1.91) |
| 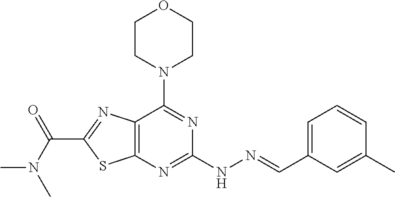 | (1.92) |
| 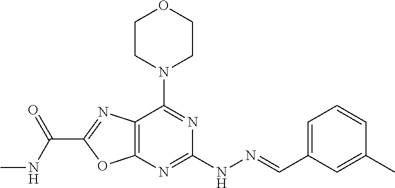 | (1.93) |
| 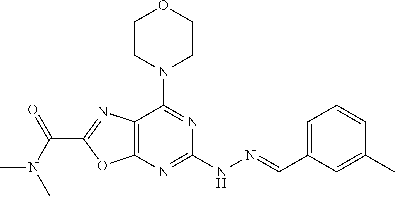 | (1.94) |
| 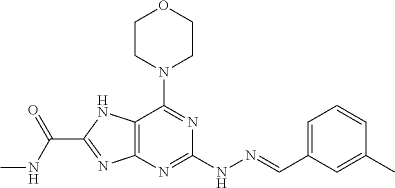 | (1.95) |
| 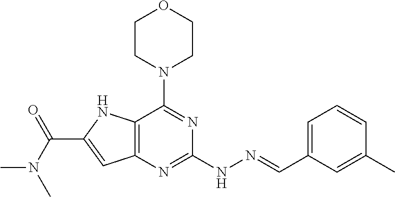 | (1.96) |
| 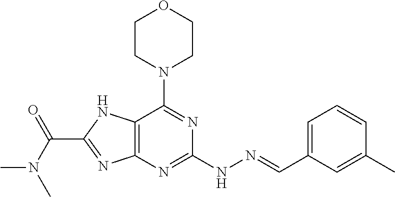 | (1.97) |
| 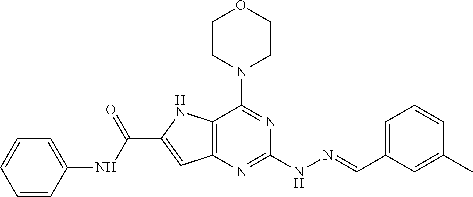 [SMILES: Cc1cccc(/C=N/Nc2nc(N3CCOCC3)c3[nH]c(C(=O)Nc4ccccc4)cc3n2)c1] | (1.98) |
| 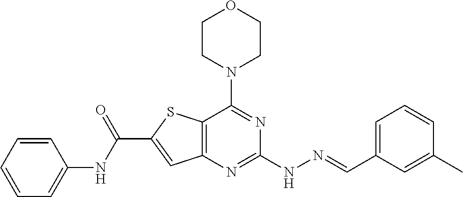 [SMILES: Cc1cccc(/C=N/Nc2nc(N3CCOCC3)c3sc(C(=O)Nc4ccccc4)cc3n2)c1] | (1.99) |
| 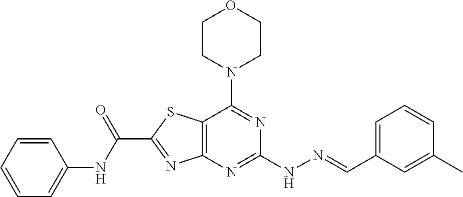 [SMILES: Cc1cccc(/C=N/Nc2nc(N3CCOCC3)c3sc(C(=O)Nc4ccccc4)nc3n2)c1] | (1.100) |
| 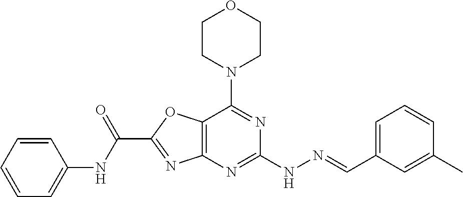 [SMILES: Cc1cccc(/C=N/Nc2nc(N3CCOCC3)c3oc(C(=O)Nc4ccccc4)nc3n2)c1] | (1.101) |
| 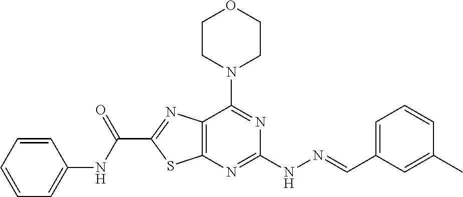 [SMILES: Cc1cccc(/C=N/Nc2nc(N3CCOCC3)c3nc(C(=O)Nc4ccccc4)sc3n2)c1] | (1.102) |
| 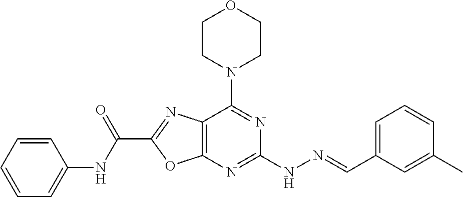 [SMILES: Cc1cccc(/C=N/Nc2nc(N3CCOCC3)c3nc(C(=O)Nc4ccccc4)oc3n2)c1] | (1.103) |
| 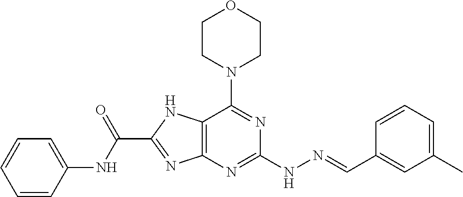 [SMILES: Cc1cccc(/C=N/Nc2nc(N3CCOCC3)c3[nH]c(C(=O)Nc4ccccc4)nc3n2)c1] | (1.104) |
| 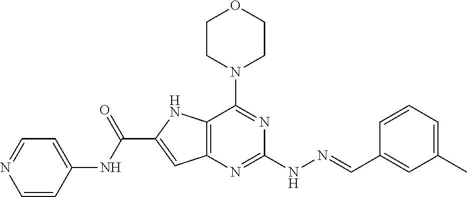 [SMILES: Cc1cccc(/C=N/Nc2nc(N3CCOCC3)c3[nH]c(C(=O)Nc4ccncc4)cc3n2)c1] | (1.105) |
| 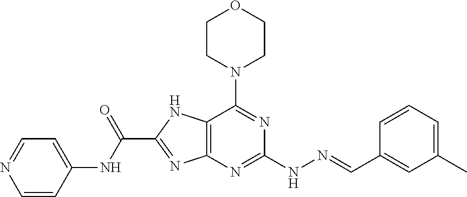 [SMILES: Cc1cccc(/C=N/Nc2nc(N3CCOCC3)c3[nH]c(C(=O)Nc4ccncc4)nc3n2)c1] | (1.106) |
| 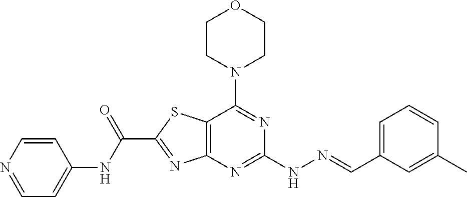 [SMILES: Cc1cccc(/C=N/Nc2nc(N3CCOCC3)c3sc(C(=O)Nc4ccncc4)nc3n2)c1] | (1.107) |
| 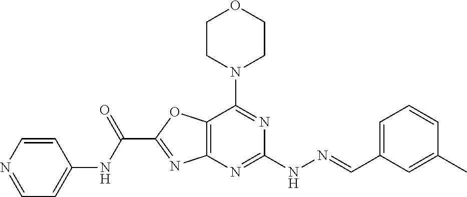 [SMILES: Cc1cccc(/C=N/Nc2nc(N3CCOCC3)c3oc(C(=O)Nc4ccncc4)nc3n2)c1] | (1.108) |
| 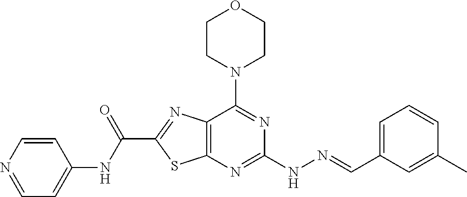 [SMILES: Cc1cccc(/C=N/Nc2nc(N3CCOCC3)c3nc(C(=O)Nc4ccncc4)sc3n2)c1] | (1.109) |
| 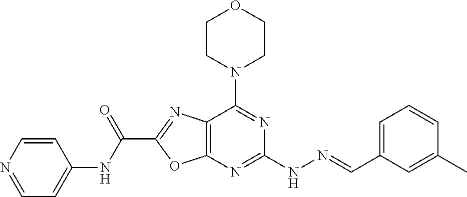 [SMILES: Cc1cccc(/C=N/Nc2nc(N3CCOCC3)c3nc(C(=O)Nc4ccncc4)oc3n2)c1] | (1.110) |
| 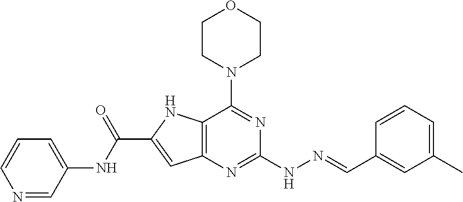 [SMILES: Cc1cccc(/C=N/Nc2nc(N3CCOCC3)c3[nH]c(C(=O)Nc4cccnc4)cc3n2)c1] | (1.111) |
| 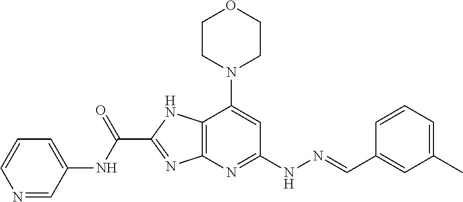 [SMILES: Cc1cccc(/C=N/Nc2cc(N3CCOCC3)c3[nH]c(C(=O)Nc4cccnc4)nc3n2)c1] | (1.112) |
| 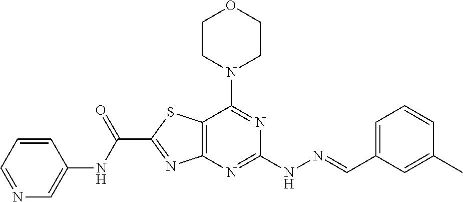 [SMILES: Cc1cccc(/C=N/Nc2nc(N3CCOCC3)c3sc(C(=O)Nc4cccnc4)nc3n2)c1] | (1.113) |
| 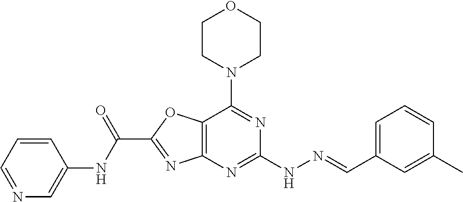 [SMILES: Cc1cccc(/C=N/Nc2nc(N3CCOCC3)c3oc(C(=O)Nc4cccnc4)nc3n2)c1] | (1.114) |
| 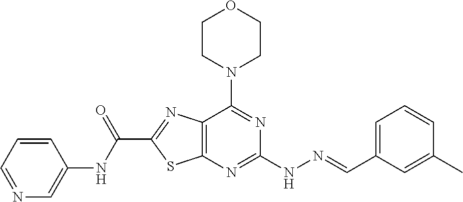 [SMILES: Cc1cccc(/C=N/Nc2nc(N3CCOCC3)c3nc(C(=O)Nc4cccnc4)sc3n2)c1] | (1.115) |
| 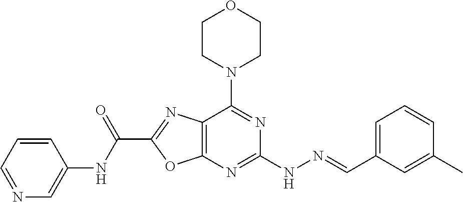 [SMILES: Cc1cccc(/C=N/Nc2nc(N3CCOCC3)c3nc(C(=O)Nc4cccnc4)oc3n2)c1] | (1.116) |
| 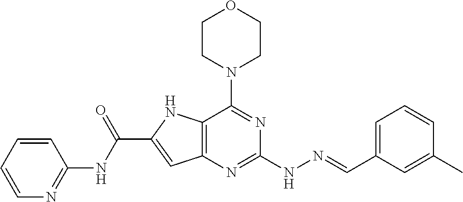 [SMILES: Cc1cccc(/C=N/Nc2nc(N3CCOCC3)c3[nH]c(C(=O)Nc4ccccn4)cc3n2)c1] | (1.117) |
| 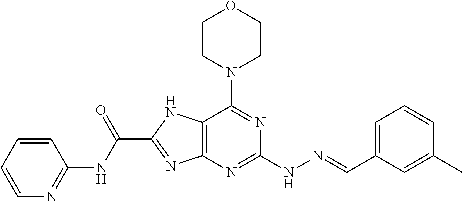 [SMILES: Cc1cccc(/C=N/Nc2nc(N3CCOCC3)c3[nH]c(C(=O)Nc4ccccn4)nc3n2)c1] | (1.118) |
| 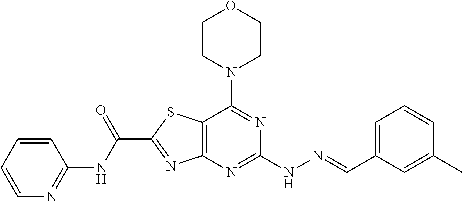 [SMILES: Cc1cccc(/C=N/Nc2nc(N3CCOCC3)c3sc(C(=O)Nc4ccccn4)nc3n2)c1] | (1.119) |
| 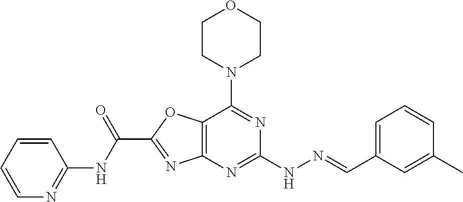 [SMILES: Cc1cccc(/C=N/Nc2nc(N3CCOCC3)c3oc(C(=O)Nc4ccccn4)nc3n2)c1] | (1.120) |
| 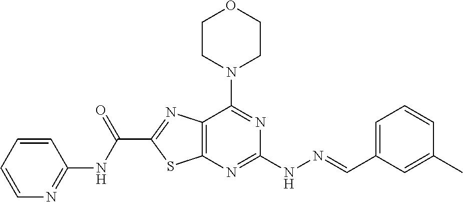 [SMILES: Cc1cccc(/C=N/Nc2nc(N3CCOCC3)c3nc(C(=O)Nc4ccccn4)sc3n2)c1] | (1.121) |
| 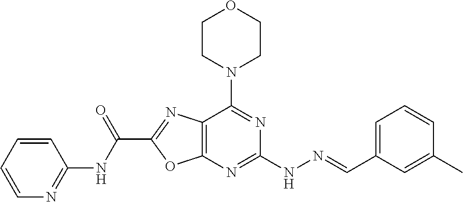 [SMILES: Cc1cccc(/C=N/Nc2nc(N3CCOCC3)c3nc(C(=O)Nc4ccccn4)oc3n2)c1] | (1.122) |
| 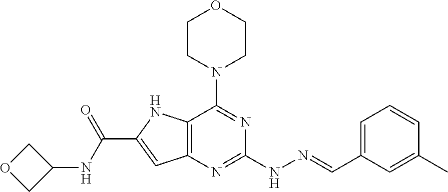 [SMILES: Cc1cccc(/C=N/Nc2nc(N3CCOCC3)c3[nH]c(C(=O)NC4COC4)cc3n2)c1] | (1.123) |
| 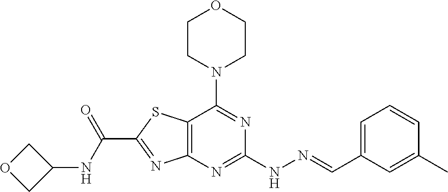 [SMILES: Cc1cccc(/C=N/Nc2nc(N3CCOCC3)c3sc(C(=O)NC4COC4)nc3n2)c1] | (1.124) |
| 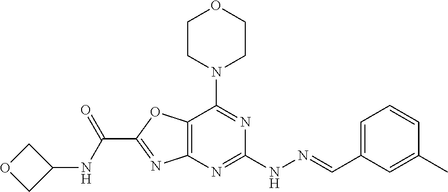 [SMILES: Cc1cccc(/C=N/Nc2nc(N3CCOCC3)c3oc(C(=O)NC4COC4)nc3n2)c1] | (1.125) |
| 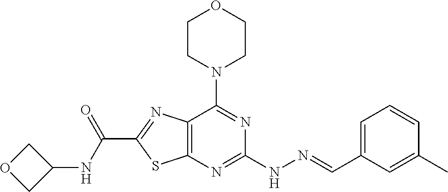 [SMILES: Cc1cccc(/C=N/Nc2nc(N3CCOCC3)c3nc(C(=O)NC4COC4)sc3n2)c1] | (1.126) |
| 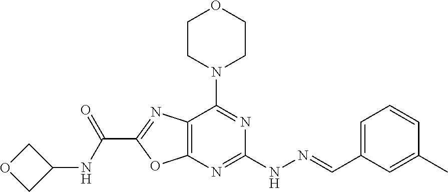 [SMILES: Cc1cccc(/C=N/Nc2nc(N3CCOCC3)c3nc(C(=O)NC4COC4)oc3n2)c1] | (1.127) |
| 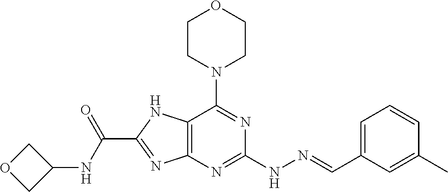 [SMILES: Cc1cccc(/C=N/Nc2nc(N3CCOCC3)c3[nH]c(C(=O)NC4COC4)nc3n2)c1] | (1.128) |
| 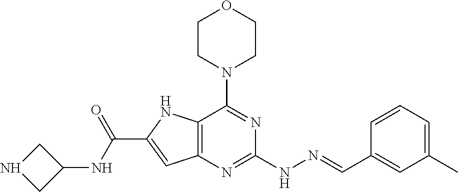 [SMILES: Cc1cccc(/C=N/Nc2nc(N3CCOCC3)c3[nH]c(C(=O)NC4CNC4)cc3n2)c1] | (1.129) |
| 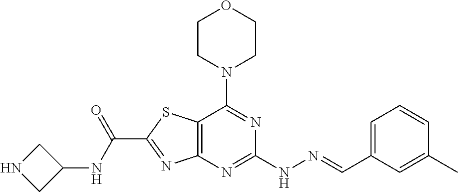 [SMILES: Cc1cccc(/C=N/Nc2nc(N3CCOCC3)c3sc(C(=O)NC4CNC4)nc3n2)c1] | (1.130) |
| 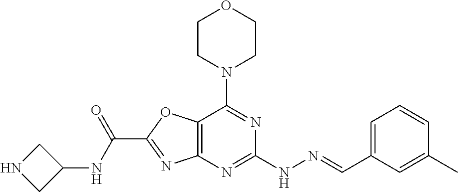 [SMILES: Cc1cccc(/C=N/Nc2nc(N3CCOCC3)c3oc(C(=O)NC4CNC4)nc3n2)c1] | (1.131) |
| 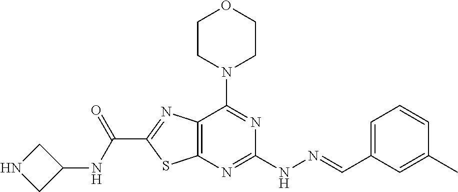 [SMILES: Cc1cccc(/C=N/Nc2nc(N3CCOCC3)c3nc(C(=O)NC4CNC4)sc3n2)c1] | (1.132) |
| 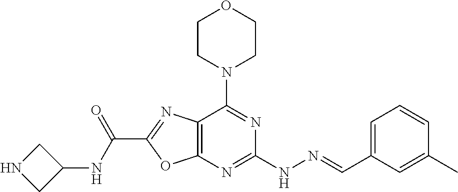 [SMILES: Cc1cccc(/C=N/Nc2nc(N3CCOCC3)c3nc(C(=O)NC4CNC4)oc3n2)c1] | (1.133) |
| 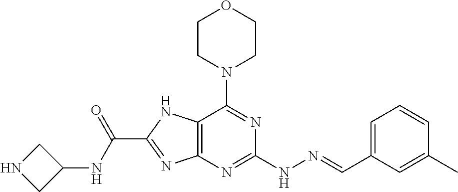 [SMILES: Cc1cccc(/C=N/Nc2nc(N3CCOCC3)c3[nH]c(C(=O)NC4CNC4)nc3n2)c1] | (1.134) |
| 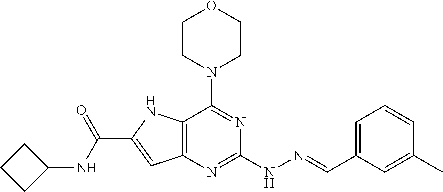 [SMILES: Cc1cccc(/C=N/Nc2nc(N3CCOCC3)c3[nH]c(C(=O)NC4CCC4)cc3n2)c1] | (1.135) |
| 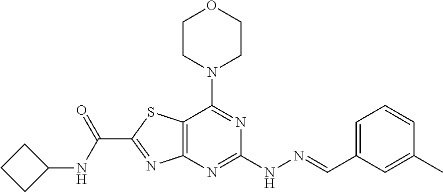 [SMILES: Cc1cccc(/C=N/Nc2nc(N3CCOCC3)c3sc(C(=O)NC4CCC4)nc3n2)c1] | (1.136) |
| 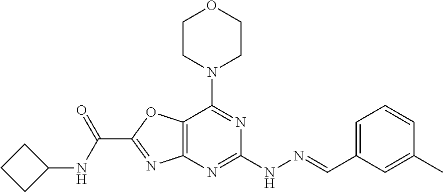 [SMILES: Cc1cccc(/C=N/Nc2nc(N3CCOCC3)c3oc(C(=O)NC4CCC4)nc3n2)c1] | (1.137) |
| 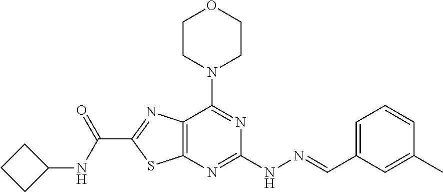 [SMILES: Cc1cccc(/C=N/Nc2nc(N3CCOCC3)c3nc(C(=O)NC4CCC4)sc3n2)c1] | (1.138) |
| 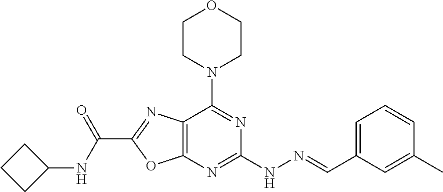 [SMILES: Cc1cccc(/C=N/Nc2nc(N3CCOCC3)c3nc(C(=O)NC4CCC4)oc3n2)c1] | (1.139) |
| 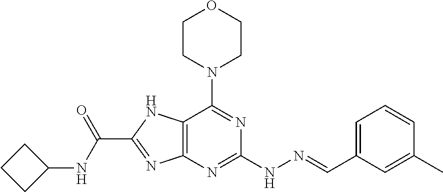 [SMILES: Cc1cccc(/C=N/Nc2nc(N3CCOCC3)c3[nH]c(C(=O)NC4CCC4)nc3n2)c1] | (1.140) |
| 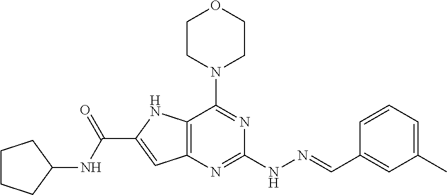 [SMILES: Cc1cccc(/C=N/Nc2nc(N3CCOCC3)c3[nH]c(C(=O)NC4CCCC4)cc3n2)c1] | (1.141) |
| 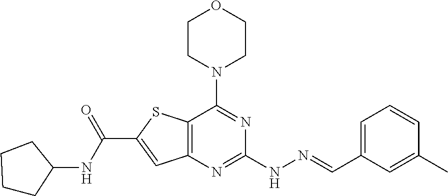 [SMILES: Cc1cccc(/C=N/Nc2nc(N3CCOCC3)c3sc(C(=O)NC4CCCC4)cc3n2)c1] | (1.142) |
| 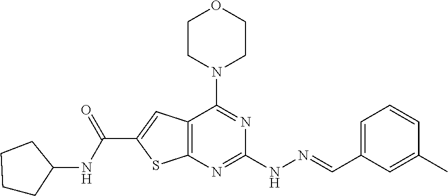 [SMILES: Cc1cccc(/C=N/Nc2nc(N3CCOCC3)c3cc(C(=O)NC4CCCC4)sc3n2)c1] | (1.143) |
| 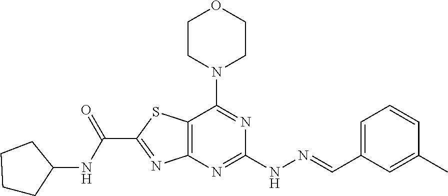 [SMILES: Cc1cccc(/C=N/Nc2nc(N3CCOCC3)c3sc(C(=O)NC4CCCC4)nc3n2)c1] | (1.144) |
| 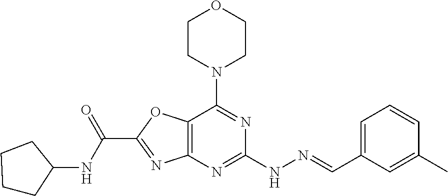 [SMILES: Cc1cccc(/C=N/Nc2nc(N3CCOCC3)c3oc(C(=O)NC4CCCC4)nc3n2)c1] | (1.145) |
| 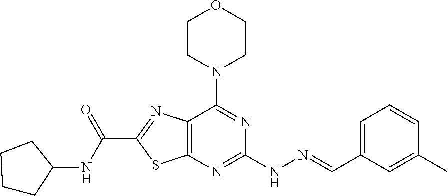 [SMILES: Cc1cccc(/C=N/Nc2nc(N3CCOCC3)c3nc(C(=O)NC4CCCC4)sc3n2)c1] | (1.146) |
| 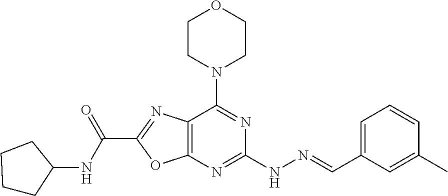 [SMILES: Cc1cccc(/C=N/Nc2nc(N3CCOCC3)c3nc(C(=O)NC4CCCC4)oc3n2)c1] | (1.147) |
| 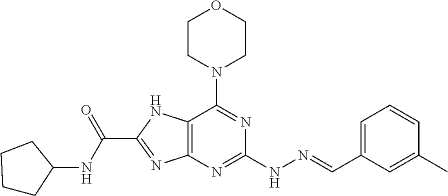 [SMILES: Cc1cccc(/C=N/Nc2nc(N3CCOCC3)c3[nH]c(C(=O)NC4CCCC4)nc3n2)c1] | (1.148) |
| 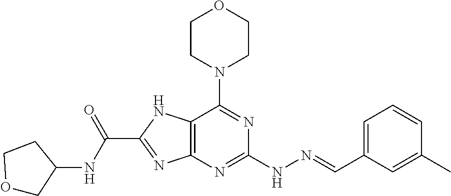 [SMILES: Cc1cccc(/C=N/Nc2nc(N3CCOCC3)c3[nH]c(C(=O)NC4CCOC4)nc3n2)c1] | (1.149) |
| 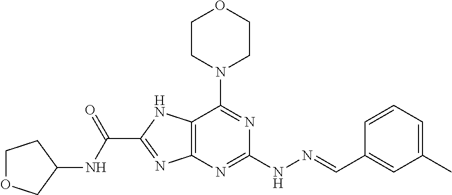 [SMILES: Cc1cccc(/C=N/Nc2nc(N3CCOCC3)c3[nH]c(C(=O)NC4CCOC4)nc3n2)c1] | (1.150) |
| 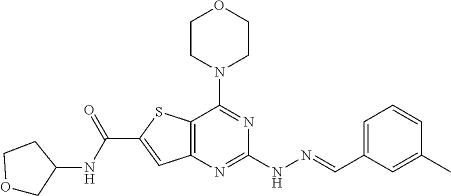 [SMILES: Cc1cccc(/C=N/Nc2nc(N3CCOCC3)c3sc(C(=O)NC4CCOC4)cc3n2)c1] | (1.151) |
| 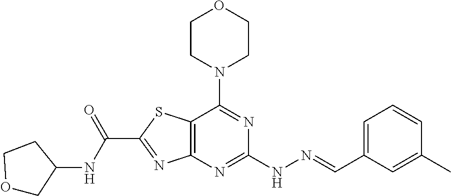 [SMILES: Cc1cccc(/C=N/Nc2nc(N3CCOCC3)c3sc(C(=O)NC4CCOC4)nc3n2)c1] | (1.152) |
| 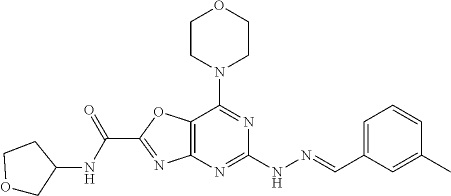 [SMILES: Cc1cccc(/C=N/Nc2nc(N3CCOCC3)c3oc(C(=O)NC4CCOC4)nc3n2)c1] | (1.153) |
| 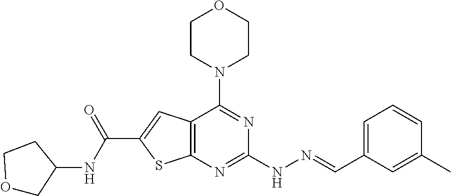 [SMILES: Cc1cccc(/C=N/Nc2nc(N3CCOCC3)c3cc(C(=O)NC4CCOC4)sc3n2)c1] | (1.154) |
| 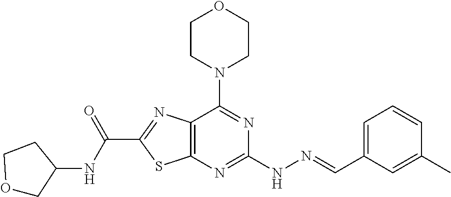 [SMILES: Cc1cccc(/C=N/Nc2nc(N3CCOCC3)c3nc(C(=O)NC4CCOC4)sc3n2)c1] | (1.155) |
| 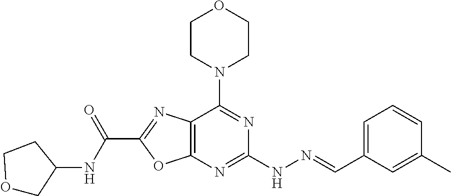 [SMILES: Cc1cccc(/C=N/Nc2nc(N3CCOCC3)c3nc(C(=O)NC4CCOC4)oc3n2)c1] | (1.156) |
| 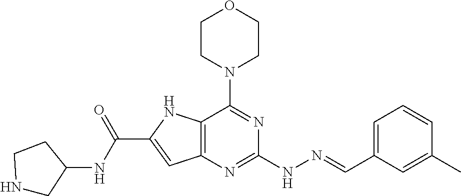 [SMILES: Cc1cccc(/C=N/Nc2nc(N3CCOCC3)c3[nH]c(C(=O)NC4CCNC4)cc3n2)c1] | (1.157) |
| 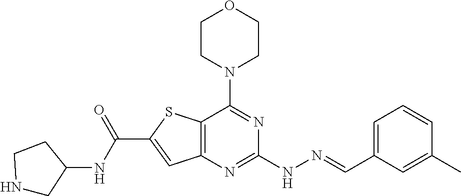 [SMILES: Cc1cccc(/C=N/Nc2nc(N3CCOCC3)c3sc(C(=O)NC4CCNC4)cc3n2)c1] | (1.158) |
| 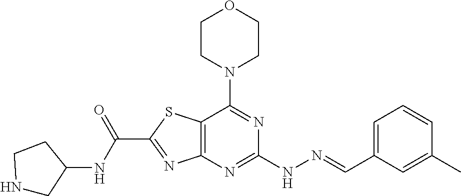 [SMILES: Cc1cccc(/C=N/Nc2nc(N3CCOCC3)c3sc(C(=O)NC4CCNC4)nc3n2)c1] | (1.159) |
| 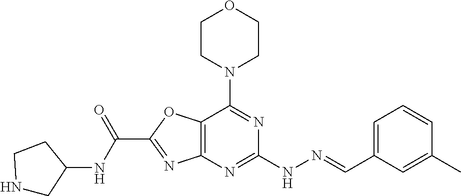 [SMILES: Cc1cccc(/C=N/Nc2nc(N3CCOCC3)c3oc(C(=O)NC4CCNC4)nc3n2)c1] | (1.160) |
| 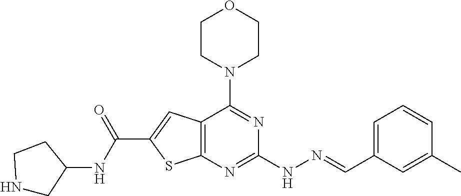 [SMILES: Cc1cccc(/C=N/Nc2nc(N3CCOCC3)c3cc(C(=O)NC4CCNC4)sc3n2)c1] | (1.161) |
| 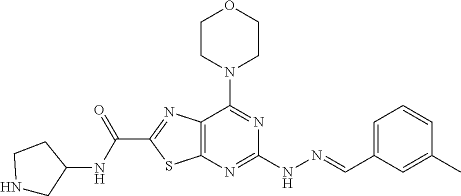 [SMILES: Cc1cccc(/C=N/Nc2nc(N3CCOCC3)c3nc(C(=O)NC4CCNC4)sc3n2)c1] | (1.162) |
| 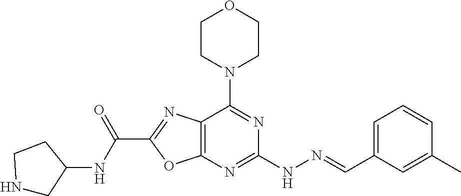 [SMILES: Cc1cccc(/C=N/Nc2nc(N3CCOCC3)c3nc(C(=O)NC4CCNC4)oc3n2)c1] | (1.163) |
| 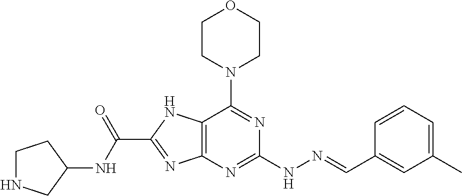 [SMILES: Cc1cccc(/C=N/Nc2nc(N3CCOCC3)c3[nH]c(C(=O)NC4CCNC4)nc3n2)c1] | (1.164) |
| 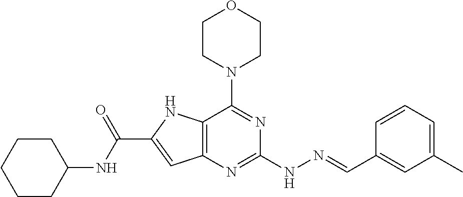 [SMILES: Cc1cccc(/C=N/Nc2nc(N3CCOCC3)c3[nH]c(C(=O)NC4CCCCC4)cc3n2)c1] | (1.165) |
| 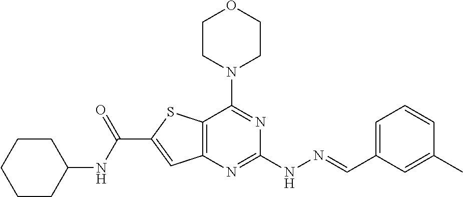 [SMILES: Cc1cccc(/C=N/Nc2nc(N3CCOCC3)c3sc(C(=O)NC4CCCCC4)cc3n2)c1] | (1.166) |
| 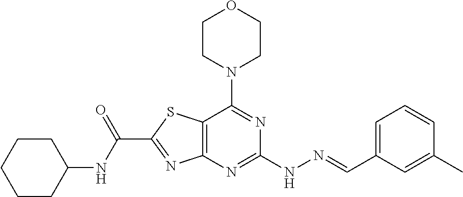 [SMILES: Cc1cccc(/C=N/Nc2nc(N3CCOCC3)c3sc(C(=O)NC4CCCCC4)nc3n2)c1] | (1.167) |
| 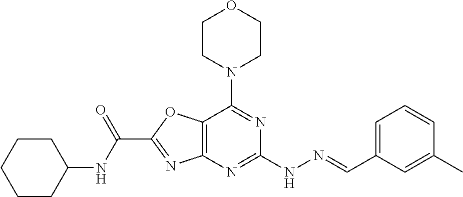 [SMILES: Cc1cccc(/C=N/Nc2nc(N3CCOCC3)c3oc(C(=O)NC4CCCCC4)nc3n2)c1] | (1.168) |
| 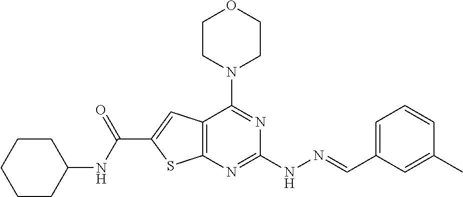 [SMILES: Cc1cccc(/C=N/Nc2nc(N3CCOCC3)c3cc(C(=O)NC4CCCCC4)sc3n2)c1] | (1.169) |
| 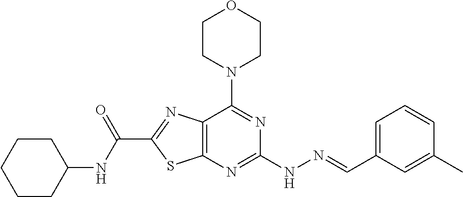 [SMILES: Cc1cccc(/C=N/Nc2nc(N3CCOCC3)c3nc(C(=O)NC4CCCCC4)sc3n2)c1] | (1.170) |
| 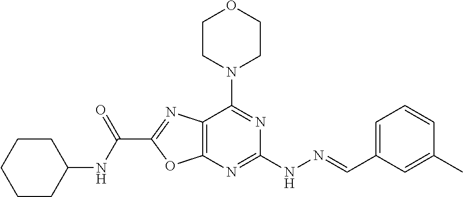 [SMILES: Cc1cccc(/C=N/Nc2nc(N3CCOCC3)c3nc(C(=O)NC4CCCCC4)oc3n2)c1] | (1.171) |
| 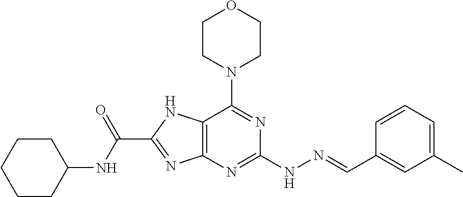 [SMILES: Cc1cccc(/C=N/Nc2nc(N3CCOCC3)c3[nH]c(C(=O)NC4CCCCC4)nc3n2)c1] | (1.172) |
| 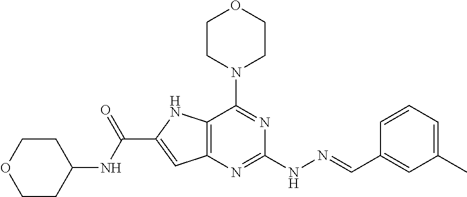 [SMILES: Cc1cccc(/C=N/Nc2nc(N3CCOCC3)c3[nH]c(C(=O)NC4CCOCC4)cc3n2)c1] | (1.173) |
| 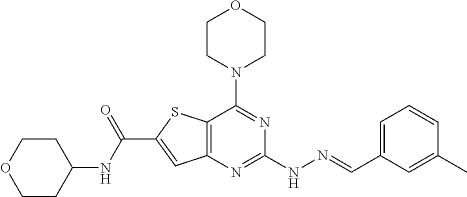 [SMILES: Cc1cccc(/C=N/Nc2nc(N3CCOCC3)c3sc(C(=O)NC4CCOCC4)cc3n2)c1] | (1.174) |
| 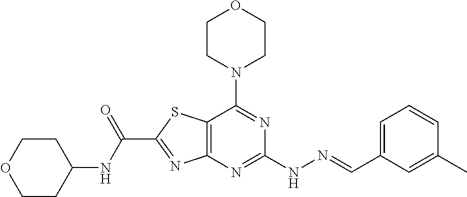 [SMILES: Cc1cccc(/C=N/Nc2nc(N3CCOCC3)c3sc(C(=O)NC4CCOCC4)nc3n2)c1] | (1.175) |
| 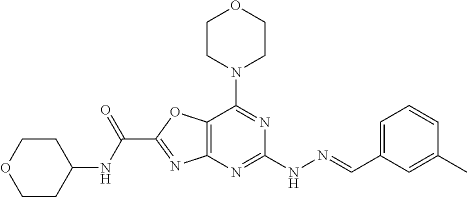 [SMILES: Cc1cccc(/C=N/Nc2nc(N3CCOCC3)c3oc(C(=O)NC4CCOCC4)nc3n2)c1] | (1.176) |
| 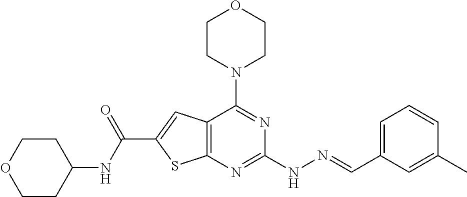 [SMILES: Cc1cccc(/C=N/Nc2nc(N3CCOCC3)c3cc(C(=O)NC4CCOCC4)sc3n2)c1] | (1.177) |
| 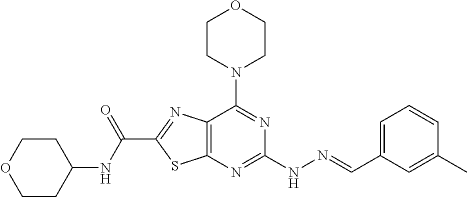 [SMILES: Cc1cccc(/C=N/Nc2nc(N3CCOCC3)c3nc(C(=O)NC4CCOCC4)sc3n2)c1] | (1.178) |
| 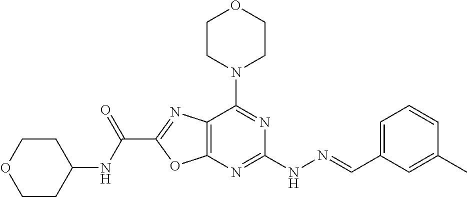 [SMILES: Cc1cccc(/C=N/Nc2nc(N3CCOCC3)c3nc(C(=O)NC4CCOCC4)oc3n2)c1] | (1.179) |
| 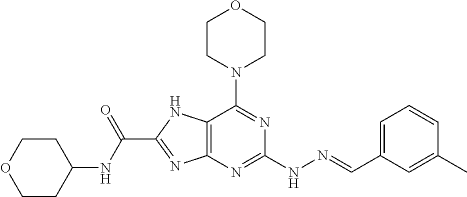 [SMILES: Cc1cccc(/C=N/Nc2nc(N3CCOCC3)c3[nH]c(C(=O)NC4CCOCC4)nc3n2)c1] | (1.180) |
| 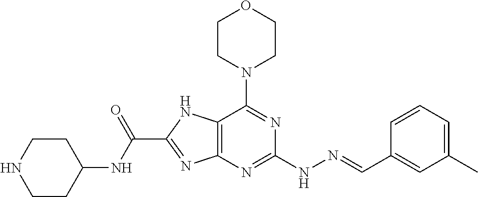 [SMILES: Cc1cccc(/C=N/Nc2nc(N3CCOCC3)c3[nH]c(C(=O)NC4CCNCC4)nc3n2)c1] | (1.181) |
| 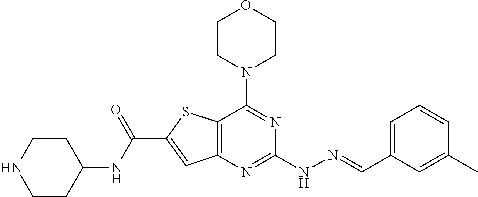 [SMILES: Cc1cccc(/C=N/Nc2nc(N3CCOCC3)c3sc(C(=O)NC4CCNCC4)cc3n2)c1] | (1.182) |
| 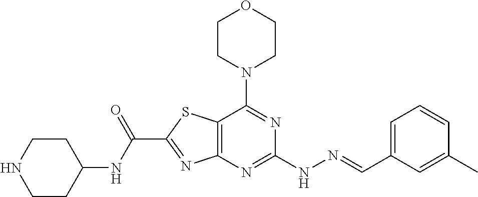 [SMILES: Cc1cccc(/C=N/Nc2nc(N3CCOCC3)c3sc(C(=O)NC4CCNCC4)nc3n2)c1] | (1.183) |
| 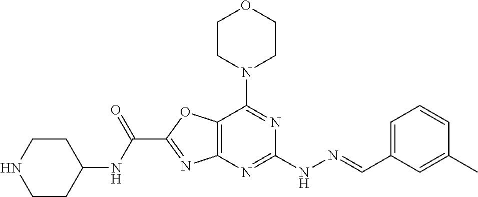 [SMILES: Cc1cccc(/C=N/Nc2nc(N3CCOCC3)c3oc(C(=O)NC4CCNCC4)nc3n2)c1] | (1.184) |
| 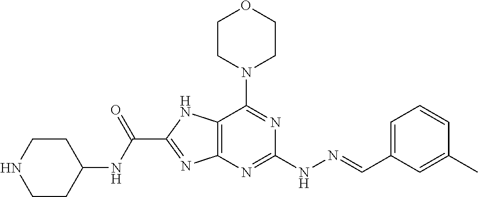 [SMILES: Cc1cccc(/C=N/Nc2nc(N3CCOCC3)c3[nH]c(C(=O)NC4CCNCC4)nc3n2)c1] | (1.185) |
| 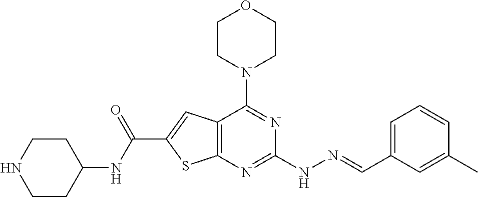 [SMILES: Cc1cccc(/C=N/Nc2nc(N3CCOCC3)c3cc(C(=O)NC4CCNCC4)sc3n2)c1] | (1.186) |
| 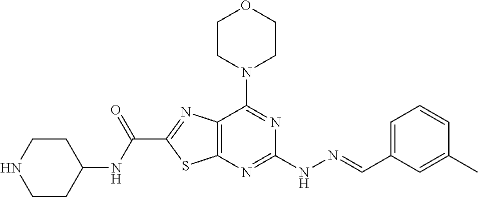 [SMILES: Cc1cccc(/C=N/Nc2nc(N3CCOCC3)c3nc(C(=O)NC4CCNCC4)sc3n2)c1] | (1.187) |
| 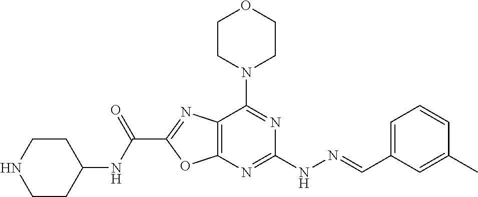 [SMILES: Cc1cccc(/C=N/Nc2nc(N3CCOCC3)c3nc(C(=O)NC4CCNCC4)oc3n2)c1] | (1.188) |
| 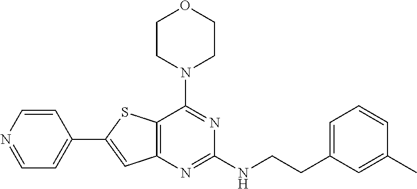 [SMILES: Cc1cccc(CCNc2nc(N3CCOCC3)c3sc(-c4ccncc4)cc3n2)c1] | (1.189) |
| 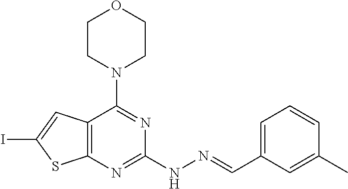 [SMILES: Cc1cccc(/C=N/Nc2nc(N3CCOCC3)c3cc(I)sc3n2)c1] | (1.190) |
| 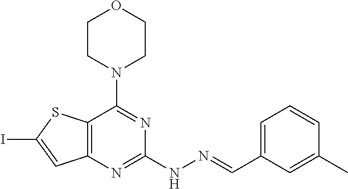 [SMILES: Cc1cccc(/C=N/Nc2nc(N3CCOCC3)c3sc(I)cc3n2)c1] | (1.191) |
| 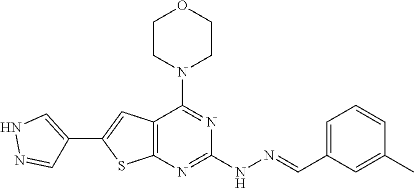 [SMILES: Cc1cccc(/C=N/Nc2nc(N3CCOCC3)c3cc(-c4cn[nH]c4)sc3n2)c1] | (1.192) |
| 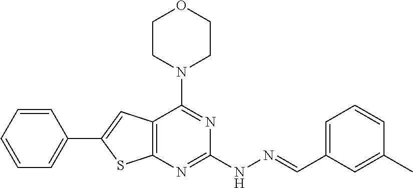 [SMILES: Cc1cccc(/C=N/Nc2nc(N3CCOCC3)c3cc(-c4ccccc4)sc3n2)c1] | (1.193) |
| 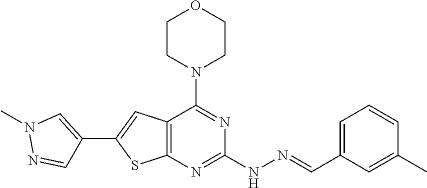 | (1.194) |
| 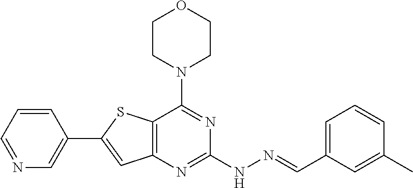 [SMILES: Cc1cccc(/C=N/Nc2nc(N3CCOCC3)c3sc(-c4cccnc4)cc3n2)c1] | (1.195) |
| 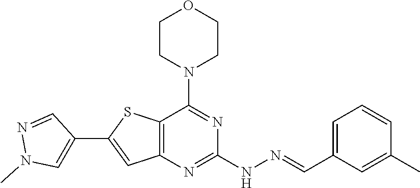 | (1.196) |
| 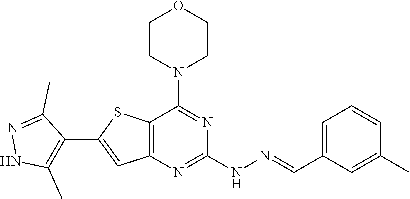 [SMILES: Cc1cccc(/C=N/Nc2nc(N3CCOCC3)c3sc(-c4c(C)n[nH]c4C)cc3n2)c1] | (1.197) |
| 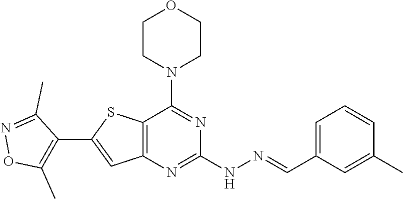 [SMILES: Cc1cccc(/C=N/Nc2nc(N3CCOCC3)c3sc(-c4c(C)noc4C)cc3n2)c1] | (1.198) |
| 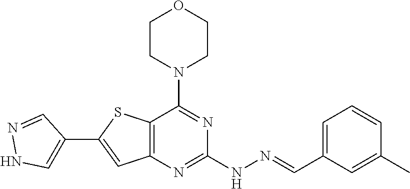 [SMILES: Cc1cccc(/C=N/Nc2nc(N3CCOCC3)c3sc(-c4cn[nH]c4)cc3n2)c1] | (1.199) |
| 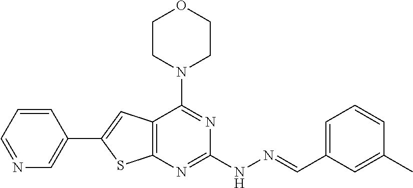 [SMILES: Cc1cccc(/C=N/Nc2nc(N3CCOCC3)c3cc(-c4cccnc4)sc3n2)c1] | (1.200) |
| 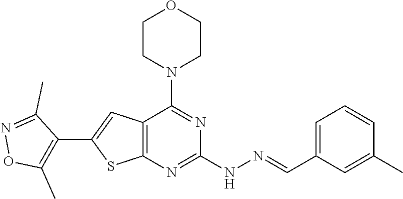 [SMILES: Cc1cccc(/C=N/Nc2nc(N3CCOCC3)c3cc(-c4c(C)noc4C)sc3n2)c1] | (1.201) |
| 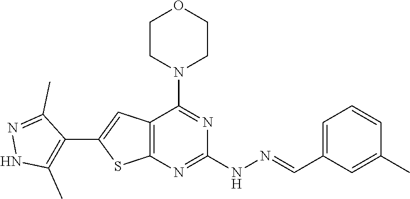 [SMILES: Cc1cccc(/C=N/Nc2nc(N3CCOCC3)c3cc(-c4c(C)n[nH]c4C)sc3n2)c1] | (1.202) |
| 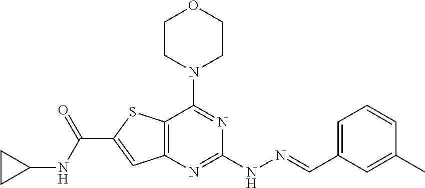 [SMILES: Cc1cccc(/C=N/Nc2nc(N3CCOCC3)c3sc(C(=O)NC4CC4)cc3n2)c1] | (1.203) |
| 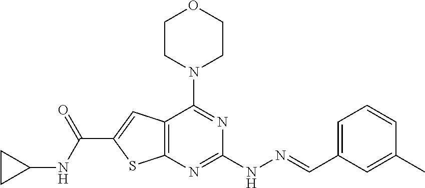 [SMILES: Cc1cccc(/C=N/Nc2nc(N3CCOCC3)c3cc(C(=O)NC4CC4)sc3n2)c1] | (1.204) |

In some embodiments, the PIKfyve inhibitor is a compound of formula (II):

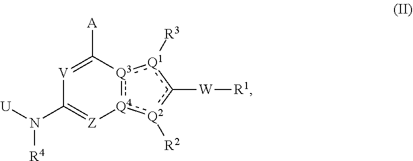

or a pharmaceutically acceptable salt or solvate thereof, wherein
R¹ is hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of Q¹, Q², Q³, and Q⁴ is independently C or N, and at least one of Q, Q¹, Q³, and Q⁴ is N;
W is a bond; O; S; (CH₂)ₙ; S(O); SO₂; NRₐ; C(O); C(O)NRₐ; NRₐC(O); SO₂NRₐ; NRₐSO₂; CRₐ═CR_{b}; C═NRₐ; or NRₐ═CR_{b}, wherein n is 1-5, and each of Rₐ and R_{b} is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of R² and R³ is optionally present depending on the valence of the atom to which each is attached, and if present, each of R² and R³ is independently hydrogen, halo, hydroxyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
R⁴ is hydrogen, optionally substituted C₁₋₆ alkyl, C₃₋₈ cycloalkyl, optionally substituted C₆₋₁₀ aryl, optionally substituted C₁₋₉ heteroaryl, or optionally substituted C₁₋₉ heterocyclyl;
U is hydrogen,

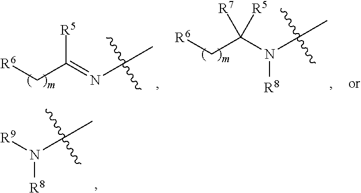

wherein m is 0-3, and each of R⁵, R⁶, R⁷, R⁸, and R^{g} is independently hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl; or
R³ and U, together with the nitrogen atom to which they are attached, form 4- to 6-membered heterocyclyl or heteroaryl optionally substituted by one or more substituents selected from hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
wherein each [symbol] is a single bond or a double bond and at least one [symbol] is a double bond; or each [symbol] is an aromatic bond;
each of V and Z is independently N or CH; and
A is optionally substituted C₃₋₈ carbocyclyl, optionally substituted C₁₋₉ heterocyclyl, or optionally substituted C₁₋₉ heteroaryl.

Exemplary compounds of formula (II) are those shown in Table 2, below, and pharmaceutically acceptable salts thereof.

TABLE 2

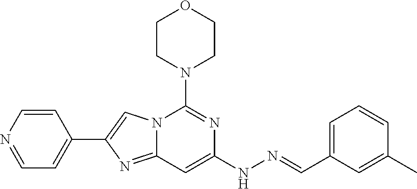 [SMILES: Cc1cccc(/C=N/Nc2cc3nc(-c4ccncc4)cn3c(N3CCOCC3)n2)c1]

(2.1)

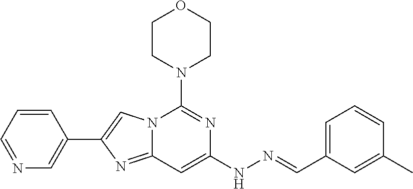 [SMILES: Cc1cccc(/C=N/Nc2cc3nc(-c4cccnc4)cn3c(N3CCOCC3)n2)c1]

(2.2)

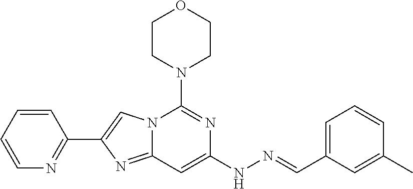 [SMILES: Cc1cccc(/C=N/Nc2cc3nc(-c4ccccn4)cn3c(N3CCOCC3)n2)c1]

(2.3)

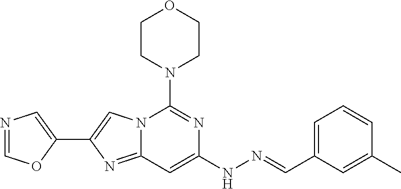 [SMILES: Cc1cccc(/C=N/Nc2cc3nc(-c4cnco4)cn3c(N3CCOCC3)n2)c1]

(2.4)

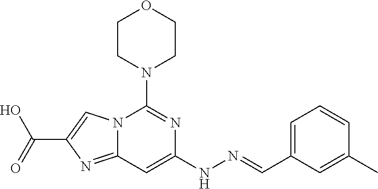 [SMILES: Cc1cccc(/C=N/Nc2cc3nc(C(=O)O)cn3c(N3CCOCC3)n2)c1]

(2.5)

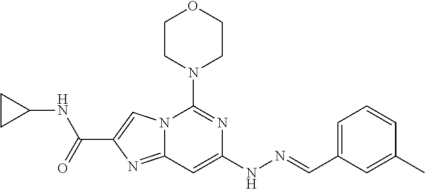 [SMILES: Cc1cccc(/C=N/Nc2cc3nc(C(=O)NC4CC4)cn3c(N3CCOCC3)n2)c1]

(2.6)

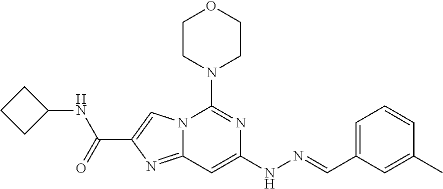 [SMILES: Cc1cccc(/C=N/Nc2cc3nc(C(=O)NC4CCC4)cn3c(N3CCOCC3)n2)c1]

(2.7)

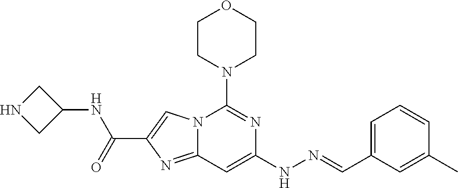 [SMILES: Cc1cccc(/C=N/Nc2cc3nc(C(=O)NC4CNC4)cn3c(N3CCOCC3)n2)c1]

(2.8)

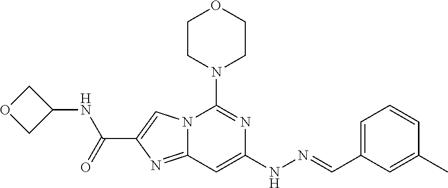 [SMILES: Cc1cccc(/C=N/Nc2cc3nc(C(=O)NC4COC4)cn3c(N3CCOCC3)n2)c1]

(2.9)

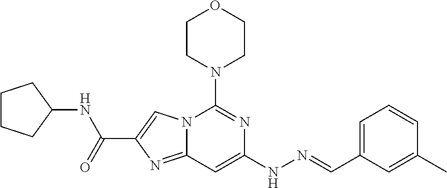 [SMILES: Cc1cccc(/C=N/Nc2cc3nc(C(=O)NC4CCCC4)cn3c(N3CCOCC3)n2)c1]

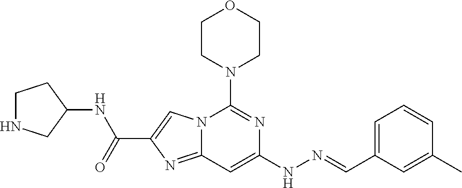 [SMILES: Cc1cccc(/C=N/Nc2cc3nc(C(=O)NC4CCNC4)cn3c(N3CCOCC3)n2)c1]

(2.10)

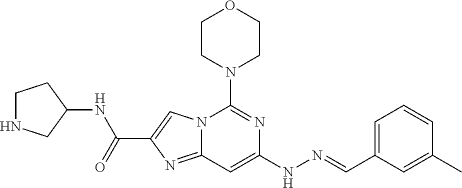 [SMILES: Cc1cccc(/C=N/Nc2cc3nc(C(=O)NC4CCNC4)cn3c(N3CCOCC3)n2)c1]

(2.11)

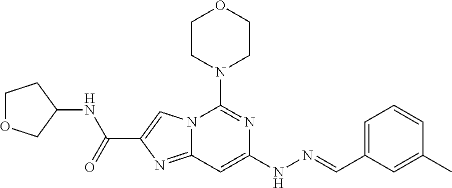 [SMILES: Cc1cccc(/C=N/Nc2cc3nc(C(=O)NC4CCOC4)cn3c(N3CCOCC3)n2)c1]

(2.12)

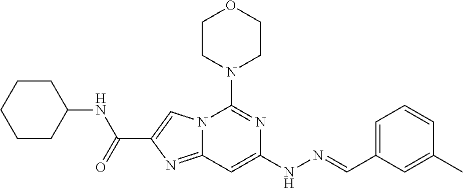 [SMILES: Cc1cccc(/C=N/Nc2cc3nc(C(=O)NC4CCCCC4)cn3c(N3CCOCC3)n2)c1]

(2.13)

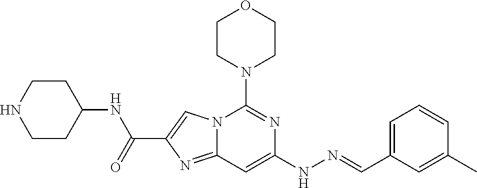 [SMILES: Cc1cccc(/C=N/Nc2cc3nc(C(=O)NC4CCNCC4)cn3c(N3CCOCC3)n2)c1]

(2.14)

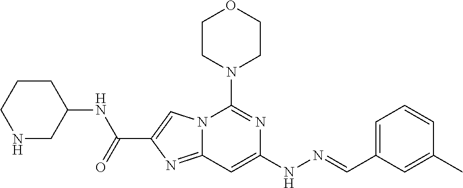 [SMILES: Cc1cccc(/C=N/Nc2cc3nc(C(=O)NC4CCCNC4)cn3c(N3CCOCC3)n2)c1]

(2.15)

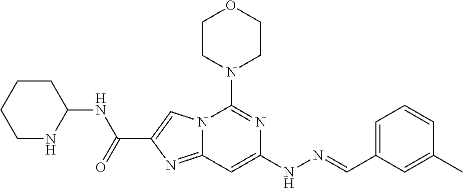 [SMILES: Cc1cccc(/C=N/Nc2cc3nc(C(=O)NC4CCCCN4)cn3c(N3CCOCC3)n2)c1]

(2.16)

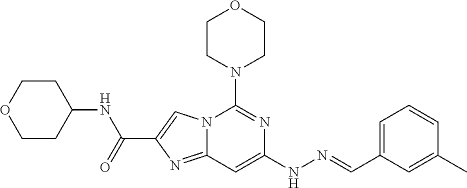 [SMILES: Cc1cccc(/C=N/Nc2cc3nc(C(=O)NC4CCOCC4)cn3c(N3CCOCC3)n2)c1]

(2.17)

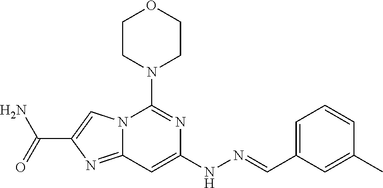 [SMILES: Cc1cccc(/C=N/Nc2cc3nc(C(N)=O)cn3c(N3CCOCC3)n2)c1]

(2.18)

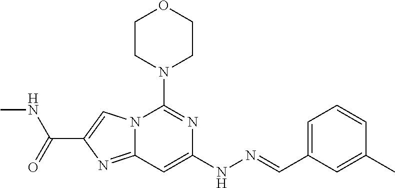

(2.19)

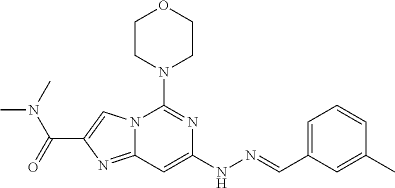

(2.20)

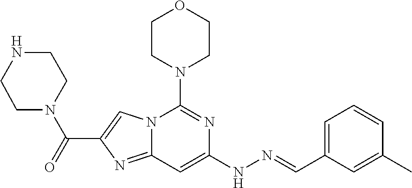 [SMILES: Cc1cccc(/C=N/Nc2cc3nc(C(=O)N4CCNCC4)cn3c(N3CCOCC3)n2)c1]

(2.21)

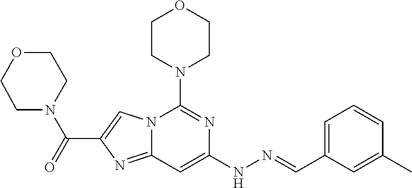 [SMILES: Cc1cccc(/C=N/Nc2cc3nc(C(=O)N4CCOCC4)cn3c(N3CCOCC3)n2)c1]

(2.22)

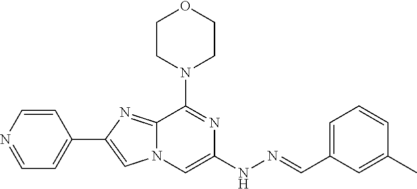 [SMILES: Cc1cccc(/C=N/Nc2cn3cc(-c4ccncc4)nc3c(N3CCOCC3)n2)c1]

(2.23)

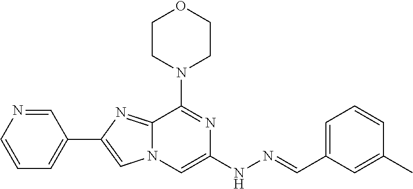 [SMILES: Cc1cccc(/C=N/Nc2cn3cc(-c4cccnc4)nc3c(N3CCOCC3)n2)c1]

(2.24)

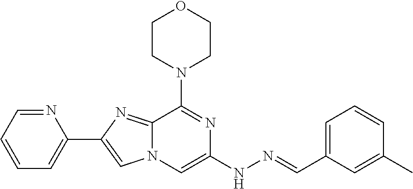 [SMILES: Cc1cccc(/C=N/Nc2cn3cc(-c4ccccn4)nc3c(N3CCOCC3)n2)c1]

(2.25)

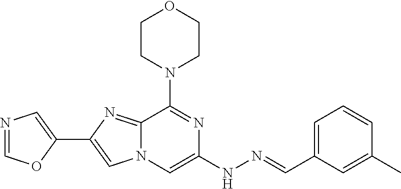 [SMILES: Cc1cccc(/C=N/Nc2cn3cc(-c4cnco4)nc3c(N3CCOCC3)n2)c1]

(2.26)

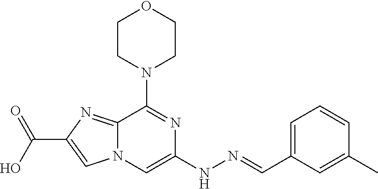 [SMILES: Cc1cccc(/C=N/Nc2cn3cc(C(=O)O)nc3c(N3CCOCC3)n2)c1]

(2.27)

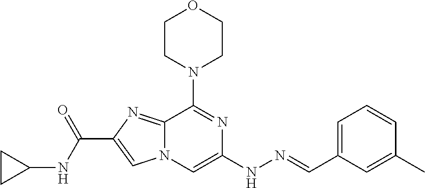 [SMILES: Cc1cccc(/C=N/Nc2cn3cc(C(=O)NC4CC4)nc3c(N3CCOCC3)n2)c1]

(2.28)

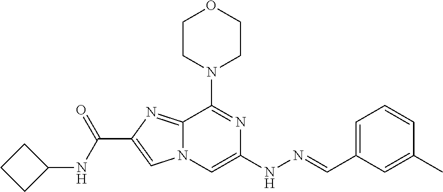 [SMILES: Cc1cccc(/C=N/Nc2cn3cc(C(=O)NC4CCC4)nc3c(N3CCOCC3)n2)c1]

(2.29)

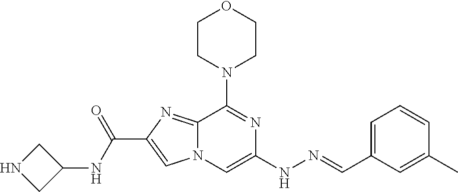 [SMILES: Cc1cccc(/C=N/Nc2cn3cc(C(=O)NC4CNC4)nc3c(N3CCOCC3)n2)c1]

(2.30)

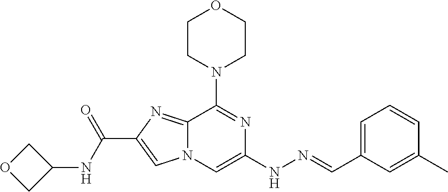 [SMILES: Cc1cccc(/C=N/Nc2cn3cc(C(=O)NC4COC4)nc3c(N3CCOCC3)n2)c1]

(2.31)

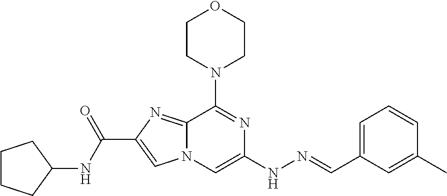 [SMILES: Cc1cccc(/C=N/Nc2cn3cc(C(=O)NC4CCCC4)nc3c(N3CCOCC3)n2)c1]

(2.32)

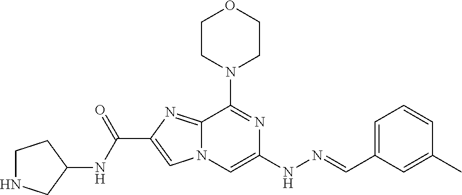 [SMILES: Cc1cccc(/C=N/Nc2cn3cc(C(=O)NC4CCNC4)nc3c(N3CCOCC3)n2)c1]

(2.33)

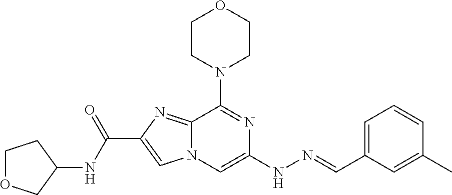 [SMILES: Cc1cccc(/C=N/Nc2cn3cc(C(=O)NC4CCOC4)nc3c(N3CCOCC3)n2)c1]

(2.34)

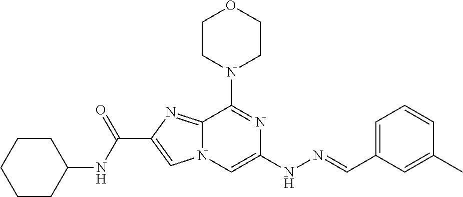 [SMILES: Cc1cccc(/C=N/Nc2cn3cc(C(=O)NC4CCCCC4)nc3c(N3CCOCC3)n2)c1]

(2.35)

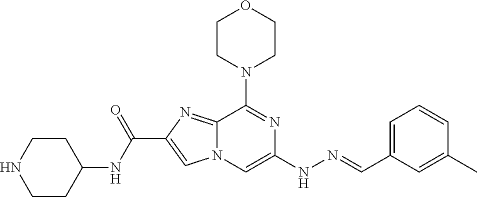 [SMILES: Cc1cccc(/C=N/Nc2cn3cc(C(=O)NC4CCNCC4)nc3c(N3CCOCC3)n2)c1]

(2.36)

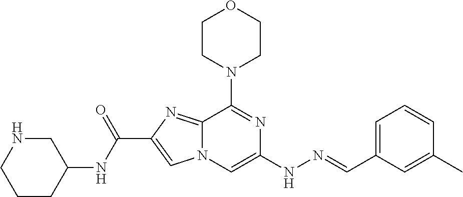 [SMILES: Cc1cccc(/C=N/Nc2cn3cc(C(=O)NC4CCCNC4)nc3c(N3CCOCC3)n2)c1]

(2.37)

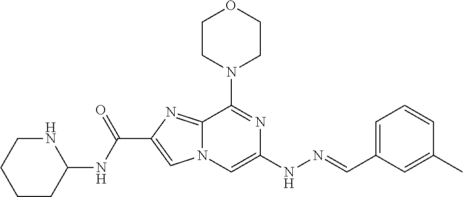 [SMILES: Cc1cccc(/C=N/Nc2cn3cc(C(=O)NC4CCCCN4)nc3c(N3CCOCC3)n2)c1]

(2.38)

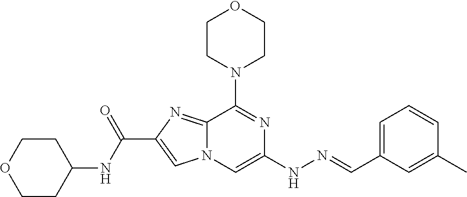 [SMILES: Cc1cccc(/C=N/Nc2cn3cc(C(=O)NC4CCOCC4)nc3c(N3CCOCC3)n2)c1]

(2.39)

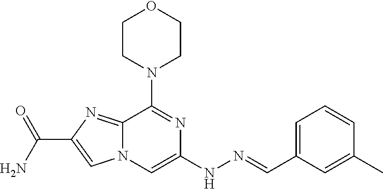 [SMILES: Cc1cccc(/C=N/Nc2cn3cc(C(N)=O)nc3c(N3CCOCC3)n2)c1]

(2.40)

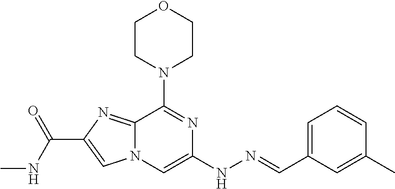

(2.41)

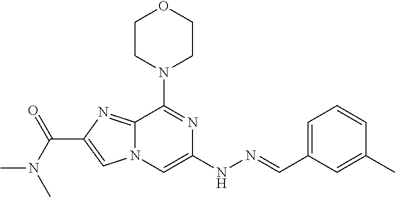

(2.42)

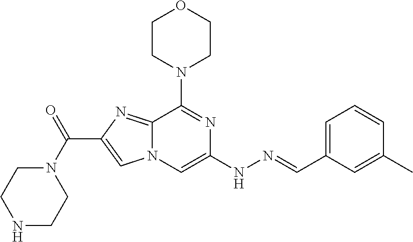 [SMILES: Cc1cccc(/C=N/Nc2cn3cc(C(=O)N4CCNCC4)nc3c(N3CCOCC3)n2)c1]

(2.43)

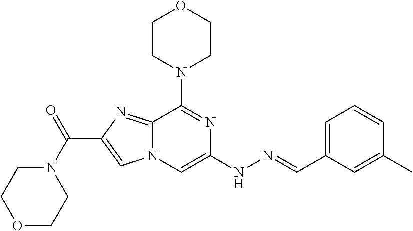 [SMILES: Cc1cccc(/C=N/Nc2cn3cc(C(=O)N4CCOCC4)nc3c(N3CCOCC3)n2)c1]

(2.44)

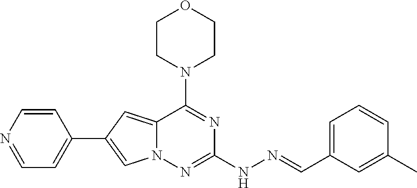 [SMILES: Cc1cccc(/C=N/Nc2nc(N3CCOCC3)c3cc(-c4ccncc4)cn3n2)c1]

(2.45)

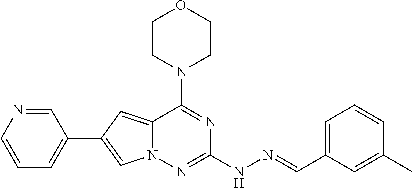 [SMILES: Cc1cccc(/C=N/Nc2nc(N3CCOCC3)c3cc(-c4cccnc4)cn3n2)c1]

(2.46)

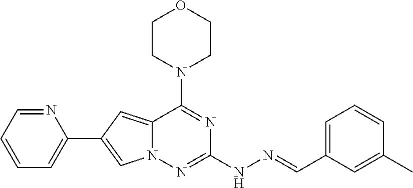 [SMILES: Cc1cccc(/C=N/Nc2nc(N3CCOCC3)c3cc(-c4ccccn4)cn3n2)c1]

(2.47)

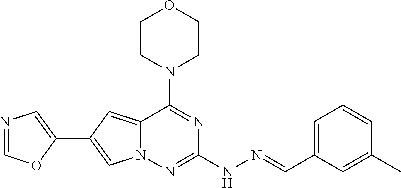 [SMILES: Cc1cccc(/C=N/Nc2nc(N3CCOCC3)c3cc(-c4cnco4)cn3n2)c1]

(2.48)

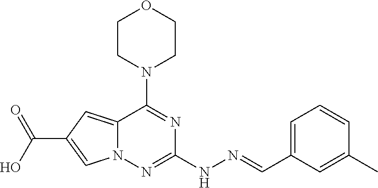 [SMILES: Cc1cccc(/C=N/Nc2nc(N3CCOCC3)c3cc(C(=O)O)cn3n2)c1]

(2.49)

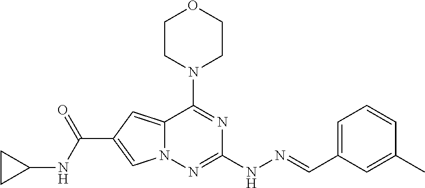 [SMILES: Cc1cccc(/C=N/Nc2nc(N3CCOCC3)c3cc(C(=O)NC4CC4)cn3n2)c1]

(2.50)

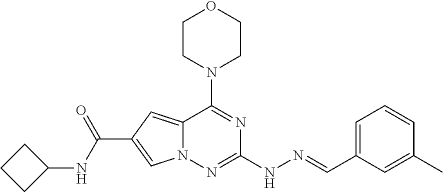 [SMILES: Cc1cccc(/C=N/Nc2nc(N3CCOCC3)c3cc(C(=O)NC4CCC4)cn3n2)c1]

(2.51)

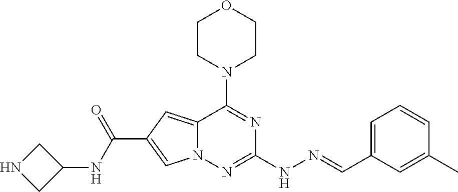 [SMILES: Cc1cccc(/C=N/Nc2nc(N3CCOCC3)c3cc(C(=O)NC4CNC4)cn3n2)c1]

(2.52)

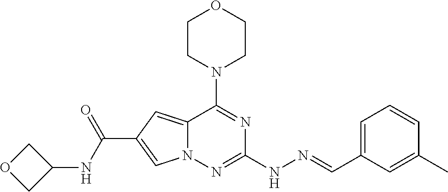 [SMILES: Cc1cccc(/C=N/Nc2nc(N3CCOCC3)c3cc(C(=O)NC4COC4)cn3n2)c1]

(2.53)

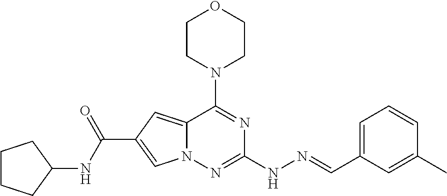 [SMILES: Cc1cccc(/C=N/Nc2nc(N3CCOCC3)c3cc(C(=O)NC4CCCC4)cn3n2)c1]

(2.54)

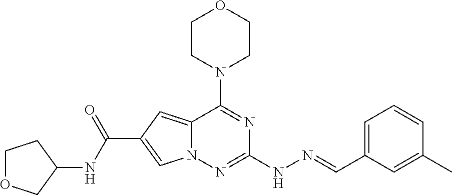 [SMILES: Cc1cccc(/C=N/Nc2nc(N3CCOCC3)c3cc(C(=O)NC4CCOC4)cn3n2)c1]

(2.55)

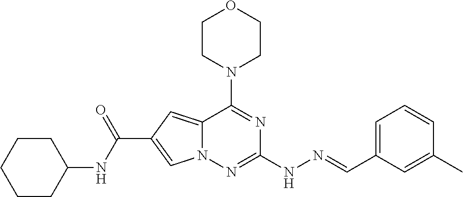 [SMILES: Cc1cccc(/C=N/Nc2nc(N3CCOCC3)c3cc(C(=O)NC4CCCCC4)cn3n2)c1]

(2.56)

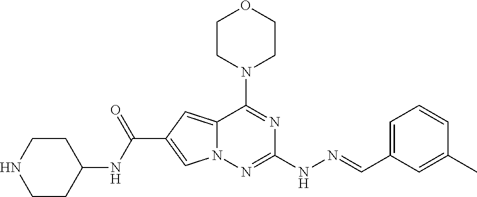 [SMILES: Cc1cccc(/C=N/Nc2nc(N3CCOCC3)c3cc(C(=O)NC4CCNCC4)cn3n2)c1]

(2.57)

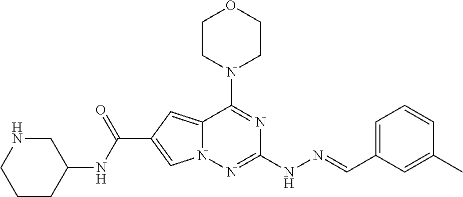 [SMILES: Cc1cccc(/C=N/Nc2nc(N3CCOCC3)c3cc(C(=O)NC4CCCNC4)cn3n2)c1]

(2.58)

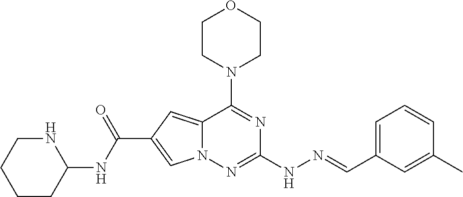 [SMILES: Cc1cccc(/C=N/Nc2nc(N3CCOCC3)c3cc(C(=O)NC4CCCCN4)cn3n2)c1]

(2.59)

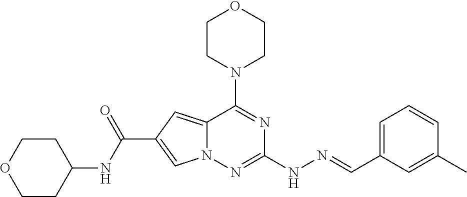 [SMILES: Cc1cccc(/C=N/Nc2nc(N3CCOCC3)c3cc(C(=O)NC4CCOCC4)cn3n2)c1]

(2.60)

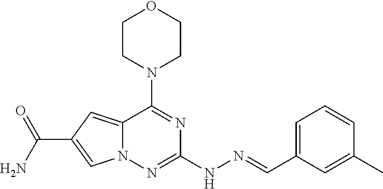 [SMILES: Cc1cccc(/C=N/Nc2nc(N3CCOCC3)c3cc(C(N)=O)cn3n2)c1]

(2.61)

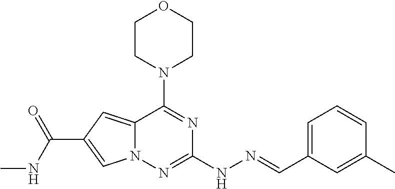

(2.62)

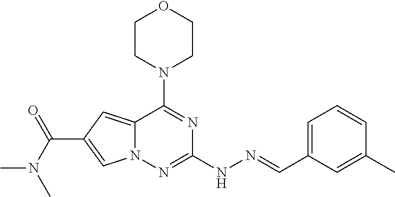

(2.63)

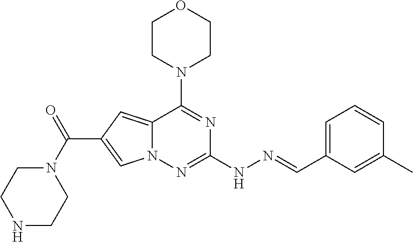 [SMILES: Cc1cccc(/C=N/Nc2nc(N3CCOCC3)c3cc(C(=O)N4CCNCC4)cn3n2)c1]

(2.64)

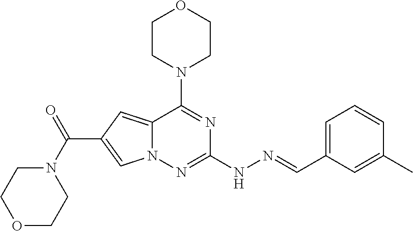 [SMILES: Cc1cccc(/C=N/Nc2nc(N3CCOCC3)c3cc(C(=O)N4CCOCC4)cn3n2)c1]

(2.65)

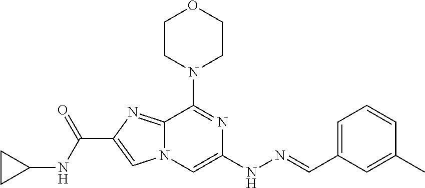 [SMILES: Cc1cccc(/C=N/Nc2cn3cc(C(=O)NC4CC4)nc3c(N3CCOCC3)n2)c1]

(2.66)

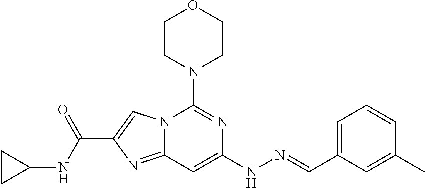 [SMILES: Cc1cccc(/C=N/Nc2cc3nc(C(=O)NC4CC4)cn3c(N3CCOCC3)n2)c1]

(2.67)

In some embodiments, the PIKfyve inhibitor is a compound of formula (III):

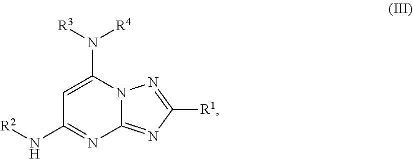

or a pharmaceutically acceptable salt thereof,
wherein
R¹ is optionally substituted C₁₋₆ alkyl, optionally substituted C₃₋₈ cycloalkyl, optionally substituted C₁₋₉ heterocyclyl, optionally substituted C₆₋₁₀ aryl, or optionally substituted C₁₋₉ heteroaryl;
R² is optionally substituted C₁₋₆ alkyl, optionally substituted C₆₋₁₀ aryl, optionally substituted C₁₋₉ heteroaryl, —N═CHRₐ, or —N═CHR_{b}R_{c}, wherein Rₐ is optionally substituted C₁₋₆ alkyl or optionally substituted C₁₋₉ heteroaryl; R_{b} is optionally substituted C₆₋₁₀ arylene; and R_{c} is hydrogen or NHSO₂Me; and
each of R³ and R⁴ is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl; or R₃ and R₄, together with the nitrogen to which they are attached, form optionally substituted C₁₋₉ heterocyclyl.

Exemplary compounds of formula (III) are those shown in Table 3, below, and pharmaceutically acceptable salts thereof.

TABLE 3

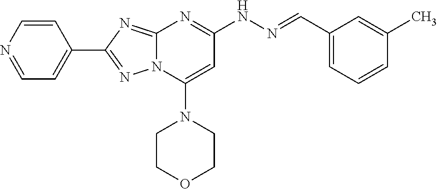 [SMILES: Cc1cccc(/C=N/Nc2cc(N3CCOCC3)n3nc(-c4ccncc4)nc3n2)c1]

(3.1)

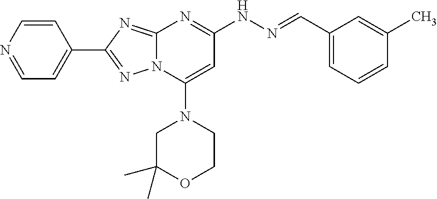 [SMILES: Cc1cccc(/C=N/Nc2cc(N3CCOC(C)(C)C3)n3nc(-c4ccncc4)nc3n2)c1]

(3.2)

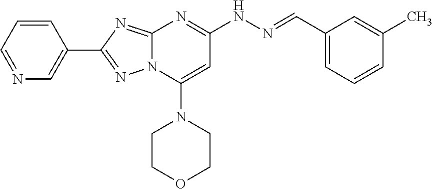 [SMILES: Cc1cccc(/C=N/Nc2cc(N3CCOCC3)n3nc(-c4cccnc4)nc3n2)c1]

(3.3)

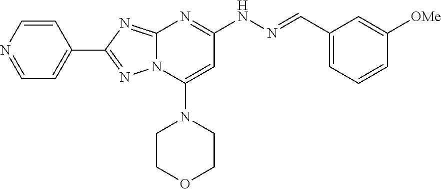 [SMILES: COc1cccc(/C=N/Nc2cc(N3CCOCC3)n3nc(-c4ccncc4)nc3n2)c1]

(3.4)

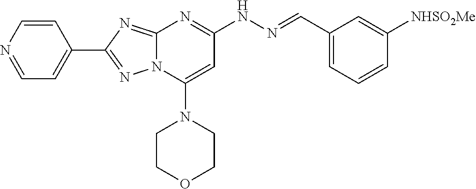

(3.5)

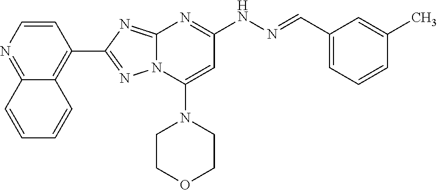 [SMILES: Cc1cccc(/C=N/Nc2cc(N3CCOCC3)n3nc(-c4ccnc5ccccc45)nc3n2)c1]

(3.6)

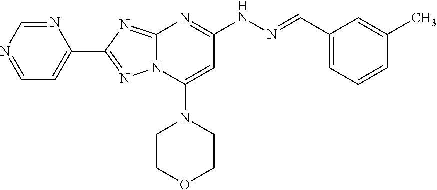 [SMILES: Cc1cccc(/C=N/Nc2cc(N3CCOCC3)n3nc(-c4ccncn4)nc3n2)c1]

(3.7)

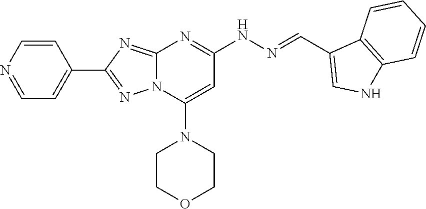 [SMILES: C(=N\Nc1cc(N2CCOCC2)n2nc(-c3ccncc3)nc2n1)/c1c[nH]c2ccccc12]

(3.8)

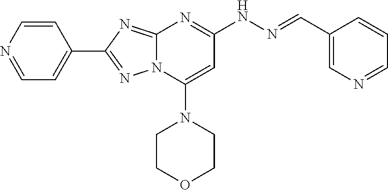 [SMILES: C(=N\Nc1cc(N2CCOCC2)n2nc(-c3ccncc3)nc2n1)/c1cccnc1]

(3.9)

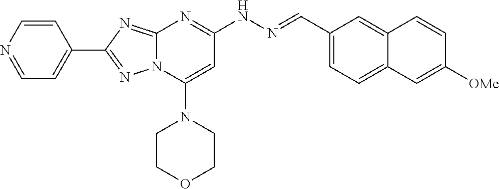 [SMILES: COc1ccc2cc(/C=N/Nc3cc(N4CCOCC4)n4nc(-c5ccncc5)nc4n3)ccc2c1]

(3.10)

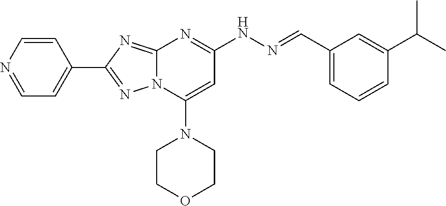 [SMILES: CC(C)c1cccc(/C=N/Nc2cc(N3CCOCC3)n3nc(-c4ccncc4)nc3n2)c1]

(3.11)

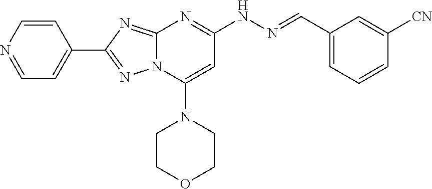 [SMILES: N#Cc1cccc(/C=N/Nc2cc(N3CCOCC3)n3nc(-c4ccncc4)nc3n2)c1]

(3.12)

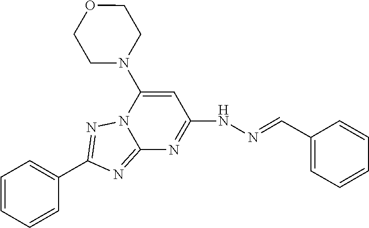 [SMILES: C(=N\Nc1cc(N2CCOCC2)n2nc(-c3ccccc3)nc2n1)/c1ccccc1]

(3.13)

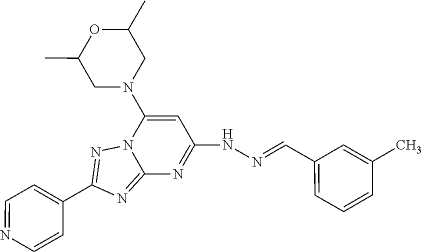 [SMILES: Cc1cccc(/C=N/Nc2cc(N3CC(C)OC(C)C3)n3nc(-c4ccncc4)nc3n2)c1]

(3.14)

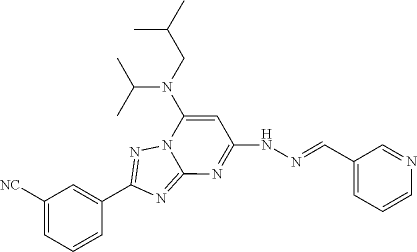 [SMILES: CC(C)CN(c1cc(N/N=C/c2cccnc2)nc2nc(-c3cccc(C#N)c3)nn12)C(C)C]

(3.15)

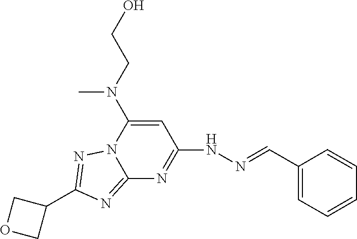 [SMILES: CN(CCO)c1cc(N/N=C/c2ccccc2)nc2nc(C3COC3)nn12]

(3.16)

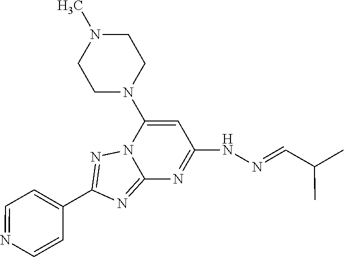 [SMILES: CC(C)/C=N/Nc1cc(N2CCN(C)CC2)n2nc(-c3ccncc3)nc2n1]

(3.17)

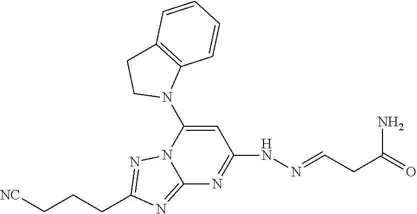 [SMILES: N#CCCCc1nc2nc(N/N=C/CC(N)=O)cc(N3CCc4ccccc43)n2n1]

(3.18)

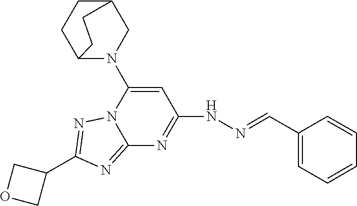 [SMILES: C(=N\Nc1cc(N2CC3CCC2CC3)n2nc(C3COC3)nc2n1)/c1ccccc1]

(3.19)

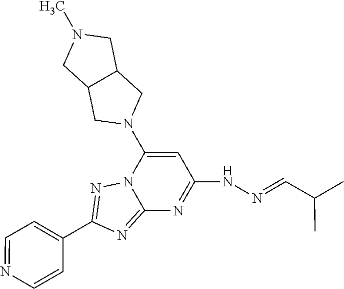 [SMILES: CC(C)/C=N/Nc1cc(N2CC3CN(C)CC3C2)n2nc(-c3ccncc3)nc2n1]

(3.20)

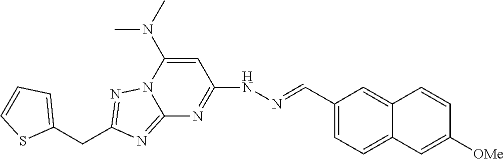 [SMILES: COc1ccc2cc(/C=N/Nc3cc(N(C)C)n4nc(Cc5cccs5)nc4n3)ccc2c1]

(3.21)

In some embodiments, the PIKfyve inhibitor is a compound shown in Table 4, below, or a pharmaceutically acceptable salt thereof.

TABLE 4

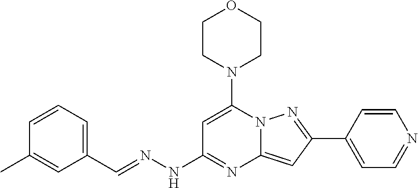 [SMILES: Cc1cccc(/C=N/Nc2cc(N3CCOCC3)n3nc(-c4ccncc4)cc3n2)c1]

(4.1)

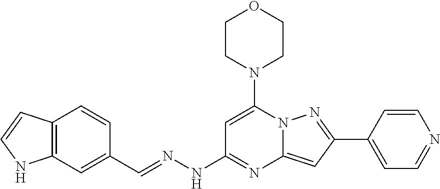 [SMILES: C(=N\Nc1cc(N2CCOCC2)n2nc(-c3ccncc3)cc2n1)/c1ccc2cc[nH]c2c1]

(4.2)

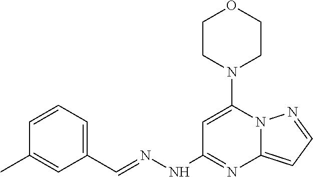 [SMILES: Cc1cccc(/C=N/Nc2cc(N3CCOCC3)n3nccc3n2)c1]

(4.3)

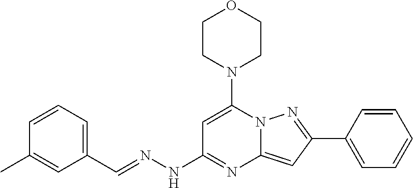 [SMILES: Cc1cccc(/C=N/Nc2cc(N3CCOCC3)n3nc(-c4ccccc4)cc3n2)c1]

(4.4)

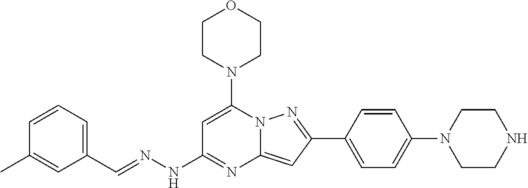 [SMILES: Cc1cccc(/C=N/Nc2cc(N3CCOCC3)n3nc(-c4ccc(N5CCNCC5)cc4)cc3n2)c1]

(4.5)

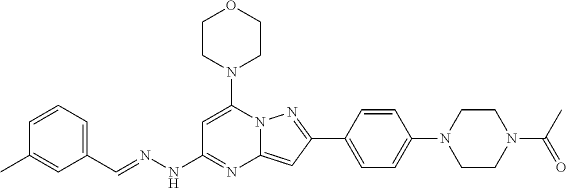 [SMILES: CC(=O)N1CCN(c2ccc(-c3cc4nc(N/N=C/c5cccc(C)c5)cc(N5CCOCC5)n4n3)cc2)CC1]

(4.6)

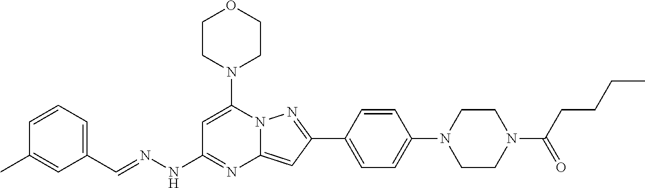 [SMILES: CCCCC(=O)N1CCN(c2ccc(-c3cc4nc(N/N=C/c5cccc(C)c5)cc(N5CCOCC5)n4n3)cc2)CC1]

(4.7)

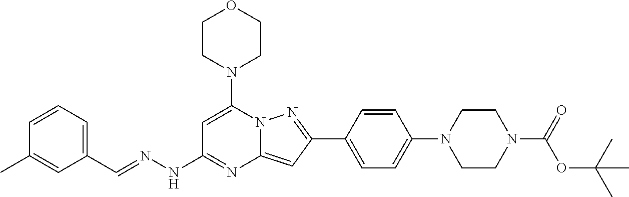 [SMILES: Cc1cccc(/C=N/Nc2cc(N3CCOCC3)n3nc(-c4ccc(N5CCN(C(=O)OC(C)(C)C)CC5)cc4)cc3n2)c1]

(4.8)

In some embodiments, the PIKfyve inhibitor is a compound shown in Table 5, below, or a pharmaceutically acceptable salt thereof.

TABLE 5

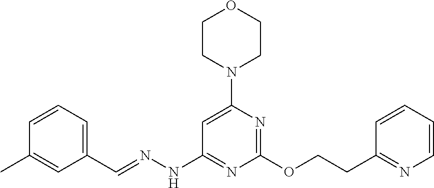 [SMILES: Cc1cccc(/C=N/Nc2cc(N3CCOCC3)nc(OCCc3ccccn3)n2)c1]

(5.1)

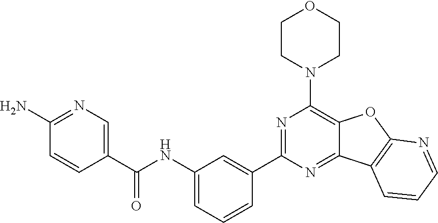 [SMILES: Nc1ccc(C(=O)Nc2cccc(-c3nc(N4CCOCC4)c4oc5ncccc5c4n3)c2)cn1]

(5.2)

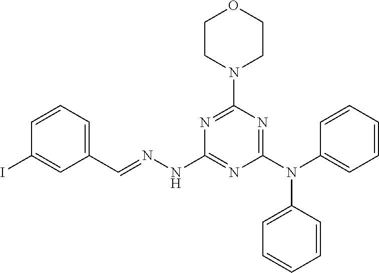 [SMILES: Ic1cccc(/C=N/Nc2nc(N3CCOCC3)nc(N(c3ccccc3)c3ccccc3)n2)c1]

(5.3)

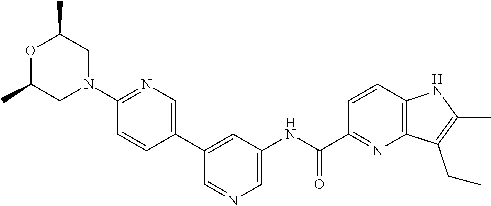

(5.4)

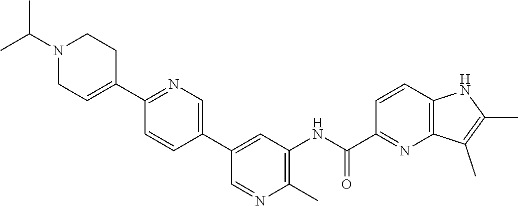 [SMILES: Cc1ncc(-c2ccc(C3=CCN(C(C)C)CC3)nc2)cc1NC(=O)c1ccc2[nH]c(C)c(C)c2n1]

(5.6)

In some embodiments, the PIKfyve inhibitor is a compound of formula (IV):

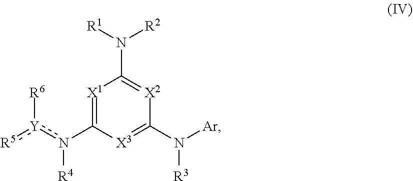

or a pharmaceutically acceptable salt thereof,
wherein
each bond denoted as [symbol] is either a single bond or a double bond, provided that the bonds denoted as [symbol] are not both simultaneously double bonds;
X¹ is selected from N and CR^{A};
X² is selected from N and CR^{A};
X³ is selected from N and CR^{A};
each R^{A} is independently selected from H, halo, C₁₋₆ alkyl, C₁₋₆ haloalkyl, C₁₋₆ alkoxy, and C₁₋₆haloalkoxy;
Ar is selected from C₆₋₁₀ aryl and 5-10 membered heteroaryl, each of which is optionally substituted with 1, 2, 3, 4, or 5 substituents independently selected from R⁷;
each R⁷ is independently selected from halo, CN, NO₂, C₁₋₆alkyl, C₁₋₆ haloalkyl, C₂₋₆ alkenyl, C₂₋₆ alkynyl, OR^{a1}, SR^{a1}, C(O)R^{b1}, C(O)NR^{c2}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} and S(O)₂NR^{c1}R^{d1};
R¹ is selected from the group consisting of H and C₁₋₆ alkyl, wherein said C₁₋₆ alkyl is optionally substituted with 1, 2, or 3 substituents independently selected from halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2};
R² is C₁₋₆ alkyl, which is optionally substituted with 1, 2, or 3 substituents independently selected from halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}; or
R¹ and R² together with the N to which they are attached form a 4-7 membered non-aromatic heterocyclyl, which is optionally substituted with 1, 2, 3, 4, or 5 substituents independently selected R⁸;
each R⁸ is independently selected from halo, C₁₋₆ alkyl, C₂₋₆ alkenyl, C₂₋₆ alkynyl, C₁₋₆ haloalkyl, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2};
R³ is selected from H, C₁₋₆ alkyl, and C₁₋₆ haloalkyl;
R⁴ is selected from H, C₁₋₆ alkyl, and C₁₋₆ haloalkyl;
Y is selected from N, C, and CR^{A};
when the bond between R⁵ and Y is a single bond, R⁵ is 5-10 membered heteroaryl,
which is optionally substituted with 1, 2, 3, 4, or 5 substituents independently selected from halo, CN, NO₂, C₁₋₆ alkyl, C₂₋₆ alkenyl, C₂₋₆ alkynyl, C₁₋₆ haloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, R^{c3}C(O)NR^{c3}R^{d3},
NR^{c3}S(O)₂R^{b3}, NR^{c3}S(O)₂NR^{c3}R^{d3}, S(O)₂R^{b3} and S(O)₂NR^{c3}R^{d3};
when the bond between R⁵ and Y is a double bond, R⁵ is CR^{B}R_{c}
R^{B} is selected from H, C₁₋₆ alkyl, and C₁₋₆ haloalkyl;
R^{c} is selected from C₆₋₁₀ aryl and 5-10 membered heteroaryl, each of which is optionally substituted with 1, 2, 3, 4, or 5 substituents independently selected from halo, CN, NO₂, C₁₋₆ alkyl, C₂₋₆ alkenyl, C₂₋₆ alkynyl, Cᵢ₋ₑ haloalkyl, OR^{a3}, SR^{a3}, C(O)R^{b3}, C(O)NR³R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR³R^{d3}, C(O)OR^{a3}, OC(O)R^{b3}, OC(O)NR³R^{d3}, NR^{c3}R^{d3}, NR^{c3}C(O)R^{b3}, NR^{c3}C(O)OR^{a3}, NR^{c3}C(O)NR³R^{d3}, NR^{c3}S(O)₂R^{b3}, NR^{c3}S(O)₂NR³R^{d3}, S(O)₂R^{b3} and S(O)₂NR^{c3}R^{d3}; or
R⁴ and R⁵ together with Y and N to which R⁴ is attached form a 5-14 membered heteroaryl, which is optionally substituted with 1, 2, 3, 4, or 5 substituents independently selected from R^{g};
each R^{g} is independently selected from halo, CN, NO₂, C₁₋₆ alkyl, C₂₋₆ alkenyl, C₂₋₆ alkynyl, Cᵢ₋ₑ haloalkyl, OR^{a3}, SR^{a3}, C(O)R^{b3}, C(O)NR³R^{d3}, C(O)OR^{a3}, OC(O)R^{b3}, OC(O)NR³R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said Cᵢ₋ₑ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3} and S(O)₂NR^{c3}R^{d3};
R⁶ is selected from H, C₁₋₆ alkyl, and C₁₋₆ haloalkyl; or R⁶ is absent;
each R^{a1}, R^{b1}, R^{a2}, R^{b2}, R^{a3}, and R^{b3} is independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are optionally substituted with 1, 2, 3, 4, or 5 substituents independently selected from R^{g};
each R^{c1}, R^{d1}, R^{c2}, R^{d2}, R^{c3}, and R^{d3} is independently selected from H, C₁₋₆ alkyl, C₁₋₄ haloalkyl, C₂₋₆ alkenyl, C₂₋₆ alkynyl, C(O)R^{b7}, C(O)NR^{c7}R^{d7}, C(O)OR^{a7}, NR^{c7}R^{d7}, S(O)R^{b7}, S(O)NR^{c7}R^{d7}, S(O)₂R^{b7}, and S(O)₂NR^{c7}R^{d7}; wherein said Cᵢ₋ₑ alkyl, C₂₋₆ alkenyl, and C₂₋₆ alkynyl is optionally substituted with 1, 2, 3, 4, or 5 substituents independently selected from R^{g}; each R^{a7}, R^{b7}, R^{c7}, and R^{d7} is in dependently selected from H, C₁₋₆ alkyl, 0-4 haloalkyl, C₂₋₆ alkenyl, C₂₋₆ alkynyl, and R^{g}, wherein said C₁₋₆ alkyl, C₂₋₆ alkenyl, and C₂₋₆ alkynyl is optionally substituted with 1, 2, 3, 4, or 5 substituents independently selected from R^{g};
or any R^{c1} and R^{d1} together with the N atom to which they are attached form a 4-, 5-, 6-, or 7-membered non-aromatic heterocyclyl group optionally substituted with 1, 2, or 3 substituents independently selected from R^{g};
or any R^{c2} and R^{d2} together with the N atom to which they are attached form a 4-, 5-, 6-, or 7-membered non-aromatic heterocyclyl group optionally substituted with 1, 2, or 3 substituents independently selected from R^{g};
or any R^{c3} and R^{d3} together with the N atom to which they are attached form a 4-, 5-, 6-, or 7-membered non-aromatic heterocyclyl group optionally substituted with 1, 2, or 3 substituents independently selected from R^{g};
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfamyl, C₁₋₆ alkylsulfonyl, carbamyl, C₁₋₆ alkylcarbamyl, di(C₁₋₆ alkyl)carbamyl, carboxy, C₁₋₆ acyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the PIKfyve inhibitor is a compound shown in Table 6, below, or a pharmaceutically acceptable salt thereof.

TABLE 6

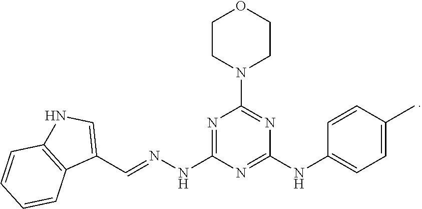 [SMILES: Cc1ccc(Nc2nc(N/N=C/c3c[nH]c4ccccc34)nc(N3CCOCC3)n2)cc1]

(6.1)

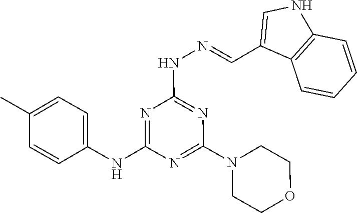 [SMILES: Cc1ccc(Nc2nc(N/N=C/c3c[nH]c4ccccc34)nc(N3CCOCC3)n2)cc1]

(6.2)

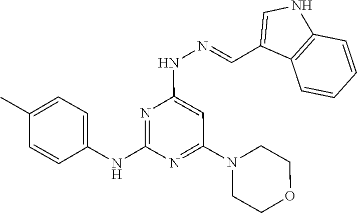 [SMILES: Cc1ccc(Nc2nc(N/N=C/c3c[nH]c4ccccc34)cc(N3CCOCC3)n2)cc1]

(6.3)

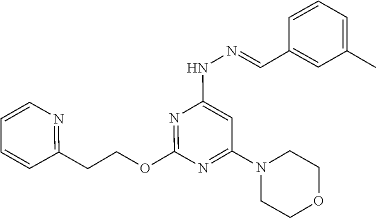 [SMILES: Cc1cccc(/C=N/Nc2cc(N3CCOCC3)nc(OCCc3ccccn3)n2)c1]

(6.4)

In some embodiments, the PIKfyve inhibitor is a compound shown in Table 7, below, or a pharmaceutically acceptable salt thereof.

TABLE 7

| | |
|---|---|
| 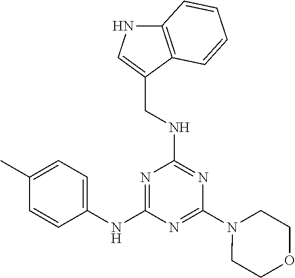 [SMILES: Cc1ccc(Nc2nc(NCc3c[nH]c4ccccc34)nc(N3CCOCC3)n2)cc1] | (7.1) |
| 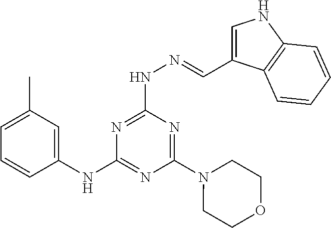 [SMILES: Cc1cccc(Nc2nc(N/N=C/c3c[nH]c4ccccc34)nc(N3CCOCC3)n2)c1] | (7.2) |
| 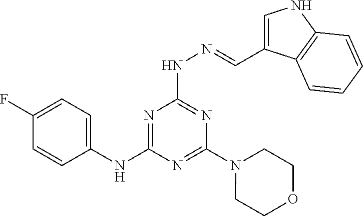 [SMILES: Fc1ccc(Nc2nc(N/N=C/c3c[nH]c4ccccc34)nc(N3CCOCC3)n2)cc1] | (7.3) |
| 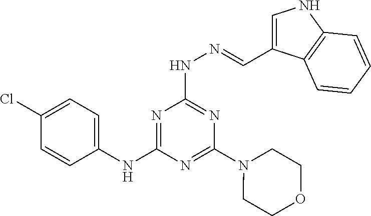 [SMILES: Clc1ccc(Nc2nc(N/N=C/c3c[nH]c4ccccc34)nc(N3CCOCC3)n2)cc1] | (7.4) |
| 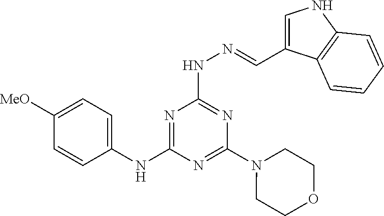 [SMILES: COc1ccc(Nc2nc(N/N=C/c3c[nH]c4ccccc34)nc(N3CCOCC3)n2)cc1] | (7.5) |
| 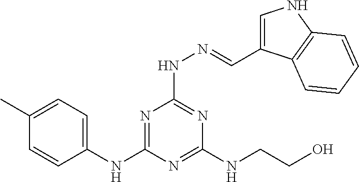 [SMILES: Cc1ccc(Nc2nc(NCCO)nc(N/N=C/c3c[nH]c4ccccc34)n2)cc1] | (7.6) |
| 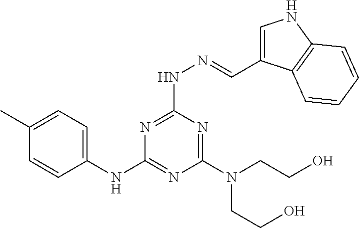 [SMILES: Cc1ccc(Nc2nc(N/N=C/c3c[nH]c4ccccc34)nc(N(CCO)CCO)n2)cc1] | (7.7) |
| 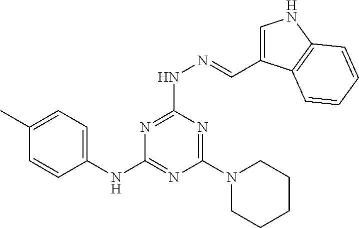 [SMILES: Cc1ccc(Nc2nc(N/N=C/c3c[nH]c4ccccc34)nc(N3CCCCC3)n2)cc1] | (7.8) |
| 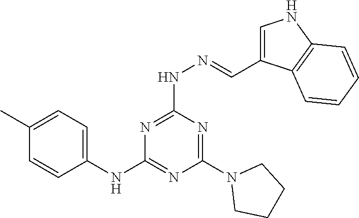 [SMILES: Cc1ccc(Nc2nc(N/N=C/c3c[nH]c4ccccc34)nc(N3CCCC3)n2)cc1] | (7.9) |
| 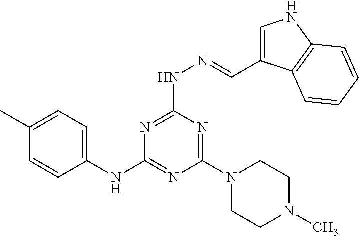 [SMILES: Cc1ccc(Nc2nc(N/N=C/c3c[nH]c4ccccc34)nc(N3CCN(C)CC3)n2)cc1] | (7.10) |
| 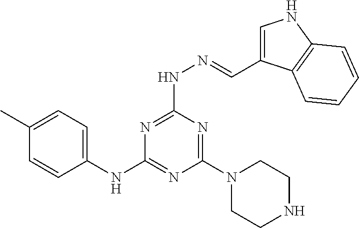 [SMILES: Cc1ccc(Nc2nc(N/N=C/c3c[nH]c4ccccc34)nc(N3CCNCC3)n2)cc1] | (7.11) |
| 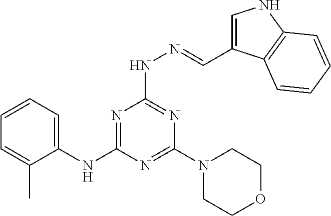 [SMILES: Cc1ccccc1Nc1nc(N/N=C/c2c[nH]c3ccccc23)nc(N2CCOCC2)n1] | (7.12) |
| 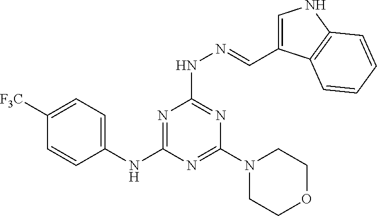 [SMILES: FC(F)(F)c1ccc(Nc2nc(N/N=C/c3c[nH]c4ccccc34)nc(N3CCOCC3)n2)cc1] | (7.13) |
| 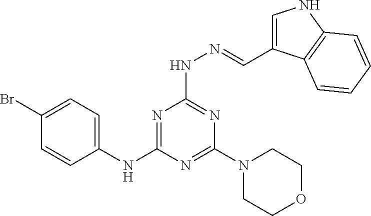 [SMILES: Brc1ccc(Nc2nc(N/N=C/c3c[nH]c4ccccc34)nc(N3CCOCC3)n2)cc1] | (7.14) |
| 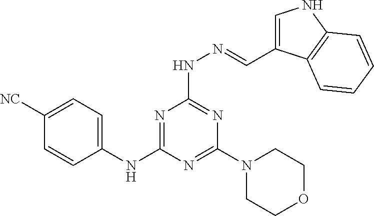 [SMILES: N#Cc1ccc(Nc2nc(N/N=C/c3c[nH]c4ccccc34)nc(N3CCOCC3)n2)cc1] | (7.15) |
| 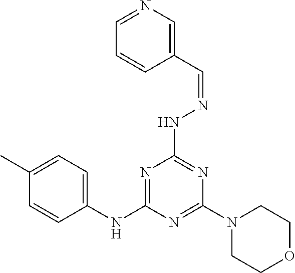 [SMILES: Cc1ccc(Nc2nc(N/N=C\c3cccnc3)nc(N3CCOCC3)n2)cc1] | (7.16) |
| 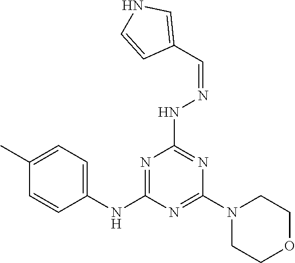 [SMILES: Cc1ccc(Nc2nc(N/N=C\c3cc[nH]c3)nc(N3CCOCC3)n2)cc1] | (7.17) |
| 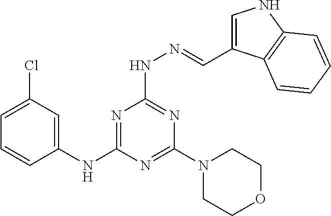 [SMILES: Clc1cccc(Nc2nc(N/N=C/c3c[nH]c4ccccc34)nc(N3CCOCC3)n2)c1] | (7.18) |
| 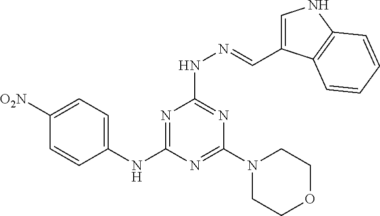 [SMILES: O=[N+]([O-])c1ccc(Nc2nc(N/N=C/c3c[nH]c4ccccc34)nc(N3CCOCC3)n2)cc1] | (7.19) |
| 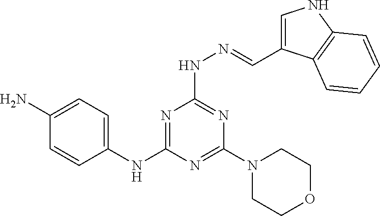 [SMILES: Nc1ccc(Nc2nc(N/N=C/c3c[nH]c4ccccc34)nc(N3CCOCC3)n2)cc1] | (7.20) |
| 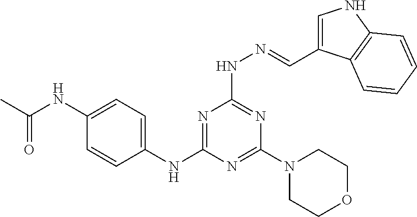 [SMILES: CC(=O)Nc1ccc(Nc2nc(N/N=C/c3c[nH]c4ccccc34)nc(N3CCOCC3)n2)cc1] | (7.21) |
| 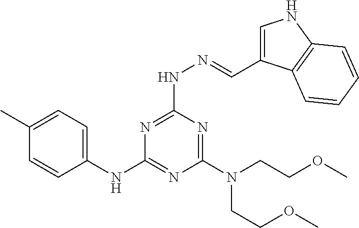 [SMILES: COCCN(CCOC)c1nc(N/N=C/c2c[nH]c3ccccc23)nc(Nc2ccc(C)cc2)n1] | (7.22) |
| 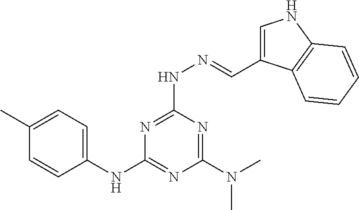 [SMILES: Cc1ccc(Nc2nc(N/N=C/c3c[nH]c4ccccc34)nc(N(C)C)n2)cc1] | (7.23) |
| 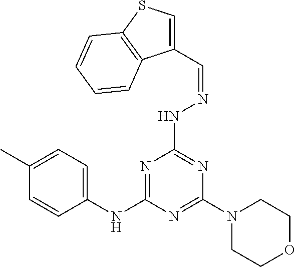 [SMILES: Cc1ccc(Nc2nc(N/N=C\c3csc4ccccc34)nc(N3CCOCC3)n2)cc1] | (7.24) |
| 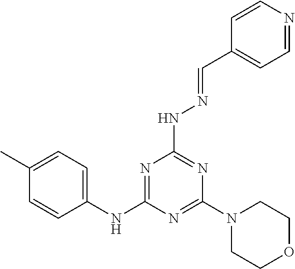 [SMILES: Cc1ccc(Nc2nc(N/N=C/c3ccncc3)nc(N3CCOCC3)n2)cc1] | (7.25) |
| 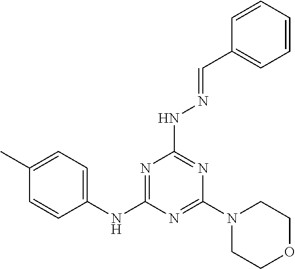 [SMILES: Cc1ccc(Nc2nc(N/N=C/c3ccccc3)nc(N3CCOCC3)n2)cc1] | (7.26) |
| 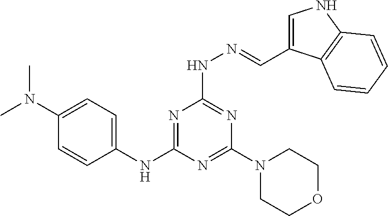 [SMILES: CN(C)c1ccc(Nc2nc(N/N=C/c3c[nH]c4ccccc34)nc(N3CCOCC3)n2)cc1] | (7.27) |
| 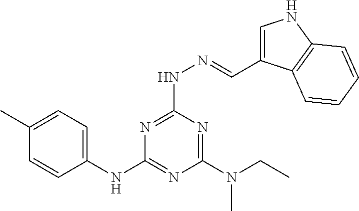 [SMILES: CCN(C)c1nc(N/N=C/c2c[nH]c3ccccc23)nc(Nc2ccc(C)cc2)n1] | (7.28) |
| 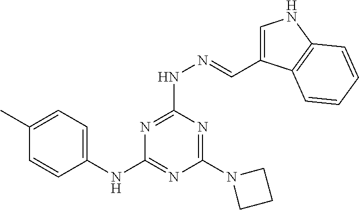 [SMILES: Cc1ccc(Nc2nc(N/N=C/c3c[nH]c4ccccc34)nc(N3CCC3)n2)cc1] | (7.29) |
| 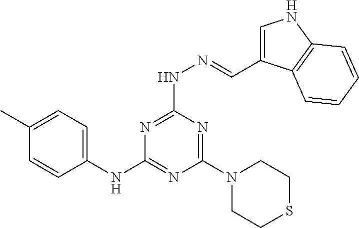 [SMILES: Cc1ccc(Nc2nc(N/N=C/c3c[nH]c4ccccc34)nc(N3CCSCC3)n2)cc1] | (7.30) |
| 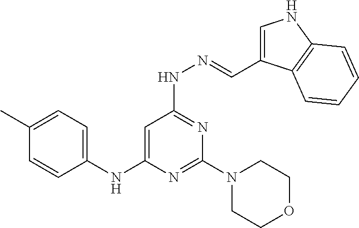 [SMILES: Cc1ccc(Nc2cc(N/N=C/c3c[nH]c4ccccc34)nc(N3CCOCC3)n2)cc1] | (7.31) |
| 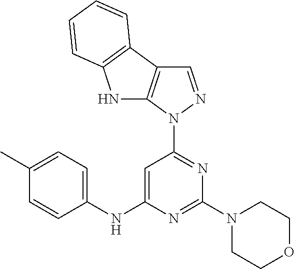 [SMILES: Cc1ccc(Nc2cc(-n3ncc4c5ccccc5[nH]c43)nc(N3CCOCC3)n2)cc1] | (7.32) |
| 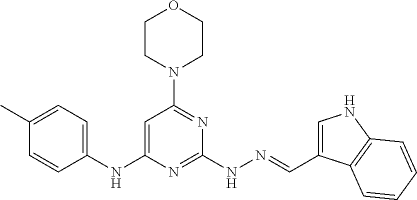 [SMILES: Cc1ccc(Nc2cc(N3CCOCC3)nc(N/N=C/c3c[nH]c4ccccc34)n2)cc1] | (7.33) |
| 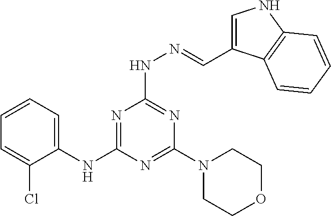 [SMILES: Clc1ccccc1Nc1nc(N/N=C/c2c[nH]c3ccccc23)nc(N2CCOCC2)n1] | (7.34) |
| 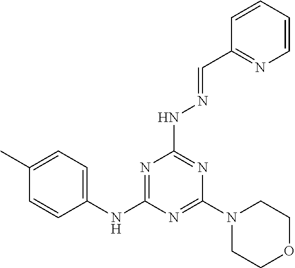 [SMILES: Cc1ccc(Nc2nc(N/N=C/c3ccccn3)nc(N3CCOCC3)n2)cc1] | (7.35) |
| 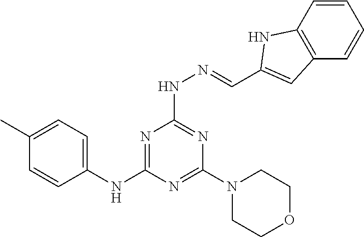 [SMILES: Cc1ccc(Nc2nc(N/N=C/c3cc4ccccc4[nH]3)nc(N3CCOCC3)n2)cc1] | (7.36) |
| 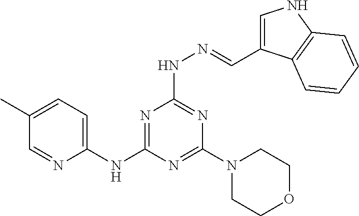 [SMILES: Cc1ccc(Nc2nc(N/N=C/c3c[nH]c4ccccc34)nc(N3CCOCC3)n2)nc1] | (7.37) |
| 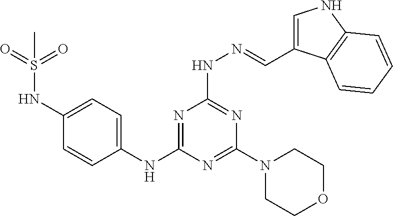 [SMILES: CS(=O)(=O)Nc1ccc(Nc2nc(N/N=C/c3c[nH]c4ccccc34)nc(N3CCOCC3)n2)cc1] | (7.38) |
| 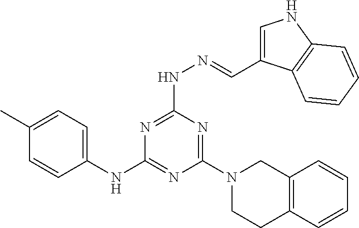 [SMILES: Cc1ccc(Nc2nc(N/N=C/c3c[nH]c4ccccc34)nc(N3CCc4ccccc4C3)n2)cc1] | (7.39) |
| 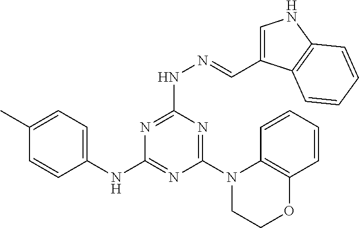 [SMILES: Cc1ccc(Nc2nc(N/N=C/c3c[nH]c4ccccc34)nc(N3CCOc4ccccc43)n2)cc1] | (7.40) |
| 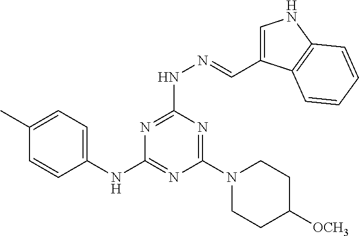 [SMILES: COC1CCN(c2nc(N/N=C/c3c[nH]c4ccccc34)nc(Nc3ccc(C)cc3)n2)CC1] | (7.41) |
| 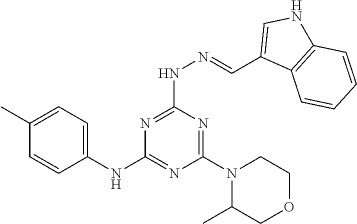 [SMILES: Cc1ccc(Nc2nc(N/N=C/c3c[nH]c4ccccc34)nc(N3CCOCC3C)n2)cc1] | (7.42) |
| 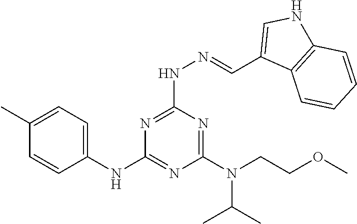 [SMILES: COCCN(c1nc(N/N=C/c2c[nH]c3ccccc23)nc(Nc2ccc(C)cc2)n1)C(C)C] | (7.43) |
| 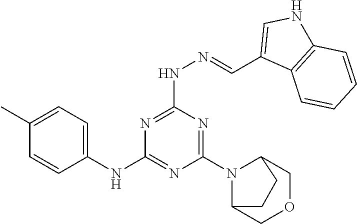 [SMILES: Cc1ccc(Nc2nc(N/N=C/c3c[nH]c4ccccc34)nc(N3C4CCC3COC4)n2)cc1] | (7.44) |
| 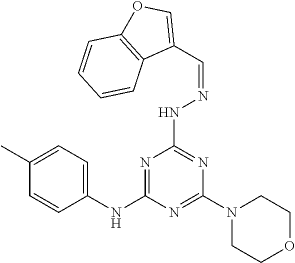 [SMILES: Cc1ccc(Nc2nc(N/N=C\c3coc4ccccc34)nc(N3CCOCC3)n2)cc1] | (7.45) |
| 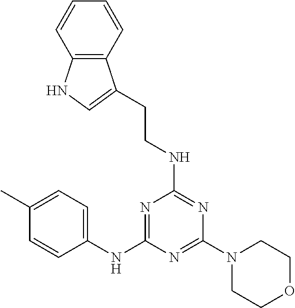 [SMILES: Cc1ccc(Nc2nc(NCCc3c[nH]c4ccccc34)nc(N3CCOCC3)n2)cc1] | (7.46) |
| 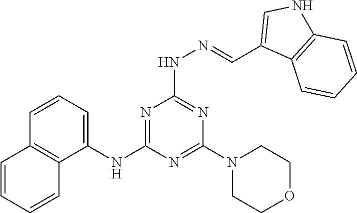 [SMILES: C(=N\Nc1nc(Nc2cccc3ccccc23)nc(N2CCOCC2)n1)/c1c[nH]c2ccccc12] | (7.47) |
| 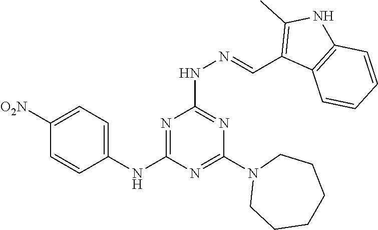 [SMILES: Cc1[nH]c2ccccc2c1/C=N/Nc1nc(Nc2ccc([N+](=O)[O-])cc2)nc(N2CCCCCC2)n1] | (7.48) |

In some embodiments, the PIKfyve inhibitor is a compound of formula (V):

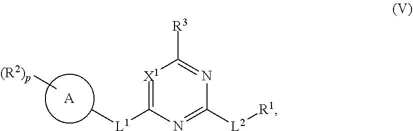

or a pharmaceutically acceptable salt thereof,
wherein
R¹ is hydroxy, optionally substituted C₁₋₆ alkyl, optionally substituted C₂₋₆ alkenyl, optionally substituted C₁₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl;
each occurrence of R² is independently optionally substituted C₁₋₆ alkyl, optionally substituted C₂₋₆ alkenyl, optionally substituted C₂₋₆ alkynyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
R³ is a nitrogen- or oxygen-containing moiety;
Ring A is (i) a 5 or 6-membered heteroaryl or 5-6 or 6-5 membered bicyclic heteroaryl, each having at least one nitrogen or oxygen ring atom, or (ii) phenyl;
L¹ is absent, C₁-C₂ alkylene, —NR^{C}—, —O—, —S—, —C(O)—, —NHC(O)—, or —C(O)NH—;
L² is —O—(CR^{a}R^{b})ₘ—, —(CR^{a}R^{b})ₘ—, —NR^{c}—(CR^{a}R^{b})ₘ—, or —S—(CR^{a}R^{b})ₘ—;
X¹ is CH, N, or CR^{C}; each occurrence of R^{a} and R^{b} are independently hydrogen, hydroxy, hydroxy(C₁₋₄)alkyl, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R_{c}, —S(O)R_{c}, —NR^{c}SO₂R^{d}, —OS(O)₂R^{d}, —OP(O)R^{d}R^{e}, or —P(O)R^{d}R^{e};
R^{c} is a hydrogen or C₁₋₆ alkyl;
each occurrence of R^{d} and R^{e} are independently hydrogen, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl;
m is 1-4; and
p is 1 or 2.

In some embodiments, the PIKfyve inhibitor is a compound shown in Table 8, below, or a pharmaceutically acceptable salt thereof.

TABLE 8

| | |
|---|---|
| 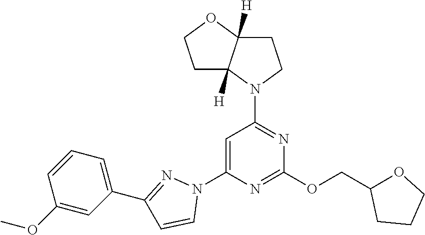 [SMILES: COc1cccc(-c2ccn(-c3cc(N4CC[C@H]5OCC[C@H]54)nc(OCC4CCCO4)n3)n2)c1] | (8.1) |
| 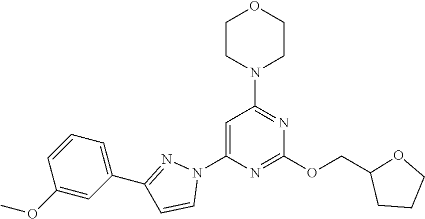 [SMILES: COc1cccc(-c2ccn(-c3cc(N4CCOCC4)nc(OCC4CCCO4)n3)n2)c1] | (8.2) |
| 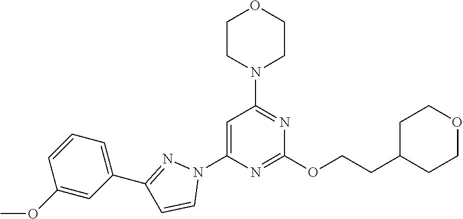 [SMILES: COc1cccc(-c2ccn(-c3cc(N4CCOCC4)nc(OCCC4CCOCC4)n3)n2)c1] | (8.3) |
| 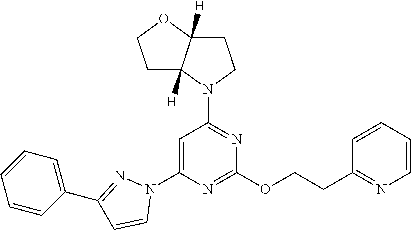 [SMILES: c1ccc(-c2ccn(-c3cc(N4CC[C@H]5OCC[C@H]54)nc(OCCc4ccccn4)n3)n2)cc1] | (8.4) |
| 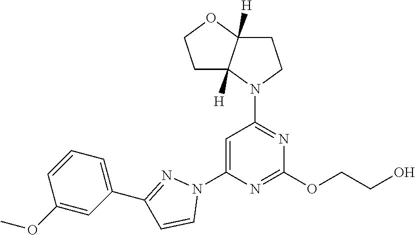 [SMILES: COc1cccc(-c2ccn(-c3cc(N4CC[C@H]5OCC[C@H]54)nc(OCCO)n3)n2)c1] | (8.5) |
| 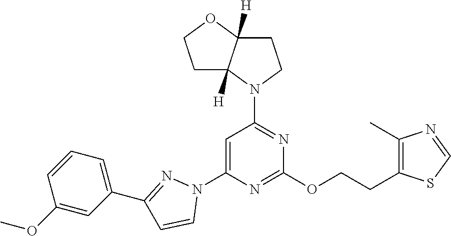 [SMILES: COc1cccc(-c2ccn(-c3cc(N4CC[C@H]5OCC[C@H]54)nc(OCCc4scnc4C)n3)n2)c1] | (8.6) |
| 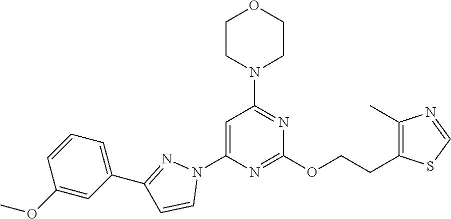 [SMILES: COc1cccc(-c2ccn(-c3cc(N4CCOCC4)nc(OCCc4scnc4C)n3)n2)c1] | (8.7) |
| 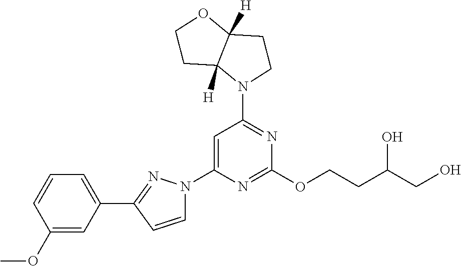 [SMILES: COc1cccc(-c2ccn(-c3cc(N4CC[C@H]5OCC[C@H]54)nc(OCCC(O)CO)n3)n2)c1] | (8.8) |
| 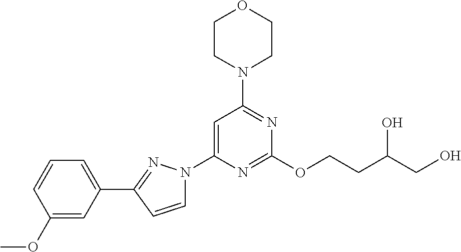 [SMILES: COc1cccc(-c2ccn(-c3cc(N4CCOCC4)nc(OCCC(O)CO)n3)n2)c1] | (8.9) |
| 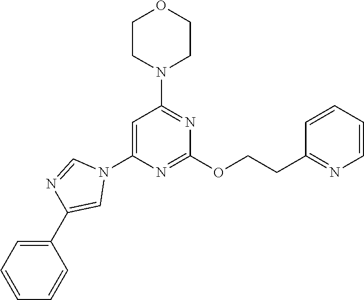 [SMILES: c1ccc(-c2cn(-c3cc(N4CCOCC4)nc(OCCc4ccccn4)n3)cn2)cc1] | (8.10) |
| 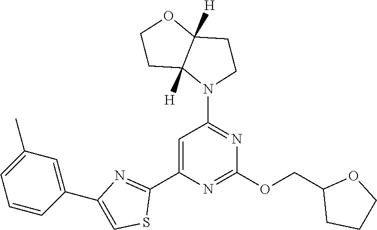 [SMILES: Cc1cccc(-c2csc(-c3cc(N4CC[C@H]5OCC[C@H]54)nc(OCC4CCCO4)n3)n2)c1] | (8.11) |
| 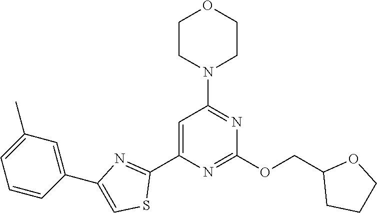 [SMILES: Cc1cccc(-c2csc(-c3cc(N4CCOCC4)nc(OCC4CCCO4)n3)n2)c1] | (8.12) |
| 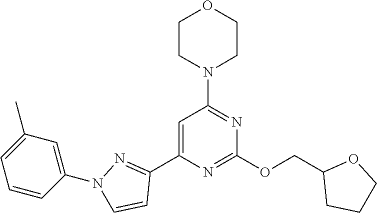 [SMILES: Cc1cccc(-n2ccc(-c3cc(N4CCOCC4)nc(OCC4CCCO4)n3)n2)c1] | (8.13) |
| 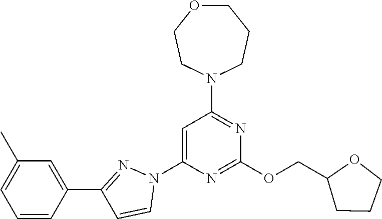 [SMILES: Cc1cccc(-c2ccn(-c3cc(N4CCCOCC4)nc(OCC4CCCO4)n3)n2)c1] | (8.14) |
| 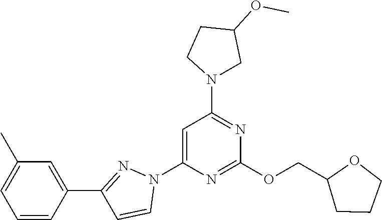 [SMILES: COC1CCN(c2cc(-n3ccc(-c4cccc(C)c4)n3)nc(OCC3CCCO3)n2)C1] | (8.15) |
| 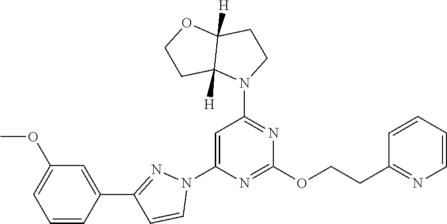 [SMILES: COc1cccc(-c2ccn(-c3cc(N4CC[C@H]5OCC[C@H]54)nc(OCCc4ccccn4)n3)n2)c1] | (8.16) |
| 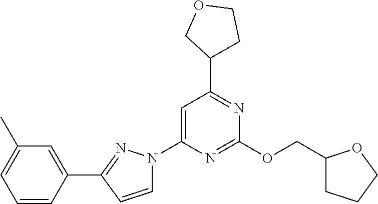 [SMILES: Cc1cccc(-c2ccn(-c3cc(C4CCOC4)nc(OCC4CCCO4)n3)n2)c1] | (8.17) |
| 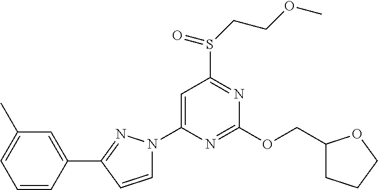 [SMILES: COCCS(=O)c1cc(-n2ccc(-c3cccc(C)c3)n2)nc(OCC2CCCO2)n1] | (8.18) |
| 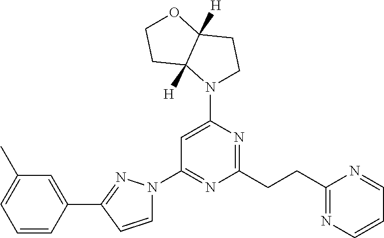 [SMILES: Cc1cccc(-c2ccn(-c3cc(N4CC[C@H]5OCC[C@H]54)nc(CCc4ncccn4)n3)n2)c1] | (8.19) |
| 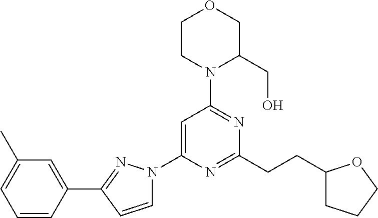 [SMILES: Cc1cccc(-c2ccn(-c3cc(N4CCOCC4CO)nc(CCC4CCCO4)n3)n2)c1] | (8.20) |

In some embodiments, the PIKfyve inhibitor is a compound of formula (VI):

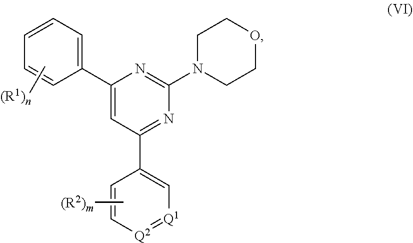

or a pharmaceutically acceptable salt thereof,
wherein
Q¹ and Q² are each independently CH or N, wherein Q¹ and Q² are not both N;
each R¹ is independently hydroxy, C₁₋₄ alkyl, or C₁₋₄ alkoxy;
n is 0, 1, or 2;
each R² is independently C₁₋₄ alkyl or C₁₋₄ alkoxy; and
m is 0 or 1.

In some embodiments, the PIKfyve inhibitor is a compound of the following structure:

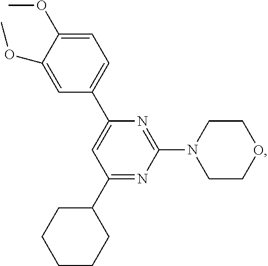 [SMILES: COc1ccc(-c2cc(C3CCCCC3)nc(N3CCOCC3)n2)cc1OC]

or a pharmaceutically acceptable salt thereof.

In some embodiments, the PIKfyve inhibitor is a compound of formula (VII):

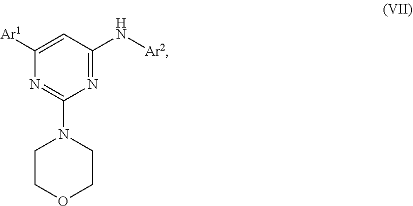

or a pharmaceutically acceptable salt thereof,
wherein
Ar¹ is phenyl or pyridyl, with each optionally independently substituted with 1 or 2 C₁₋₄ alkoxy; Ar² is phenyl, pyridyl, or pyrimidyl with each optionally independently substituted with halo, C₁₋₄ alkyl, C₁₋₄ alkoxy, or C(O)NR^{2a}R^{2b}; and
R^{2a} and R² are each independently H or C₁₋₄ alkyl

In some embodiments, the PIKfyve inhibitor is a compound shown in Table 9, below, or a pharmaceutically acceptable salt thereof.

TABLE 9

| | |
|---|---|
| 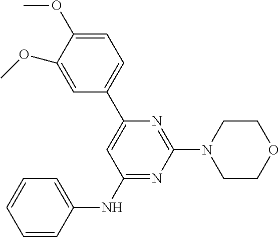 [SMILES: COc1ccc(-c2cc(Nc3ccccc3)nc(N3CCOCC3)n2)cc1OC] | (9.1) |
| 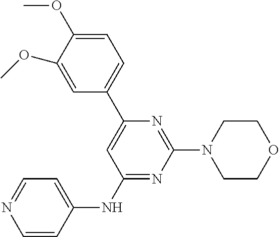 [SMILES: COc1ccc(-c2cc(Nc3ccncc3)nc(N3CCOCC3)n2)cc1OC] | (9.2) |
| 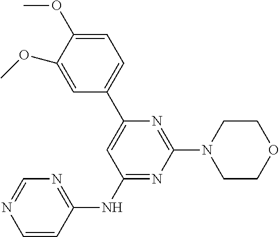 [SMILES: COc1ccc(-c2cc(Nc3ccncn3)nc(N3CCOCC3)n2)cc1OC] | (9.3) |
| 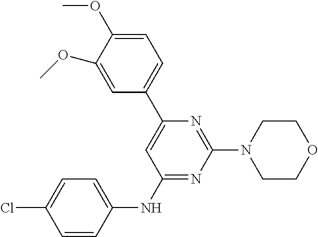 [SMILES: COc1ccc(-c2cc(Nc3ccc(Cl)cc3)nc(N3CCOCC3)n2)cc1OC] | (9.4) |
| 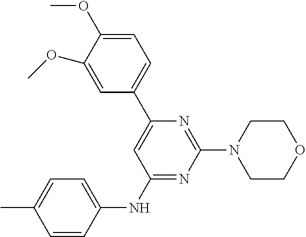 [SMILES: COc1ccc(-c2cc(Nc3ccc(C)cc3)nc(N3CCOCC3)n2)cc1OC] | (9.5) |
| 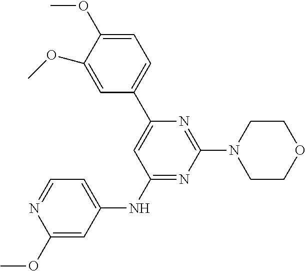 [SMILES: COc1cc(Nc2cc(-c3ccc(OC)c(OC)c3)nc(N3CCOCC3)n2)ccn1] | (9.6) |
| 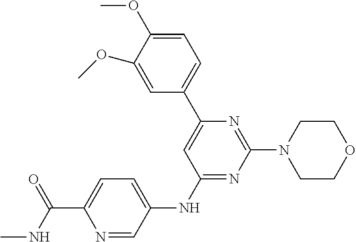 [SMILES: CNC(=O)c1ccc(Nc2cc(-c3ccc(OC)c(OC)c3)nc(N3CCOCC3)n2)cn1] | (9.7) |
| 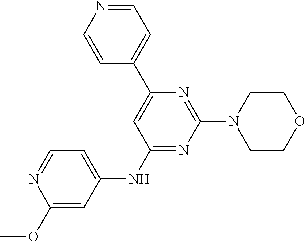 [SMILES: COc1cc(Nc2cc(-c3ccncc3)nc(N3CCOCC3)n2)ccn1] | (9.8) |
| 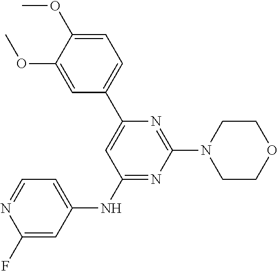 [SMILES: COc1ccc(-c2cc(Nc3ccnc(F)c3)nc(N3CCOCC3)n2)cc1OC] | (9.9) |
| 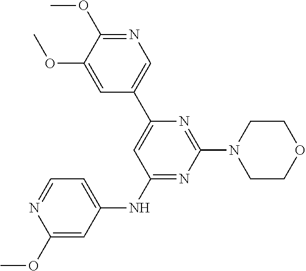 [SMILES: COc1cc(Nc2cc(-c3cnc(OC)c(OC)c3)nc(N3CCOCC3)n2)ccn1] | (9.10) |

In some embodiments, the PIKfyve inhibitor is a compound of formula (VIII):

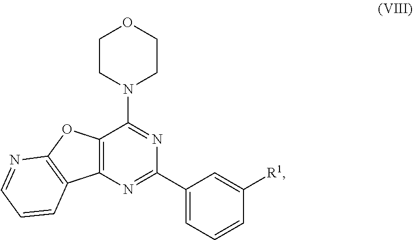

or a pharmaceutically acceptable salt thereof,
wherein
R¹ is hydroxy, C₁₋₄ alkoxy, or H(CO)R^{1a}; and
R^{1a} is phenyl or pyridyl, optionally substituted with amino, alkylamino, or dialkylamino.

In some embodiments, the PIKfyve inhibitor is a compound shown in Table 10, below, or a pharmaceutically acceptable salt thereof.

TABLE 10

| | |
|---|---|
| 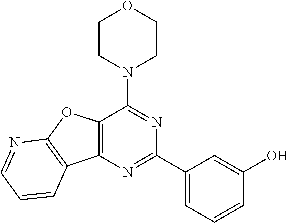 [SMILES: Oc1cccc(-c2nc(N3CCOCC3)c3oc4ncccc4c3n2)c1] | (10.1) |
| 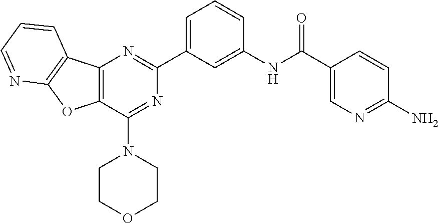 [SMILES: Nc1ccc(C(=O)Nc2cccc(-c3nc(N4CCOCC4)c4oc5ncccc5c4n3)c2)cn1] | (10.2) |

In some embodiments, the PIKfyve inhibitor is a compound of formula (IX):

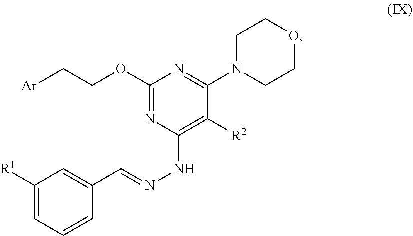

or a pharmaceutically acceptable salt thereof,
wherein
Ar is phenyl or pyridyl, with each optionally independently substituted with 1 or 2 alkyl, aminoalkyl, (alkylamino)alkyl, or (dialkylamino)alkyl;
R¹ is hydrogen or alkyl; and
R² is hydrogen or halo.

In some embodiments, the PIKfyve inhibitor is a compound shown in Table 11, below, or a pharmaceutically acceptable salt thereof.

TABLE 11

| | |
|---|---|
| 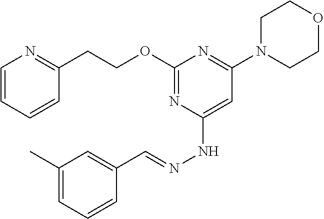 [SMILES: Cc1cccc(/C=N/Nc2cc(N3CCOCC3)nc(OCCc3ccccn3)n2)c1] | (11.1) |
| 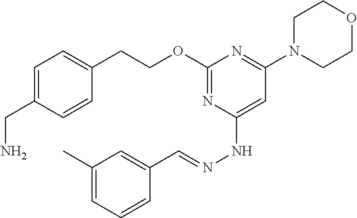 [SMILES: Cc1cccc(/C=N/Nc2cc(N3CCOCC3)nc(OCCc3ccc(CN)cc3)n2)c1] | (11.2) |
| 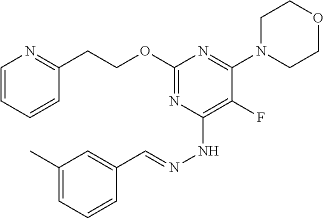 [SMILES: Cc1cccc(/C=N/Nc2nc(OCCc3ccccn3)nc(N3CCOCC3)c2F)c1] | (11.3) |

In some embodiments, the PIKfyve inhibitor is a compound of formula (X):

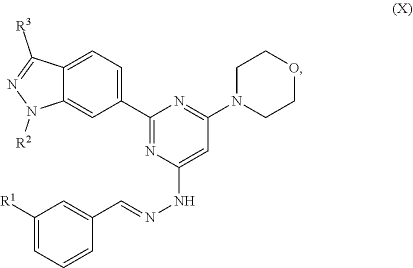

or a pharmaceutically acceptable salt thereof,
wherein

R¹ and R² are each independently hydrogen or C₁₋₄ alkyl; R³ is hydrogen or C₁₋₃ alkyl substituted with morpholinyl.

In some embodiments, the PIKfyve inhibitor is a compound shown in Table 12, below, or a pharmaceutically acceptable salt thereof.

TABLE 12

| | |
|---|---|
| 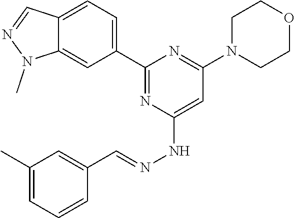 [SMILES: Cc1cccc(/C=N/Nc2cc(N3CCOCC3)nc(-c3ccc4cnn(C)c4c3)n2)c1] | (12.1) |
| 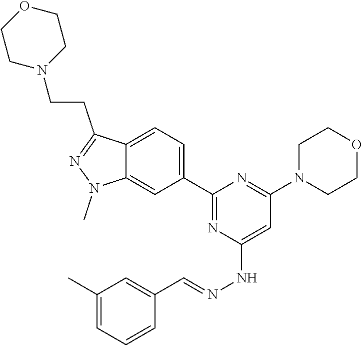 [SMILES: Cc1cccc(/C=N/Nc2cc(N3CCOCC3)nc(-c3ccc4c(CCN5CCOCC5)nn(C)c4c3)n2)c1] | (12.2) |
| 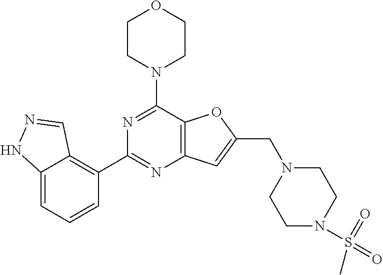 [SMILES: CS(=O)(=O)N1CCN(Cc2cc3nc(-c4cccc5[nH]ncc45)nc(N4CCOCC4)c3o2)CC1] | (12.3) |
| 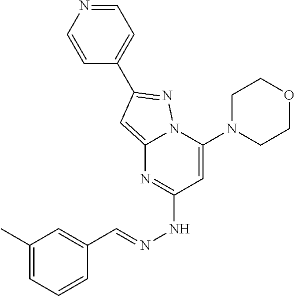 [SMILES: Cc1cccc(/C=N/Nc2cc(N3CCOCC3)n3nc(-c4ccncc4)cc3n2)c1] | (12.4) |

In some embodiments, the PIKfyve inhibitor is a compound of formula (XI):

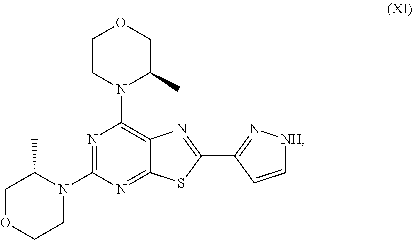 [SMILES: C[C@@H]1COCCN1c1nc(N2CCOC[C@@H]2C)nc2sc(-c3cc[nH]n3)nc12]

or a pharmaceutically acceptable salt thereof,
wherein

X is selected from

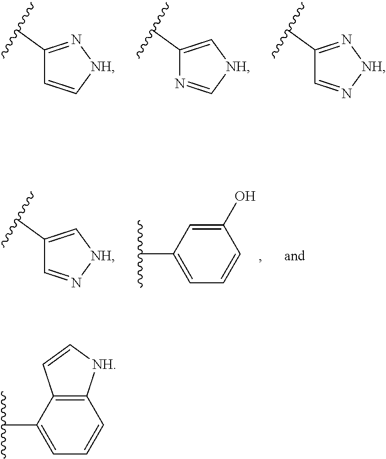

In some embodiments, the PIKfyve inhibitor is

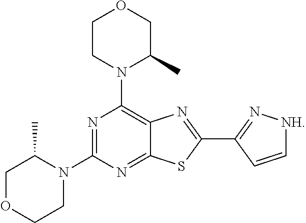 [SMILES: C[C@@H]1COCCN1c1nc(N2CCOC[C@@H]2C)nc2sc(-c3cc[nH]n3)nc12]

In some embodiments, the PIKfyve inhibitor is an antibody or antigen-binding fragment thereof, such as one that specifically binds to PIKfyve and/or inhibits PIKfyve catalytic activity. In some embodiments, the antibody or antigen-binding fragment thereof is a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv. In some embodiments, the antibody has an isotype selected from IgG, IgA, IgM, IgD, and IgE.

In some embodiments, the PIKfyve inhibitor is an interfering RNA molecule, such as a short interfering RNA (siRNA), micro RNA (miRNA), or short hairpin RNA (shRNA). The interfering RNA may suppress expression of a PIKfyve mRNA transcript, for example, by way of (i) annealing to a PIKfyve mRNA or pre-mRNA transcript, thereby forming a nucleic acid duplex; and (ii) promoting nuclease-mediated degradation of the PIKfyve mRNA or pre-mRNA transcript and/or (iii) slowing, inhibiting, or preventing the translation of a PIKfyve mRNA transcript, such as by sterically precluding the formation of a functional ribosome-RNA transcript complex or otherwise attenuating formation of a functional protein product from the target RNA transcript.

In some embodiments, the interfering RNA molecule, such as the siRNA, miRNA, or shRNA, contains an antisense portion that anneals to a segment of a PIKfyve RNA transcript (e.g., mRNA or pre-mRNA transcript), such as a portion that anneals to a segment of a PIKfyve RNA transcript having a nucleic acid sequence that is at least 85% identical to the nucleic acid sequence of NCBI Reference Sequence Nos. NM_015040.4 (e.g., 85%, 86%, 87%, 88%, 89%, 90%, 91%, 92%, 93%, 94%, 95%, 96%, 97%, 98%, 99%, 99.9%, or 100% identical to the nucleic acid sequence of NCBI Reference Sequence Nos. NM_015040.4).

In some embodiments, the interfering RNA molecule, such as the siRNA, miRNA, or shRNA, contains a sense portion having at least 85% sequence identity to the nucleic acid sequence of a segment of NCBI Reference Sequence Nos. NM_015040.4 (e.g., 85%, 86%, 87%, 88%, 89%, 90%, 91%, 92%, 93%, 94%, 95%<96%, 97%, 98%, 99%, 99.9%, or 100% identical to the nucleic acid sequence of a segment of NCBI Reference Sequence Nos. NM_015040.4).

In some embodiments, the neurological disorder is amyotrophic lateral sclerosis, and following administration of the PIKfyve inhibitor to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient.

In another aspect, the invention features a kit containing a PIKfyve inhibitor. The kit may further contain a package insert, such as one that instructs a user of the kit to perform the method of any of the above aspects or embodiments of the invention. The PIKfyve inhibitor in the kit may be a small molecule, antibody, antigen-binding fragment thereof, or interfering RNA molecule, such as a small molecule, antibody, antigen-binding fragment thereof, or interfering RNA molecule described above and herein.

### Definitions

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1 H— and 3H-imidazole, 1 H-, 2H- and 4H-1,2,4-triazole, 1 H- and 2H-isoindole, and 1 H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

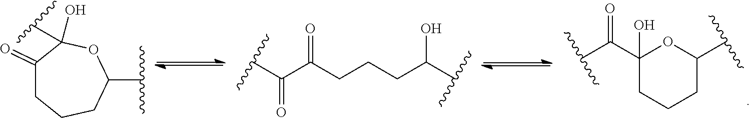 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physicochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc.). In some embodiments, such entities may be utilized in any form, including in any solid form. In some embodiments, such entities are utilized in a particular form, e.g., in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C4 alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkylamino,” as used herein, represents —NHR, where R is alkyl.

The term “alkoxy,” as used herein, represents —OR, where R is alkyl.

The term “alkoxycarbonyl,” as used herein, represents —COOR, where R is alkyl.

The term “alkylaminocarbonylamino,” as used herein, represents —NHCONHR, where R is alkyl.

The term “alkylcarbamyl,” as used herein, represents —CONHR, where R is alkyl.

The term “alkylsulfamyl,” as used herein, represents a group of the following structure:

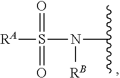

where R^{A} is alkyl, and R^{B} is hydrogen or alkyl.

The term “alkylsulfonyl,” as used herein, represents a group of the following structure:

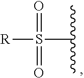

where R is alkyl.

The term “alkylcarbonylamino,” as used herein, represents —NH—CO—R, where R is alkyl.

The term “alkylsulfonylamino,” as used herein, represents —NH—SO₂—R, where R is alkyl.

The term “alkylaminosulfonyl,” as used herein, represents —SO₂NHR, where R is alkyl.

The term “alkylaminosulfonylamino,” as used herein, represents —NHSO₂NHR, where R is alkyl.

The term “alkylthio,” as used herein, represents —SR, where R is alkyl.

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aminocarbonylamino,” as used herein, represents —NHCONH₂.

The term “aminosulfonyl,” as used herein, represents —SO₂NH₂.

The term “aminosulfonylamino,” as used herein, represents —NHSO₂NH₂.

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆₋₁₀ aryl, C₁-C₁₀ alkyl C₆₋₁₀ aryl, or C₁-C₂₀ alkyl C₆₋₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “carbamyl,” as used herein, represents —CONH₂.

The term “carboxy,” as used herein, represents —COOH.

The term “cyano,” as used herein, represents a CN group.

The term “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl. A polycyclic cycloalkyl may be fused, bridged, or spiro cycloalkyl.

The term “dialkylamino,” as used herein, represents —NR₂, where each R is independently alkyl.

The term “dialkylaminocarbonyl,” as used herein, represents —CONR₂, where each R is independently alkyl.

The term “dialkylaminocarbonylamino,” as used herein, represents —NHCONR₂, where each R is independently alkyl.

The term “dialkylaminosulfonyl,” as used herein, represents —SO₂NR₂, where each R is independently alkyl.

The term “dialkylaminosulfonylamino,” as used herein, represents —NHSO₂NR₂, where each R is independently alkyl.

The term “dialkylcarbamyl,” as used herein, represents —CONR₂, where each R is independently alkyl.

The term “halo,” as used herein, means a fluorine (fluoro), chlorine (chloro), bromine (bromo), or iodine (iodo) radical.

The term “haloalkoxy,” as used herein, refers to an alkoxy group substituted with one or more halogen (e.g., fluorine).

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, three, or four ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C1-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. A heterocyclyl group may be aromatic or non-aromatic. An aromatic heterocyclyl is also referred to as heteroaryl. A polycyclic heterocyclyl may be fused, bridged, or spiro heterocyclyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro.” as used herein, represents an NO₂ group.

The term “oxo,” as used herein, represents divalent oxygen group (═O), as typically found in carbonyl and sulfone groups.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halo (e.g., fluoro), hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, orthiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained, for example, by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Additional Definitions

As used herein, the term “about” refers to a value that is within 10% above or below the value being described. For instance, a value of “about 5 mg” refers to a quantity that is from 4.5 mg to 5.5 mg.

As used herein, the term “affinity” refers to the strength of a binding interaction between two molecules, such as a ligand and a receptor. The term “Kᵢ”, as used herein, is intended to refer to the inhibition constant of an antagonist for a particular molecule of interest, and is expressed as a molar concentration (M). Kᵢ values for antagonist-target interactions can be determined, e.g., using methods established in the art. The term “K_{d}”, as used herein, is intended to refer to the dissociation constant, which can be obtained, e.g., from the ratio of the rate constant for the dissociation of the two molecules (k_{d}) to the rate constant for the association of the two molecules (kₐ) and is expressed as a molar concentration (M). K_{d} values for receptor-ligand interactions can be determined, e.g., using methods established in the art. Methods that can be used to determine the K_{d} of a receptor-ligand interaction include surface plasmon resonance, e.g., through the use of a biosensor system such as a BIACORE® system.

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a PIKfyve inhibitor described herein, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the PIKfyve inhibitor, such as an improvement in the subject's ALSFRS or ALSFRS-R score within one or more days, weeks, or months following administration of the PIKfyve inhibitor (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (ii) an increase in the subject's slow vital capacity following administration of the PIKfyve inhibitor, such as an increase in the subject's slow vital capacity within one or more days, weeks, or months following administration of the PIKfyve inhibitor (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (iii) a reduction in decremental responses exhibited by the subject upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); and (vi) a decrease in the frequency and/or severity of muscle cramps exhibited by the subject, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject).

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 13, below.

TABLE 13
Representative physicochemical properties of naturally-occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “FYVE-type zinc finger containing phosphoinositide kinase” and its abbreviation, “PIKfyve,” are used interchangeably. These terms refer to the enzyme that catalyzes phosphorylation of phosphatidylinositol 3-phosphate to produce phosphatidylinositol 3,5-bisphosphate, for example, in human subjects. The terms refer not only to wild-type forms of PIKfyve, but also to variants of wild-type PIKfyve proteins and nucleic acids encoding the same. The gene encoding PIKfyve can be accessed under NCBI Reference Sequence No. NG_021188.1. Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_689884.1, and NP_001171471.1.

For example, the terms “FYVE-type zinc finger containing phosphoinositide kinase” and its abbreviation, “PIKfyve,” as used herein include forms of the human PIKfyve protein that have an amino acid sequence that is at least 85% identical to the amino acid sequence of NCBI Reference Sequence No. NP_055855.2 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence Nos. NP_055855.2) and/or forms of the human PIKfyve protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type PIKfyve protein. Similarly, these terms include, for example, forms of the human PIKfyve gene that encode an mRNA transcript having a nucleic acid sequence that is at least 85% identical to the nucleic acid sequence of NCBI Reference Sequence No. NM_015040.4 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence Nos. NM_015040.4).

As used herein, the term “PIKfyve inhibitor” refers to substances, such as small molecules, peptides, and biologic agents (e.g., antibodies and antigen-binding fragments thereof), that suppress the activity of the PIKfyve enzyme. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances, such as small molecules, peptides, and biologic agents (e.g., antibodies and antigen-binding fragments thereof), that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” also encompasses substances that reduce the concentration and/or stability of PIKfyve mRNA transcripts in vivo, as well as those that suppress the translation of functional PIKfyve enzyme. Examples of inhibitors of this type are interfering RNA molecules, such as short interfering RNA (siRNA), micro RNA (miRNA), and short hairpin RNA (shRNA). Additional examples of “PIKfyve inhibitors” are substances, such as small molecules, peptides, and biologic agents (e.g., antibodies and antigen-binding fragments thereof), that attenuate the transcription of an endogenous gene encoding PIKfyve.

As used herein, the term “dose” refers to the quantity of a therapeutic agent, such as a PIKfyve inhibitor described herein (e.g., an inhibitory small molecule, antibody, antigen-binding fragment thereof, or interfering RNA molecule described herein) that is administered to a subject for the treatment of a disorder or condition, such as to treat or prevent a neurological disorder in a subject (e.g., a human subject). A therapeutic agent as described herein may be administered in a single dose or in multiple doses for the treatment of a particular indication. In each case, the therapeutic agent may be administered using one or more unit dosage forms of the therapeutic agent. For instance, a single dose of 1 mg of a therapeutic agent may be administered using, e.g., two 0.5 mg unit dosage forms of the therapeutic agent, four 0.25 mg unit dosage forms of the therapeutic agent, one single 1 mg unit dosage form of the therapeutic agent, and the like.

As used herein, the term “endogenous” describes a molecule (e.g., a metabolite, polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a small molecule, polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-cell that can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells are described, for example, in Takahashi and Yamanaka, Cell 126:663 (2006), the disclosure of which is incorporated herein by reference as it pertains to the structure and functionality of iPS cells.

As used herein, the term “interfering RNA” refers to a RNA, such as a short interfering RNA (siRNA), micro RNA (miRNA), or short hairpin RNA (shRNA) that suppresses the expression of a target RNA transcript, for example, by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient, such as a human patient having a neurological disorder described herein, in the form of, for example, a single- or double-stranded oligonucleotide, or in the form of a vector (e.g., a viral vector) containing a transgene encoding the interfering RNA. Exemplary interfering RNA platforms are described, for example, in Lam et al., Molecular Therapy—Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100multiplied by(the fraction X/Y)

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by(the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein in the context of administration of a therapeutic agent, the term “periodically” refers to administration of the agent two or more times over the course of a treatment period (e.g., two or more times daily, weekly, monthly, or yearly).

As used herein, the term “pharmaceutical composition” means a mixture containing a therapeutic compound to be administered to a patient, such as a mammal, e.g., a human, in order to prevent, treat or control a particular disease or condition affecting the mammal, such as a neurological disorder described herein.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

As used herein in the context of therapeutic treatment, the terms “provide” and “providing” refer to the delivery of a therapeutic agent to a subject (e.g., a mammalian subject, such as a human) in need of treatment, such as a subject experiencing or at risk of developing a neurological disorder described herein. A therapeutic agent may be provided to a subject in need thereof, for instance, by direct administration of the therapeutic agent to the subject, or by administration of a prodrug that is converted in vivo to the therapeutic agent upon administration of the prodrug to the subject. Exemplary prodrugs include, without limitation, esters, phosphates, and other chemical functionalities susceptible to hydrolysis upon administration to a subject. Prodrugs include those known in the art, such as those described, for instance, in Vig et al., Adv. Drug Deliv. Rev. 65:1370-1385 (2013), and Huttunen et al., Pharmacol. Rev. 63:750-771 (2011), the disclosures of each of which are incorporated herein by reference in their entirety.

As used herein, the term “neuromuscular disorder” refers to a disease impairing the ability of one or more neurons to control the activity of an associated muscle. Examples of neuromuscular disorders are amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome, among others.

As used herein, the term “repeat region” refers to segments within a gene of interest or an RNA transcript thereof containing nucleic acid repeats, such as the poly GGGGCC (SEQ ID NO: 5) sequence in the human c9orf72 gene (or the poly GGGGCC sequence in the RNA transcript thereof). A repeat region is considered to be an “expanded repeat region,” a “repeat expansion,” or the like, if the number of nucleotide repeats in the repeat region exceeds the quantity of repeats ordinarily found in the repeat region of a wild-type form of the gene or RNA transcript thereof. For example, the human c9orf72 gene typically contains from 2 to 19 GGGGCC repeats. “Expanded repeat regions,” “repeat expansions,” and “hexanucleotide repeat expansions” (or “HREs”) in the context of the c9orf72 gene or an RNA transcript thereof thus refer to repeat regions containing greater than 19 GGGGCC repeats, such as from about 20 to about 2,000 GGGGCC hexanucleotide repeats (e.g., about 50 GGGGCC hexanucleotide repeats, about 60 GGGGCC hexanucleotide repeats, about 70 hexanucleotide repeats, 80 hexanucleotide repeats, 90 hexanucleotide repeats, 100 hexanucleotide repeats, 110 hexanucleotide repeats, 120 hexanucleotide repeats, 130 hexanucleotide repeats, 140 hexanucleotide repeats, 150 hexanucleotide repeats, 160 hexanucleotide repeats, 170 hexanucleotide repeats, 180 hexanucleotide repeats, 190 hexanucleotide repeats, 200 hexanucleotide repeats, 210 hexanucleotide repeats, 220 hexanucleotide repeats, 230 hexanucleotide repeats, 240 hexanucleotide repeats, 250 hexanucleotide repeats, 260 hexanucleotide repeats, 270 hexanucleotide repeats, 280 hexanucleotide repeats, 290 hexanucleotide repeats, 300 hexanucleotide repeats, 310 hexanucleotide repeats, 320 hexanucleotide repeats, 330 hexanucleotide repeats, 340 hexanucleotide repeats, 350 hexanucleotide repeats, 360 hexanucleotide repeats, 370 hexanucleotide repeats, 380 hexanucleotide repeats, 390 hexanucleotide repeats, 400 hexanucleotide repeats, 410 hexanucleotide repeats, 420 hexanucleotide repeats, 430 hexanucleotide repeats, 440 hexanucleotide repeats, 450 hexanucleotide repeats, 460 hexanucleotide repeats, 470 hexanucleotide repeats, 480 hexanucleotide repeats, 490 hexanucleotide repeats, 500 hexanucleotide repeats, 510 hexanucleotide repeats, 520 hexanucleotide repeats, 530 hexanucleotide repeats, 540 hexanucleotide repeats, 550 hexanucleotide repeats, 560 hexanucleotide repeats, 570 hexanucleotide repeats, 580 hexanucleotide repeats, 590 hexanucleotide repeats, 600 hexanucleotide repeats, 610 hexanucleotide repeats, 620 hexanucleotide repeats, 630 hexanucleotide repeats, 640 hexanucleotide repeats, 650 hexanucleotide repeats, 660 hexanucleotide repeats, 670 hexanucleotide repeats, 680 hexanucleotide repeats, 690 hexanucleotide repeats, 700 hexanucleotide repeats, 710 hexanucleotide repeats, 720 hexanucleotide repeats, 730 hexanucleotide repeats, 740 hexanucleotide repeats, 750 hexanucleotide repeats, 760 hexanucleotide repeats, 770 hexanucleotide repeats, 780 hexanucleotide repeats, 790 hexanucleotide repeats, 800 hexanucleotide repeats, 810 hexanucleotide repeats, 820 hexanucleotide repeats, 830 hexanucleotide repeats, 840 hexanucleotide repeats, 850 hexanucleotide repeats, 860 hexanucleotide repeats, 870 hexanucleotide repeats, 880 hexanucleotide repeats, 890 hexanucleotide repeats, 900 hexanucleotide repeats, 910 hexanucleotide repeats, 920 hexanucleotide repeats, 930 hexanucleotide repeats, 940 hexanucleotide repeats, 950 hexanucleotide repeats, 960 hexanucleotide repeats, 970 hexanucleotide repeats, 980 hexanucleotide repeats, 990 hexanucleotide repeats, 1,000 hexanucleotide repeats, 1,100 hexanucleotide repeats, 1,200 hexanucleotide repeats, 1,300 hexanucleotide repeats, 1,400 hexanucleotide repeats, 1,500 hexanucleotide repeats, 1,600 hexanucleotide repeats, 1,700 hexanucleotide repeats, 1,800 hexanucleotide repeats, 1,900 hexanucleotide repeats, or 2,000 hexanucleotide repeats, among others).

As used herein, the term “sample” refers to a specimen (e.g., blood, blood component (e.g., serum or plasma), urine, saliva, amniotic fluid, cerebrospinal fluid, tissue (e.g., placental or myometrial), pancreatic fluid, chorionic villus sample, and cells) isolated from a patient.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein, e.g., with a K_{D} of less than 100 nM. For example, a ligand that specifically binds to a protein may bind to the protein with a K_{D} of up to 100 nM (e.g., between 1 pM and 100 nM). A ligand that does not exhibit specific binding to a protein or a domain thereof will exhibit a K_{D} of greater than 100 nM (e.g., greater than 200 nM, 300 nM, 400 nM, 500 nM, 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are used interchangeably and refer to an organism, such as a mammal (e.g., a human) that receives therapy for the treatment or prevention of a neurological disease described herein, for example, for amyotrophic lateral sclerosis. Patients that may receive therapy, or that are considered to be in need of therapy, for the treatment or prevention of a neurological disease described herein include subjects (e.g., human subjects) that have been diagnosed as having the neurological disease and/or that exhibit one or more symptoms of the disease, as well as those at risk of developing the disease. In the context of a neurological disorder described herein, such as amyotrophic lateral sclerosis, examples of patients that may be treated using the compositions and methods of the present disclosure are those that are at risk of developing the disease, as well as those that are classified as having clinically definite, clinically probable, clinically probable (laboratory-supported), or clinically possible amyotrophic lateral sclerosis according to the El-Escorial diagnostic criteria for this disease. A patient may be diagnosed as having a neurological disorder, for example, by way of (i) electrodiagnostic tests including electromyography (EMG) and nerve conduction velocity (NCV); (ii) blood and urine studies, including high resolution serum protein electrophoresis, thyroid and parathyroid hormone levels, and 24-hour urine collection for heavy metals; (iii) spinal tap; x-rays, including magnetic resonance imaging; (iv) myelogram of cervical spine; (v) muscle and/or nerve biopsy; and/or (vi) thorough neurological evaluation.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from A315T, Q331K, M337V, D169G, G294A, G294V, Q343R, G295S, N345K, R361 S, N390D, A382T, and G376D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided herein as SEQ ID NOs: 3 and 4, which correspond to NCBI Reference Sequence NOs. NM_007375.3 and NP_031401.1, respectively. These sequences are shown in Table 14 below.

TABLE 14
Amino acid and nucleic acid sequences
of wild-type human TDP-43

| SEQ ID | |
|---|---|
| NO. | Sequence |
| 1 | MSEYIRVTEDENDEPIEIPSEDDGTVLLST |
| | VTAQFPGACGLRYRNPVSQCMRGVRLVEGI |
| | LHAPDAGWGNLVYVVNYPKDNKRKMDETDA |
| | SSAVKVKRAVQKTSDLIVLGLPWKTTEQDL |
| | KEYFSTFGEVLMVQVKKDLKTGHSKGFGFV |
| | RFTEYETQVKVMSQRHMIDGRWCDCKLPNS |
| | KQSQDEPLRSRKVFVGRCTEDMTEDELREF |
| | FSQYGDVMDVFIPKPFRAFAFVTFADDQIA |
| | QSLCGEDLIIKGISVHISNAEPKHNSNRQL |
| | ERSGRFGGNPGGFGNQGGFGNSRGGGAGLG |
| | NNQGSNMGGGMNFGAFSINPAMMAAAQAAL |
| | QSSWGMMGMLASQQNQSGPSGNNQNQGNMQ |
| | REPNQAFGSGNNSYSGSNSGAAIGWGSASN |
| | AGSGSGFNGGFGSSMDSKSSGWGM |
| 2 | GGUGGGCGGGGGGAGGAGGCGGCCCUAGCG |
| | CCAUUUUGUGGGAGCGAAGCGGUGGCUGGG |
| | CUGCGCUUGGGUCCGUCGCUGCUUCGGUGU |
| | CCCUGUCGGGCUUCCCAGCAGCGGCCUAGC |
| | GGGAAAAGUAAAAGAUGUCUGAAUAUAUUC |
| | GGGUAACCGAAGAUGAGAACGAUGAGCCCA |
| | UUGAAAUACCAUCGGAAGACGAUGGGACGG |
| | UGCUGCUCUCCACGGUUACAGCCCAGUUUC |
| | CAGGGGCGUGUGGGCUUCGCUACAGGAAUC |
| | CAGUGUCUCAGUGUAUGAGAGGUGUCCGGC |
| | UGGUAGAAGGAAUUCUGCAUGCCCCAGAUG |
| | CUGGCUGGGGAAAUCUGGUGUAUGUUGUCA |
| | ACUAUCCAAAAGAUAACAAAAGAAAAAUGG |
| | AUGAGACAGAUGCUUCAUCAGCAGUGAAAG |
| | UGAAAAGAGCAGUCCAGAAAACAUCCGAUU |
| | UAAUAGUGUUGGGUCUCCCAUGGAAAACAA |
| | CCGAACAGGACCUGAAAGAGUAUUUUAGUA |
| | CCUUUGGAGAAGUUCUUAUGGUGCAGGUCA |
| | AGAAAGAUCUUAAGACUGGUCAUUCAAAGG |
| | GGUUUGGCUUUGUUCGUUUUACGGAAUAUG |
| | AAACACAAGUGAAAGUAAUGUCACAGCGAC |
| | AUAUGAUAGAUGGACGAUGGUGUGACUGCA |
| | AACUUCCUAAUUCUAAGCAAAGCCAAGAUG |
| | AGCCUUUGAGAAGCAGAAAAGUGUUUGUGG |
| | GGCGCUGUACAGAGGACAUGACUGAGGAUG |
| | AGCUGCGGGAGUUCUUCUCUCAGUACGGGG |
| | AUGUGAUGGAUGUCUUCAUCCCCAAGCCAU |
| | UCAGGGCCUUUGCCUUUGUUACAUUUGCAG |
| | AUGAUCAGAUUGCGCAGUCUCUUUGUGGAG |
| | AGGACUUGAUCAUUAAAGGAAUCAGCGUUC |
| | AUAUAUCCAAUGCCGAACCUAAGCACAAUA |
| | GCAAUAGACAGUUAGAAAGAAGUGGAAGAU |
| | UUGGUGGUAAUCCAGGUGGCUUUGGGAAUC |
| | AGGGUGGAUUUGGUAAUAGCAGAGGGGGUG |
| | GAGCUGGUUUGGGAAACAAUCAAGGUAGUA |
| | AUAUGGGUGGUGGGAUGAACUUUGGUGCGU |
| | UCAGCAUUAAUCCAGCCAUGAUGGCUGCCG |
| | CCCAGGCAGCACUACAGAGCAGUUGGGGUA |
| | UGAUGGGCAUGUUAGCCAGCCAGCAGAACC |
| | AGUCAGGCCCAUCGGGUAAUAACCAAAACC |
| | AAGGCAACAUGCAGAGGGAGCCAAACCAGG |
| | CCUUCGGUUCUGGAAAUAACUCUUAUAGUG |
| | GCUCUAAUUCUGGUGCAGCAAUUGGUUGGG |
| | GAUCAGCAUCCAAUGCAGGGUCGGGCAGUG |
| | GUUUUAAUGGAGGCUUUGGCUCAAGCAUGG |
| | AUUCUAAGUCUUCUGGCUGGGGAAUGUAGA |
| | CAGUGGGGUUGUGGUUGGUUGGUAUAGAAU |
| | GGUGGGAAUUCAAAUUUUUCUAAACUCAUG |
| | GUAAGUAUAUUGUAAAAUACAUAUGUACUA |
| | AGAAUUUUCAAAAUUGGUUUGUUCAGUGUG |
| | GAGUAUAUUCAGCAGUAUUUUUGACAUUUU |
| | UCUUUAGAAAAAGGAAGAGCUAAAGGAAUU |
| | UUAUAAGUUUUGUUACAUGAAAGGUUGAAA |
| | UAUUGAGUGGUUGAAAGUGAACUGCUGUUU |
| | GCCUGAUUGGUAAACCAACACACUACAAUU |
| | GAUAUCAAAAGGUUUCUCCUGUAAUAUUUU |
| | AUCCCUGGACUUGUCAAGUGAAUUCUUUGC |
| | AUGUUCAAAACGGAAACCAUUGAUUAGAAC |
| | UACAUUCUUUACCCCUUGUUUUAAUUUGAA |
| | CCCCACCAUAUGGAUUUUUUUCCUUAAGAA |
| | AAUCUCCUUUUAGGAGAUCAUGGUGUCACA |
| | GUGUUUGGUUCUUUUGUUUUGUUUUUUAAC |
| | ACUUGUCUCCCCUCAUACACAAAAGUACAA |
| | UAUGAAGCCUUCAUUUAAUCUCUGCAGUUC |
| | AUCUCAUUUCAAAUGUUUAUGGAAGAAGCA |
| | CUUCAUUGAAAGUAGUGCUGUAAAUAUUCU |
| | GCCAUAGGAAUACUGUCUACAUGCUUUCUC |
| | AUUCAAGAAUUCGUCAUCACGCAUCACAGG |
| | CCGCGUCUUUGACGGUGGGUGUCCCAUUUU |
| | UAUCCGCUACUCUUUAUUUCAUGGAGUCGU |
| | AUCAACGCUAUGAACGCAAGGCUGUGAUAU |
| | GGAACCAGAAGGCUGUCUGAACUUUUGAAA |
| | CCUUGUGUGGGAUUGAUGGUGGUGCCGAGG |
| | CAUGAAAGGCUAGUAUGAGCGAGAAAAGGA |
| | GAGAGCGCGUGCAGAGACUUGGUGGUGCAU |
| | AAUGGAUAUUUUUUAACUUGGCGAGAUGUG |
| | UCUCUCAAUCCUGUGGCUUUGGUGAGAGAG |
| | UGUGCAGAGAGCAAUGAUAGCAAAUAAUGU |
| | ACGAAUGUUUUUUGCAUUCAAAGGACAUCC |
| | ACAUCUGUUGGAAGACUUUUAAGUGAGUUU |
| | UUGUUCUUAGAUAACCCACAUUAGAUGAAU |
| | GUGUUAAGUGAAAUGAUACUUGUACUCCCC |
| | CUACCCCUUUGUCAACUGCUGUGAAUGCUG |
| | UAUGGUGUGUGUUCUCUUCUGUUACUGAUA |
| | UGUAAGUGUGGCAAUGUGAACUGAAGCUGA |
| | UGGGCUGAGAACAUGGACUGAGCUUGUGGU |
| | GUGCUUUGCAGGAGGACUUGAAGCAGAGUU |
| | CACCAGUGAGCUCAGGUGUCUCAAAGAAGG |
| | GUGGAAGUUCUAAUGUCUGUUAGCUACCCA |
| | UAAGAAUGCUGUUUGCUGCAGUUCUGUGUC |
| | CUGUGCUUGGAUGCUUUUUAUAAGAGUUGU |
| | CAUUGUUGGAAAUUCUUAAAUAAAACUGAU |
| | UUAAAUAAUAUGUGUCUUUGUUUUGCAGCC |
| | CUGAAUGCAAAGAAUUCAUAGCAGUUAAUU |
| | CCCCUUUUUUGACCCUUUUGAGAUGGAACU |
| | UUCAUAAAGUUUCUUGGCAGUAGUUUAUUU |
| | UGCUUCAAAUAAACUUAUUUGAAAAGUUGU |
| | CUCAAGUCAAAUGGAUUCAUCACCUGUCAU |
| | GCAUUGACACCUGAUACCCAGACUUAAUUG |
| | GUAUUUGUUCUUGCAUUGGCCAAAGUGAAA |
| | AUUUUUUUUUUUCUUUUGAAAUCUAGUUUU |
| | GAAUAAGUCUGGGUGACCGCACCUAAAAUG |
| | GUAAGCAGUACCCUCCGGCUUUUUCUUAGU |
| | GCCUCUGUGCAUUUGGGUGAUGUUCUAUUU |
| | ACAUGGCCUGUGUAAAUCUCCAUUGGGAAG |
| | UCAUGCCUUCUAAAAAGAUUCUUAUUUGGG |
| | GGAGUGGGCAAAAUGUUGAUUAUUUUCUAA |
| | UGCUUUGUAGCAAAGCAUAUCAAUUGAAAA |
| | GGGAAUAUCAGCACCUUCCUAGUUUGGGAU |
| | UUGAAAAGUGGAAUUAAUUGCAGUAGGGAU |
| | AAAGUAGAAGAAACCACAAAUUAUCUUGUG |
| | CCUGAAAUCCAUUAAGAGGCCUGAUAGCUU |
| | UAAGAAUUAGGGUGGGUUGUCUGUCUGGAA |
| | GUGUUAAGUGGAAUGGGCUUUGUCCUCCAG |
| | GAGGUGGGGGAAUGUGGUAACAUUGAAUAC |
| | AGUUGAAUAAAAUCGCUUACAAAACUCACA |
| | CUCUCACAAUGCAUUGUUAAGUAUGUAAAA |
| | GCAAUAACAUUGAUUCUCUGUUGUACUUUU |
| | UUGUAACUAAUUCUGUGAGAGUUGAGCUCA |
| | UUUUCUAGUUGGAAGAAUGUGAUAUUUGUU |
| | GUGUUGGUAGUUUACCUAAUGCCCUUACCU |
| | AAUUAGAUUAUGAUAAAUAGGUUUGUCAUU |
| | UUGCAAGUUACAUAAACAUUUAUCAAUGAA |
| | GUCAUCCUUUAGACUUGUAAUCGCCACAUU |
| | GUUUCAUUAUUCAGUUUCCUCUGUAAAGGG |
| | AUCUUGAGUUGUUUUAAUUUUUUUUUUCUG |
| | CAUCUGAAUCUGCAUGAUUUCCAAACCCUG |
| | UACCAUCUGAAUUUUGCAUUUUAGCACUUG |
| | CACUAUUACUCAGCAGCAGUAACAUGGUAA |
| | CACUUAAAAUGGUACUCGGGGACCUCCAAA |
| | GACUAAACUGACAAGCCUUCAAGGAGCCCA |
| | GGGGUAAGUUAACUUGUCAACGGCAUGGUU |
| | UAAUCCCUUCUUUACACUUGUGUAAAUUUC |
| | AGUUACUGGUCAUAGAAGGCUUUCAAUGUU |
| | GAGUGGCCUUUUAUUAACAUGUUUAUGGUA |
| | CUGCAUAGAUACGGGUAUUUAUUUUACCCU |
| | AAGAAGAUUUUGAAGUUUAAAAGUACUUAA |
| | ACUAUUUGGCAAAGAUUUGUUUUUAAAAAU |
| | CUAUUUGGUCAAUCUAAAUGCAUUCAUUCU |
| | AAAAAAUUUUUUGAACCAGAUAAAUAAAAU |
| | UUUUUUUUGACACCACAAAAAAAAAAAAAA |
| | AAAAAA |

The terms “TAR-DNA binding protein-43” and “TDP-43” as used herein include, for example, forms of the human TDP-43 protein that have an amino acid sequence that is at least 85% identical to the amino acid sequence of SEQ ID NO: 1 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from A315T, Q331K, M337V, D169G, G294A, G294V, Q343R, G295S, N345K, R361S, N390D, A382T, and G376D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of SEQ ID NO: 2 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 2).

As used herein in the context of a PIKfyve inhibitor, such as an inhibitory small molecule, antibody, antigen-binding fragment thereof, or interfering RNA molecule described herein, the term “therapeutically effective amount” refers to a quantity of the inhibitor that, optionally when administered in combination with one another agent, achieves a beneficial treatment outcome for a subject that has or is at risk of developing a neurological disease described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. For example, the term “therapeutically effective amount” of a PIKfyve inhibitor described herein includes amounts of the inhibitor that, optionally when administered in combination with another agent, is capable of achieving (i) an improvement in the subject's condition as assessed using the amyotrophic lateral sclerosis functional rating scale (ALSFRS) or the revised ALSFRS (ALSFRS-R) following administration of the PIKfyve inhibitor, such as an improvement in the subject's ALSFRS or ALSFRS-R score within one or more days, weeks, or months following administration of the PIKfyve inhibitor (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (ii) an increase in the subject's slow vital capacity following administration of the PIKfyve inhibitor, such as an increase in the subject's slow vital capacity within one or more days, weeks, or months following administration of the PIKfyve inhibitor (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (iii) a reduction in decremental responses exhibited by the subject upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); and/or (vi) a decrease in the frequency and/or severity of muscle cramps exhibited by the subject, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject).

As used herein in the context of a neurological disorder, the terms “treat” or “treatment” refer to therapeutic treatment, in which the object is to slow, delay, or halt the progression or development of a neurological disorder, e.g., in a human subject. Successful treatment of a subject using a PIKfyve inhibitor as described herein (e.g., using a PIKfyve inhibitory small molecule, antibody, antigen-binding fragment thereof, or interfering RNA molecule described herein) may manifest in a variety of ways. Desired treatment outcomes that may be achieved using the compositions and methods described herein include, without limitation, (i) an improvement in the subject's condition as assessed using the amyotrophic lateral sclerosis functional rating scale (ALSFRS) or the revised ALSFRS (ALSFRS-R) following administration of the PIKfyve inhibitor, such as an improvement in the subject's ALSFRS or ALSFRS-R score within one or more days, weeks, or months following administration of the PIKfyve inhibitor (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (ii) an increase in the subject's slow vital capacity following administration of the PIKfyve inhibitor, such as an increase in the subject's slow vital capacity within one or more days, weeks, or months following administration of the PIKfyve inhibitor (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (iii) a reduction in decremental responses exhibited by the subject upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); (vi) a decrease in the frequency and/or severity of muscle cramps exhibited by the subject, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject); and (vii) a decrease in TDP-43 aggregation exhibited by the patient, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the subject.

As used herein, the term “treatment period” refers to a duration of time over which a patient may be administered a therapeutic agent, such as a PIKfyve inhibitor as described herein, so as to treat or prevent a neurological disorder. Treatment periods as described herein may have a duration of several hours, days, weeks, months, or years.

As used herein, the term “pharmaceutically acceptable salt” refers to a salt, such as a salt of a compound described herein, that retains the desired biological activity of the non-ionized parent compound from which the salt is formed. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. The compounds can also be administered as pharmaceutically acceptable quaternary salts, such as quaternary ammonium salts of the formula —NR,R′,R″ ⁺Z⁻, wherein each of R, R′, and R″ may independently be, for example, hydrogen, alkyl, benzyl, C₁-C₆—alkyl, C₂-C₆-alkenyl, C₂-C₆-alkynyl, C₁-C₆-alkyl aryl, C₁-C₆-alkyl heteroaryl, cycloalkyl, heterocycloalkyl, or the like, and Z is a counterion, such as chloride, bromide, iodide, —O-alkyl, toluenesulfonate, methyl sulfonate, sulfonate, phosphate, carboxylate (such as benzoate, succinate, acetate, glycolate, maleate, malate, fumarate, citrate, tartrate, ascorbate, cinnamoate, mandeloate, and diphenylacetate), or the like.

As used herein in the context of a PIKfyve inhibitor, the term “variant” refers to an agent containing one or more modifications relative to a reference agent and that (i) retains an ability to inhibit PIKfyve and/or (ii) is converted in vivo into an agent that inhibits PIKfyve. In the context of small molecule PIKfyve inhibitors, structural variants of a reference compound include those that differ from the reference compound by the inclusion and/or location of one or more substituents, as well as variants that are isomers of a reference compound, such as structural isomers (e.g., regioisomers) or stereoisomers (e.g., enantiomers or diastereomers), as well as prodrugs of a reference compound. In the context of an antibody or antigen-binding fragment thereof, a variant may contain one or more amino acid substitutions, such as one or more conservative amino acid substitutions, relative to the parent antibody or antigen-binding fragment thereof. In the context of an interfering RNA molecule, a variant may contain one or more nucleic acid substitutions relative to a parent interfering RNA molecule.

As used herein, the term “antibody” (Ab) refers to an immunoglobulin molecule that specifically binds to, or is immunologically reactive with, a particular antigen, and includes polyclonal, monoclonal, genetically engineered, and otherwise modified forms of antibodies, including, but not limited to, chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments. In some embodiments, two or more portions of an immunoglobulin molecule are covalently bound to one another, e.g., via an amide bond, a thioether bond, a carbon-carbon bond, a disulfide bridge, or by a linker, such as a linker described herein or known in the art. Antibodies also include antibody-like protein scaffolds, such as the tenth fibronectin type III domain (¹⁰Fn3), which contains BC, DE, and FG structural loops similar in structure and solvent accessibility to antibody complementarity-determining regions (CDRs). The tertiary structure of the ¹⁰Fn3 domain resembles that of the variable region of the IgG heavy chain, and one of skill in the art can graft, e.g., the CDRs of a reference antibody onto the fibronectin scaffold by replacing residues of the BC, DE, and FG loops of ¹⁰Fn3 with residues from the CDR-H1, CDR-H2, or CDR-H3 regions, respectively, of the reference antibody.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated CDR; and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “bispecific antibodies” refers to monoclonal, often human or humanized antibodies that have binding specificities for at least two different antigens.

As used herein, the term “chimeric” antibody refers to an antibody having variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, 1985, Science 229(4719): 1202-7; Oi et al, 1986, BioTechniques 4:214-221; Gillies et al, 1985, J. Immunol. Methods 125:191-202; U.S. Pat. Nos. 5,807,715; 4,816,567; and 4,816,397; incorporated herein by reference.

As used herein, the term “complementarity-determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may comprising modifications in these hybrid hypervariable positions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structure, peptides configured in this way typically trimerize so as to position the V_{H} and V_{L} domains of neighboring peptide chains spatially proximal to one another to permit proper folding (see Holliger et al., Proc. Natl. Acad. Sci. USA 90:6444-48, 1993; incorporated herein by reference).

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al, mAbs 4(6):653-663, 2012; incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen. Multi-specific antibodies can have structures similar to full immunoglobulin molecules and include Fc regions, for example IgG Fc regions. Such structures can include, but not limited to, IgG-Fv, IgG-(scFv)₂, DVD-Ig, (scFv)₂-(scFv)₂-Fc and (scFv)₂-Fc-(scFv)₂. In case of IgG-(scFv)₂, the scFv can be attached to either the N-terminal or the C— terminal end of either the heavy chain or the light chain. Exemplary multi-specific molecules have been reviewed by Kontermann, 2012, mAbs 4(2):182-197, Yazaki et al, 2013, Protein Engineering, Design & Selection 26(3):187-193, and Grote et al, 2012, in Proetzel & Ebersbach (eds.), Antibody Methods and Protocols, Methods in Molecular Biology vol. 901, chapter 16:247-263; incorporated herein by reference. In some embodiments, antibody fragments can be components of multi-specific molecules without Fc regions, based on fragments of IgG or DVD or scFv. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, 2003, Nature Medicine 9:129-134; incorporated herein by reference.

As used herein, the term “primatized antibody” refers to an antibody comprising framework regions from primate-derived antibodies and other regions, such as CDRs and/or constant regions, from antibodies of a non-primate source. Methods for producing primatized antibodies are known in the art. See e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The V_{L} and V_{H} domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

### BRIEF DESCRIPTION OF THE FIGURES

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene was deleted through homologous recombination with a G418 resistance cassette (fab1::G418R) (FIG. 2). PIKFYVE was cloned downstream of the GPD promoter harbored on a URA3-containing plasmid and introduced into the fab1::G418R ura3 strain. The pGAL1-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, among others. Particularly, the disclosure provides inhibitors of FYVE-type zinc finger containing phosphoinositide kinase (PIKfyve) that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in vivo. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation. The compositions and methods described herein thus provide the additional clinical benefit of enabling the identification of patients that are likely to respond to PIKfyve inhibitor therapy, as well as processes for treating these patients accordingly.

For example, a patient may be identified as having or at risk of developing TDP-43 aggregation by way of an in vitro biopsy assay. In this context, a patient's propensity for TDP-43 aggregation may be assessed by analyzing the morphology and gene expression patterns of neuronal cells obtained by differentiation of induced pluripotent stem cells (iPSCs) derived from the patient. For example, to assess the patient's propensity of developing TDP-43 aggregation, a sample of somatic cells may be isolated from the patient and reprogrammed into iPSCs. The somatic cells may be, for example, hematopoietic cells. The isolated somatic cells may reprogrammed into iPSCs by contacting the cells with one or more agents that increase expression and/or activity of Oct4, Sox2, cMyc, and/or Klf4. Upon reprograming the somatic cells into iPSCs, the iPSCs may then be differentiated into motor neurons. Methods for differentiating iPSCs into motor neurons are described, for example, in Fujimori et al., Nature Medicine 24:1579-1589 (2018); Fujimori et al., Mol. Brain 9:88 (2016); Fujimori et al., Stem Cell Reports 9:1675-1691 (2017); and Matsumoto et al., Stem Cell Reports 6:422-435 (2016), the disclosures of each of which are incorporated herein by reference. Once the iPSCs are differentiated into motor neurons, the motor neurons may be monitored for changes in morphology and gene expression that are consistent with TDP-43 aggregation and the onset of neurological disorders.

For example, (e.g., in embodiments in which the neurological disorder is amyotrophic lateral sclerosis), the patient's propensity to develop TDP-43 aggregation can be assessed by analyzing the time-dependent neurite outgrowth patterns of motor neurons obtained by differentiation of iPSCs reprogrammed from mature hematopoietic cells isolated from the patient. In some embodiments, TDP-43 aggregation is signaled by a finding that neurites on such motor neurons exhibit a decrease in size and/or a decrease in their rate of growth after a period of time following differentiation in vitro. For example, TDP-43 aggregation may be signaled by a finding that neurites on such motor neurons exhibit a decrease in size and/or a decrease in their rate of growth after from about 10 days to 100 days following differentiation (e.g., after from about 20 days to about 60 days following differentiation, after from about 21 days to about 59 days following differentiation, after from about 22 days to about 58 days following differentiation, after from about 23 days to about 57 days following differentiation, after from about 24 days to about 56 days following differentiation, after from about 25 days to about 55 days following differentiation, after from about 26 days to about 54 days following differentiation, after from about 27 days to about 53 days following differentiation, after from about 28 days to about 52 days following differentiation, after from about 29 days to about 51 days following differentiation, after from about 30 days to about 50 days following differentiation, after from about 31 days to about 49 days following differentiation, after from about 32 days to about 48 days following differentiation, after from about 33 days to about 47 days following differentiation, after from about 34 days to about 46 days following differentiation, after from about 35 days to about 45 days following differentiation, after from about 36 days to about 44 days following differentiation, after from about 37 days to about 43 days following differentiation, after from about 38 days to about 42 days following differentiation, after from about 39 days to about 41 days following differentiation, after from about 35 days to about 40 days following differentiation, or after from about 40 days to about 45 days following differentiation).

In some embodiments (e.g., when the neurological disorder is amyotrophic lateral sclerosis), TDP-43 aggregation is signaled by a finding that motor neurons obtained by differentiation from iPSCs reprogrammed from somatic cells (e.g., hematopoietic cells) isolated from the patient begin to undergo apoptosis after a period of time following differentiation in vitro. Apoptosis of such motor neurons may be assessed, for example, by monitoring the presence of leaked lactate dehydrogenase (LDH) and/or cleaved caspase-3 (CC3) in a sample of the motor neurons. In this context, a finding that the concentration of leaked LDH has increased and/or that the relative quantity of CC3+ neurons has increased is indicative of apoptosis. TDP-43 aggregation may be signaled by a finding that such motor neurons exhibit an increase in leaked LDH concentration and/or an increase in CC3 expression after from about 10 days to 100 days following differentiation (e.g., after from about 20 days to about 60 days following differentiation, after from about 21 days to about 59 days following differentiation, after from about 22 days to about 58 days following differentiation, after from about 23 days to about 57 days following differentiation, after from about 24 days to about 56 days following differentiation, after from about 25 days to about 55 days following differentiation, after from about 26 days to about 54 days following differentiation, after from about 27 days to about 53 days following differentiation, after from about 28 days to about 52 days following differentiation, after from about 29 days to about 51 days following differentiation, after from about 30 days to about 50 days following differentiation, after from about 31 days to about 49 days following differentiation, after from about 32 days to about 48 days following differentiation, after from about 33 days to about 47 days following differentiation, after from about 34 days to about 46 days following differentiation, after from about 35 days to about 45 days following differentiation, after from about 36 days to about 44 days following differentiation, after from about 37 days to about 43 days following differentiation, after from about 38 days to about 42 days following differentiation, after from about 39 days to about 41 days following differentiation, after from about 35 days to about 40 days following differentiation, or after from about 40 days to about 45 days following differentiation).

Additionally or alternatively, using the compositions and methods of the disclosure, a patient may be identified as likely to benefit from treatment with a PIKfyve inhibitor on the basis of TDP-43 expression. In this context, the patient is determined to be likely to benefit with PIKfyve inhibitor therapy if the patient expresses a mutant form of TDP-43 having a mutation associated with TDP-43 aggregation. The mutation in TDP-43 may be, for example, one or more of A315T, Q331 K, M337V, D169G, G294A, G294V, Q343R, G295S, N345K, R361S, N390D, A382T, and G376D.

The sections that follow provide a description of exemplary PIKfyve inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### Small Molecule PIKfyve Inhibitors

PIKfyve inhibitors useful in conjunction with the compositions and methods of the disclosure include compounds of formula (I):

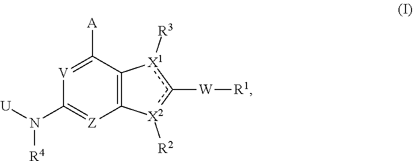

or a pharmaceutically acceptable salt or solvate thereof, wherein
R¹ is hydrogen, halo, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
each of X¹ and X² is independently selected from O, S, N, and C;
W is a bond; O; S; (CH₂)ₙ; S(O); SO₂; NRₐ; C(O); C(O)NRₐ; NRₐC(O); SO₂NRₐ; NRₐSO₂; CRₐ═CR_{b}; C═NRₐ; or NRₐ═CR_{b}, wherein n is 1-5 and each of Rₐ and R_{b} is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of R² and R³ is optionally present depending on the valence of the atom to which each is attached, and if present, each of R² and R³ is independently hydrogen, halo, hydroxyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
R⁴ is hydrogen, optionally substituted C₁₋₆ alkyl, C₃₋₈ cycloalkyl, optionally substituted C₆₋₁₀ aryl, optionally substituted C₁₋₉ heteroaryl, and optionally substituted C₁₋₉ heterocyclyl;
U is hydrogen,

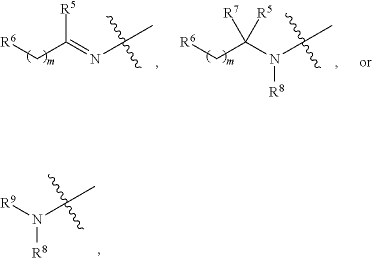

wherein m is 0-3, and each of R⁵, R⁶, R⁷, R⁸, and R^{g} is independently hydrogen, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl; or
R₃ and U, together with the nitrogen atom to which they are attached, form 4- to 6-membered heterocyclyl or heteroaryl optionally substituted by one or more substituents selected from hydrogen, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
one of the two [symbol] is a single bond, and the other [symbol] is a double bond; or each of the two [symbol] are aromatic bonds;
each of V and Z is independently N or C_{H}; and
A is optionally substituted C₃₋₈ carbocyclyl, optionally substituted C₁₋₉ heterocyclyl, and optionally substituted C₁₋₉ heteroaryl.

Exemplary compounds of formula (I) are those shown in Table 1, above, and pharmaceutically acceptable salts thereof.

In some embodiments, the PIKfyve inhibitor is a compound of formula (II):

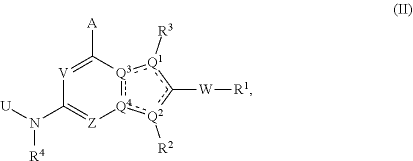

or a pharmaceutically acceptable salt or solvate thereof, wherein
R¹ is hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of Q¹, Q², Q³, and Q⁴ is independently C or N, and at least one of Q, Q¹, Q³, and Q⁴ is N;
W is a bond; O; S; (CH₂)ₙ; S(O); SO₂; NRₐ; C(O); C(O)NRₐ; NRₐC(O); SO₂NRₐ; NRₐSO₂; CRₐ═CR_{b}; C═NRₐ; or NRₐ═CR_{b}, wherein n is 1-5, and each of Rₐ and R^{b} is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of R² and R³ is optionally present depending on the valence of the atom to which each is attached, and if present, each of R² and R³ is independently hydrogen, halo, hydroxyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
R⁴ is hydrogen, optionally substituted C₁₋₆ alkyl, C₃₋₈ cycloalkyl, optionally substituted C₆₋₁₀ aryl, optionally substituted C₁₋₉ heteroaryl, or optionally substituted C₁₋₉ heterocyclyl;
U is hydrogen,

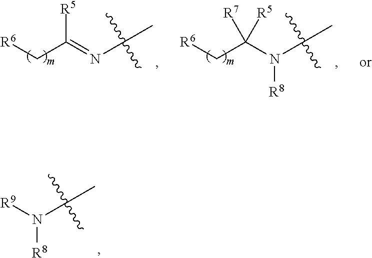

wherein m is 0-3, and each of R⁵, R⁶, R⁷, R⁸, and R^{g} is independently hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl; or
R³ and U, together with the nitrogen atom to which they are attached, form 4- to 6-membered heterocyclyl or heteroaryl optionally substituted by one or more substituents selected from hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
wherein each [symbol] is a single bond or a double bond and at least one [symbol] is a double bond; or each [symbol] is an aromatic bond;
each of V and Z is independently N or C_{H}; and
A is optionally substituted C₃₋₈ carbocyclyl, optionally substituted C₁₋₉ heterocyclyl, or optionally substituted C₁₋₉ heteroaryl.

Exemplary compounds of formula (II) are those shown in Table 2, above, and pharmaceutically acceptable salts thereof.

In some embodiments, the PIKfyve inhibitor is a compound of formula (III):

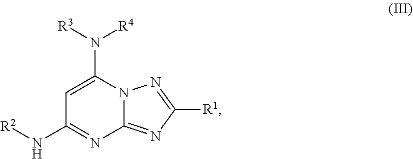

or a pharmaceutically acceptable salt thereof,
wherein
R¹ is optionally substituted C₁₋₆ alkyl, optionally substituted C₃₋₈ cycloalkyl, optionally substituted C₁₋₉ heterocyclyl, optionally substituted C₆₋₁₀ aryl, or optionally substituted C₁₋₉ heteroaryl;
R² is optionally substituted C₁₋₆ alkyl, optionally substituted C₆₋₁₀ aryl, optionally substituted C₁₋₉ heteroaryl, —N═CHRₐ, or —N═CHR_{b}R_{c}, wherein Rₐ is optionally substituted C₁₋₆ alkyl or optionally substituted C₁₋₉ heteroaryl; R_{b} is optionally substituted C₆₋₁₀ arylene; and R_{c} is hydrogen or NHSO₂Me; and
each of R³ and R⁴ is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl; or R₃ and R⁴, together with the nitrogen to which they are attached, form optionally substituted C₁₋₉ heterocyclyl.

Exemplary compounds of formula (III) are those shown in Table 3, above, and pharmaceutically acceptable salts thereof.

In some embodiments, the PIKfyve inhibitor is a compound shown in Table 4 or 5, above, or a pharmaceutically acceptable salt thereof.

In some embodiments, the PIKfyve inhibitor is

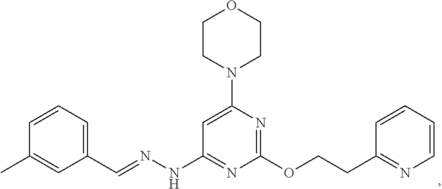 [SMILES: Cc1cccc(/C=N/Nc2cc(N3CCOCC3)nc(OCCc3ccccn3)n2)c1]

or a pharmaceutically acceptable salt thereof.

In some embodiments, the PIKfyve inhibitor is a compound of formula (IV):

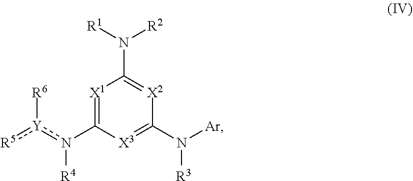

or a pharmaceutically acceptable salt thereof,
wherein
each bond denoted as [symbol] is either a single bond or a double bond, provided that the bonds denoted as [symbol] are not both simultaneously double bonds;
X¹ is selected from N and CR^{A};
X² is selected from N and CR^{A};
X³ is selected from N and CR^{A};
each R^{A} is independently selected from H, halo, C₁₋₆ alkyl, C₁₋₆ haloalkyl, C₁₋₆ alkoxy, and C₁₋₆ haloalkoxy;
Ar is selected from C₆₋₁₀ aryl and 5-10 membered heteroaryl, each of which is optionally substituted with 1, 2, 3, 4, or 5 substituents independently selected from R⁷;
each R⁷ is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c2}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} and S(O)₂NR^{c1}R^{d1};
R¹ is selected from the group consisting of H and C₁₋₆ alkyl, wherein said C₁₋₆ alkyl is optionally substituted with 1, 2, or 3 substituents independently selected from halo, CN, NO₂, OR^{a2}, C(O)R^{b2}, C(O)NR^{c2}R^{d2}. C(O)OR^{a2}, NR^{c2}R^{d2}. NR^{c2}C(O)R^{b2}, NR^{c2}C(O)OR^{a2}, NR^{c2}C(O)NR^{c2}R^{d2}. NR^{c2}S(O)₂R^{b2}, NR^{c2}S(O)₂NR^{c2}R^{d2}, S(O)₂R^{b2}, and S(O)₂NR^{c2}R^{d2};
R² is C₁₋₆ alkyl, which is optionally substituted with 1, 2, or 3 substituents independently selected from halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}; or
R¹ and R² together with the N to which they are attached form a 4-7 membered non-aromatic heterocyclyl, which is optionally substituted with 1, 2, 3, 4, or 5 substituents independently selected R⁸;
each R⁸ is independently selected from halo, C₁₋₆ alkyl, C₂₋₆ alkenyl, C₂₋₆ alkynyl, C₁₋₆ haloalkyl, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2};
R³ is selected from H, C₁₋₆ alkyl, and C₁₋₆ haloalkyl;
R⁴ is selected from H, C₁₋₆ alkyl, and C₁₋₆ haloalkyl;
Y is selected from N, C, and CR^{A};
when the bond between R⁵ and Y is a single bond, R⁵ is 5-10 membered heteroaryl, which is optionally substituted with 1, 2, 3, 4, or 5 substituents independently selected from halo, CN, NO₂, C₁₋₆ alkyl, C₂₋₆ alkenyl, C₂₋₆ alkynyl, C₁₋₆ haloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, R^{c3}C(O)NR^{c3}R^{d3},
NR^{c3}S(O)₂R^{b3}, NR^{c3}S(O)₂NR^{c3}R^{d3}, S(O)₂R^{b3} and S(O)₂NR^{c3}R^{d3};
when the bond between R⁵ and Y is a double bond, R⁵ is CR^{B}R_{c}
R^{B} is selected from H, C₁₋₆ alkyl, and C₁₋₆ haloalkyl;
R^{c} is selected from C₆₋₁₀ aryl and 5-10 membered heteroaryl, each of which is optionally substituted with 1, 2, 3, 4, or 5 substituents independently selected from halo, CN, NO₂, C₁₋₆ alkyl, C₂₋₆ alkenyl, C₂₋₆ alkynyl, Cᵢ₋ₑ haloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{C}3C(O)NR³R^{d3}, NR^{c3}S(O)₂R^{b3}, NR^{c3}S(O)₂NR^{c3}R^{d3}, S(O)₂R^{b3} and S(O)₂NR^{c3}R^{d3}; or
R⁴ and R⁵ together with Y and N to which R⁴ is attached form a 5-14 membered heteroaryl, which is optionally substituted with 1, 2, 3, 4, or 5 substituents independently selected from R^{g};
each R^{g} is independently selected from halo, CN, NO₂, C₁₋₆ alkyl, C₂₋₆ alkenyl, C₂₋₆ alkynyl, Cᵢ₋ₑ haloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said Cᵢ₋ₑ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3} and S(O)₂NR^{c3}R^{d3};
R⁶ is selected from H, C₁₋₆ alkyl, and C₁₋₆ haloalkyl; or R⁶ is absent;
each R^{a1}, R^{b1}, Rₐ2, R^{b2}, R^{a3}, and R^{b3} is independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are optionally substituted with 1, 2, 3, 4, or 5 substituents independently selected from R^{g};
each R^{c1}, R^{d1}, R^{C2}, R^{d2}, R^{C3}, and R^{d3} is independently selected from H, C₁₋₆ alkyl, C₁₋₄ haloalkyl, C₂₋₆ alkenyl, C₂₋₆ alkynyl, C(O)R^{b7}, C(O)NR^{c7}R^{d7}, C(O)OR^{a7}, NR^{c7}R^{d7}, S(O)R^{b7}, S(O)NR^{c7}R^{d7}, S(O)₂R^{b7}, and S(O)₂NR^{c7}R^{d7}; wherein said Cᵢ₋ₑ alkyl, C₂₋₆ alkenyl, and C₂₋₆ alkynyl is optionally substituted with 1, 2, 3, 4, or 5 substituents independently selected from R^{g}; each R^{a7}, R^{b7}, R^{c7}, and R^{d7} is in dependently selected from H, C₁₋₆ alkyl, 04 haloalkyl, C₂₋₆ alkenyl, C₂₋₆ alkynyl, and R^{g}, wherein said C₁₋₆ alkyl, C₂₋₆ alkenyl, and C₂₋₆ alkynyl is optionally substituted with 1, 2, 3, 4, or 5 substituents independently selected from R^{g};
or any R^{c1} and R^{d1} together with the N atom to which they are attached form a 4-, 5-, 6-, or 7-membered non-aromatic heterocyclyl group optionally substituted with 1, 2, or 3 substituents independently selected from R^{g};
or any R^{c2} and R^{d2} together with the N atom to which they are attached form a 4-, 5-, 6-, or 7-membered non-aromatic heterocyclyl group optionally substituted with 1, 2, or 3 substituents independently selected from R^{g};
or any R^{o3} and R^{d3} together with the N atom to which they are attached form a 4-, 5-, 6-, or 7-membered non-aromatic heterocyclyl group optionally substituted with 1, 2, or 3 substituents independently selected from R^{g};
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfamyl, C₁₋₆ alkylsulfonyl, carbamyl, C₁₋₆ alkylcarbamyl, di(C₁₋₆ alkyl)carbamyl, carboxy, C₁₋₆ acyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the PIKfyve inhibitor is a compound shown in Table 6 or 7, above, or a pharmaceutically acceptable salt thereof.

In some embodiments, the PIKfyve inhibitor is a compound of formula (V):

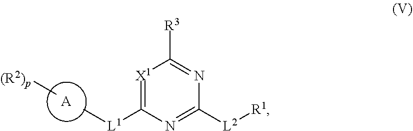

or a pharmaceutically acceptable salt thereof,
wherein
R¹ is hydroxy, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl;
each occurrence of R² is independently optionally substituted C₁₋₆ alkyl, optionally substituted C₂₋₆ alkenyl, optionally substituted C₂₋₆ alkynyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
R³ is a nitrogen- or oxygen-containing moiety;
Ring A is (i) a 5 or 6-membered heteroaryl or 5-6 or 6-5 membered bicyclic heteroaryl, each having at least one nitrogen or oxygen ring atom, or (ii) phenyl;
L¹ is absent, C1-C2 alkylene, —NR^{C}—, —O—, —S—, —C(O)—, —NHC(O)—, or —C(O)NH—;
L² is —O—(CR^{a}R^{b})ₘ—, —(CR^{a}R^{b})ₘ—, —NR^{c}—(CR^{a}R^{b})ₘ—, or —S—(CR^{a}R^{b})ₘ—;
X¹ is C_{H}, N, or CR^{C}; each occurrence of Rₐ and R^{b} are independently hydrogen, hydroxy, hydroxy(Cᵢ₋₄)alkyl, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R_{c}, —S(O)R_{c}, —NR^{c}SO₂R^{d},—OS(O)₂R^{d}, —OP(O)R^{d}R^{e}, or —P(O)R^{d}R^{e};
R^{c} is a hydrogen or C₁₋₆ alkyl;
each occurrence of R^{d} and R^{e} are independently hydrogen, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl;
m is 1-4; and
p is 1 or 2.

In some embodiment, the PIKfyve inhibitor is a compound shown in Table 8, above, or a pharmaceutically acceptable salt thereof.

In some embodiments, the PIKfyve inhibitor is a compound of formula (VI):

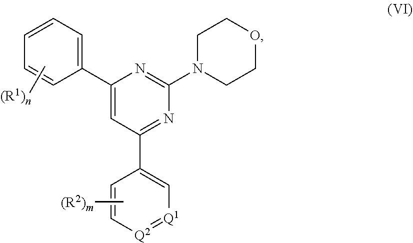

or a pharmaceutically acceptable salt thereof,
wherein
Q¹ and Q² are each independently CH or N, wherein Q¹ and Q² are not both N;
each R¹ is independently hydroxy, C₁₋₄ alkyl, or C₁₋₄ alkoxy;
n is 0, 1, or 2;
each R² is independently C₁₋₄ alkyl or C₁₋₄ alkoxy; and
m is 0 or 1.

In some embodiments, the PIKfyve inhibitor is a compound of the following structure:

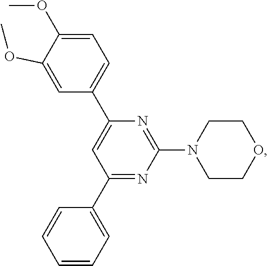 [SMILES: COc1ccc(-c2cc(-c3ccccc3)nc(N3CCOCC3)n2)cc1OC]

or a pharmaceutically acceptable salt thereof.

In some embodiments, the PIKfyve inhibitor is a compound of formula (VII):

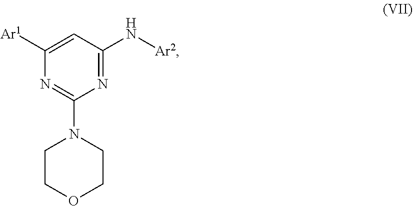

or a pharmaceutically acceptable salt thereof,
wherein
Ar¹ is phenyl or pyridyl, with each optionally independently substituted with 1 or 2 C₁₋₄ alkoxy; Ar² is phenyl, pyridyl, or pyrimidyl with each optionally independently substituted with halo, C₁₋₄ alkyl, C₁₋₄ alkoxy, or C(O)NR^{2a}R^{2b}; and
R^{2a} and R² are each independently H or C₁₋₄ alkyl.

In some embodiments, the PIKfyve inhibitor is a compound shown in Table 9, above, or a pharmaceutically acceptable salt thereof.

In some embodiments, the PIKfyve inhibitor is a compound of formula (VIII):

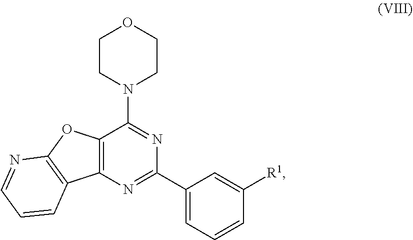

or a pharmaceutically acceptable salt thereof,
wherein
R¹ is hydroxy, C₁₋₄ alkoxy, or H(CO)R^{1a}; and
R^{1a} is phenyl or pyridyl, optionally substituted with amino, alkylamino, or dialkylamino.

In some embodiments, the PIKfyve inhibitor is a compound shown in Table 10, above, or a pharmaceutically acceptable salt thereof.

In some embodiments, the PIKfyve inhibitor is a compound of formula (IX):

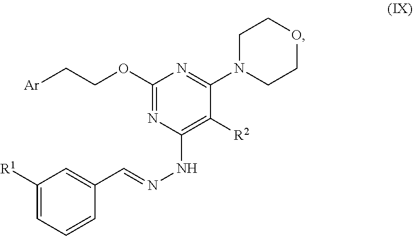

or a pharmaceutically acceptable salt thereof,
wherein
Ar is phenyl or pyridyl, with each optionally independently substituted with 1 or 2 alkyl, aminoalkyl, (alkylamino)alkyl, or (dialkylamino)alkyl;
R¹ is hydrogen or alkyl; and
R² is hydrogen or halo.

In some embodiments, the PIKfyve inhibitor is a compound shown in Table 11, above, or a pharmaceutically acceptable salt thereof.

In some embodiments, the PIKfyve inhibitor is a compound of formula (X):

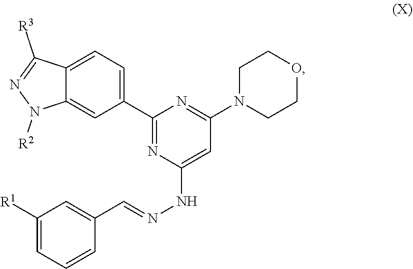

or a pharmaceutically acceptable salt thereof,
wherein
R¹ and R² are each independently hydrogen or C₁₋₄ alkyl; R³ is hydrogen or C₁₋₃ alkyl substituted with morpholinyl.

In some embodiments, the PIKfyve inhibitor is a compound shown in Table 12, above, or a pharmaceutically acceptable salt thereof.

In some embodiments, the PIKfyve inhibitor is a compound of the following structure:

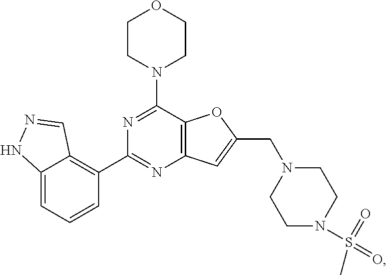 [SMILES: CS(=O)(=O)N1CCN(Cc2cc3nc(-c4cccc5[nH]ncc45)nc(N4CCOCC4)c3o2)CC1]

or a pharmaceutically acceptable salt thereof.

In some embodiments, the PIKfyve inhibitor is a compound of the following structure:

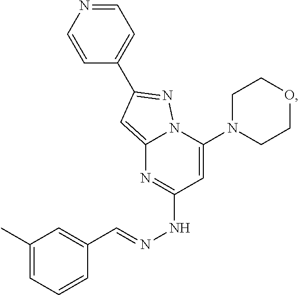 [SMILES: Cc1cccc(/C=N/Nc2cc(N3CCOCC3)n3nc(-c4ccncc4)cc3n2)c1]

or a pharmaceutically acceptable salt thereof.

In some embodiments, the PIKfyve inhibitor is a compound of formula (XI):

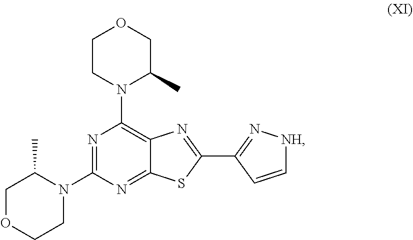 [SMILES: C[C@@H]1COCCN1c1nc(N2CCOC[C@@H]2C)nc2sc(-c3cc[nH]n3)nc12]

or a pharmaceutically acceptable salt thereof,
wherein

X is selected from

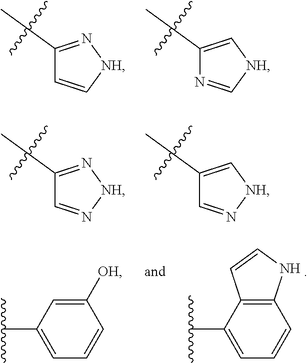

In some embodiments, the PIKfyve inhibitor is

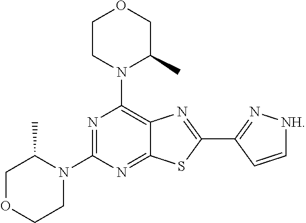 [SMILES: C[C@@H]1COCCN1c1nc(N2CCOC[C@@H]2C)nc2sc(-c3cc[nH]n3)nc12]

### Antibody Inhibitors of PIKfyve

PIKfyve inhibitors useful in conjunction with the compositions and methods described herein include antibodies and antigen-binding fragments thereof, such as those that specifically bind to PIKfyve and/or inhibit PIKfyve catalytic activity. In some embodiments, the antibody or antigen-binding fragment thereof is a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv. In some embodiments, the antibody has an isotype selected from IgG, IgA, IgM, IgD, and IgE.

### Interfering RNA Inhibitors of PIKfyve

PIKfyve inhibitors useful in conjunction with the compositions and methods described herein include interfering RNA molecules, such as short interfering RNA (siRNA) molecules, micro RNA (miRNA) molecules, or short hairpin RNA (shRNA) molecules. The interfering RNA may suppress expression of a PIKfyve mRNA transcript, for example, by way of (i) annealing to a PIKfyve mRNA or pre-mRNA transcript, thereby forming a nucleic acid duplex; and (ii) promoting nuclease-mediated degradation of the PIKfyve mRNA or pre-mRNA transcript and/or (iii) slowing, inhibiting, or preventing the translation of a PIKfyve mRNA transcript, such as by sterically precluding the formation of a functional ribosome-RNA transcript complex or otherwise attenuating formation of a functional protein product from the target RNA transcript.

In some embodiments, the interfering RNA molecule, such as the siRNA, miRNA, or shRNA, contains an antisense portion that anneals to a segment of a PIKfyve RNA transcript (e.g., mRNA or pre-mRNA transcript), such as a portion that anneals to a segment of a PIKfyve RNA transcript having a nucleic acid sequence that is at least 85% identical to the nucleic acid sequence of NCBI Reference Sequence Nos. NM_015040.4 (e.g., 85%, 86%, 87%, 88%, 89%, 90%, 91%, 92%, 93%, 94%, 95%<96%, 97%, 98%, 99%, 99.9%, or 100% identical to the nucleic acid sequence of NCBI Reference Sequence Nos. NM_015040.4).

In some embodiments, the interfering RNA molecule, such as the siRNA, miRNA, or shRNA, contains a sense portion having at least 85% sequence identity to the nucleic acid sequence of a segment of NCBI Reference Sequence Nos. NM_015040.4 (e.g., 85%, 86%, 87%, 88%, 89%, 90%, 91%, 92%, 93%, 94%, 95%<96%, 97%, 98%, 99%, 99.9%, or 100% identical to the nucleic acid sequence of a segment of NCBI Reference Sequence Nos. NM_015040.4).

Interfering RNAs as described herein may be provided to a patient, such as a human patient having a neurological disorder described herein, in the form of, for example, a single- or double-stranded oligonucleotide, or in the form of a vector (e.g., a viral vector) containing a transgene encoding the interfering RNA. Exemplary interfering RNA platforms are described, for example, in Lam et al., Molecular Therapy—Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

### Methods of Treatment

### Suppression of PIKfyve Activity and TDP-43 Aggregation to Treat Neurological Disorders

Using the compositions and methods described herein, a patient suffering from a neurological disorder may be administered a PIKfyve inhibitor, such as a small molecule, antibody, antigen-binding fragment thereof, or interfering RNA molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome.

The present disclosure is based, in part, on the discovery that PIKfyve inhibitors, such as the agents described herein, are capable of attenuating TDP-43 aggregation in vivo. TDP-43-promoted aggregation and toxicity have been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from A315T, Q331K, M337V, D169G, G294A, G294V, Q343R, G295S, N345K, R361 S, N390D, A382T, and G376D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

### Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from A315T, Q331 K, M337V, D169G, G294A, G294V, Q343R, G295S, N345K, R361S, N390D, A382T, and G376D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient.

### Routes of Administration and Dosing

PIKfyve inhibitors (e.g., inhibitory small molecules, antibodies, antigen-binding fragments thereof, and interfering RNA molecules) described herein may be administered to a patient (e.g., a human patient having one or more neurological disorders described herein) by a variety of routes. Exemplary routes of administration are oral, transdermal, subcutaneous, intranasal, intravenous, intramuscular, intraocular, parenteral, topical, intrathecal, and intracerebroventricular administration. The most suitable route for administration in any given case will depend on the particular agent being administered, the patient, pharmaceutical formulation methods, administration methods (e.g., administration kinetics), the patient's age, body weight, sex, severity of the diseases being treated, the patient's diet, and the patient's excretion rate, among other factors.

Therapeutic compositions can be administered with medical devices known in the art. For example, therapeutic compositions described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of implants and modules useful in conjunction with the routes of administration described herein are those described in U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference as they pertain to devices suitable for administration of a therapeutic agent to a patient (e.g., a human patient). Various other such implants, delivery systems, and modules are known to those skilled in the art.

### Pharmaceutical Compositions

The PIKfyve inhibitors (e.g., small molecules, antibodies, antigen-binding fragments thereof, and interfering RNA molecules described herein) suitable for use with the compositions and methods described herein can be formulated into pharmaceutical compositions for administration to a patient, such as a human patient exhibiting or at risk of developing TDP-43 aggregation, in a biologically compatible form suitable for administration in vivo. A pharmaceutical composition containing, for example, a PIKfyve inhibitor described herein, such as a small molecule, an antibody, antigen-binding fragment thereof, or interfering RNA molecule described herein, may additionally contain a suitable diluent, carrier, or excipient. PIKfyve inhibitors can be formulated for administration to a subject, for example, by way of any one or more of the routes of administration described above. Under ordinary conditions of storage and use, a pharmaceutical composition may contain a preservative, e.g., to prevent the growth of microorganisms. Procedures and ingredients for the selection and preparation of suitable formulations are described, for example, in Remington: The Science and Practice of Pharmacy (2012, 22^{nd} ed.) and in The United States Pharmacopeia: The National Formulary (2015, USP 38 NF 33).

Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a patient in need of treatment.

A pharmaceutical composition may be administered to a patient, e.g., a human patient, alone or in combination with one or more pharmaceutically acceptable carriers, e.g., as described herein, the proportion of which may be determined by the solubility of the compound, the chemical nature of the compound, and/or the chosen route of administration, among other factors.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Example 1. Viability Assay to Assess TDP-43 Toxicity in FAB1 TDP-43 and PIKFYVE TDP-43 Yeast Cells

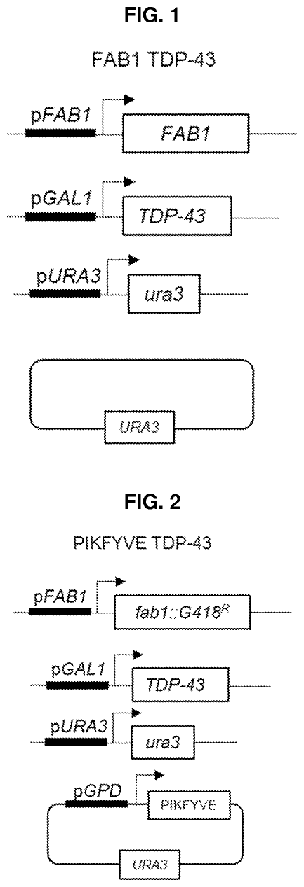

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::Klura3;pdr1::NATMX; fab1::G418R, his3;leu2;ura3;met15;LYS2+) (FIG. 2). Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab1::G418R pAG416GPD-PIKFYVE yeast strain (FIG. 1). Transformed yeast were plated on CSM-ura containing 2% glucose and 200 μg/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 μL dispensed into each well of a flat bottom 96-well plates. Compounds formulated in 100% dimethyl sulfoxide (DMSO) were serially diluted in DMSO and 1.5 μL diluted compound transferred to the 96-well plates using a multichannel pipet. Wells containing DMSO alone were also evaluated as controls for compound effects. Tested concentrations ranged from 15 μM to 0.11 μM. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 μL of 10 mM PI was added to 10 mL of CSM-ura (raffinose or galactose). The final PI solution (50 μL/well) was dispensed into each well of a new round bottom 96-well plate. The overnight 96-well assay plate was then mixed with a multichannel pipet and 50 μL transferred to the PI-containing plate. This plate was then incubated for 30 minutes at 30° C. in the dark. A benchtop flow cytometer (Miltenyi MACSquant) was then used to assess red fluorescence (B2 channel), forward scatter, and side scatter (with following settings: gentle mix, high flow rate, fast measurement, 10,000 events). Intensity histograms were then gated for “PI-positive” or “PI-negative” using the raffinose and galactose cultures treated with DMSO as controls. The DMSO controls for raffinose or galactose-containing cultures were used to determine the window of increased cell death and this difference set to 100. All compounds were similarly gated and then compared to this maximal window to establish the percent reduction in PI-positive cells. IC50 values were then calculated for compounds that demonstrated a concentration-dependent enhancement of viability by fitting a logistic regression curve.

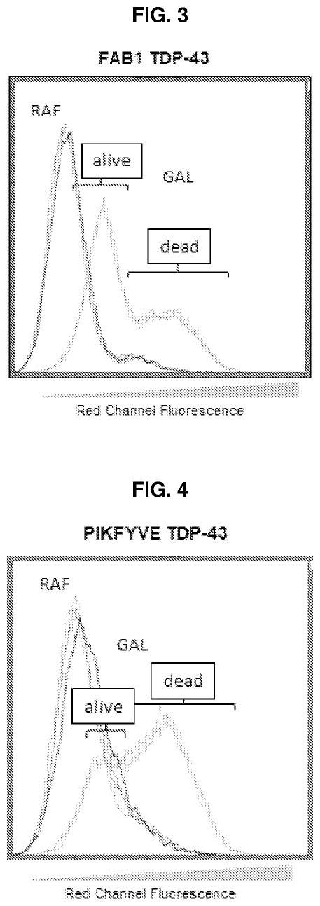

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKFYVE TDP-43 Model. The biochemical PIKFyve inhibition assays were run by Carna Biosciences according to proprietary methodology based on the Promega ADP-Glo™ Kinase assay. A full-length human PIKfyve [1-2098(end) amino acids and S696N, L932S, Q995L,T998S, S1033A and Q1 183K of accession number NP_055855.2] was expressed as N-terminal GST-fusion protein (265 kDa) using baculovirus expression system. GST-PIKfyve was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data were analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

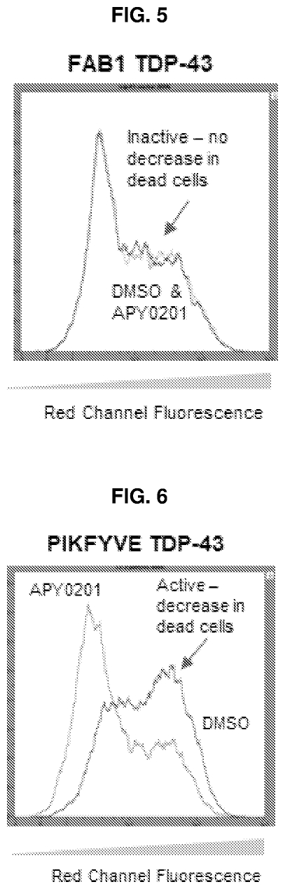

Activity of APY0201, a known PIKfyve inhibitor, was assessed in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKfyve inhibition ameliorated TDP-43 toxicity. Concentrations ranging from 0.5 mM to less than 100 nM afforded increased viability.

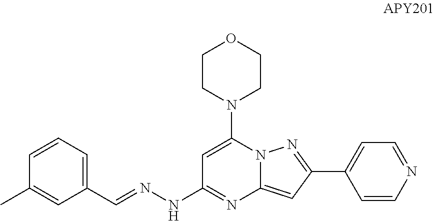 [SMILES: Cc1cccc(/C=N/Nc2cc(N3CCOCC3)n3nc(-c4ccncc4)cc3n2)c1]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

TABLE 15

| | | FAB1 | PIKFYVE |
|---|---|---|---|
| | PIKfyve | TDP-43 | TDP-43 |
| | IC₅₀ | (active/ | (active/ |
| Structure | (nM) | inactive) | inactive) |
| 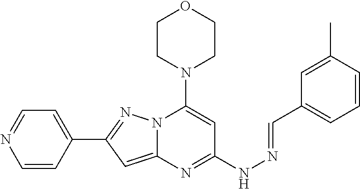 [SMILES: Cc1cccc(/C=N/Nc2cc(N3CCOCC3)n3nc(-c4ccncc4)cc3n2)c1] | 7.5 | Inactive | Active |
| 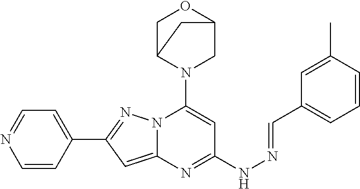 [SMILES: Cc1cccc(/C=N/Nc2cc(N3CC4CC3CO4)n3nc(-c4ccncc4)cc3n2)c1] | 12 | Inactive | Active |
| 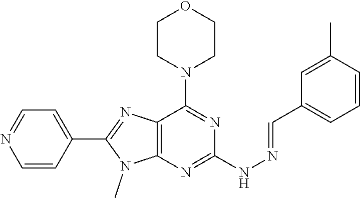 [SMILES: Cc1cccc(/C=N/Nc2nc(N3CCOCC3)c3nc(-c4ccncc4)n(C)c3n2)c1] | 4.9 | Inactive | Active |
| 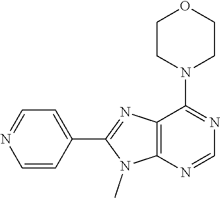 [SMILES: Cn1c(-c2ccncc2)nc2c(N3CCOCC3)ncnc21] | 640 | Inactive | Inactive |
| 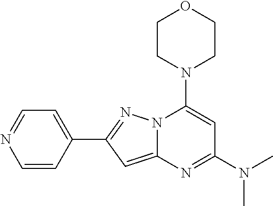 [SMILES: CN(C)c1cc(N2CCOCC2)n2nc(-c3ccncc3)cc2n1] | 2007 | Inactive | Inactive |
| 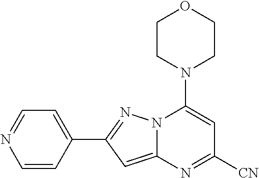 [SMILES: N#Cc1cc(N2CCOCC2)n2nc(-c3ccncc3)cc2n1] | >10000 | Inactive | Inactive |

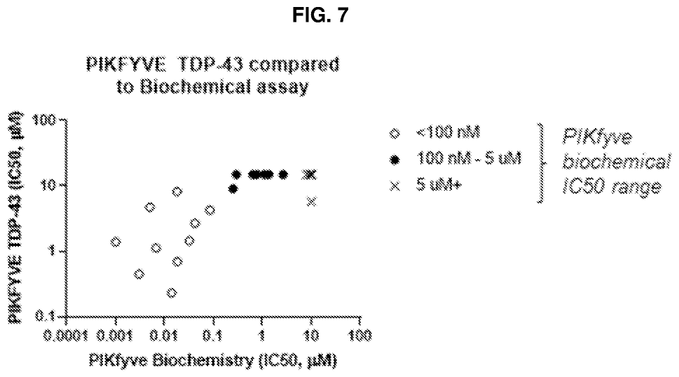

Biochemical and Efficacy Assays. A larger set of PIKfyve inhibitors were evaluated in both a PIKfyve kinase domain binding assay (nanobret) and in the PIKFYVE TDP-43 yeast strain. IC50 values (pM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

TABLE 16

| | PIKFYVE | PIKFYVE |
|---|---|---|
| | Biochemistry | TDP-43 |
| Structure | (IC₅₀, μM) | (IC₅₀, μM) |
| 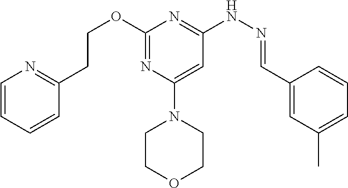 [SMILES: Cc1cccc(/C=N/Nc2cc(N3CCOCC3)nc(OCCc3ccccn3)n2)c1] | 0.003 | 0.450 |
| 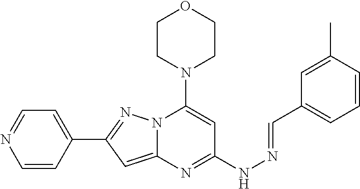 [SMILES: Cc1cccc(/C=N/Nc2cc(N3CCOCC3)n3nc(-c4ccncc4)cc3n2)c1] | 0.001 | 1.390 |
| 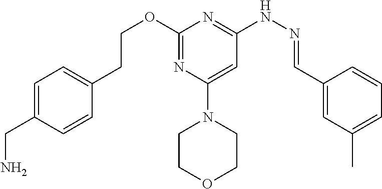 [SMILES: Cc1cccc(/C=N/Nc2cc(N3CCOCC3)nc(OCCc3ccc(CN)cc3)n2)c1] | 0.007 | 1.120 |
| 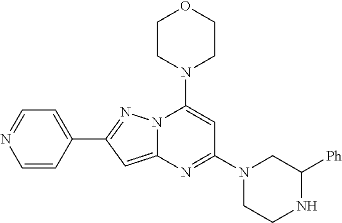 [SMILES: c1ccc(C2CN(c3cc(N4CCOCC4)n4nc(-c5ccncc5)cc4n3)CCN2)cc1] | 2.660 | >15 |
| 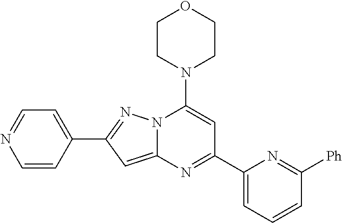 [SMILES: c1ccc(-c2cccc(-c3cc(N4CCOCC4)n4nc(-c5ccncc5)cc4n3)n2)cc1] | 0.014 | 0.230 |
| 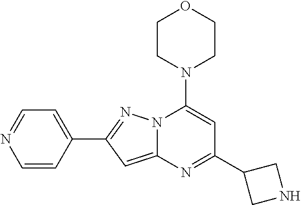 [SMILES: c1cc(-c2cc3nc(C4CNC4)cc(N4CCOCC4)n3n2)ccn1] | 8.020 | >15 |
| 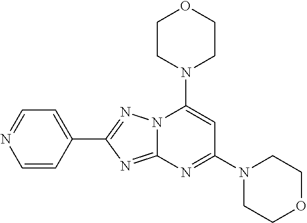 [SMILES: c1cc(-c2nc3nc(N4CCOCC4)cc(N4CCOCC4)n3n2)ccn1] | 9.200 | >15 |
| 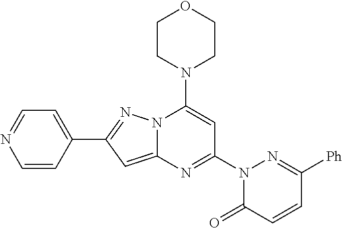 [SMILES: O=c1ccc(-c2ccccc2)nn1-c1cc(N2CCOCC2)n2nc(-c3ccncc3)cc2n1] | 0.295 | >15 |
| 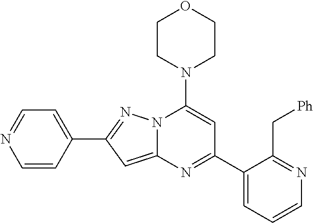 [SMILES: c1ccc(Cc2ncccc2-c2cc(N3CCOCC3)n3nc(-c4ccncc4)cc3n2)cc1] | 1.090 | >15 |
| 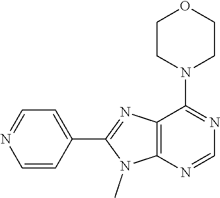 [SMILES: Cn1c(-c2ccncc2)nc2c(N3CCOCC3)ncnc21] | 0.640 | >15 |
| 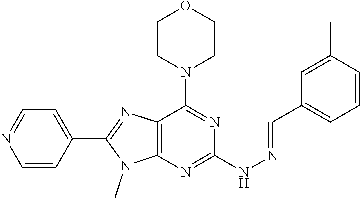 [SMILES: Cc1cccc(/C=N/Nc2nc(N3CCOCC3)c3nc(-c4ccncc4)n(C)c3n2)c1] | 0.005 | 4.720 |
| 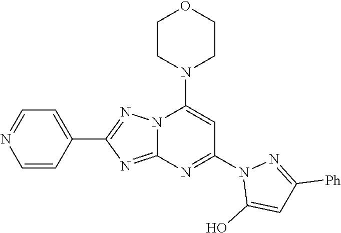 [SMILES: Oc1cc(-c2ccccc2)nn1-c1cc(N2CCOCC2)n2nc(-c3ccncc3)nc2n1] | 0.018 | 0.693 |
| 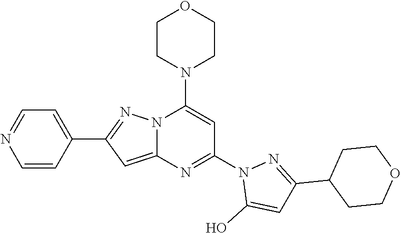 [SMILES: Oc1cc(C2CCOCC2)nn1-c1cc(N2CCOCC2)n2nc(-c3ccncc3)cc2n1] | 0.253 | 9.105 |
| 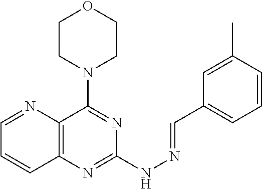 [SMILES: Cc1cccc(/C=N/Nc2nc(N3CCOCC3)c3ncccc3n2)c1] | 0.018 | 8.214 |
| 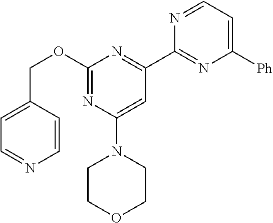 [SMILES: c1ccc(-c2ccnc(-c3cc(N4CCOCC4)nc(OCc4ccncc4)n3)n2)cc1] | 0.032 | 1.447 |
| 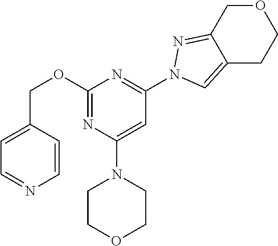 [SMILES: c1cc(COc2nc(N3CCOCC3)cc(-n3cc4c(n3)COCC4)n2)ccn1] | 1.343 | >15 |
| 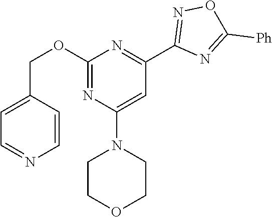 [SMILES: c1ccc(-c2nc(-c3cc(N4CCOCC4)nc(OCc4ccncc4)n3)no2)cc1] | >10 | >15 |
| 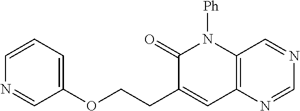 [SMILES: O=c1c(CCOc2cccnc2)cc2ncncc2n1-c1ccccc1] | >10 | >15 |
| 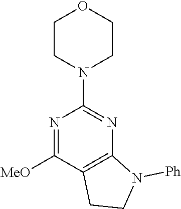 [SMILES: COc1nc(N2CCOCC2)nc2c1CCN2c1ccccc1] | 0.085 | 4.273 |
| 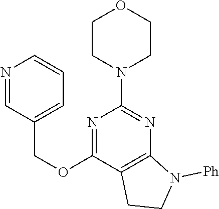 [SMILES: c1ccc(N2CCc3c(OCc4cccnc4)nc(N4CCOCC4)nc32)cc1] | 0.042 | 2.685 |
| 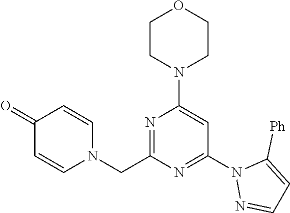 [SMILES: O=c1ccn(Cc2nc(N3CCOCC3)cc(-n3nccc3-c3ccccc3)n2)cc1] | >10 | >15 |
| 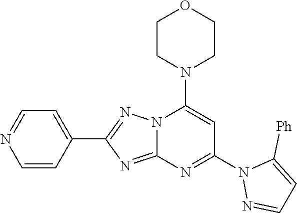 [SMILES: c1ccc(-c2ccnn2-c2cc(N3CCOCC3)n3nc(-c4ccncc4)nc3n2)cc1] | 0.767 | >15 |
| 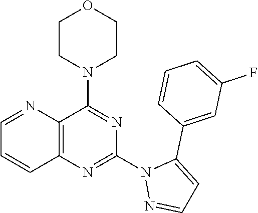 [SMILES: Fc1cccc(-c2ccnn2-c2nc(N3CCOCC3)c3ncccc3n2)c1] | >10 | 5.754 |

### Example 2. Use of a PIKfyve Inhibitor for the Treatment or Prevention of a Neurological Disorder in a Human Patient

Using the compositions and methods described herein, a patient suffering from or at risk of developing a neurological disorder, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, or hereditary inclusion body myopathy, may be administered a PIKfyve inhibitor so as to treat the disease, alleviate one or more symptoms of the disease, or slow or prevent the onset of the disease. The PIKfyve inhibitor may be, for example, a small molecule that specifically binds to an/or inhibits the enzymatic activity of PIKfyve, an antibody or antigen-binding fragment thereof that specifically binds to and/or inhibits the activity of PIKfyve, or substance that reduces expression of functional PIKfyve, such as an interfering RNA molecule (for example, a siRNA, miRNA, or shRNA molecule described herein).

Prior to treatment, the patient may be subjected to one or more analytical tests in order to determine their initial quality of life, muscle strength, muscle function, slow vital capacity, decremental responses exhibited upon repetitive nerve stimulation, among other parameters that describe the patient's initial disease state. The patient may then be administered a PIKfyve inhibitor, such as by way of oral, transdermal, subcutaneous, intranasal, intravenous, intramuscular, intraocular, parenteral, topical, intrathecal, and/or intracerebroventricular administration. The PIKfyve inhibitor may be administered to the patient in combination with one or more pharmaceutically acceptable excipients, carriers, or diluents. The PIKfyve inhibitor may be administered to the patient once or a plurality of times, such as periodically over the course of a treatment period of one or more days, weeks, months, or years.

To determine the responsiveness of the patient to PIKfyve inhibitor therapy, a physician may perform one or more tests in order to evaluate whether the patient exhibits any of the following indications of clinical benefit:
(i) an improvement in condition as assessed using the amyotrophic lateral sclerosis functional rating scale (ALSFRS) or the revised ALSFRS (ALSFRS-R);
(ii) an increase in slow vital capacity, such as an increase in the patient's slow vital capacity within one or more days, weeks, or months following administration of the PIKfyve inhibitor;
(iii) a reduction in decremental responses exhibited by the patient upon repetitive nerve stimulation, such as a reduction that is observed within one or more days, weeks, or months following administration of the PIKfyve inhibitor;
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment);
(v) an improvement in quality of life, as assessed, for example, using the amyotrophic lateral sclerosis-specific quality of life (ALS-specific QOL) questionnaire;
(vi) a decrease in the frequency and/or severity of muscle cramps, such as a decrease in cramp frequency and/or severity within one or more days, weeks, or months following administration of the PIKfyve inhibitor; and/or
(vii) a decrease in TDP-43 aggregation, such as a decrease in TDP-43 aggregation within one or more days, weeks, or months following administration of the PIKfyve inhibitor.

### Example 3. Determining the Likelihood of a Patient to Respond to PIKfyve Inhibitor Therapy

Using the compositions and methods described herein, one may determine the propensity of a patient (e.g., a human patient) suffering from a neurological disease to respond to PIKfyve inhibitor therapy. For example, the patient may be identified as likely to benefit from treatment with a PIKfyve inhibitor by determining that the patient is susceptible to TDP-43 aggregation. The susceptibility of the patient to developing TDP-43 aggregation may be determined, e.g., by analyzing the morphology of neuronal cells obtained by differentiation of induced pluripotent stem cells (iPSCs) derived from the patient. For example, a sample of somatic cells (e.g., hematopoietic cells) may be isolated from the patient and reprogrammed into iPSCs. The isolated somatic cells may reprogrammed into iPSCs by transfecting the cells to express one or more of genes Oct4, Sox2, cMyc, and/or Klf4. Upon reprograming the somatic cells into iPSCs, the iPSCs may then be differentiated into motor neurons, for example, using methods described herein and known in the art. Once the iPSCs are differentiated into motor neurons, the motor neurons may be monitored for changes in morphology that serve as a proxy for TDP-43 aggregation.

For example, the patient's propensity to develop TDP-43 aggregation can be assessed by analyzing the time-dependent neurite outgrowth patterns of motor neurons obtained by differentiation of iPSCs reprogrammed from mature hematopoietic cells isolated from the patient. In some embodiments, TDP-43 aggregation is signaled by a finding that neurites on such motor neurons exhibit a decrease in size and/or a decrease in their rate of growth after a period of time following differentiation in vitro. For example, TDP-43 aggregation may be signaled by a finding that neurites on such motor neurons exhibit a decrease in size and/or a decrease in their rate of growth after from about 10 days to 100 days following differentiation.

Upon determining that the patient is prone to TDP-43 aggregation, and is thus likely to respond to treatment with a PIKfyve inhibitor, the patient may be administered one or more PIKfyve inhibitors, for example, as described in Example Two, above. The inhibitor of PIKfyve may be a small molecule. In some embodiments, the PIKfyve inhibitor is an anti-PIKfyve antibody or antigen-binding fragment thereof, or a compound, such as an interfering RNA molecule, that attenuates PIKfyve expression.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating a neurological disorder in a human patient, the method comprising administering to the patient a therapeutically effective amount of a PIKfyve inhibitor, wherein the patient does not express a mutant form of c9orf72 comprising an expanded GGGGCC hexanucleotide repeat.

2. A method of treating a neurological disorder in a human patient, the method comprising:
(i) determining that the patient is susceptible to developing TAR-DNA binding protein (TDP)-43 aggregation; and
(ii) administering to the patient a therapeutically effective amount of a PIKfyve inhibitor.

3. (canceled)

4. A method of treating a neurological disorder in a human patient, the method comprising:
(i) determining that the patient expresses a mutant form of TDP-43 having a mutation associated with TDP-43 aggregation; and
(ii) administering to the patient a therapeutically effective amount of a PIKfyve inhibitor.

5-9. (canceled)

10. The method of claim 1, wherein the neurological disorder is a neuromuscular disorder.

11. The method of claim 10, wherein the neuromuscular disorder is selected from the group consisting of amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome.

12. (canceled)

13. The method of claim 1, wherein the neurological disorder is selected from the group consisting of frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, inclusion body myopathy with early-onset Paget disease and frontotemporal dementia, sporadic inclusion body myositis, myofibrillar myopathy, dementia pugilistica, chronic traumatic encephalopathy, Alexander disease, and hereditary inclusion body myopathy.

14. The method of claim 1, wherein the PIKfyve inhibitor is a small molecule that binds to and/or inhibits PIKfyve enzymatic activity.

15. The method of claim 14, wherein the PIKfyve inhibitor is a compound represented by formula (I) 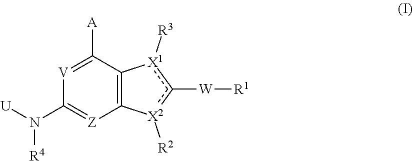
or a pharmaceutically acceptable salt or solvate thereof, wherein
R¹ is hydrogen, halo, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
each of X¹ and X² is independently selected from O, S, N, and C;
W is a bond; O; S; (CH₂)ₙ; S(O); SO₂; NRₐ; C(O); C(O)NRₐ; NRₐC(O); SO₂NRₐ; NRₐSO₂; CRₐ═CR_{b}; C═NRₐ; or NRₐ═CR_{b}, wherein n is 1-5 and each of Rₐ and R_{b} is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of R² and R³ is optionally present depending on the valence of the atom to which each is attached, and if present, each of R² and R³ is independently hydrogen, halo, hydroxyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
R⁴ is hydrogen, optionally substituted C₁₋₆ alkyl, C₃₋₈ cycloalkyl, optionally substituted C₆₋₁₀ aryl, optionally substituted C₁₋₉ heteroaryl, and optionally substituted C₁₋₉ heterocyclyl;
U is hydrogen, 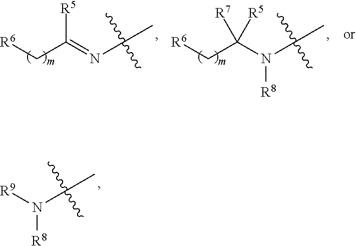
wherein m is 0-3, and each of R⁵, R⁶, R⁷, R⁸, and R^{g} is independently hydrogen, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl; or
R₃ and U, together with the nitrogen atom to which they are attached, form 4- to 6-membered heterocyclyl or heteroaryl optionally substituted by one or more substituents selected from hydrogen, optionally substituted C₁₋₆ alkyl, optionally substituted C₆₋₁₀ aryl, optionally substituted C₁₋₉ heteroaryl, optionally substituted C₃₋₈ cycloalkyl, and optionally substituted C₁₋₉ heterocyclyl;
one of the two [symbol] is a single bond, and the other [symbol] is a double bond; or each of the two [symbol] are aromatic bonds;
each of V and Z is independently N or C_{H}; and
A is optionally substituted C₃₋₈ carbocyclyl, optionally substituted C₁₋₉ heterocyclyl, and optionally substituted C₁₋₉ heteroaryl.

16. (canceled)

17. The method of claim 14, wherein the PIKfyve inhibitor is a compound represented by formula (II) 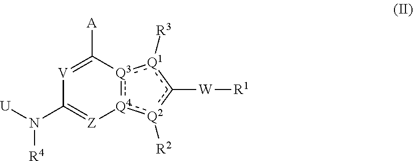
or a pharmaceutically acceptable salt or solvate thereof, wherein
R¹ is hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of Q¹, Q², Q³, and Q⁴ is independently C or N, and at least one of Q¹, Q², Q³, and Q⁴ is N;
W is a bond; O; S; (CH₂)ₙ; S(O); SO₂; NRₐ; C(O); C(O)NRₐ; NRₐC(O); SO₂NRₐ; NRₐSO₂; CRₐ═CR_{b}; C═NRₐ; or NRₐ═CR_{b}, wherein n is 1-5, and each of Rₐ and R_{b} is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
each of R² and R³ is optionally present depending on the valence of the atom to which each is attached, and if present, each of R² and R³ is independently hydrogen, halo, hydroxyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
R⁴ is hydrogen, optionally substituted C₁₋₆ alkyl, C₃₋₈ cycloalkyl, optionally substituted C₆₋₁₀ aryl, optionally substituted C₁₋₉ heteroaryl, or optionally substituted C₁₋₉ heterocyclyl;
U is hydrogen, 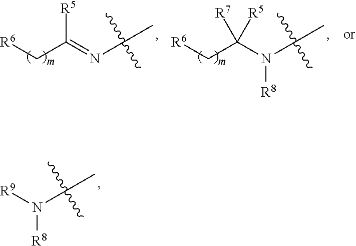
wherein m is 0-3, and each of R⁵, R⁶, R⁷, R⁸, and R^{g} is independently hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl; or
R³ and U, together with the nitrogen atom to which they are attached, form 4- to 6-membered heterocyclyl or heteroaryl optionally substituted by one or more substituents selected from hydrogen, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
wherein each [symbol] is a single bond or a double bond and at least one [symbol] is a double bond; or each [symbol] is an aromatic bond;
each of V and Z is independently N or C_{H}; and
A is optionally substituted C₃₋₈ carbocyclyl, optionally substituted C₁₋₉ heterocyclyl, or optionally substituted C₁₋₉ heteroaryl.

18. (canceled)

19. The method of claim 14, wherein the PIKfyve inhibitor is a compound represented by formula (III) 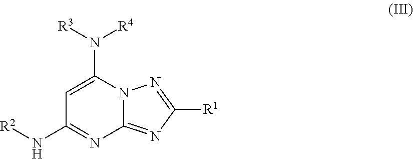
or a pharmaceutically acceptable salt thereof,
wherein
R¹ is optionally substituted C₁₋₆ alkyl, optionally substituted C₃₋₈ cycloalkyl, optionally substituted C₁₋₉ heterocyclyl, optionally substituted C₆₋₁₀ aryl, or optionally substituted C₁₋₉ heteroaryl;
R² is optionally substituted C₁₋₆ alkyl, optionally substituted C₆₋₁₀ aryl, optionally substituted C₁₋₉ heteroaryl, —N═CHRₐ, or —N═CHR_{b}R_{c}, wherein Rₐ is optionally substituted C₁₋₆ alkyl or optionally substituted C₁₋₉ heteroaryl; R_{b} is optionally substituted C₆₋₁₀ arylene; and R_{c} is hydrogen or NHSO₂Me; and
each of R³ and R⁴ is independently H, optionally substituted C₁₋₆ alkyl, optionally substituted C₆₋₁₀ aryl, optionally substituted C₁₋₉ heteroaryl, optionally substituted C₃₋₈ cycloalkyl, or optionally substituted C₁₋₉ heterocyclyl; or R³ and R⁴, together with the nitrogen to which they are attached, form optionally substituted C₁₋₉ heterocyclyl.

20-22. (canceled)

23. The method of claim 14, wherein the PIKfyve inhibitor is a compound represented by formula (IV) 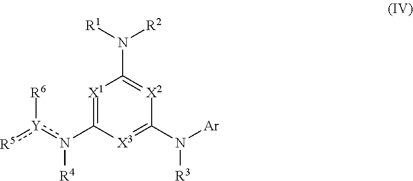
or a pharmaceutically acceptable salt thereof,
wherein
each bond denoted as [symbol] is either a single bond or a double bond, provided that the bonds denoted as [symbol] are not both simultaneously double bonds;
X¹ is selected from N and CR^{A};
X² is selected from N and CR^{A};
X³ is selected from N and CR^{A};
each R^{A} is independently selected from H, halo, C₁₋₆ alkyl, C₁₋₆ haloalkyl, C₁₋₆ alkoxy, and C₁₋₆ haloalkoxy;
Ar is selected from C₆₋₁₀ aryl and 5-10 membered heteroaryl, each of which is optionally substituted with 1, 2, 3, 4, or 5 substituents independently selected from R⁷;
each R⁷ is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c2}R^{d1}, C(O)OR^{a1}, OC(O)R^{b1}, OC(O)NR^{c1}R^{d1} NR^{c1}R^{d1}, NR^{c1}C(O)R^{b1}, NR^{c1}(O)OR^{a1} NR^{c1}C(O)NR^{c1}Rₐ₁, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1} NR^{c1}C(O)R^{b1}, NR^{c1}(O)OR^{a1}, NR^{c1}C(O)NR^{c1}R^{c1}, NR^{c1}S(O)₂R^{b1}, NR^{c1}S(O)₂, NR^{c1}R^{a1}, S(O)₂R^{b1} and S(O)₂NR^{c1}R^{d1};
R¹ is selected from the group consisting of H and C₁₋₆ alkyl, wherein said C₁₋₆ alkyl is optionally substituted with 1, 2, or 3 substituents independently selected from halo, CN, NO₂, OR^{a2}, C(O)R^{b2}, C(O)NR^{c2}R^{d2}, C(O)OR^{a2}, NR^{c2}R^{d2} NR^{c2}C(O)R^{b2} NR^{c2}C(O)OR^{a2} NR^{c2}C(O)NR^{c2}R^{d2} NR^{c2}S(O)₂R^{b2}, NR^{c2}S(O)₂NR^{c2}R^{d2}, S(O)₂R^{b2}, and S(O)₂NR^{c2}R^{d2};
R² is C₁₋₆ alkyl, which is optionally substituted with 1, 2, or 3 substituents independently selected from halo, CN, NO₂, OR^{a2}, C(O)R^{b2}, C(O)NR^{c2}R^{d2}, C(O)OR^{a2}, NR^{c2}R^{d2} NR^{c2}C(O)R^{b2}NR^{c2}C(O)OR^{a2} NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}; or
R¹ and R² together with the N to which they are attached form a 4-7 membered non-aromatic heterocyclyl, which is optionally substituted with 1, 2, 3, 4, or 5 substituents independently selected R⁸;
each R⁸ is independently selected from halo, C₁₋₆ alkyl, C₂₋₆ alkenyl, C₂₋₆ alkynyl, C₁₋₆ haloalkyl, CN, NO₂, OR^{a2}, C(O)R^{b2}, C(O)NR^{c2}R^{d2}, C(O)OR^{a2}, NR^{c2}R^{d2}, NR^{c2}C(O)R^{b2} NR^{c2}C(O)OR^{a2} NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2};
R³ is selected from H, C₁₋₆ alkyl, and C₁₋₆ haloalkyl;
R⁴ is selected from H, C₁₋₆ alkyl, and C₁₋₆ haloalkyl;
Y is selected from N, C, and CR^{A};
when the bond between R⁵ and Y is a single bond, R⁵ is 5-10 membered heteroaryl,
which is optionally substituted with 1, 2, 3, 4, or 5 substituents independently selected from halo, CN, NO₂, C₁₋₆ alkyl, C₂₋₆ alkenyl, C₂₋₆ alkynyl, C₁₋₆ haloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3}, NR^{c3}C(O)OR^{a3} NR^{c3}C(O)NR^{c3}R^{d3} NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3} NR^{c3}C(O)OR^{a3}, R^{c3}C(O)NR^{c3}R^{d3},
NR^{c3}S(O)₂R^{b3}, NR^{c3}S(O)₂NR^{c3}R^{d3}, S(O)₂R^{b3} and S(O)₂NR^{c3}R^{d3};
when the bond between R⁵ and Y is a double bond, R⁵ is CR^{B}R^{c};
R^{B} is selected from H, C₁₋₆ alkyl, and C₁₋₆ haloalkyl;
R^{c} is selected from C₆₋₁₀ aryl and 5-10 membered heteroaryl, each of which is optionally substituted with 1, 2, 3, 4, or 5 substituents independently selected from halo, CN, NO₂, C₁₋₆ alkyl, C₂₋₆ alkenyl, C₂₋₆ alkynyl, Cᵢ₋ₑ haloalkyl, OR a, SR^{a3}, C(O)R^{b3}, C(O)NR³R^{d3}, C(O)OR^{a3}OC(O)R^{b3}, OC(O)NR^{c3}R^{d3}, NR^{c3}R^{d3} NR^{c3}C(O)R^{b3}, NR^{c3}C(O)OR^{a3}, NR^{c3}C(O)NR³R^{d3}, NR^{c3}S(O)₂R^{b3}, NR^{c3}S(O)₂NR^{c3}R^{d3}, S(O)₂R^{b3}, and S(O)₂NR^{c3}R^{d3}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR³R^{d3}, C(O)OR^{a3}, OC(O)R^{b3}, OC(O)NR^{c3}R^{d3}, NR^{c3}R^{d3} NR^{c3}C(O)R^{b3} NR^{c3}C(O)OR^{a3}, NR^{c3}C(O)NR^{c3}R^{d3} NR^{c3}S(O)₂R^{b3}, NR^{c3}S(O)₂NR^{c3}R^{d3} S(O)₂R^{b3} and S(O)₂NR^{c3}R^{d3}; or
R⁴ and R⁵ together with Y and N to which R⁴ is attached form a 5-14 membered heteroaryl, which is optionally substituted with 1, 2, 3, 4, or 5 substituents independently selected from R^{g};
each R^{g} is independently selected from halo, CN, NO₂, C₁₋₆ alkyl, C₂₋₆ alkenyl, C₂₋₆ alkynyl, Cᵢ₋ₑ haloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3} NR^{c3}C(O)OR^{a3}, NR^{c3}C(O)NR^{c3}R^{d3} NR^{c3}S(O)₂R^{b3}, NR^{c3}S(O)₂NR^{c3}R^{d3} S(O)₂R^{b3}, and S(O)₂NR^{c3}R^{d3}; wherein said Cᵢ₋ₑ alkyl, C₂₋₆ alkenyl, and C₂₋₆ alkynyl are each optionally substituted with 1, 2 or 3 substituents independently selected from halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR³R^{d3} NR^{c3}C(O)R^{b3}, NR³C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3} and S(O)₂NR^{c3}R^{d3};
R⁶ is selected from H, C₁₋₆ alkyl, and C₁₋₆ haloalkyl; or R⁶ is absent;
each R^{a1}, R^{b1}, R^{a2}, R^{b2}, R^{a3}, and R^{b3} is independently selected from H, C₁₋₆ alkyl, C₁₋₄ haloalkyl, C₂₋₆ alkenyl, and C₂₋₆ alkynyl, wherein said C₁₋₆ alkyl, C₂₋₆ alkenyl, and C₂₋₆ alkynyl are optionally substituted with 1, 2, 3, 4, or 5 substituents independently selected from R^{g};
each R^{c1}, R^{d1}, R^{c2}, R^{d2}, R^{c3}, and R^{d3} is independently selected from H, C₁₋₆ alkyl, C₁₋₄ haloalkyl, C₂₋₆ alkenyl, C₂₋₆ alkynyl, C(O)R^{b7}, C(O)NR^{c7}R^{d7}, C(O)OR^{a7}, NR^{c7}R^{d7}, S(O)R^{b7}, S(O)NR^{c7}R^{d7}, S(O)₂R^{b7}, and S(O)₂NR^{c7}R^{d7}; wherein said Cᵢ₋ₑ alkyl, C₂₋₆ alkenyl, and C₂₋₆ alkynyl is optionally substituted with 1, 2, 3, 4, or 5 substituents independently selected from R^{g}; each R^{a7}, R^{b7}, R^{c7}, and R^{d7} is in dependently selected from H, C₁₋₆ alkyl, C₁₋₄ haloalkyl, C₂₋₆ alkenyl, C₂₋₆ alkynyl, and R^{g}, wherein said C₁₋₆ alkyl, C₂₋₆ alkenyl, and C₂₋₆ alkynyl is optionally substituted with 1, 2, 3, 4, or 5 substituents independently selected from R^{g};
or any R^{c1} and R^{d1} together with the N atom to which they are attached form a 4-, 5-, 6-, or 7-membered non-aromatic heterocyclyl group optionally substituted with 1, 2, or 3 substituents independently selected from R^{g};
or any R^{c2} and R^{d2} together with the N atom to which they are attached form a 4-, 5-, 6-, or 7-membered non-aromatic heterocyclyl group optionally substituted with 1, 2, or 3 substituents independently selected from R^{g};
or any R^{c3} and R^{d3} together with the N atom to which they are attached form a 4-, 5-, 6-, or 7-membered non-aromatic heterocyclyl group optionally substituted with 1, 2, or 3 substituents independently selected from R^{g};
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, thio, C₁₋₆ alkylthio, C₁₋₆ alkylsulfamyl, C₁₋₆ alkylsulfonyl, carbamyl, C₁₋₆ alkylcarbamyl, di(C₁₋₆ alkyl)carbamyl, carboxy, C₁₋₆ acyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

24-25. (canceled)

26. The method of claim 14, wherein the PIKfyve inhibitor is a compound represented by formula (V) 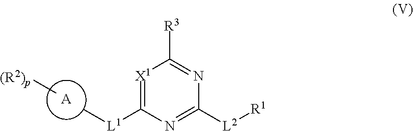
or a pharmaceutically acceptable salt thereof,
wherein
R¹ is hydroxy, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl;
each occurrence of R² is independently optionally substituted C₁₋₆ alkyl, optionally substituted C₂₋₆ alkenyl, optionally substituted C₂₋₆ alkynyl, optionally substituted C₆₋₁₀ aryl, optionally substituted C₁₋₉ heteroaryl, optionally substituted C₃₋₈ cycloalkyl, or optionally substituted C₁₋₉ heterocyclyl;
R³ is a nitrogen- or oxygen-containing moiety;
Ring A is (i) a 5 or 6-membered heteroaryl or 5-6 or 6-5 membered bicyclic heteroaryl, each having at least one nitrogen or oxygen ring atom, or (ii) phenyl;
L¹ is absent, C1-C2 alkylene, —NR^{C}—, —O—, —S—, —C(O)—, —NHC(O)—, or —C(O)NH—;
L² is —O—(CR^{a}R^{b})ₘ—, —(CR^{a}R^{b})ₘ—, —NR^{c}—(CR^{a}R^{b})ₘ—, or —S—(CR^{a}R^{b})ₘ—;
X¹ is C_{H}, N, or CR^{C}; each occurrence of R^{a} and R^{b} are independently hydrogen, hydroxy, hydroxy(Cᵢ₋₄)alkyl, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R, —NR^{c}SO₂R^{d}, —OS(O)₂R^{d}, —OP(O)R^{d}R^{e}, or —P(O)R^{d}R^{e};
R^{c} is a hydrogen or C₁₋₆ alkyl;
each occurrence of R^{d} and R^{e} are independently hydrogen, optionally substituted C₁₋₆ alkyl, optionally substituted C₂₋₆ alkenyl, optionally substituted C₂₋₆ alkynyl, optionally substituted C₁₋₆ alkoxy, optionally substituted C₆₋₁₀ aryl, optionally substituted C₁₋₉ heteroaryl, optionally substituted C₃₋₈ cycloalkyl, or optionally substituted C₁₋₉ heterocyclyl;
m is 1-4; and
p is 1 or 2.

27. (canceled)

28. The method of claim 14, wherein the PIKfyve inhibitor is a compound represented by formula (VI) 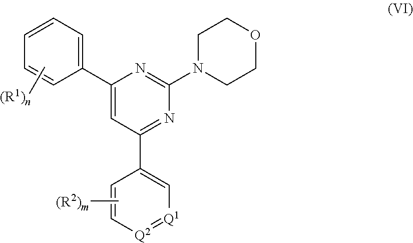
or a pharmaceutically acceptable salt thereof,
wherein
Q¹ and Q² are each independently C_{H} or N, wherein Q¹ and Q² are not both N;
each R¹ is independently hydroxy, C₁₋₄ alkyl, or C₁₋₄ alkoxy;
n is 0, 1, or 2;
each R² is independently C₁₋₄ alkyl or C₁₋₄ alkoxy; and
m is 0 or 1.

29. The method of claim 14, wherein the PIKfyve inhibitor is 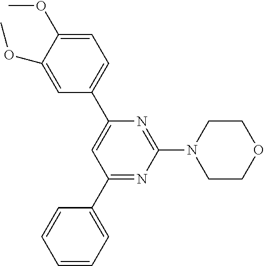 [SMILES: COc1ccc(-c2cc(-c3ccccc3)nc(N3CCOCC3)n2)cc1OC]
or a pharmaceutically acceptable salt thereof.

30. The method of claim 14, wherein the PIKfyve inhibitor is a compound represented by formula (VII) 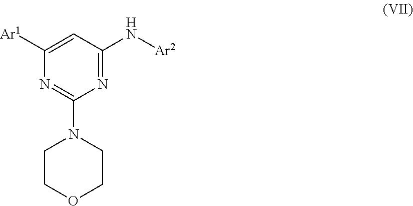
or a pharmaceutically acceptable salt thereof,
wherein
Ar¹ is phenyl or pyridyl, with each optionally independently substituted with 1 or 2 C₁₋₄ alkoxy;
Ar² is phenyl, pyridyl, or pyrimidyl with each optionally independently substituted with halo, C₁₋₄ alkyl, C₁₋₄ alkoxy, or C(O)NR^{2a}R^{2b}; and
R^{2a} and R² are each independently H or C₁₋₄ alkyl.

31. (canceled)

32. The method of claim 14, wherein the PIKfyve inhibitor is a compound represented by formula (VIII) 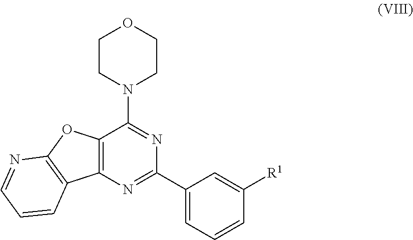
or a pharmaceutically acceptable salt thereof,
wherein
R¹ is hydroxy, C₁₋₄ alkoxy, or H(CO)R^{1a}; and
R^{1a} is phenyl or pyridyl, optionally substituted with amino, alkylamino, or dialkylamino.

33. (canceled)

34. The method of claim 14, wherein the PIKfyve inhibitor is a compound represented by formula (IX) 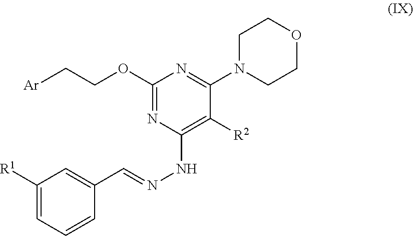
or a pharmaceutically acceptable salt thereof,
wherein
Ar is phenyl or pyridyl, with each optionally independently substituted with 1 or 2 alkyl, aminoalkyl, (alkylamino)alkyl, or (dialkylamino)alkyl;
R¹ is hydrogen or alkyl; and
R² is hydrogen or halo.

35. (canceled)

36. The method of claim 14, wherein the PIKfyve inhibitor is a compound represented by formula (X) 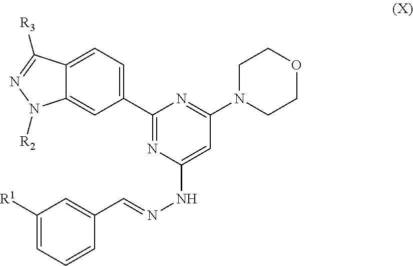
or a pharmaceutically acceptable salt thereof,
wherein
R¹ and R² are each independently hydrogen or C₁₋₄ alkyl; R³ is hydrogen or C₁₋₃ alkyl substituted with morpholinyl.

37. (canceled)

38. The method of claim 14, wherein the PIKfyve inhibitor is a compound represented by formula (XI) 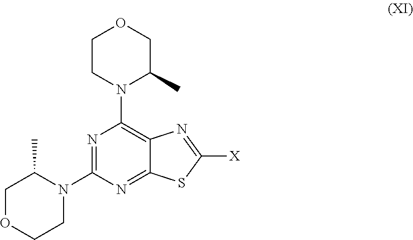
or a pharmaceutically acceptable salt thereof,
wherein
X is selected from

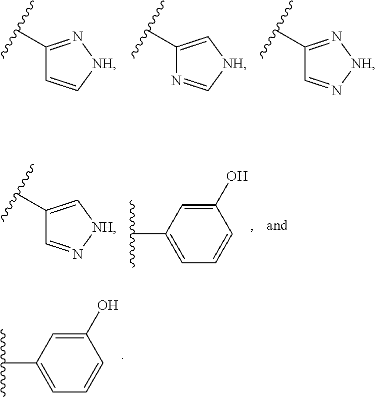

39. The method of claim 14, wherein the PIKfyve inhibitor is

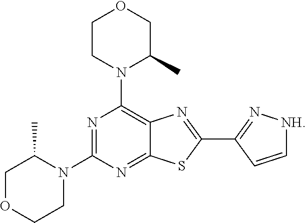 [SMILES: C[C@@H]1COCCN1c1nc(N2CCOC[C@@H]2C)nc2sc(-c3cc[nH]n3)nc12]

40-44. (canceled)


SEQUENCE LISTING (13 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - RNA, 4236 nt, Homo sapiens
ggugggcggggggaggaggcggcccuagcgccauuuugugggagcgaagcgguggcugggcugcgcuuggguccgucgcugcuucggugucccugucgggcuucccagcagcggccuagcgggaaaaguaaaagaugucugaauauauucggguaaccgaagaugagaacgaugagcccauugaaauaccaucggaagacgaugggacggugcugcucuccacgguuacagcccaguuuccaggggcgugugggcuucgcuacaggaauccagugucucaguguaugagagguguccggcugguagaaggaauucugcaugccccagaugcuggcuggggaaaucugguguauguugucaacuauccaaaagauaacaaaagaaaaauggaugagacagaugcuucaucagcagugaaagugaaaagagcaguccagaaaacauccgauuuaauaguguugggucucccauggaaaacaaccgaacaggaccugaaagaguauuuuaguaccuuuggagaaguucuuauggugcaggucaagaaagaucuuaagacuggucauucaaagggguuuggcuuuguucguuuuacggaauaugaaacacaagugaaaguaaugucacagcgacauaugauagauggacgauggugugacugcaaacuuccuaauucuaagcaaagccaagaugagccuuugagaagcagaaaaguguuuguggggcgcuguacagaggacaugacugaggaugagcugcgggaguucuucucucaguacggggaugugauggaugucuucauccccaagccauucagggccuuugccuuuguuacauuugcagaugaucagauugcgcagucucuuuguggagaggacuugaucauuaaaggaaucagcguucauauauccaaugccgaaccuaagcacaauagcaauagacaguuagaaagaaguggaagauuuggugguaauccagguggcuuugggaaucaggguggauuugguaauagcagaggggguggagcugguuugggaaacaaucaagguaguaauauggguggugggaugaacuuuggugcguucagcauuaauccagccaugauggcugccgcccaggcagcacuacagagcaguugggguaugaugggcauguuagccagccagcagaaccagucaggcccaucggguaauaaccaaaaccaaggcaacaugcagagggagccaaaccaggccuucgguucuggaaauaacucuuauaguggcucuaauucuggugcagcaauugguuggggaucagcauccaaugcagggucgggcagugguuuuaauggaggcuuuggcucaagcauggauucuaagucuucuggcuggggaauguagacagugggguugugguugguugguauagaauggugggaauucaaauuuuucuaaacucaugguaaguauauuguaaaauacauauguacuaagaauuuucaaaauugguuuguucaguguggaguauauucagcaguauuuuugacauuuuucuuuagaaaaaggaagagcuaaaggaauuuuauaaguuuuguuacaugaaagguugaaauauugagugguugaaagugaacugcuguuugccugauugguaaaccaacacacuacaauugauaucaaaagguuucuccuguaauauuuuaucccuggacuugucaagugaauucuuugcauguucaaaacggaaaccauugauuagaacuacauucuuuaccccuuguuuuaauuugaaccccaccauauggauuuuuuuccuuaagaaaaucuccuuuuaggagaucauggugucacaguguuugguucuuuuguuuuguuuuuuaacacuugucuccccucauacacaaaaguacaauaugaagccuucauuuaaucucugcaguucaucucauuucaaauguuuauggaagaagcacuucauugaaaguagugcuguaaauauucugccauaggaauacugucuacaugcuuucucauucaagaauucgucaucacgcaucacaggccgcgucuuugacggugggugucccauuuuuauccgcuacucuuuauuucauggagucguaucaacgcuaugaacgcaaggcugugauauggaaccagaaggcugucugaacuuuugaaaccuugugugggauugaugguggugccgaggcaugaaaggcuaguaugagcgagaaaaggagagagcgcgugcagagacuugguggugcauaauggauauuuuuuaacuuggcgagaugugucucucaauccuguggcuuuggugagagagugugcagagagcaaugauagcaaauaauguacgaauguuuuuugcauucaaaggacauccacaucuguuggaagacuuuuaagugaguuuuuguucuuagauaacccacauuagaugaauguguuaagugaaaugauacuuguacucccccuaccccuuugucaacugcugugaaugcuguaugguguguguucucuucuguuacugauauguaaguguggcaaugugaacugaagcugaugggcugagaacauggacugagcuuguggugugcuuugcaggaggacuugaagcagaguucaccagugagcucaggugucucaaagaaggguggaaguucuaaugucuguuagcuacccauaagaaugcuguuugcugcaguucuguguccugugcuuggaugcuuuuuauaagaguugucauuguuggaaauucuuaaauaaaacugauuuaaauaauaugugucuuuguuuugcagcccugaaugcaaagaauucauagcaguuaauuccccuuuuuugacccuuuugagauggaacuuucauaaaguuucuuggcaguaguuuauuuugcuucaaauaaacuuauuugaaaaguugucucaagucaaauggauucaucaccugucaugcauugacaccugauacccagacuuaauugguauuuguucuugcauuggccaaagugaaaauuuuuuuuuuucuuuugaaaucuaguuuugaauaagucugggugaccgcaccuaaaaugguaagcaguacccuccggcuuuuucuuagugccucugugcauuugggugauguucuauuuacauggccuguguaaaucuccauugggaagucaugccuucuaaaaagauucuuauuugggggagugggcaaaauguugauuauuuucuaaugcuuuguagcaaagcauaucaauugaaaagggaauaucagcaccuuccuaguuugggauuugaaaaguggaauuaauugcaguagggauaaaguagaagaaaccacaaauuaucuugugccugaaauccauuaagaggccugauagcuuuaagaauuaggguggguugucugucuggaaguguuaaguggaaugggcuuuguccuccaggaggugggggaaugugguaacauugaauacaguugaauaaaaucgcuuacaaaacucacacucucacaaugcauuguuaaguauguaaaagcaauaacauugauucucuguuguacuuuuuuguaacuaauucugugagaguugagcucauuuucuaguuggaagaaugugauauuuguuguguugguaguuuaccuaaugcccuuaccuaauuagauuaugauaaauagguuugucauuuugcaaguuacauaaacauuuaucaaugaagucauccuuuagacuuguaaucgccacauuguuucauuauucaguuuccucuguaaagggaucuugaguuguuuuaauuuuuuuuuucugcaucugaaucugcaugauuuccaaacccuguaccaucugaauuuugcauuuuagcacuugcacuauuacucagcagcaguaacaugguaacacuuaaaaugguacucggggaccuccaaagacuaaacugacaagccuucaaggagcccagggguaaguuaacuugucaacggcaugguuuaaucccuucuuuacacuuguguaaauuucaguuacuggucauagaaggcuuucaauguugaguggccuuuuauuaacauguuuaugguacugcauagauacggguauuuauuuuacccuaagaagauuuugaaguuuaaaaguacuuaaacuauuuggcaaagauuuguuuuuaaaaaucuauuuggucaaucuaaaugcauucauucuaaaaaauuuuuugaaccagauaaauaaaauuuuuuuuugacaccacaaaaaaaaaaaaaaaaaaaa

SEQ ID NO: 3 - RNA, 4236 nt, Homo sapiens; identical to SEQ ID NO: 2

SEQ ID NO: 4 - protein, 414 aa, Homo sapiens; identical to SEQ ID NO: 1

SEQ ID NO: 5 - DNA, 6 bp, Homo sapiens
ggggcc

SEQ ID NO: 6 - RNA, 6 nt, Homo sapiens
ggggcc

SEQ ID NO: 7 - RNA, 9908 nt, Homo sapiens
gagaagccgcatcaaccatgtaagcagcttcgcttcctgccgcaaccgtccgcggcctgaggagcccaccgccgctctcgggggccgacttccgggggctgagccgttgaagcggaggctggggcggggggcagccggcgcggccggggcaggaggcgcagactcatgaaatggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattagacacaagaaggaaagcagaacctacctttggaggtcatgaccctcgtacagctgttcagcttcgaagcctcagcacagtattaaaacgcctcaaggaaatcatggaggggaaaagccaggatagtgacctgaaacaatactggatgccagatagccaatgtaaagagtgctatgactgtagtgagaaatttacaacctttaggcgcagacaccattgccgactatgtgggcagattttctgcagtcgttgctgtaatcaagaaatccctggaaaatttatgggctatacaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaagtttgattcatccagattcctcaaatactcctctttcaacaagacttgtatctgtgcaagaggatgctgggaaatctcctgctcgaaatagatcagccagcattactaacctgtcactggatagatctggttctcctatggtaccttcatatgagacatctgtcagtccccaggctaaccgaacatatgttaggacagagaccactgaggatgaacgcaaaattcttctggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaagggcacaagctatagcaattggacaagcaatggttgatggacgttggctggattgtgttagtcatcacgaccagcttttcagagatgagtatgcgctgtatagaccactgcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctaattctgccagtcctagcaagcgcacatcagtcagcagtttccagtccacagtggacagtgactcagccgcttctatcagcctgaacgtggagctggacaacgtgaacttccatatcaagaagccctccaagtacccacatgtgccccctcaccctgctgaccaaaaagagtatttgatttctgacactggaggacaacagctctcaataagtgacgctttcatcaaagaatccttatttaatcgccgagtagaggaaaaatccaaagagctgcctttcacacctttgggctggcatcataacaacctggagctcctgagggaggagaatggggagaaacaagccatggagaggttgctttcagctaatcataaccacatgatggcactactccagcagttgctccatagtgactcactgtcatcatcttggagggacatcatcgtgtcattggtctgccaggttgttcagacagtccgacctgatgtcaagaaccaggatgatgacatggatatccgtcagtttgtccacatcaaaaaaatcccaggtggaaagaagtttgattctgtggttgtcaatggctttgtttgtaccaagaacattgcacataaaaagatgagttcttgtattaaaaaccccaaaattcttctgttgaagtgttccattgagtatctctacagagaagaaactaagtttacttgcattgatcctattgtgcttcaggaaagggaattcttgaagaattatgtccagcgaatagttgatgttcgacccaccttggttcttgttgagaaaacagtgtctcggattgcccaggacatgttattggaacatggcattactttggtcattaatgtaaagtcacaagttttggaacgaatcagtcgaatgacccaaggtgatttagtgatgtcaatggaccagctgcttacgaaaccacacctgggcacttgtcacaaattttatatgcagatatttcagttgcctaatgaacaaaccaagacactgatgttttttgaaggttgtccacagcacctaggctgtacaatcaagctaagaggaggctctgattatgagctggctcgagttaaggagatcctaatatttatgatctgtgttgcttatcattctcaactagaaatatcctttctcatggatgaatttgctatgcctcccacattaatgcaaaacccttcattccattccctgattgagggacgagggcatgagggggctgtccaagagcagtacggtggaggttccatcccctgggatcctgacatccctcctgagtctctgccctgtgatgatagcagtttgctggaattgaggattgtgtttgagaagggtgagcaggaaaataaaaatcttccgcaggctgttgcctctgtgaagcatcaagaacatagcacaacagcttgcccggcgggtctcccttgtgctttctttgcacctgtaccggaatcattgttgccactccctgtggatgaccaacaagatgctttaggcagcgagcagccagagactttgcagcaaacagttgtgctgcaggatcccaaaagccagataagagcctttagagaccctctacaggatgacactggattatatgttactgaggaagtcacctcctctgaagataaacgaaagacttattctttggcctttaagcaggaattaaaagatgtgatcctctgtatctccccagtaatcacattccgagaaccctttcttttaactgaaaaggggatgagatgctctacccgagattattttgcagagcaggtttactggtctcctctcctcaataaagaattcaaagaaatggagaacaggaggaagaaacagctgctcagggatctctctggacttcagggcatgaatggaagtattcaggccaagtctattcaagtcttaccctcacatgagctagtgagcactagaattgctgagcatctgggcgatagccagagcttgggtagaatgctggccgattatcgagccagaggaggaagaattcagcccaaaaattcagacccttttgctcattcaaaggatgcatcaagtacttcaagtggccaatcaggaagcaaaaatgagggtgatgaagagagagggcttattctgagtgatgctgtgtggtcaacaaaggtggactgtctgaatcccattaatcaccagagactttgtgtgctcttcagcagctcttctgcccagtccagcaatgctcctagtgcctgtgtcagtccttggattgtaacaatggaattttatggaaagaatgatcttacattaggaatatttttagagagatactgtttcaggccttcttatcagtgtccaagcatgttctgtgatacccccatggtacatcatattcggcgctttgttcatggccaaggctgtgtgcagataatcctgaaggagttggattctccagtacctggatatcagcatacaattcttacatattcctggtgtagaatctgcaaacaggtaacaccagttgttgctctttccaatgagtcctggtctatgtcatttgcaaaataccttgaacttaggttttatgggcaccagtatactcgcagagccaacgctgagccctgtggtcactccatccatcatgattatcaccagtatttctcctataaccagatggtggcgtctttcagttattctcccattcggcttcttgaagtatgtgttccactccccaaaatattcattaagcgtcaggccccattaaaagtgtcccttcttcaggatctgaaggacttctttcaaaaagtttcacaggtatatgttgccattgatgaaagacttgcatctttgaaaactgatacatttagtaaaacaagagaggaaaaaatggaagatatttttgcacagaaagagatggaagaaggtgagttcaagaactggattgagaagatgcaagcaaggctcatgtcttcctctgtagatacccctcagcaactgcagtcggtctttgagtcactcattgccaagaaacaaagtctctgtgaagtgctgcaagcttggaataacaggttgcaggaccttttccaacaggaaaagggtagaaagagaccttcagttcctccaagtcctggaagactgagacaaggggaagaaagcaagataagtgcgatggatgcatctccacggaatatttctccaggacttcagaatggagaaaaagaggatcgcttcttaacaactttgtccagccagagctccaccagttctactcatctccaattgcctacgccacctgaagtcatgtctgaacagtcagtgggagggccccctgagctagatacagccagcagttccgaagatgtgtttgatgggcatttgctgggatccacagacagccaagtgaaggaaaagtcaaccatgaaagccatctttgcaaatttgcttccaggaaatagctataatcctattccatttccttttgatccagataaacactacttaatgtatgaacatgaacgagtgcccattgcagtctgcgagaaggaacccagctccatcattgcttttgctctcagttgtaaagaataccgaaatgccttagaggaattgtctaaagcgactcagtggaacagtgccgaagaagggcttccaacaaatagtacttcagatagcagaccaaagagtagcagccctatcagattacctgaaatgagtggaggacagacaaatcgtacaacagaaacagaaccacaaccaaccaaaaaggcttctggaatgttgtccttcttcagagggacagcagggaaaagccccgatctctcttcccagaagagagagaccttacgtggagcagatagtgcttactaccaggttgggcagacgggcaaggaggggaccgagaatcaaggcgttgagcctcaagatgaagtagatggaggagatacacaaaagaagcaactcataaatcctcatgtggaacttcaattttcagatgctaatgccaagttttactgtcggctctactatgcgggagagtttcataagatgcgtgaagtgattctggacagcagtgaagaagatttcattcgttccctctcccactcatcaccctggcaggcccggggaggcaaatcaggagctgccttctatgcaactgaggatgatagatttattttgaagcaaatgcctcgtctggaagtccagtccttcctcgactttgcaccacattacttcaattatattacaaatgctgttcaacaaaagaggcccacggcgttggccaaaattcttggagtttacagaattggttataagaactctcagaacaacactgagaagaagttagatctccttgtcatggaaaatcttttctacgggagaaagatggcacaggtttttgatttgaagggctctcttaggaatcggaatgtaaaaactgacactggaaaagagagttgtgatgtggtcctgctagatgaaaatctcctaaagatggttcgagacaaccctctatatattcgttctcattccaaagctgtgctgagaacctcgatccatagtgactcccatttcctttctagccacctcattatagattattctttgctggttgggcgagatgatactagcaatgagctagtagttggaattatagattatattcgaacatttacatgggacaaaaagcttgagatggttgtgaaatcaacaggaattttaggtggacaaggtaaaatgccaacagtggtgtctccggagttgtacaggactaggttttgtgaggcaatggacaagtatttcctaatggtaccagaccactggacaggcttgggtctgaattgctgaaatcaagcacatattttgaaatggactgtgaaggaaaaggggacaggaacaaaggaccaaaaataagctacatgttttatttcttcatcgtgttcaccactgtatgccaaggcttttcagttctgtggctgtttagactgtccgtaatggaatggtaaaactccatgaatttgcactttggtttttgatacctgtggagctgtctgtaggttgggaagtggcatgaaaattttcttaagctaaaatacagacatgtttcaaagggctaaagttggagatgagtagatagggtgaaaaatgggttaaatttgctagcttaattgttttaagaagaaaacagtgtctcataaattgactatcctggcatcacatttaacatgttatctacttagaaagcatttgtagagctgctgaatttgttttgtgtttttctgtaataatttaatgttacttattatcagaatttctgaaacctttacaaaaattctgatttattccattaatggccggttaaacacgtgggcatttattgttttattgaggaatttgacttaaactgggaatcctgtcatgttgtttatctttccagcttgcctgtttttgagtatgtttgatgtttttaaaattttgtcttctctgtggaagacaggaggctacagcaattaactttaagcctccttttagagatatttttaaaacttgtttaaaatttttgtgcaattcatatattaaattgcacttacttgcatacgctcatattctagggttttttctctatttttagggtatcatagtaaatcattagtaaatgagtctgtagttactaaaccctaatggaataattattaatgaaagatttttgaaatataaaaaataaattaggcccaatccaagaaattgagtgagaaggaaacacttgttttattcacagaggtaaagtgtcttttcaatataaccagcaatttaggtggcatctataaaataaaaaatttctactgtggacatccccttttccaactttctacataatggctagttctgactactaagaaatgttaagaaataggccaagtgcggtggctcacgcctataatcctatcactttgggaggcctaggcaggcagatcacctgaggtcaggagttcaagaccagcctggccaacatgacgaaaccccatctctactaaaaagacaaaaaattagctgggcatggtggcatatacctgtaaccccaactacttgggtggctgaggcaggagaattgtttgaacctgggaggcggaggctgcagtgagctgggattacaccattgcattccagcctgggcaacagagcaagactgtctcaaaaaacaaaacgaaacaaaaaaagaaagttattcttagtaaggaacttcttgtttaatagcatttttgtttattttaaaaagtgatcagaagtagtaaactatctttgaggaaatactgtaaccccagaatatttcctcttgacttctttttgtaacaaggataatttagggatttataaagttgtaaggatttcactgtttttggactgcctataataatagcacattaaccttcacataataagaaatctggacaagttcagttacacagtatgatgaatacttgaattaggaacattgtggaaaatttgctttagagaatcaaggcagtagtttggtatttggtgcttattaaaaatgtggtttgttttgaactggaagcaagttgaccaaggacttatgactaatgtgatgctaagttccacttggccccttttaaaaacgtgtatgtgccttttgaagatacacaaaacactgaggattttagttttgaaatcaaagactattaaaggagctgtacagaggtaaaaaaataaatgtggaacattattaacttactttgtgtctaggaacaatggattttgtatctgatttaaaatgccaacactgttttgtctctgttcattttttctgtgaggatacttaaggttattattcctgtctgtttcctgtactcccctagtcatgagcacttgaagtacaaggtgtctccccctaggtgcaattaggttgtttctttgtttttagtttcaattctatgtgcatagcaggaatgctccacaggaatggcttctgacaataatctgtcctgttgattttgttttccttgcccatgacttgaacaactgtgttttaaagtactgtagtctagtaggtaactttgtggcaaaaattttcaatataatacattctgaaacaatagttgctgccttgcaaaggtaatctctcattttaaaattggacagtattaatgaaggggaaatatacaatttatttctattgagtggtagaactatatgtctggtcccttgctgctcttgtttaggccactatcatagatatatttcaaatattgtactactcagtgttaagtattgaatgactgtttccctttccttcaaggcctagagtatattctgaaaatttaggaatgaggaagaaatcttaatacttccttccttaacatacaacatgagtcccgagaataattgatagtagcaagagaaaactatgtcagtaacatgttgctttgtataaaaatcttatttataaatgtgaagctttttgatgccatcaaaacttattaaaaaataggatttacttttttctaattctgacctaagaaaaataatgagaacaagctgttgcaagctcttttgtagtctattgaatattttatagatattcaaaatttcctacaaactataattttttccatgatttagcagtgagtgattttctagctttggctcttattaggtattgtaaatagtagggttatatcgatatcagcttttgtgatggcattgtggtcatcagcttcatgacattttacccatttgcagtgatcctgtgtaaaactgccaaggaaagtaattacctgtaggagtttgctgagcttgaagagtgaaaactgttgtgaatgagcctgatcataaaacggaccaggccattcattattcctcaagtgttaatatactgacttatgcagtattcaaaccatctagtgcaatgtttttgtttttgttttttttttggtaacacaggtgcagtgtattatagaaaaaataaaaactacaatcattagcagttttaatactgctgtgtcagttttgtaaaaaatgtacattatgtcttttgacatgttgaattttaaactagggaaatgacattgtaaatcatagtagcctcttttaatttaatatgaaaaatgccactatattgaaagtacttaatgtattgtatatatttctctactttggttctagctattttatatgattgacatgttatttaaaagataactgccttgaacttttggagacttgtactgtaaataaagaaatcttaacaataaactcagaatctactta

SEQ ID NO: 8 - DNA, 99485 bp, Homo sapiens
tcaagaaattctcctgcctcagcctcctgagtagctgggactacaggcatgcgccaccatgcctggctaatttttctatttttagtagagatggggtttcagcatgttggccaggctggtctcgaactcctgacctcatgatctgcccacctctgcctcccaaagtgctgggattacaggcgtgagccaccacacctggcaggttacatgatcttatacataaaaaaccctaaagactgcaccaaaaaactattagaataaacaaatttagtaatgttgtaggatataaaatcagtatacaaaaatgagtactgtttccaaatattaacagtgaactatctgaaagagaaatcaataaaacaatcctatttgcaatagctacaaaaatatattaaaatacttagaaataaatacgggccaggtgtggtggctcacacctgtaatcccagcactttgggaggccaaggcaggcgaagcacgaggtcaggggtttgagaccagcctggccaacatggtgaaaccccgtctctactaaaaatacaaaaaaattagctgggcgtagtggtgggcacctgtaatcccagctactcaggaggctgaggcaggagaattgcttgaacctgggaggcggaggttgcagtgagccgagatggccccactgcactccagcccgggtgacacagtgagactccatctcaaaaaaaaaaaaaaaaaaaagaaagaaatttaacagaagtaaaagacccgcacactgaaacctataaaacattgatgaaagaaactgaagaagacagaaataaataaaaagacatctcatgtttatgggttgaaagaattaatattgttaaaatgtccaaagtgatccacagattcaatgcaatccctataaaaattccaatgactttttcgcacagctagaaaaaaaagtctaaaattcctatggaatgacaaaagaggctgaatagctaaagaattcttgagcaaaaagaacaaagctagaagcatcatactgcttgatctcaaaatacactacaaagctacagtaatcagaacagcatggtactggcagagaaacagacaaaccaactgaacagaataaagagtctatcagttaataggatgctcatttttaaaaaaaaataaaaagtgaaaaaaagaataaagagtccagaagtaaatttacccatttacagtcaattgttcaataaggggacaagaatacactatagggaaaggatattctcttcaataagtagtaatgggtaaatgggatatccatatgcaggagaatgaaattagaggcttatctcataccatatataataatcaactcaaataaattgaagacgttatgtgtaccccctgaaactgtaaaaaacgagagggaaaagctccatgacattggtctgagtagtgattttttagatatgaccccaaaagcacaggctacaaaagccaaatagacaaatgggattacataaaactgagaagtttctacagagcaagggttacaatagacagactgaagagacaacagagaacaggagcaaatatttgccagccatacatctgataaagggataatatccaaaatatataaggaactcaaacaactcaatagtaagaaagcaaataatctgattataaaatgggcaaaggatgtgaatagacatttctcaaaagaggacatacaaatggccaacaggtatatgaaaaaaagttcaacgtaattaatcatcagacaaatgcaaattaaaaccacaatgagagatatcacttcacacctattacaatggctatatcaaaaagacaaaagataacaagattggagagaaaatagagaaaaggaaacccttgcacactgttggtaggaatgcaaattagtacagccattatggaaaacagtatggaagttgccaaaaaatttaaaaataaaactatcatgtgatccagccatcccactacagggtatatatccaaaggaaataaaatcactattttttcttttattgaaattaaaaaaaatctttttatagagatggggtcttgctatgttgaccaggctggtgttgaactcctggcttcaagcaatccttccatcttggcctcccaaagtgctaggattacaggcgcgagccaccgtgtccagccaaaatcactatttttaagaggtatctgcactcccatctttattgcagcattattcacaatagccaagatatgaaatcaacctaagtgtccatcaatgaacaactgaataaagaaaatgtggggctgggcactgtggctcacgccggtaatcccagcactttgggaggctgaggcaggtggatcacgaggtcaggggttcaagaccagcctggccaagacagtgaaaccccgtctctactaaaaatacaaaaattagccaggtgcagtgacaggcgcctgtaatcccagctactcgggaggctgaggcaggagaataggggtggggtggaggtcgggggggttggggcagagggtggagattgcagtaagcccagattgcaccactgtactccagccttggtgacagactccatgtcaaaaaaataaaataaaatgtggtatatatatacggtggaacattattcagccttaaaaaataaggaaattctgtcttttgtgacaacatggatgaacctggggacattatgttaagtgaaataaaccagacacagaaatataccacaagatctcatatatatggaatttaaaaatgttgaactcatagaagtagtgggtagaatggtggttgccaggggtaaggggtggcggagagagaaaaagaacaaaaagtaagatgtgctcaaaaggtaatggaggcatgtcaaatggactcaggagtctgctggaaggggctcacaccagccaaggcggggacacctggagcacagaaatgaatagtggcagtaatgaactataacatgttggaaaaaaatgagtccaaagtcatactaaccaatataaagaaagaaagaaaaagatggaacgctattagttgcagtaaaattcaactaatacatacagaggaatagaagtataatatcatcgaacaataattgattcagcaagaatcctaaatctgtgcttgaatgaaagtttttgaggaggatatatacacagacaaataatctctctacagataaaattcatctggaattaattttggatgtctagtgtgaagtgagaatctaaaatgatttcttttgcaaaaacagtagcccagttttcaatctcttttccacagaaattaaactgtaagctaatatagatattagacggtaagcttcttgaggacatggactgtgtaatgttcacatatatttttattttttaggctagtcaagtgaagcagtgggatacgttcataattctatccttagtgtagtgcttctataactaacacactgcaggttaaattagggactcaaaaatatttgcggagtgcactttttttttttttttttaaaccatagatggagtttctacatacactgcagtcgtcgtggctgggcaatttattcttttcatatgatctctgtacctatttcatactttattgtaggtttatgaaatattaacatagggtgataattacgagggccttttttgcttatcttaaatttttaaaaaaaatttttttaaaggaaggcagagacttgagcatctttatgggctgaggaggcagagtcattggaaaaggggaggttgcagatacggtgcaagctcccagacaggagagggtgaattgggggccaaaaggattgagctggggaagaagaggccccttcctcagagagatgagggagaaaggcagaccagggcagatgtggatgtaggtaaatgttgtggtgtgtggggaattagcttgttaagggtcacgcccagataccttgggatttttcacgtgggtataaaatatgattctctgatgagaatgatggtcaaggtaatgtgggggtcgtggtggtggtctgacgcagcagtaataaggaatgggagaagctgactacagactcccagaaagattgctgaagggcccagctgaggatggtgactgggatttgcactggactcaacctgcaggtttcgatggtgctttccaactccaggaaaactgggctgggatttgacaaaactcttgaggaaaaggactcacagaatttaaaactgcatttccctttaggtttcgtctgctttccatcctcccctaccaagccccacaactgcggcgagcatgttttctgcagttaactgcactgcgctctcagcaggacagaaatgacgaataactctagccaagctgaacaagatagggcttgggcttggtaactttatttgcatcagcatttgcaacacaacacctaaatgaaaaaattaactataaagccgacatcacttactctgggtcagggaactgtgccattcgctttaagtgcatttacttaatctcaaaaaagtattaagaagtgagtccttttattaatccgactttacacaccagaggtctaaggcctaaataaatgagcaatttgcttaagatcatatggctggttattggtggagtcatgcaagtctggaaacagagtcatcgctaactccggttcagtgctacacgagtgaactgtctacaacaaagcaataacatattatgagtgcccacagtatcgaacacatacggtaaccgcagtccagccagcacccctgcagtgcccagagcaccaagcacccatccgttgaccccgcatgcgtgacgctcacgcgcgcagtccccctggcttcttccaggaaggctgaagcggcctaatctcggccaaaagatcaggacgactcgatctgggctggactccttctgcctgagcatctgatccgagcgtcggccgggctgggaggagccggcccccgggaaagatttcggactgggggacaggaaaacagagggcggagtcttgggggggggaagcgagaagccgcatcaaccatgtaagcagcttcgcttcctgccgcaaccgtccgcggcctgaggagcccaccgccgctctcgggggccgacttccgggggctgagccgttgaagcggaggctggggcggggggcagccggcgcggccggggcaggaggcgcaggtaagggagggggcctggagcttggccggctggggctcagcatgggaaggaggccggtatggggagctccagggccagagctgaggtgaaagtgagaaccagggctccgcagtcgggggctagggcctggctgccggctgagcaaggaagggcagagaatctggctttagaattagcttgtcctccctattccccggctgccttgtctggcgagtggggacggaagccgggtggggcccttgagatttccttggaaacatttgaagttcctttgcctctctctcgacccttacgcctgaacttaattaggaggggaagagtacagttacttttccctctgagtgcatgagttctgagtttgtcagaggcgcatccgtaggctggaaaagaaagttcatgagctgcagaaacgataccaaccgtgggggttcaaataagtgtttgtctttttaaatgcttgcctctgctatttgagacgattccatttcaacttaggttttggagtttcttgaggaggagcattatttcccaattagtgtttaccctttgcttagaatgttcccaacaaaaggatgagaatggaatgtgttggaaacttttaggacttaggggttttctgcccaatcagaggttattcctaagacagtcaagaaatataagaagtgctagaattctgtataaagcaagttattcgtgtgtatctgatgtgtatacatgattcattatctactattgacgcagtgcatcctgtgttgtaaaatatgtccagtgttgacaatagaggtagtcgcttggtataatgtacttatttctgatttatttgagccccttttttggcactccaaaactcccactctgtcagttttcttacaaaaaatgttctttcttttgtgtttgtgacaaagattgtgtaggttaaatctcttctgtggagcaaggctaatttcctgtgttctccctttttctgctttctggatcctgctctggttttcctttaagcgtgttggtcaaaaattgaacaagaaatctttgttgatagaagctaacatttccaatattcagctttcctaaattattacattgccagttttttttttttttttttttgagagacagagtctcgctctgtcccccaggttggagtgcagtggcgcgatctcctccgactgcaacctccgcctcccagggttcaggcgattctcctgcctcagcctcctgagtagctagattgcaggcgcgcgacaccacactcggctaatttttgtatttttaggggagacggggtttcaccatgttagcgaagctggttttgaattcctgacctcaggcgattcacccgcctcggcctcccaaggtgctgggattacaggcgtgagccaccgggcgggggtctccctccctcgcccaggctggagtgaagtggtgtgatctcggcttactgcagtctccgcctcccaggctcaagggattctcctgcctcggcctcccaaagtgctgggatcacaggtgtgagcagccacgtccggcctttactgactggcttctgatagtttgtagtaggtctgcatatattagtagccttaattagaaatggtgaagtattggtgatatatcagaaggctttgggtaggtaggttaataggactgattaccactcaggaaagagtcctaatttatattttaaagcgttgttaagctgctaaagagtgagagttataagataattttttcagggccctagtgtcattatgtaattattagggttaactatatgattgggtttatatttcagagactacacttgctaagtgttttcatgggaatttttatcttcttatttccaaagccgagtataatttttgtgtagataacctttagaagtaattctaagactctttaggcagaatgtcatctctgcctctgacattacttgtgtcaaagtagaatgtaggctgttttccaggggtggttgttacggccatcacattatcgtttctctccagagctcttttttttttttttttttttttccctaaataaaatagagatgaagtctcgctatgttgactaggctggtctggaactcctggcctcaagcgatcctcccgtctcagcctcctaaagtgctgggattacagacatgaggccacagtgcctggcctctttttttttttaatataaaaaaaacacaaacttttttttgagtgtatacttattcagttatcagcttacttcccaacactttttgctgaattgtcatgacacttcagaaggattgttgatgattattctcagtggaagatattagaaaccttccttccccttcttcttccttttctccttcctctttccttccccttcctcttcctctttttcttccccccccaataaacatgtattgccaaaattctgtttaagtcagtggtttcatttctgccttaatttattaattaaattattgatcagacaaagccttaagtagttagagaacgcggataggctaaaagggaagtgtgaagagctacaaaagtaattttgatttaggtaatacttttggggtgagacttggggtatagtgacagttctactaatgtctgtgtgacccccaagaaagttgttttaggttcagagtaccttggttgcctcaaaggtaaaatgtcataaagccagctgtgtgtgccttccaggattgtaagagtaaaatgagatggtagctatgattttgaatgtgaaaagcattttaccgagactgtaatgtcctctttgtaaagtggagactagattataaactatagtatatatattgttttctccaagggaggaaccttgtctttattttgaaacagtaggtagcctcctggtggctggaacatgcttctgcctgcagggggcccattgtctgggccctagggatctacagagctcagtaggcttaggaagtgtttcaggaaggcctcctagttgatgctgtcctcatgtcctgccagaacagtctccatctcctcctcagccatcttccctcccagggtggtgaggacttgtctgagctctgctcccgtgactttgctgttcccctccttgactaacacatgaagcccctccaggtagtcctcatatgtgccttggttctgcttcttggctgctgcctggagcatgggcaggaaagtcttgaagtccacatgccgggacttcaggtcatcactcttggggttccccaggaccttcatcaccttggcactggtgaggttctggtccagggcccccatcatgtccccaaactggctgtatgggatcttgccatcccccactcagttaaacagcttgaaggccaccttgaactcctccagctgatccttgttaaacttgatcaccactttggagagatcaactgggggctccttggttttctcaggggcctgggggatgggatagctggctcagctttggtcttggctggaggggcccatgctgctgctggcttggcagcacctttggagatggagggccctgctggttcctcccagaatatccttgggaggcatgatgtctggtggccaaaggacagtgtgcggatgggggcacccatctcttgcagggtccgtgtctgggccagactacagtattttttttttttttttttttttgtggatggagttttgctcttattgcccaggctggagtgcagtggcgtgatcggctcgctgtaacctccgcctcctgggttccagcgattctcctgcctcagcctcctgagtagctgggattacaggtacccgccaccatgtccagctaatttttgtatttttggtagagatggggtttcaccatgttggccaggctggtctcgaactcctgacttcaggtgatccgtccacttcggcctcccaaagtgctgggattataggtgtgagccaccgtgcctggaccagtctacattattttaatgattagagttatccatggttcttctctttgaaatataaatcaatatggcagaaaatagtacttttctgtaacttccatctggtgatgtggttagctttattgctttggttgaaatatgattttttggttatttagaaacaaagccctttaagaagatcttcatcttaatgtctatgaattgaggcttaaagggaggaaaaaccaggttcatgtgtttaggagcaaagatagtggggaacaacttgttgatttgagtagttgtcataggtcatgaagtgtagcttgaggtaattgttaaagttttgtagacatgtcattagagaatgactttcatgtcctcgtcttagaaatataatgatttttatattactttttaaaatataaaagttgaggccaggcatggtggctcatgcctgtaatcccagcactttgggaggctgaggcgggtggattacctgaagtcaggagttggagaccagcctggccaacatggtgaaaccccatctctactgaaaatacaaaaaattaactgggcgtggtgactgcgcacctatagtcccagctactcaggaggctgaggcaggaggatcacttgaacctgggaggtggaggttgcagtgagccaggatcacgccactgcactccagtctgggcgacagagcaagactccatctccaaaaaaaaaaaaaaaaagttgaaataaggaaaataagactgaagaatttatgtgtgtgttgtataactagcttccccccataactttaaaaagctgttgtgtatcccttcttccgaaacatcttaaaatgaaagagctagaaatgatgtgaaaatgcttatttttcttctgcctgctcacattaatgctttacctccaaatttttattaatttaaatcttttctataatttgaatagtaatgtttaaaattgagttggctatcagatcatagttgtagatgattggggctcttatgatttagatagcagtgttttcagtcctttggaatatatgaagctagatttatttactgtttgttttcatagtctgacttttcacagaataggattttctggatatttcgatgctgtttggaatcaacttttgagcttcctgaggaatttagatttttgcttgtttcttttgtttttcagactcatgaaatggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaaggtaagacttattaaagataagaggtttgattcaggtctgattgtttgaaatgctcctttgtctccagtggctcaccatgatcatatagtggttagtactctgttcagctttaatgctgggtctgtaagaaagtgaaatattaacaagcgatagctttaatcgtaggtctccagggtttactgctgaagagaaagctagaattttcactgtaatatcattaattattggtaactaaaggtctggattaaaagctaaatgttaggccaggcgtgatgcctcacacctgtaatcccagcactttgggaggctgaggcgggtggatcacgaggtcaggagatcaagaccatcctggctaacacggtgaaatcccgtctctactaaaaatacaaaaaaattagccgggcatggtggcaggcgcctgtagtcccagctactcaggaggctgaggcaggagaatggcgtgaacccaggaggcggggcttgcagtgagccgagattgcgcgactgtactccagcctgggtgacagagcgtgactccatctcaaaaaataaaaataaaataaaaaactaaatgttaaaaggagatttcttttaatagagaaagtagtcgtctttttttgttattctttttttcttaatatgctttaagttagtccatagaatggactttgttcttttgggggttaatagctaaaatatttaaagcaatgaaactgaaaggaacagtacatgggaggaaaggtttatatttttcaaaaattggaagctgtcagtaggcttcttgttgacttaaaactgttagactaaggtgtacatgtaatgacatcaaatggaaagatatttttaaaatgagctctttttaagtaatatgtcttttacttgggacaagaattaaaaaacatggcctatatcttttgtcttttgtttgtctgtattacttgaagtatgttagaaagtaattcttgttgttgtatagaatttggagaggttaatagtgttaattaactcagtagaacctttaataatattaactgaattttatttatgatcatgtttaaagtgctatgaaaaccctttagtacatttttaagacaatgatacttaaaaaaaaaaactattttttagaagagttttagatttacagaaaaattgggaagatagaatttccgtatactcccatacccagttttcctcatattaacattttacattagtctggtacatttgttacaattaatgaactgttattgatagattattaactaaattacgtattttatttagatttcttgagtttttaccagttgttctttttatattctaggatcccatttgtcataccacattacacttagttgtcataagacaataaaatttctatttagcaaatgtttattgggtacctcctttgtactgggaactgttttgatagtgtaagggatctgtccagtaaacagacccaaaaaatccctgccttcatatgaagcttgcattttgtttcctttagtatgctcaagaaatttctaatgaaaagtgaaacaaataggagatcatatataccaagggtgtgattattggcctgtaattttgcctccttctttgaattttctttttttaaaatctaggctgggcacagtggcctgtgcctgtaatttcagcactttggaaggctgaggtgggaggattgcttgaggccaggagttcagtaccagcctgggcaacatagggagaacctgcttgtatttaaaattaaataaatgaaaaaggaaaaaaaaaatttttagttacgcatataatacatgcatacattgttgcctgaaaattttcatctacttccttctcattgctgaactctcagtctttaaataatgccaagcgttcccttgctacagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattaggtaaaacagtgttcttatttcattcccttctatatgctagatttttccacagtcattgtgtttcctaggttgtttatagcaggacttaactttcctgctatttggaatagtaagatactatttgaaatagtcaggtactgtttagagtaaataaaactcttacctcggtagtgaaagaatttattgacacctgaaaaaattcttaaacgtctcattaaactttaatttaggcaaggcacagatttcttgactattttttgattaattacttgcttgggtttcagcagacaacagtttgcaacatccccaggagaacacaggttagtttccactttgtgaatgaattctgcatctgttcttggggtgctagtgaaatgtgatatgcatgcctctggctgcagaactctgtcaactccattattgggctgccacttgctcttggccttctgtccttgttggggtgcaaactgtcagtgtggctgaataagctgagctcctctcatctgtctgttttccattctggtctactacacagtcatctccttcattttcatgctcttctgatgagtatgggctggggattggtgtcagaattgagagattagaaaaagagaggtggtttatgttttatttctgtcagatttttcaacttggagtggtttgcctaagtcaaggtgacaaaggaagtgggaggtggcatgatagaggggaagggatgcagcattgggaattagatgccccagaacaccagatctcgtctggtgtggctgatcttcccccagatagtggtggttggcagaaggatattttattgtgagttcagacttggtcagccagaacaaagagaaactgagcaaaagtagagtttctacccgaggactggcacagagctggctgcatcacctgcacaggttttctagggcagtgagcctcagccagggttacatcagccatttggagggaagaatcagaagatggggagtgggagagggagaaagaaaggagctctccttttattccagataatctctattttaacttttaacctcaataaactctccttagctcttgaatctgactcacaacaacactctttttgttgagggagctgttttgactaaatcccatgctgaattgagtgtaggatttttttatttctaaaatcaagttagactaaaatgtaatattttaggagttacaaattggcagcctgccaaaccttgccattggtattatgatcaacttgaactttaatatctccaggtggagcctttactgtctagtttccttactgtccttctcaagtctgcttgattatttgattctatcttgaaggcatttgagcttcagaagcctggtgttatttggccagtcaagttcttttccctattaaaagggctgtaattctataatttactagctttccgaccttaggaaagttatttaattttctaatgttcattttgcaagagaagcagctaaggctatttgcctaagggcatagagctagaccagattttctctctgggtttctttcatcttcagccaacatctgtgattctcttggttccaagaagggtctcactcatcctttttcagcggatgatgctattactactttcttatattcctaattggctaaatgccagggacttcacaagtttttgcctgctgcacggaagcacagccattatgtttccagtagcttcatctgtcttctttttcaatttgtagatcatggggagttgttttttgttttgagactcaagtttagttattctaatacagtagcaacacatctgaggtatgtgctaaaattaagatataactttatagaaagataaaagttgccaaagttttctaaaataggtacaattttggtagttacttcagtgattacattatcttgggaaattctttgtaaactttagatcccaacctctcatttctttgtaattcttttttttccctcccagccaaaacagcacctatctagcaaatggtgagtttgaggaagattcagagtggggtattttccagtttactttgccataggttatcagtttgatgggtttaagctaataggatgttcatcctttttgtcaaaacgtctttgcttcatgcttaggttttgtaactgccagaaataggcagaaacttcagttgtaattttggtctaaattaaggaaataataagtgacaactttggtgggcataccctattttggccatgaggctggaatttgggcttattagtgatagatttggcttcagtattgctatgtcaggtataaatgatcctttcctatttggagctgtggggttggggaagtgacagggaaggataggattggagttttagcaattttctggcagttttaatttgcttggacataggtcaggttttaccatggcaaaatttgccatcctggatgttgtgaagtctgtcagaatctttgaaggctgggctattaaacttctttctgatcatcttctgtatgagactttggatatatggtctacagatatagacagaactgaactgtttgatcctattgcttacctgtttgactttagagaccaagctaagttaaccctccttgattgctttttcttctgctaaatttctagcatgcatttattataggttggaccaaagggcttaaggagtcattaatgaattttggaccatttaagtgatgttcccattccaggcctgaaaaccatcaatggtctcagacaatgcttgtcttaggcagccttgactcgcttgtaaaacagcattttacatgagaagaaatactttcagtagcctcttacccttataaagacttgttttaactctcaattcatataaccgggtgggagaacataattatatgccaacaataagaggcagtttccatcacatatataccttgttgtatatagatactagggactgttaacaaggttgctttttttttaatccaactcagacacaagaaggaaagcagaacctacctttggaggtcatgaccctcgtacagctgttcagcttcgaagcctcagcacagtattaaaacgcctcaaggaaatcatggaggggaaaagccaggtactgtctagaggctttggaagattacttattaagcgcttatggggatcatgccatacctaatgaaaatggcttaatgccactgaaagaaagcttaattagcgcttcctccagctgtgagcaccctatttttatacataagttcttaattttattgtagtctaatttatcagtttttttcccgttatccttagtgcttttcgcgttctgtttaagaagtgtttatctagttcagggcagtagatattctcctatggtttcttcaagaagctttattatttctgtcaagtataggggtcaggcttccatttttcctcatggatgtccagttggatatagtaccattgtttttgaaaagaccatctttcactgattgcattataaattccaccgtaagtccatatatgtgtgagggtgtgtcagggctctctttttctgttccatacctttatttttttctatccttgtaccagtaccctgtctaaaattattgtagcattaagaataagtcttgatggctggtaatattaagtccttcaatttgttcttcttcaagattgtcttggttattttgacctttcgtattcccatatgaatttttgaattagtttgttttctgcaaaagaaacctttgaggatttcatttgccattgcatattaataatcagtattttcaagaagccttcacacatttttattgttataggatagtgacctgaaacaatactggatgccagatagccaatgtaaagagtgctatgactgtagtgagaaatttacaacctttaggcgcagacaccattgccgactatgtgggcagattttctgcagtcgttgctgtaatcaagaaatccctggaaaatttatgggctatacaggtaaatgcattttattgccagtttacaatttttaaaggtcataactataagacacttataatacaggatagtgttaagccaattttcattttgggttttatgtgtaagacatggggacacaaaggtgagggagacagctttgcttttaagaactttcctattgaataatacagtagatgggacttatcataaataactagagtaaaagtgtatttattgtagatgctagcaaaaaactataggaacattaaaatttattagacaagatcgttagagtagattactaatttaaatcactatgagagttttcattttaaaaggacagatcttccttgacgtaattacatgacactgtgctttggcagtgaattgtgtcttactcagattcctccataaggaacatagtcaaatttcagcagtacagaaaatttaagttatttcttgaaacagaaaaaactgaacaatggtatgttgatttatacagtgatttgggagtagaagatagtcagaaattacctgaaaaacaaaactcctaacatctctctcatgttatttttgttattgtgaagatctttctgtctgctcatttttttttccacctaaactactacagagttcagaatcccattatctgtcacccagaaccccattagcttttacaacagcttttttgtttgaaaattacttgtatttctttttttttttggtaataatttcaacttttattttagattcatgtttgttacaggggtatatttagtgatgctgagattttgcatatgactgatcctgtcacccaggtagtgagtatggtatccagtagttagtttttcaaaccttgctcctcttcctccctccctgctctagtagtccccttgtctattgttgccatctttatgtcctcatgtactcagtgtttagctcccacttgtaagtgagagcttgcggcatttggttttctgttctgtgttaatttacttaggataatggcctccagctgcatccatattgctgcaaaggacataattttgttctttttttatggctgcatagtattccatggtgtatatgtaccacattttctttatccatttcaccattggtggcacccaggttgattctgtatttgctattataaaaagtgttacaatgaacatacgagtacatgtgccttttgatagaatgatttattttcctttggatatatacccagtagtgggattcctgaatcaaatggtagcagttctgttttaagctctttgagaaatcttcaaaccgtcttccacagtggctgaactaatttacattcccatcagcagtgtataagcattctcttttctctgcagctttaccagcattcgttttttggctttttaataacagccattctgattggtgtgagatggtatcttattgtggttttgatttgcatttcactaatgatcattgatgtggaacattttttcatgtttgttggccacttgtaggtctttttttgagaagtgtctgttcatgtcttttgcccaatttttaatggggtcatttgttttttacttgttgaattacgtttcttatatattgtggatattagatctttgtcagatgcatagtttgcaagtattttctcctattctgtggcttgtttgtttactctgttgatagtatcttttgccatgcagaagcactttagtttaattaggtcccacatgtcaatttttgtttttgttgcagttgcttttgaggacttagtcataaattgtttcccaaggctgatgtccagaatcatgcttcataggttttcttctaggactttcatagtttgaggtcttatatttaaatctttaatccatcttgaattaattttcgtatatgatgaaacataggggccagttttattcttttgcatatggctagccagctattccagtgccatttattgaatagagagtcctttccctgttgttcatttttgttgactttgttgaagatcaaatgtctgtaagtgtgtgattttatttctggtttctttgttctgttccattggcttatgtgcctgtttttgtaccagtaccactgctgtttttgttactgtagcctattcctatgaaactaccaatgtcgtttttcatagaattagaaaaaactattcaaaaattcatatagaaccaaaaaagagcctgaatagccaaaacaatcctaagcaaaaagaacaaggcaggaggcatcacatttcctgacttcaaattgtaatctaaggctacagtgacaagattacagcttttaaaatacattttttttacctttagtttctccctacctctaattcatatttcatatcctagtcagcattctttgtaaaatgataatttgatcatgtcagggtattttcctgcataatactctttcagagctcccattaccctcaggataaaacctttcttgttaatatggtcggtatgttagcttcctaggtctgccataacacagtaccacaggctgtgtgtcttggaacaacagaaatgtatgtctcatagttctggaggccagaagtaccaaaatacagatcagggaacaaatatagggattctgctggttgtctattctaactgtatgttctagaactggggaaataaccatagagtgagtttggggactcactaaactcacagtgagatggacctgagctaaagcttcactgatcacctgacctctgtaagtccctactctgacaggcagaccagagtgatgttttgggtctcctggaattagtgtcagttgagagtaggtgtctaataacccctgacaaatatgatcatttccttctcctcagtgtccagtcgccttggtaaaagcataggtctctttggggatggctggtagaaagattaacggtgtacatttctggcagtgtagtggggttcttcttcagaatgaccaatcccctcttcattcaaggagttctggatcatctgtaagtggctcaagtctgggaattgagtgaaggatcgtatgactctgttttcatgattcaagttggacttacagctacttgatatggagttgtttcccttatacaaatcaagtaagaattttgttgactgcccatctacttcacttgtggagacattatgatcaactagccaatgccatacatctcttaggctattctgattattgctttgattctgatgtctattatagtaaccatgcccaccttgtctttggcgactaaagtgctgcctattggcccctgccaccctgggatccagtgatccccattgcatttaggtttcctgacccagtagcagtagttcctgctgtaatttctgatctacaaagaagagcaaccacaaagctctttaaggattcggggcctcccctcacaaatttatttctcatagtcgtggttgaaaggttatgtccttcttgggtgagtcagcaagtcttacatggtaaaactacttttaacatttcaatctcccctaacctttggatgccttcttctataatataatagggcatttctgatatttcaaccccatttagtgtaagccaccttttggtctatgtttcagccagtcatctaaacagactgttggagacccttttaaccccttgagctgcaacaataaatatagaatctgcttagtgggcccatgtcagtaaattcagcttgatccaactttatattctttccactattatcccataccttcattatccattcccacatgtatttccttggtttttatctgtataaattggaaaaatcatgtagttcttctggagtgtagtgcaccttctcatgggtcacagtttgtatctcacctttcagggcctacctgagcttgagttcctttataggtctggtagcaaagaggagtagtaggagtagatcctgagtaaaattagcagtgtcttgcaaggcagctaactcaggggaggtcattacagcctgtctgatcaaagccaggttaatctcctcaaatgaggtggaaagactgctgctgctggcaaagaagactcagaattttggtgtttaacatccccagctccatcaggatcttcttacatatttccattccttcccaatcaacaccctcactttaatagcagacaaacttcaaggttggtgattcagtttgtgtttcagttcagtcacttgcaggatgagagtgttgttctgatttttaacaatcttagcactgaacctgcaggagataaggatttctttcaaggaagacctagaaactttcagataatttatacagttcttgagctgggaatttgaatccctaaggttatccatttctttttttacttttgtctaatgcaattaggaggaaccagcccatgtcattatagttgttagcttgacaaaaccattctaagatattaaatacttggtcactcagaactttgccttttataagtattaatatttgattaggagtatctgtaggtgatattttgcatgtctttgttgccacattacacaatggactatcagtgttctctttactactggaaatagagtcattagtgcctttaaatctaatcagcttagagaaccaattccagaaatgtaagaaccaattcagaaaacttacccttaagattctgttgctctaaagccattcttggtaccaaaatatgtattaaggttctccagagaaacagaaccagtaggatacacaaacacacatttatatagagagaaatttatgtttaacaattggtcaagttattttgaggactggcaagtctgaaattctcagggcaggccagtaggctggaaactcctacaggatttctgtcttgcagtgttgagacagaatttctttttcttcagaaaactttagtctttgtccttaaggacttcaactgattagatgagacccactcacattctagaggacaatctgctttacttaaagtcagctgattgtaaatattaatcacatccaaaaaaaataaccttcacagcatctcagcttgaccaaataactaatctagccaagttgacgcataaggttaatcacaaatgggataatatagtggtctccaacctttttggcaccagggaccggttttgtggcagacagtttttccatggactggtgtgggggacgggggatggttttaggacgaaactattccaccttaggtaatcaggcattagattctcgtaaggaacgtgcaatctagatccctcacatgcgcagttcaccatagggttcacgcttctataagaatctaatgccaccgctgatctgacaggaggtggagctcaggaggtaatgcccgtttgtgtatggcttgtgtgctgcttgctcgccactcacctcctgctgtgcggctcggttcctaacaggctacggacggggagtagtacatgtccctgggattggggacccctggggtaatatatgctaggcacagttatacattgtgaccctgaataaatacaaataactagaaaaatgtctagtacatggaaagggatcaatgaacagtaaactattgttattgatggtaatagcaatagtatctgcatctgtcatgcagctttttggttatatgtgtgttgcctccagtggcctatgagttttttgatggcagggctgtatctcatcaatctttgtattcccatcacaccatataggcatgcgcttaataggctcttgctgatgaactttcttcatgaaatgaaaacttttatgattaggcttggctctgtttattgattttacaattttagttctaatagctgactggttaggcgagatttcctttatagtgtaagaaacaaatgaagtctttgttgtagtaaattgaaagtctttctgcagggagtaatttcacttcttttgtttttttttttggagactgagtcttgctctgttgcccaggctggagtgcagtggcacgaccacagctcagctcactgcagcctcagcttcctgggctcaagcaatcctcctacctctccgcctcccaagtagcttggactacagggtgcatgccaccatgccctggctagttttgtattttttattgatgtggggtctcaccatgtagcctaggctggtctcaaactcctgtgctcaagcagtccgcctgccttggcctcccaaagtgttgggattacgggtgtgcaccaccatgcctggcctacatgcataatttaataaaactgagtactgtgtaatagtagtcttaaaatgaaaaagtaaaaggccattttggtagtgattatggtaaaaacataatacagttaacttttagaagtcttttattttgtatcacttcagttttctaatattagtgggaaaattttaggtggaattattaaacaacatgttattagtaaggtttaattattttacctccttttttaatacctaaaatgttacagaatcactcaaaacaatgttatagaaaaatagagtagaaatgaaaacttctatttggacaggactgcaccctcaaaaaatacatatttagaaatattaaccttgtttgtcctctcaagaatatttcatttctatccttttttttttttttttcccagacagtttcgcttttattgcccaggctggagtgcagtggcacagtcttggctcactgcaacctctgcctcccaggttcaagcgattctcccaccgcagcctcccgagtagctgggattacaggcacccgccgccatgcctggctaatttttgtatttttagtagagacagggtttcaccatgttggccaggctggtcttggactactgacctcaggtgatttatccgtctcagtctcccaaagtgctgggattacaggcgtgagccaccgtgcccggccttaacttttttttataataattcttttatagtttttactctcatatattatactgcatgtaagcccattttctgttagtttgcttttgaaattctttggagggtactcttcagggcttcacaataaagaccttaataatattttcctccttaattaatttgcctatggaaaatatgttgtttccatttgtgttggtttatttattctattaaaaaatgttattccttgaaactcatgtctctgttagatttcttttttaagattttattttatatatgtttatatttgtatagagacagggtctcggtatgttggccaggttgttcttgagctcctgccctcaaacagtcctcccgccctcaagcagtccttctgccttggccccccaaagtgctgggattacaggtgtgagccagtgtgcctgtcctagatcccttatttcacttttaaaacctggaaaaaaatactcttttaagcaaaattttctggtatcattgggatatacattttgttttgttttgttttgttttattttatttatttatttattttgagacggagtttcgttcttgttgcccaggctggagtgcaatggcacagtcttggctcactgcaacctccgcctcccgggttcaagcaattctcctgcctcaccctcctgagtagctgggattacaggtatgcgttaccatgcccggctaatttttgtatttttagtagagatggggtttcaccatgttggccaggctagtcttgaactcctgacctcaggtgatctgccgcctcggcctcccaaagtgttgggattataggcgtgagccaccacgtctggcctggtatgttacattttaaactcattataaatgtttatggtagtttggacacctggatgaatttatcataatgatggctctttttttttcttttaacttctaacttttgatttttagtgccagaattcaaaatggcccttaaatatatataatagagctaacaaaattatttcactttttaaaatgtctttttttgagagtctgtacttgaagacatttccgttattagatgaactgttgacccaaggagttaaaaaagttacgttaagcaatatgttactgctatcttttccttcaatttccttgtttttgtttttgtttttttctcctaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaaggtattgtcccttaaatataattttatttagagttgaaaaatatcgatagttgtctgacctttgagtgctttcagttaacacttaccctcttagaattatttcctgtcagttcactgtgttctcctttttctttggcttgcctccagaatgtttgcagatgtttatggatagtgatgatattggctaattagaatgtaagctccatgagaggagactttacatctttcttgtcactgttctaccccaggcaccaagaacagcgcatggcatgtatataatcttgataatatttgttgaacaggcaacaaattgagatttttaatttaattaatgaacagaagtcattgagattattaatttctaatgtagccttctgtgagaagaagaatgcctgtacaatatgtattcatatttccaagtgaaattagattttttttccttctgaaggtatatgtcatgtttttctgtaagtatactgcactgctacttcatgtttttttgtaagtatactgcactgcttggtagtagagggtgctaactcttggaagttacctggaattgtttgaattagagtcttagtttcatcagtttctgatttttaaaaattattagttattatttttgagacggccttgctctgtcacccaggctggagtgtagtggcgtgatcatagctcagggcagcctcgaatcccctctgccttagcctcctgagtagctgggacaacaggcgcacaccactgtgcttggctgatttttttttttttttaagagatggggtcttgccatgttgaccaggctgtcttgaactcctggcttcaagcgatcctcccacctcagcttcccaaagtgctgagattacaggcatgagccactcactgcacctggcctctgattattttctttagctgaattattttgttaagcataataccaaatctggccgttacaaagagcctatagttttaagtaacatctctctgataagttaatttctcagttaacccttaaggaattgagaaattagtatgtaatgaatttaaatagaagtaaaaaatgaattaattacagctacattcaatatgtatgaatctcagggacataaagtggagtagaaaagaaaatcatctaatcatgtcaccgggaaaatttaattaatataaaatttaaaaatttaaaaatacacaaaactactacactgtctagggactgaaatgtggtaacacttaaagacgatagatatgaaagttataacagtggtttcactgagggaagggaagggattaggactgggtaggggaatttcacagtcagtggtagtgtactttttctttttcttttttttttttttttgagacagagttttttttgctcttgttgcccaggttggagtgcaatggcacgatctcggctcactgcaaccttcttttcctgggttcaagcgattctcctgcctcaacctcctgagtagctggacttacaggtgcccaccaccatgcctggctaatttttctgtttttagtagggacggggtttcatcatgttggccaggctggtcttgaactcctgacctcaggtgatctacccgcctcggcctcccaaagggctgggattacaggtgtgagccaccgcgcctggccagagtttctttttcttacattagtttttatttatttatttattttgagatggagtctcgctctgtcacccaggctggagtgcagtggcgtgatcttgactcactgcaacctccgcctcccaggttcaagtgattcttgtgtcttagcctcccaattagctgggattacaggtgtgtgccaccatgcccggctaagttttgtattttcagtagagatgggttttgccatgttggccaggctggtctcgaattcctaggctccagtgatttgcccgcctcccaaagtggtggcattataggcatgagccaccatgcccagccttttcttacattgcttcatgaatactttggtgtttatggcattatgatttttttataccttacatatattttataaattttaaaaattgattctatgtttaattaaacaatatgaagtatagagtttttttagtttttgcttactttagttagtaatgctttttagttgtgacaggccctgtcgtaatgggcagatgtgatttaacaaaacaaatcaggtaagaaagctccctgagagatgtacaatagatgtcaaaggtagaatgattgttaaattttaggttctgtgctaattgcttaatgttcattctcttttagtcagcatatctctgtggtaggcactattttctccttttgacagatgtggatgctgaggtttagcgagattaagtaatgaacccaaggtcatgtagctagtaagaggtcgagcctgcacttggacctaggtttgtctgactctatggtgctattctagtgggttatggggactgagaataaaatatttgaactcaagatttccctccaaaatcaatttcatcttgtattaataatgttagacgtttcaaaggcacccatccaactaactttatttacattccatatacttaaaaactttttaaaattgtcatttcggcattttatattcttaaaagccatgcactgcaaaaactatcattcataccatttacaccctaattaaaggaagaacaaaaatgacacataatctcattattgcaaggaaaaagattaaatctgcttttaatgttaaagcgcctcattgaaattcccttttactgtcctgaattatttcagtattttcctattttctttatagtttgattcatccagattcctcaaatactcctctttcaacaagacttgtatctgtgcaagaggatgctgggaaatctcctgctcgaaataggtaaactgacaaatgaaaacactgtgctctctgatgtttatttcttttcatgctaataagaagaacaaacggataaaaagggtgggattgattgtcatatacttccgtagctctaaaagctgaattaaactatcattttctttttgctattcagcccttgtttccattctctcagtgactttccagtattctccagtggcttccgggtttggaacctacaccatcctttctcttctggttaaataagacgtgaaggatgtgagggaaggagccatgtggacattttgtggaggagtgtcccaagaaaacaggaataacaaattcctgagaggaaatacacttggcaaggaataacaaggaatcctatgataatggagtagagcagtcttagagttaattataaggactctgtcatttactctgagtgagatgggaagctgttggagagttttacattttttgtcgttaatttttaattgctttgaaaaaatgtcaaacatgcagaaaagttgcgagaatagaaaaatatatcttcatgtatttttcattaagatttactaatttatattttgtccctttgctttctcattttttctttttttaatttttatttatcttattattatttttgagacggagtctcactctctcgccaggctggagtgcggtggtgtgatctcagctcactgcaagctctgcctcctgggttcatgccattctcctgcctcagcttcctgagtagctgggactacaggcgcccatcaccatgcccagctaatttttttgtatttttagtagagacggggtttcaccgtgttagccaggatggtcttgatctcctgacctcatgatccacccacctcggcctcccaaagtgttgggattacaggcatgagccaccgagcccagcctgttattttttagtaaaatacagagatggggtttcactttgttgttcaggatggtcttgaacttctgacctcaagcgatcttcccacctcggactcctaaagtgctgagattacaggtgtgagccactgcatctggccatcgttttttatattgtatatacatgtatgcatattttctgaaccatctgagagtaagttgcagacatcattatcctttatatatttttattaagtttattttttatagcagttttaaggttcatacaaaaaatgagcagaaagtacagagagttcccctatagcccctgtccccacacatgcacaatcttcatgactatcggtgtcctgcactagagtggcatattgtattcagtggacctactttgacacatgatcatcgtctaaaagttaatagttaacattagaattcactcttggtgttgaatattctatggatttggacaaatgtataatgacgtgcgtctgtcattgttgtatcctacggaatagttcctctgcccaaaaagtctactgggcttctcctatttatcccttcctctccccaacccatgacaatcattaatcttttttactgtctccatagttttacttttcccagaatgccatatagttggaattatacagtgtgtagacttttcggataggcttctttcacctactaatacgcatttaagttttctctatgtctttccatagcttgatagctcatttctaagtgatgaataatatcccattggctggatacactatagtttatctactcacctactgaaggacatcttgattgcttctaagttttatcagttacgagcaaggctgttataacatctgagtataggtttttttgtgggtgtaattttcaattcatatggataaataccaaggagcatgattgctgagtcctatggtaagattatgtttaattttgtgaggcactgctagacttttttccaaagtggttttaccattttgcattcctaccagcaatgaatgagagttcctgttgtttcacatcctcatcacatttggtgttgtcagttttggattttggcagttttaatgggtgtgtagtattatctcatgtgattgacttttgtatattaatcttgtatcctgccaccttgctataatcacttactaatttcaggagttcgttagttcttttggattttctatataaacagtcttgtcatcgaacaaagttttgtttcttccttcccaatcagtgtactttttatttccttttcctgtcttatttcattagctaaaacttctagtccaatgttgaaaaggagtagtgagaggggacatcctttccttgttcctgatcttagtgggaaagctttgagtttctcaccattaagtatgatgttagctgtatgttgtttttcgtagatactctttatcatgttggggaagtttcctctattcctagtttactgagagtttttataatgaaggagtgttggattttgtcaaatgctttttctgtatcaatgacatgattacataattattcttctgtaacttgatgtgatgggttatattaattgatttttgaatgttgaaccagccttgcatgcctgggataaattccatttggtcgtggtttatatagttttttatacattgttggattggatttgttactgttttgttgaggataatagtgtatggtttccttttcttgtcatgtctttgtctggttttggtattagggaaatcctggcctcacagactgagttagaaagtatttcctctgattctatcttcaggaaaattttgtggagaattgacataatttatttcttaaatgtttggtagaattcaccagtgaatgcatctgggcctggtgctttctgttttggaaagttaccagttattgattcaatttctttaagagaaataggtctgtttcgattttcatttctgatattagtactttgtgttttctcccttttttttctcagcctggctggatacttactgattttattgatcttttcaaagaaccagctttggctttgttgattttctctattgacttcttctctaatctttattattatttttcctgtgctcactttggatttaatatgctttttgacaaacactttgttccctaaggtggaagcttagatgattgattttagatcttctttttaaatgtatgcgcttcgtgctatgaatttcctgctaagcactgctttctgttgcattccacgaatttagttaggttgtgttttcattttcatttagttacaaatatttaaaaatgtttctttatattcttctttgactcatgtatttttttttacaagtgtgttgtttcatctccaaggattttgggattttgtagctgtctttctgttaatgatttctagtttgattccatgttggttgttgaaggcagacattgtatgatttctactcttaaattttttatggtgtgttttatggcccagaatgtggtgtgttttggtgaatgttccatgttacttgagaggaatgtgcattctgttgttgaattaaacagtctataagatattcattatatccacctgattaatggtgttgttgagttcagctatgttgttattgattttctctctgctggatgtatccatctctgatagaggggttaaagtctccagctataataatgaattcgtccatttctcctgtagttctatcagtttttgcctcatatattttggtactctgttgttaggagcgagcatacatattaaggattgttctgtcttttggaatattgatttctttatcattatgtaatttccctttttatctttgattgctctgaagtctgttctgcctgaaaattaatatagttactcctgctcttttttttattagcgttaacatggtttatctttctccatctatttccttttgttcatgtatcctgttttgatttcatttaaaaatttaaaaattaatttttaaaattgacacataataattgtgcatatttatggggtaaatagtgacattttgacccttcacttattttttaatctgtatgtgtctttgtatttaaattaggtttcttgtagacaatgtatttttgggtcttggttttgatccattctaaccatctctgtcttttagttggtgtatttagactattgacatttaaggtgattactgatgtagttagagtaatccctgccgtatttgttactgttttctacttgttgcccttgttcttcctgtttttgtcttctgtaccttttccacctttatgtatttaattgagcattttatgttattccattttctctcctttttaaaggatacgagttacattctttttttttttttaatgattgccctagagtttgcaatgtaacttacaactaatccacatttactttcaaataatactatactacttcacaggtaatgcaaataccttataacaaaatactcccaatttttccctcccatctgttgtattactggcattcatttcacttacatataagcatgcgtacatacataattgaatatattgttgctattattttgaagaaatagttatctgttacatcaactaagagtaagaaaaagaaaagtttttactttaacttattccttctttgatgcttttctttgtgtattaatagatgtaagtttctgacctatatcatattccttctctctgccttttcagcattttttgcaaggcaggtctactggcaaaaaattccctcagttttcatttgtctaagaaaggctttacttctccttcacttttgaagggtatcatcagggtacagaattgtaggttgatggcttttttctctcaacatcttaaatatttcatttcattctttttgcttgcgtggcttctgaggagaagtcagatgtatttattatctttgatactctacaggtaaggtttttttttttttttctttaaatagagatggggtcttgctgtgttgcccaggctggtctcaaactcctggcctcaagcgatcctcttgctttggcttcccaaagtgcagggattacaggtgtgagccaccatgcttggtctttaaggtggtctttttcaagatctttttctttatttttgactttctgccatttgaatacacttagttttgcctatgtgtagtttttttgacatttatccacgcttgtgttctctgagcttcctggacctgtggcttggcatctgacattaatttgtgggaaattctcagtgattatggcttcaaatattgcttgtgttcctttctctctttcttcttttttcggtattcccattacatttatattataccttttgtagttgtcccacatttcttggatattgtttgttcagtcttttttctatttgcttttcagttttggcagtttccgttgagatagcctcaagttcaaagattctttccttacctctgtctagtctactaagagtccatcaaaggcactcttctttaatgttatggtgtttttgatctcttgcatttcctttttgcttctttcttagaatatccatctctctgcttatattatctgttcttgcctgttgtttacttttcgcattaacacccttagcatattaattatagtttaaaaacaattcctagactgataattcccacattcatgttgagcctgtttcggatgcttgttcagtgtctttgaactgtgttttttggctttcattatgccttgtgattttttgttgaaaggtggacatgctgtactgggtaaaaggaactatggtaaataggcctttagtgatgtagtgataggatgtagggggaggttaagtggtctctagtcctgtgaatcactgtcacaggtgagcctgtgcccctgaactgtaaactttatcagggcttttcgtttttttccccttaggtgagacagaatggctggagggggctggagttggggatttttcttcccccatgtggaaggctagaaggggctgtggttggggatttccttctccctgggtaggttaagctctgatataactccagcaggttaggctctggtaaaatagtttctcctcagggcaggacttgttatgaagaacagaatgctctgacgtatttttaagttctttattctctccctctgctggaagccagaagccggagggaatttttctgacattcactgtgaggacgtggtagatctggaagtgaaactcccaaaagcatccccactacccccatgactgggttcacctagaatttgtatctctggtttgtctacactgagcttcaaggaatttgtcaatttgattttaggttttcctaccctattactggttcccatggaggtttctgctccagtgagttgtgatcctctgtatctgcttgtctgtctgcagtatttggagcaggatttggcttgtgacctgacttctctgatggacctaagaacagttgatgatttttctctttgttcaggttttagttgttgagatggagtgaagatgctcacgtgctagaccagaagtagaaagttctattttttacttttgaatacttaagtgtttttcctcagaacaatcatgttctcttacataacaaatttaggaaattgtaacagtggtacatttctattatgtaatatactgtaggtattctgattttgccaattgtcctaataatgtttttttgtactgattttccccccaatctgtgatgcagttcagaatcatattgaacttatttgtcatatgtcttggttttctttactctgtaacagtatcttagtctttctttgtgaagaggatagaccagttgtttagtaaaatgcccttcaatttaagtttatcaaaagcttcctatgattagatttgggctgtggagttttggggcagtaatactccatgagtgctacatccttctcaatctgttatgttataaaggacacaatacaagtttatcccattggtgttgttgagttgatctctcacatgttaggtttctccactgtaaagttactattttatcctttgtaattggtaagtaatatgtgggggagatactttgaaactgcatgaataccctatatcttactaatctttcagtcaatagtttcagcattcatttatgatacttgttgaaatgcaatgttattatgtttgcaaaatggtgatttttctaagtctatcattgcttttacacttattagttgacattctactctgaaaagctttccgttgttggagagttttgggtggagtgtggaaggatgacatgacattttttttttttagacagtctcgctttgttgcccaggctggagtgcagtggcgcaatttcacctcattgcaacctctgcctcctaggttcaagcaattcttgtgcctcagtctcccgctagcctgggattacaggcacctgctagcatgcctggctaatttttgtatttttaatagagacagagtctcaccatgttggccaggctggtctcaaactcctgacctcaggtgatccacctgcctcggcctcccaaagtgctgggattataggtgtgagccaccacgcctggacgaaacagacaaattctaaatatattttgattgtagagctaataggatttgctaatcaactggatgtaggcttcaacagaaagacatcaaaggtgactcaggtctttgacctgagcagttggaggggtggaattaccaactgggaggaacagtttggttttagatgtgttgaatttgagatatcttctagacatccaagtgagatgtcaaatagttggatatatgagtccagaatttaggggagggctacaggctagagatgtaaacttggatgttgtcatcatgtgtatggtatttatgaacttggatggagatcatcaagagaatgagtgtgaaaagggaggaaataaggactaaggactgagattggagaaatcaagagaaaccagaaaaggagacggggagaggccagtgaggggacagggaaaccaggagggtatttatcttattagtcaagtggagaaagtttttcaaggaaagggagttatgctgctgatatgttaagtaagatgaggagtgaaaactgactattggatttaacaacaacattggtaatcttgataggcagtctcagtgggttgtttggggtttaagagataatggaagaattactggagatgagagttttgctgtaaagtagagggaaatgcagccgagggatgtggagtcaggagatggctttttaaaaagatggggaaaattacagtattcctgttggcatgtgtagaagtgtagggatacctgtgctaatttacagtctatctccatgtgttttgaattaatgctaagactgatgtaagtctttaggaaagacgtaatttagtcaatgaatgctgtctctaaatataattttaatcagtattcttgctactaggctatttattacagcttttgacattaaaattgactaattttcttttattcttgttgccactatattagaatatgtgcaaggatggttaaaatgatagagaagtagaatgtatggataatgcacccattggaaaagagattaaattaagaaataatagcaagctttcactgaaagactatgccaagaaataaataaccaattatttttaaaaattgcttatcggctatttgatatcagtgatttcctgttaactgacactttgctaaacctgtgaatactttcttaaattatttaaaaaatatttttatctttatactggggaaatattggtttttcaattgacacctaattctaggtggcagccctgatacatattaggcgagctactttaggcagattacttaatcccttattcttgatttctttatctgtaaaatagagctagtaatacatattccataggatgctgtgaagattaaatgtgggaagatacagatactgccctaatgaatgagttacatatatgtagtctaacatatcataggtgcttttgccttttatcctttatgtggaggctaaaattgggtttaatttttttctagatagaagccaactaaatttctagttaccctttcttttaaataaattaaagtttccttgatgctattggtattccacttctaagtcatttgtttggtaactttaccaagagtcaaaattaaggatatgtatgaatactgatattatgagaagaaacaaatttcttgtaatttttctactgacaaaatagcataatgtaataatatttgaggacaatattttaaatataggtctactagtgagaagaaaggaattctttttttggtcataacattttgcttatttgggcgtcaaaattggtgttcccttcatcagattgattgcattgcagatatttaactataaaaccttcttagttcatgggctatacagaaagaggaagaaggctagatttggcctgtgggccatagtttcctgactctgatctgcaagattatttctgcatttaatttcattctttatatttgttattaaaaaaaaaatccactaacttacagcattttaacaagaagactttgtatctattccctttgaaatattactgatatatgaactatgtaattgttttcctgtaatctttccataactactttcttttaaacttatttccaagtaattttagacacacatagaattgcaaaatatagacttacatagggttgcaaatatcgtacagagagtttctgggtatcctttaccaaatttctcccaatgataatatcatgcataaccatcataagtacttttaaaagtacccttaacattggtaaataagcatttatttaaaaatgaattttctctgtaattctgtttattgaagaagaatttacacctgtgatttcacttcttgtttttatttccagatcagccagcattactaacctgtcactggatagatctggttctcctatggtaccttcatatgagacatctgtcagtccccaggctaaccgaacatatgttaggacagagaccactgaggatgaacgcaaaattcttctggtactggtccagtatctttgacccattgctagttttgagcatagagaatgttcatgtgactgagtctttttttttctttttgagacacggtcttggtgtgctgcccaggctggggtgcagtggtgtgatcttggctccctgcaacctctgcctctcagattcaagcgattctggtgccttagtctcctgagtagctgggattacaggcgcatgccactacacctaattttttgtatttttagtagagacatggtttcgccctgttggctaggctggtctcaaactcctgacctcaagtgatctgcccgccttggctcccagagtgctgggattacaggtgtgagccactgcacccagcctgtcactgagtctttttaataagaaatatataagtgaaatagcatatcagttaaacagaagtgcttagcttcagttttactaggatcacctgagagatgggtcaagtattctatactcattgcattaatattttcttttctttctttctttattattattattttttttggagatggagtctcgctctgtcaccaggctggagtgcagtggcgcaatctcggctcactgcaacctccgcctcccaggttccagcgattctcctgcctcagcctcctgagtagctgggactacaggcatgtgccaccacactgagctaatttttgtatttttagtagagacggggtttcacagtgttggtcaggatggtctcaatctcctgacctcgtgatctccctgccttggcctcacaaagtgctgggattacaggcgtgagccaccgcgcctggccagtattttctttacccttttatctggtaaaatcagaattcagttacctatgtaataagagagcttaactcttagatcccatattgctatttaagctttgaagcatgtttgttatacacatgtaacacttttcagacatttaatactatatgtttatatgaaataatcagttgcttattgaatttatctgtgcataaaatactgtgttcaattctaagagtgactcagagaaatggaaggtatggctgtgtcctcaagcttataattcaaaagtagaaataagagccaggtgtggtggcatgcacgtgtagtcccagctacttgggaggctagggtgagaggactcttgaggccaggggtttgaggctgtagggtgctgtgatcatggctgtgaatagccactgcactccagcctgggcaacatagcaagaccttgtctctaagacaacaaacaaaccagcaaacccacaaaaggtagaaataagatcagtgggaagatccttaatagtggaagtagtaatactttaataataaaaaagtgtcaaataaataaatgactatatgtcatgtaagaattcaaaaaaacagaaagctcattactaatgagggaatcagaaaaaggcttaaactgaaggacttgaactggaccttgagagatggaatcattttggataggcagtgaggaaggtgaatcttatttaaaggtgtgaagaaagatacagaggtaagaataagcatgacattttccatggccagaagtaggattttttaattgggagagagtttaagtaggagtctgggggcaatgagaaaggaaagttatttgggactacctggtggaaggcaatgaattctgtacttacaggtgtagatttgaaactttagttgctgaggaacattcagaatttttttgagcagggagtaatttactgaagctcttctttttaaaaaactcatctgtcagttgggtacacagtgagaatctggtggagtgaaagcctctaggaacagttaccttaggttagtgtgattaactgttgtctccctctgccttgccccatctccaaagaagaatacataaataggagattgataatatgtgccattgactgaaaaaagaagtatttggaatagttggttttcaagagaaaaagttatgtttggaattagatgagcagagttcaagctgttcccacgtatatgacatttgtattaaattgtgtgagttagctaccataaagaaatccaagttaggtttccttttatgagtctaaaaatcacaggaaaacatctgaaaaggtatatagcatttttctcaagggtaactacttgctgttaatgtgattgcaggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaaggtattctgatcttagaaggtttcaatattatttgtttattttgaatgagaaaacatatttgaagatagtaagagttacagatttctttgtagagttagggtgctctttgttttatgtaattgatggtttagggtgctaattacttaaccctcctccaatctgagttttaaccagcagagaacagatgcaaggagtacaaggagccaaataggatcttaatttaccttattaaaagcagaagaactctctagcaaaggaggattagagaaggtagctggaacctaaggccaaagaattccccagctactgttcactaatctagacaggttgtaacctgtgttgagtatttgatccataagctatgcctgtcgggtcctagcttgcctcacaaatcatttataaaaaagagtaacagacaggtcctgcttttcaaatacatgtatcccctagcttttactattttctcagattagctactcctcctctcatgggaagaaaaaggtacgtaatgaagtaaagagagaaatagctttaagcagtaaagccctggttgcaattttggctgtatatattttttagctttgtgcttttagtcacattattcaatctctgagacttcttcaccatgaggaaaattgtgtggtcctgtgaataaactgtgggggatgaaataatagtggctgaagtgctttataaattgtgaaatgccatataatgtatggtgtcattaagtgtctatttcatatatatatatacacacacacacattttatggctgtatctgtaatatttggtattgtcttattaaaactaagctccttgaaggtaggagcttttctcaacattttaaaatctgttaagtatagggttgtgcgtgcattgaatactcagtcaatgcttgctgttattaaaattaggttttgttgggactgccttaccagtagtttcttctcctttactatagtacctggaagaatcttttagtttgctttaactctctcgtttcgtcctttacttatccaagggccatttgaaattctaactctgattagaactgaaaaaaaatattttaaggttgtgtcttatctggtacttaactattctgactgacttttaaaacttttcatttttgtgggtcttgattcagggcacaagctatagcaattggacaagcaatggttgatggacgttggctggattgtgttagtcatcacgaccagcttttcagagatgagtatgcgctgtatagaccactgcaggtacttttcagtgtttactgccaattagaatgtagcaagcttattttatagtctttcttcatcagtgggaataaaaagtagtttattagaagatgatttttctaacattttttcctgctgtttgaggaaagttaaaactttttttacttggtaggcagcagtgatgaaaaagctgccattagaatcaaacattttattgtttactttaaatttgatcagacaataaaaaaggaaaaagataatcaaacacttttttcagttggtgtggtagttcttaaatttgggtttatggtctttatagttgcttatcatttaactttaatggtttttggttttattaggcctatgcttttagaactgagagtataaataataaaatgaaaaattttatggcatctattgaaatcattttgactctagaactattgtataactagtgatgatggctgggtacggtggctcacgcttgtaatcccagcactttgggaggctgaggtgggtgcatcacctgaggtcaggagatcgagaccatcctggctaacatggtgaaatcccgtctctactaaaaatacaaaaaaattagccaggcatggtggtggacacctgtggttccagctacttgggaggctgagacaggagaatgacgtgaacctgggaggtggagcttgcagtgagccgagattgcgccactgcactccagcctgggcgacaaagcaaggctccgtctcaaaacaaacacaaacaaacgaaaaacaatggtatactgatgaatatatgtttttttactttagggaagtggaaattatttgaagatttaacttcaaaaaaagatatactttcactcttggtttatgtaatttgatacaatatataggttatatatatacacacacacacatatataaacatatatatacatatacacacacaggcacacacacacacacatatatatatagctgaccgttaagaattagggatactgaccccctgtatagttgaaaattcacggatgtttgacttccccaaaacttaactactaatagcttagtgttgactggaatctttattgataacagacaattaatacatattttgtatgttgtgtgtattatataccatattcttacaataaagctagagaaacatgaagaaaatcataaggaagagaaactatacttactattcattaagtggaaatggatcatcacaaacgtcttcatcctcgtttcgtgttgagtgggctgaggaagagtgaggaaaagaagagattgcttttgctgtctcaggggtagcagaggcagaagaaaatccacatataagtagaccaatgcagtttaaacccttgttcaaaggtcagctgtgtaatgtttagtaacacctttgtgaactaaaattcgaaagtcaatggttagaagagacaaatgactccaaatattggtgtggcatgattgattaaaagttattttgcaagatgtgttctggagagaattgccccagagctaaaataaattctaagctatcttcactctcaggactaaggcatgaaatgaaacactataaaatctgtatcaaaaaaatgttttgttcttaatatactacttaagtcttctagttaacgaaaatcacaattttaaagagtggaattgtcagtatgattctggtttataacttggtttttgttaatttgtttatttttataaaattcaattgttaggttcatagaaatattggactacaatttatttatagtgtgacagcctcaacattgttgccattgaaggccaattgcttggatcctgtcttcatcctttcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctagtgagtttaactttctaacattttagttttgatgggtcattgctgtgtatgtatgataaaatgattatcttgtttgttgaaataattcagctttataatttatggctccaactaaatggaaacatctgatagcagtctttgtcttagatacagtattttcttaaaccaacttgttaggttgacatttctagattttaccatagtctatagtttgtagcagcttaaaaataactgaactaataatatggcagcagacaactttctcatctcttcactataatatcagactttctctctgcagaacctccaatatatttcttcattttaaaaattgattttatatttaacttgttcaaattttattaagtctacacagaagtacatgaaaatactgtatccattttggtattgcacattgggcttgtatattaaaaaaacatttttctcctttcctccaataccctgataaaacccatttttactcccctcctctccctatatttctttccccttcccaacactaaaagattctgccagtcctagcaagcgcacatcagtcagcagtttccagtccacagtggacagtgactcagccgcttctatcagcctgaacgtggagctggacaacgtgaacttccatatcaagaagccctccaagtacccacatgtgccccctcaccctgctgaccaaaaaggtaggaggtagtcaccatctggaatccaaacacaggctggacagctgggccataggatctcatgccagcctgtccttctggcttcctctgtcccacgtggctgagggtatttggtgtgggttttgttctaccctttctcatttctcccacacctcctccccataccttttttggttgattccttttatttcttgttttctccctcagcaccaccatgcccagtgtaaagcttgtagtctttaaaaggaactacatctgactgctcttcccataatgtgcaacttccaaggtggcatttctttgcatttataacatggatgttctgctctattcttttctctttcttcatttaacattttaaagcctatttgcctttgccgttacccagtaacataatatttcactaaacccattaagatgcttgaactagattgggggaaaatcattcacacattccaaaatatctccataataattcataatgcttattatgtatattctatatttattaaaacaatttcttctctaatgtaaaatgaattcattatttttagctatcttatttgatgctttagtctactggctgaaaaataatagtgaccatattcaaaagatatttggtgtaaaaggagaaagaggtaaacattactaaaatctaacatagcagcacataagaaatgcctgtttttaaaatggatgatgaggtttactttattgggtataattaaaagccaatttaaattatagtcctatcatgggattgtgtaaaagtgaataaataaaaaatacatgagtaaataaataaatgtgggattttgttatgttgtaataactgaaaaatcattattataaagatgaagcactttattccatgattttacctggagtttgtattttaaacatataactgtctaaagtggaattgtctccttacttagatctgaaatgcttgcctaagttttgcgcactctatttcgagcctaagaatatgatcagaaaacagaagcttttgaaccaaacatttatactaaaatgatttgatacaagtttatatttctccaacaaatttctcatagttgtcagtattagctattatcaagatgtggtaaaatggtactgaagagttatagtaataaaaaggaatatgttatttaaaaataagtagagaaggtgtgttaatgagacttgactagcattgaacatgagtaagatagacatctttatggaaatcctcaaaacagaaattggaaaaatgatggagaatatttgggtgagtatgaaaggagggaaaagcagatatgacacttgtgtgagaattaactatagatggtggtcacttggaggatattatgaaggttttgaatataaacttacagatgtgggtcaggggcacacagatgatgaacatctgttctctggacacatctgttggcaatcacattttgctgtaagcagaatgtgaagtatacttttgacttggtgatgatttcatctcttggaatgttaaagaagttttgaaaatcttgaagaaagtgacctgtcatcatagaatttgtaatagggaaggatagactgctgagcacagtcagatacttacttttgaaaacctaacttgagaacattcggagtggaatggaacaggaggctccaaaaatgacattgttatggtgcaaccccagattgtatctccaaggaaattgacaggagaagcacctgcagttgacaaaaatatgaccccttttgaacctccctgatttcaatcctttttttttttttttctaagagacagggtctgactctgttgcccaggctggagtgcagtggcgcgatcttggctcactgcaacctccgcctcgtgggttcaagtgattctcccacctcagcctcctgagtagctgggattaccagtgcgcaccaccatgcctggctaattttttgtatttttagtaaaggtggggttttgccatgttggccaggctggtcttgaactcctggccccaagtgattcacctgccttagctttcctaagtgctgggattacaggcataagccactgtgcctggccctcaatcctttaaaaagattatctatcaaagttgtaagtggcaactttcgtaggagaatgaatctgaaagagggggcagcacttttaagaaaaatgacagtctaatggaagtccagcttgccctgaaggttggagaaaatgccaaggatatctgagaaagatttaaagtttggtagaaagaaaaactaggccagttatttgggaaagtttatgtaatattgattgatgactaaaagaaggcttctttttcctccccgctctttagactaactttatcaggaaagaatacacatggttaagaggaaagtgaagttcaggatattgaagagatagagaattaatggttttttaaataagtctgttgccaggtttaggcaaactgtatgccaggttgttgaaagactttggatctgggatcatggagccagagtttgttatctttgaagagtttcgaagagtaggagagagtctagatgactatagtgatagatacattttttttttttttttaaagatgggttcttgatttgactttggaagctggcaaaatgtagagaagtgtagtaagcagaatgtattgtggtatttagaaaagggattggtggtgactgggccaccagcattggttaagcaaactaagtcgttccaaaacaaccccatttccattttttatggtgatacaagattgataaatcagaagactataatagatactatctttgttgatgttaataagacatttaatgatctctcaataattttgtagacaaagaggacaatttcgtacttgaaatatgttaaaagaggcaaaggcaatggtcatacttgtttaaagcacaggctttggagtctgtatgagctttatttttaaaattgtaaattgacaaattataattgtatgtatttatgggatataaaaagatgttacaaagtatgtataaaatatggaatgattaaggaataaggatttgaaatgatagatatgtttattaaaccaaactaatgaacatattcatcatttcaaatcctttttgttggggcagggtgcagtggctcatgcctgtaatcccagcaagttgggtggctgaggcaggcggattgcttgagtctaggagttcaagaccagtttaggcaatgtggcaaaaccccgtctctacaaaaaatacaaaaaaattagccgggtgtggtggtgtgcgcctgtggtcccagctatttgggaggctgaggtgggaggattgcttgagcttggcaggttgaggctgcagtgagcgatgattttgccactgcagtccagcctcggtgacagagtgagactttatctaaaaaaaaaaagaaaaaaaattccatattattcttagtggtgagaacctttgaaatttactcttagcaattttgaaatatacaatacactattattaactgtagtcatcatgctgtacagtatatctcaaaaacttattcctcctgtctgaatgaaactttttaccctttgactagcatctccccattcccttcactccctcaacctctggtaaccaccattctacttagtatgagcttttaatatgaaaaacacaaagagtgtagtgtatctggtgaatgtaagagcttatgataacactagtagttggtttttttttttttttgagacgaaattttgctcttattgtccaggctggagtgcaatggcctggtcttggctcactgcaacctccgccttccgggttaaagtgattctcctgtcttagcctcccaagtagctgggattacaggctcctgccaccatgcccggctaatttttgtatttttagtagagacagggtttcaccctgttggtcaggctggtctctaactcctgatctcaggcagtccgccaacctaggcctcccaaagtgctgggattacaggcgtgagccaccgcacccggcccactactagtattaacacgtggatttgtagcttgttgtacaaaatattgaaaggataattaataatttgatgtcaacaggaagtggatgctcctaggttatgttatagggacttgactctatatttagagttgttcacttttttttttggtcagcacttttaatgtttttttaggtccacaaataattaaaatctggaaagaatggctagcgtgttggatagcagaattgacatttaaagatctttattttctcaagcaatagattgagactaacaagatgaaagttaacacagttaaacataaaagaataaggcttaacatgtttactattagtactttggagaaagtcactcttaataatttgtgtgataaagatctaagttttagttatttgcaaacttaatagtgtgatgtgccaaccccgcactccctggcactccctagggcaataatagatttcattagtggccatataatgtctagaatgaggatccaatttactgttttgtgttcagttctcatttatgaatagaacgaggttgccaggatcctaagacatctgaaaaccatgctaaggaaagctggactatttaaaatggaaaaaagacgtggtgtttcctttaggtatctgaagagtacctgaaagaaattatatttattttgcatatggctgaaggaagaactagattcagtagttgggagtagtgaacttttgccactgtgcatggtaagtgtccaggttgtgtctgaatgtccacctgtcatggttattgaggacagatgcctgcattgggtgatgcgttatatagacttggggacttctaagttttcttttaattctaaatctgtgaactgaaaaatctagatctagaattaaaaagaaattataatcctttagcctactcagtgagaatattacagaggaggttccagcattaggaagaagataattattaaattgacctcttaggtcatttgcagtcttataatttgaagactttaaaagtctcttaataaatattactggctttcacagttctctcttagtttttaacatactaacgagttattctttttacattttttcttgtgaagttgaagcgtaatagtttgctgagtgttatagtcaagatgaacactgattatcagaaaataaagatttccagggagactggtagttacgtgaaaaaataatagaattcttttgcctctggttttcattccctttgtataaatacaaatgggaatggtttttatctttacattgaaattaaaggtagtacttattaaatattttcttatggattgtcctatattgtttcaggctgaggaaaattggatttataaaattggttgaaatatctgtactttaaaaaatactacttatgctttctgtctgaagtgagaaaaccctattttcagaagctcgtaaaactggcttaatttttcattattggtcttggagaattgtacagttaaaatatattttattatgtattacaagcaggattctgaactattcttggaaagttagcaagtaaaacctagagaagaaggtgaaaatcacggcttccgtaaatctctagatgtagttttccgtgtataggaaaacatgggatagaggagcaagttaaacacaccatgagaaaacaatggattgtactccgaatgtgggacattgcacagaacatttgacctgggttcttttaaattcatgttatgaaaagaaaaaaagacaggagatctgttctagattaaaagagaatgaagagatacaggaaccaaatgccatgtgtgaacttgattgcatcgaggcttaagaagaaaaactgtggattaaaaacattttggagacagttggataaacttgagtatgggctaattctgtgctattaagacattgttaattttctcaaatatgtaaataattttgtggctatagagaagaatacctttatttttatgagagttgtgctgatgtcctagtggtaaagtgtcatgatgtgtgtaactttgaatgatcggggggaagaaacatacacatatatggcaaatccttaacaattgttgaactcaggtggatggtatacagcattttctgtaggtttgaatttttttcataactaaatggggaaaatccaccatttctggagtctcatatagatacaattaagctttagtgtgagaaggctggtaaagaacaatacattaattgaactaataggatttggtgttactcattcctgtgagggaagagtagaaataaagtgtaacatctatcttgcccacatactcgattttattgtatttggcatagtgcacttgaactttctaaataaaatattttctttgtttcctggttggaaggatgagcatattggcttataaacatagtaacttttaaagcccagttgtttataaatgtacttggagaaaattttgtggagatcctttaccgtaaaggagtttacatgcatcatgttttaatttgcatagtggaaagaacctcatttgagctatgcttggtcacagacctagagaaagttcacggggaagtaaagagtaagtttcacttgtaagaaaagtatttaatatattaaaagcattcagagtgtgttcagttaatgtctagcaaaaatttggcagtgtttgatataatttttgaaaaacaaaaacacttatttggaactaatacaaatactgatgagttttatctttattcctgccaatgtcacttctgtattaatttgttttatagcttatattctgaaatttgaaaaaagtttatattgttttatcaggtgggttctgtatgaactgctttgtgcataggcaaagtgattatttgttaaatcatcaagttacatgtgaaaagaactgtaatcttttaggatggtcgtacaagtatctgaagatttaattttcataaaccggttggttttggtaagagaaaaacatttctctatctgacatttgaattaaggagaattgttttcacggctctattgtgttattaatgcaagtctgcttaagtttagtatgaagtgcatagaacaagtataaagaggcatttttacccaaagctttcttgaatttgccgggcagtgatgggtttagtatgagtaatattttgtgtgtgggcgtattctctatataattatgctgacatgttatagtcatatgtctctacttttgttcctcctctctgttgtctccctggtatgcagagtatttgatttctgacactggaggacaacagctctcaataagtgacgctttcatcaaaggtaattttataaaaagctgtatgtggaatggtgtctctttttttcatgaggatatacttagggcttagcacattgattagcacagagaaggtgctgaaaatatatttgattatggtggtatcatgggaacacagtttttaatatatataatgatttagagaatgttgagaaccaagaatgtaaacttggacttggtttgcgagaaggaggtctctgatgtgaaaagtaattgtttaaaaacttttaagtagaatttgaacttaattcttttaaagacatagtttacacttaactgtattgtgaatataatattaagtattgatataaatatcatcaaggattgtcaaatttgtcaaaaagcccacaggcagatctccagttaaggatgctgcttgccctgtggagtgaggaagagaagcaggcagtagaggttaaagaactaactctgatgataaatttcttttggacacaaaaggagattagccttctcctattccttccctacttagcttagcttagcttcctagagtaaagacattcttagtccatagtactccactggttaggcctcagataatgggagaaaatttctcttccttcttggctcccacggtttcagatcaaagagagaaggcaagcgggtacaaggagccactgcagcttatctgatagcttggcagtgcttctaaattcatctccaagcatctggttcggctagaagtttacagagtggccctttatgtaattgaaatgtgcaattcttgtacttcacatgtcatggacatatactttactgtgtaatctgctgggctgtttggtggaagtttttttgtttgtttgttttgcttttaagaggggacaggttagagaatttttgtaagcatttcccacttcttttctttccacttatttggtcagattattcaacagttatttagtgaacatatttttttgttaagcatttattatttattttttgtcttatctattttcattcatcctttaaaaaatttaaagattttacacaaattctacataaatgctatttattatgttttaatatttatgtttttgagtctgtcttgctaaaattttgttaaaaaacccctgtgccaatattgaaagagaaacactattgaggattgtctgaatagactttctacactgtcatctgttgttttgtgatatctgagctgagcttctttcttgaaaattcatcactctcgctatcatgtgttcttttgccagcattcagatttctgttgaatgcagttttcagtttgtgatttttttttttttttttttgagatggagtctcgctctgtcactcaggttggagtgcagtggtgcgatcttggctcactgcaatctctgcctcctaggttcaagtgattctcctgcctcagcctcccttgtagctgggattacaggcgtgtgccaacacgcccagctaatttttgtgtttttagcagagatggagtttcgccatgttggccaggctggtctcgaactcctgacctcaggtgatctgcccacctcggcctcccaaagtgttggaattacaggcgtgagctaccacgcgcggcccagtttgtgattttaaaggggaaattaacaccctcgtttttaaaaattaaaaggagaagcaaaagtttcattttatttgcttttcagaagctgtgcttataattattggcttgagagtcttagttacgtacatttgattttcacagttctgtttctgattttggctgttgccaattactgctcttcttaattcttgcatttctgtatttttagtgtattttatgtatttgtaagtattttcttatgcattttatttatatgtttattacatgttcttgcctttttacaccaataatttatgttggctaacttaaaacatttataaagacaaagggcaatttctaaatatatgagtagatggtataaaacaggacaatgaagtaataacttcttattttatattgtacttagaatccttatttaatcgccgagtagaggaaaaatccaaagagctgcctttcacacctttgggctggcatcataacaacctggagctcctgagggaggagaatggggagaaacaagccatggagaggttgctgtaaggcaatgaaatttttaattttaatttttcacttttattatcttcagtgacttgataagcatatgcatcattcctcttaaaaagggaggtctttttctttttccttagaaagtaaaaaaatactaaggttacttaggaaatacagtctattttgacttgtgattaaatttcttataaaaccaagagccccggctgggtgcagtggctcacgcctgtaatcccagcactttgggaggctgaggcgggaggatcacaagctcagaaggttgagaccagcctggccaacatggtaaaaccccgtctctactaataatacaaaaattagccaggcgtggtggcgctggcctgtaatcccaactactcagaggctgagacaggaggattgcttgaactcaagaggcggaggttgcagtgagctgagatcgcgccactgcactccaacctaggcaacagagcaagacttcgtctcagagaaaaaaaacaaaaacaaaagccccaaactgtgcttgtctgtccccattttacgtgttaaaatttcatttcagtcaattgcaggggacttcttagtggaagatattcagatttcatttatatgcaaaatccttaaggtacagatataatgaaagaatgatattgacctagaaaatcttatctgtgagccgtaattgaaatgtttatttctttgtaggctgtttgttttatcatcagaattattgtctctggcagtattaactatgcaaacttctttcttataatatttttgtagttcagctaatcataaccacatgatggcactactccagcagttgctccatagtgactcactgtcatcatcttggagggacatcatcgtgtcattggtctgccaggttgttcagacagtccgacctgatgtcaagaaccaggatgatgacatggatatccgtcagtttgtccacatcaaaaaagtgagctctgctaatgttttactaatgctaggaactgatgaggcaggactgattgtaatttttgtcttaatggtaatctgaaaagagtcattaccctgaggggtgctaggtgaggagtaggaggttttgctgtctgctttgttagcagcgcacttaggagtcatggaagccacgcataggaagtgaaaggtcaatggaaaggatgagttatatcggagatgagttgggaacccaaacagagataggttcttcctttcttgcttcctcttcttgacaagtcagctatgagaaagcctgttctcacagattgtgatcactgtttggaggaaaagcagtgggtaatttgctttatttagatcaagcttgtcccactcgtggcctgtgggctgcatgcagcccaagactgctttgaatctggcccaacaccaattcataaacttcttaaaacattatgacattttttgcgatttttttttttaacttttttatgtctctttctttttttttaattattattatactttaagttttagggtacatgtgcacaatgtgcgggttagttacatatgtatacatgtgccatgctggtgtgctgcacccattaactcgtcatttagcattaggtatatcacctaatgctatccctcacccctccccccaccccacaacagtccccagagtgtgatgttccccttcctgtgtccatgtgttctcattgttcaattcccatctatgagtgagaacatgtggtgtttggttttttgtccttgcgatagtttactgagaatgatgatttccaatttcatccacgtccctacaaaggacatgaactcacccttttttatggttgcatagcattccatggtgtgtatgtgtcacattttcttaatccagtctatcattattggacatttgggttggttccaagtctttgctattgtgaatagtgccgcaataaacataatgtgtgcatgtgtctttatagcagcatgatttatagtcctttgggtatatacccagtaatgggatggctgggtcaaatggtatttctagttctagatccctgaggaatcaccacactgacttccacaatggttgaactagtttacagtcccaccaacagtgtaaaagtgttcctatttctccacatcctctccagcacctgttgtttcctgactttttaatgattgccattctaactggtgtgagatggtatctcattgtggttttgatttgcatttctctgatgaaaaaaccactttaaagttcgtatggaaccaaaaaagagcccgcatcgccaagtcaatcctaagccaaaagaacaaagctggaggcatcacgctacctgacttcaaactatactacaaggctacagtaaccaaaacagcgtggtactggtaccaaaatagagatatagatcaatggaacagaacagaaccctcagaaataacactgcatatctacaactatccgatctttgacaaacctgagaaaaacaagcaatggggaaaggattccctatttaataaatggtgctgggaaaactgcctaaccatatgtagaaagctgaaactggattccttccttacatcttatacaaaaattaattcaagatggattaaagacttaaacgttagacctaaaaccataaaaaccctagaagaaaacgtaggcattaccattcaggacataggcatgggcaaggacttcatgtctaaaacaccaaaagcaacggcaacaaaagccaaaattgacaaatgggatctaattaaactaaagagcttctgcacagcaaaagaaactaccatcagagtgaacaggcaacctacaaaatgggagaaaattttcgcaacctactcatctgacaaagggctaatatccacaatctacaatgaactcaaacaaatttacaagaaaaaaacaaccccatcaaaaagtgggcaaaggatatgaacagacacttctcaaaagaagacatttatgcagccaaaagacacatgaaaaaatgctcatcatcactggccatcagagaaatgcaaatcaaaaccacaatgcgattttttttttaagctcatcagctgttattagtggattttatgtgtggcccaatacaattcttcttccagtgaggcccagggaagtcaaaagattggacacccctgatttagatagtaatggagtcctgtgtgtgctttttccctatgatatttaacttaaacaatagtgttaactgccacatggcactagtttattcagtcagtgagtactttagagtcctagggtagtacattagagaacaggggatgggaaaacagagtcatgcagagatgccattgatcgttggttgaaaggaagtattatttcctgaaattttttgttcttatttgattacataatagtttaaaaaaaatggaaagaaataatagcgtacatctaactagattctaagcatgttatcattgaattttattgttttcttaattgttccattagatcccaggtggaaagaagtttgattctgtggttgtcaatggctttgtttgtaccaagaacattgcacataaaaaggtaatgtgattcagttcagcagtgaagttcagcaaaccatggctgtgtgcttaggtaacaagttggggcaccttagttatggaagaaatggacaatggacaatgaaaggcagctgtgtctgccataggggtgaaatttggttggttcagggaagatttcccagagctgaggatgaatggattggaatttgaataatgggaattcaccagacagaaggaggaaggggagaggatcaggcttctagattaacagaatgataagatactggcattatttatctcctgatcctgttacgtcttagggagacagggtttctctagtaggctggagtccttccagaggcttagcattctaaaactgatagctcttctagtgctcggctacagaactgtcagtcttcttctgttatccttctcaccttatatctttgtagcactagcacaaaggagtggagtgagataacctggggtattaaggagactctgagttcctaaggtgtaatatgggcaatgtagatgaagctggaggaagcaggcaagggctttgtatgtcattaaaggtcttgaactttattctgaaatcttttgattctcagtgtagtgtacatacattgagatcacatgtggaccttttaaaaatgtctgttcttaacctaacccctagaaattctcattcagtaggttaagggcaatgggagtgtatggatgttagggaaatggtggtactgtatttaaagtaggcactaggctgagtgtgtaataccagcactttgggaggccgaggctggcagatcacgatgtcaggattttgagagcagcctggccatatggtgaaaccacatctgtactaaaaatacaaaagttagccggcgtggtggcgtgtgcctgtagtctcagctactcgggaggctgaggcagaagaatctcttgaaccttggaggcgaaggttgcagtgagctgagatcacgccactgcagtgcagtctgggcaacagagcgagactccgtctcaaaaaaaaaaaaaaaaaaaaagtaggcacttcaggtgtttccagtgtcatttgttggtccttgcattttgagataccatttatctatagtagtgttctcaaaccttgctgtgcttcagaatcaccagcggaaacattcaaaacagagataccctggccccacttctggggatactgattttgttacagcaagagctgaggcagatttctagcctgagcaaccagatggatgaactctagtcttgttttcactttttttctaagttgagcttaatatagttttttctaaggcattaagattcctgaaaattattagtgaccatagataaataaaaatatacttggcttgagttttgtacttatcgcgtattttattgcaataattcgacatcagtcaatatttctacacagagtcttactaaaaccacacattaccctttaaaggagaactttaaaccaaattaaatgatatttgttgaattaagaggtaaatatgttgtgccttctcagaaagtgaggaaaatttgtatgtgggtgtggtggaaacatatgcatgaatgactgtcttgccccactgattagagatagtccttgatttgtagttgctaaaaagagtcctgaattgagtttcccagctggtgagtcactgttttcggaactgtttgccagtttggggtccataaacttggtcagcctgaaacctcaattgctattctgcctgtggctttaagcacacgattagggtatattatttgggtctctgcttcttgagatgctttcagattttctttttgaacttcatgtttcctatttcagcttgccaccttaatgattgtttggaaatttattctattagcaatatgttcaggaggcattttaattcttttctccctcttctcctcttcaattttagacagactaaaatatgcttttaaagttgcttatttcttggagtttgtgaagtaacctgtcattaaatactgctccctccgtgcacactcagggcatgatgtctcttcttacaacatcagaaataaaaaaaaaagttaaagagtatagaagatcaaagtagtattaagaaagaaaagccattttttgaagaattttgcattgttctgaacatattatttgctttgaaaaaatttaataccgtaaaagaactaaatgagaatatattaatactaatatgagatacattaagttagtaggaattatgaacaatatagatttaaagttataattaaaggagggctgtaaaatgcaaacctttaaacactttaacaaactgttcattttttgtagatgagttcttgtattaaaaaccccaaaattcttctgttgaagtgttccattgagtatctctacagagaagaaactaagtttacttgcattgatcctattgtgcttcaggtaagaatttactactgaaaataataatttagagggatgtttgtgtaccatgatgcttataaactcttaatgattttgacttaattctagctaattaaattggataggcaaacttgatagcttaccaaaacctctgtcactcattatttttaaagtttcatagtattaaaggtaacatgtgactgttcaaggatataaggaagaaaaagtgaaaatccaaatactaatttccctttcccacttaatccactgatttactgtgggaatttctcgaacttaccagatttctaatggaatctctggtttgtaatacagtctatgctactaaaacatatttctagaacatcaattagcttatatgtagttggcaaacagaataatgtcagatgaaagttgtgttggtttctcagtggtttttcctaagcaaggaggcccagaatagctgcattaggttataaccctcagctggaaaggaaaacagtctcagctcagctgctacacaggcagaaacccttccagctctattttaagaaagtaaaagtgagcttttgcttgcagggctctctccccagggaggcacacccgcatctctgtgtggttcttggtagctttgctgctcagagctccatttctagttgtagcgttgcaatagtttttgtgcatttaattttacttgaggcagcctcatgccatatgaagctctctgcctgggctctccatgattcctcttaaggtaccaattattatttttattagtgctgaaggtataaagttgcataggttttaagttttctgctccaactccttttctcccataaatactgttaattttactttgtggtcattcaaagtggaagcttttagggaatgtgtatattatattatagtggaaacacttatgctgggttccagaaacatttaggaaaagcattacttgaaaattcctaggtaaatacatttttttcaagagtggtaggagcaatcacttttgtactgatttcctagataacaataagagaaaatgagttgtagtaaaagaaacagtgctttataaaactctgctgttattacttaagaactctgtgacctggggctgttttctctttggaattcagctgtgctatcagtaaaaatgatgggttttgactagacattttagtagagaactctaaagaaataaagggaaatacttcttttttctttttcttttcttttgtttttttttttttttttttgagacgaagtctcgctcttgtcccccaggctggagcgcaatggtgcaatcttggctcactgaaacccctgcctcccaggttcaagctattctcctgcctcagcctctcgagtagctgggattacaggtgcctgccaccatgcctggctaatttttgtatttttttagtagagacggggtttcatcatgttggccaggctggtcttgagctccagatgtcaggtgatccgcccgaggcgtcccaaaatgctgggattacaggcgtgagccacaatattcatttcttttgaaattaagcctttgtgataaaattctattagcattatgtttgagatctttaactgtagctgttctaggtattatatgatctgttgggaaattggaattaggaagtagctgaaatagcatctgttagagttatttattcagagatccagaaaaagggataactcatgttggtttcagaatgtagaggagagaattcataaccatactaaaacaacttagggatgggcatagtggctgacacttgtgattacacttttggagctgaaggcaggaggattgcttgaggccaggagttcaagaccagcctgggtaacatagtgagactctgttcctacaaaaaatagaaaaaattaggcaggcatggtgatgtgtgcctgtagtcctagaagggtgaagcaggaggattgcttgagcccaggactttgaggtcacagtgagctatgacggcacctctgcactccagcctgggtaacagaatgagaccctgtctcttaaaaaaaaaaaaaaaaaaaaaaaacttagaaattgcttttattttgggagttgtgaggttaggtttttttgttgttcttgtttttttttaacctctgaactagctaattggattcttggcttttaaattaagaattgaaaatctttcaaggaaagttagttggagaatggaatttctcctgaaactcttgtcagcataatcaagtttagaatgatgaatgagattcttactgtgtttgcattaatttgcttacagacttatttgtggatgaagaaaatttagtctaagattgataatagtcaagctatcttgatatatatatatatattttttttattacactttaagttctagggtacatgtgcacaatgtgcaggtttgttacatatgtatacatatgccatgttcatgtgttgcacccattaacttgtcatttacattaggtatatctcctaatgctatccctcccccctcccccaccccatgacaggcccccgtgtatgatgttccccttcctgtgtccaggtgttctcaatgttgaattcccacctatgagtgagaacatgcggtgtttggttttttgtccttgtgatagtttgctgagaatgttggtttccagcttcatccatgtccctacaaaggacatgaactcatccttttttatggctgcagagtattccatggtgtgtatgtgccacattttcttttttatttttattttattttttattttattattattagtagtagtatactttaagttttagggtacatgtgcacaatgtgcaggttagttacatatgtatacatgtgccgtgctggtgtgctgtacccattaactcgtcatttagcattaggtatatctcctaatgctattcctcccccctccccccaccccacaacagtccccagagtgtgatgttccccttcctgtgtccatgtgttctcattgttcaattcccatgtatgagtgagaacatgtggtgtttggttttttgtccttgcgatagtttactgagaatgatgatttacaatttcatccacgtccctacaaaggacatgaactcatccttttctctggctgcagagtattccatggtgtgtatgtgccacattttcttaatccagtctatcattgttggacatttgggttggttccaagtctttgctattgtgaatagtgccgcaataaacatacttgtgcatgtgtctttatagcagcatgatttataatcctttgggtatatacccagtaatgggatggctgggtcaaatggtatttctagttctagatccctgaggaatcaccacactgtcttccacaatgttgaactagtttacagtcccaccaacagtgtaaaagtgttcctatttctccacatcctctccagcacatgttgtttcctgactttttaatgattgccattctatctggtgtgagatggtatctcattgtggttttgatttgcatttctctgatggccagtgatgatgagcattttttcatgtgtctgttgggtgcataaatgtcttcttttgagaagtgtctgttcatgtgcttcgcccactttttgatggggttgtttgtttttttcttgtaaatttgtttgagttttttgtagattctggatattagccctttgtcagatgagtagattgcaaaaattttctcccattctgtaggttgcctgttcactctgatgatattttaatatgtccagatactgcattttaccaggtgtaatatttctgatttatcttacttaggaaagggaattcttgaagaattatgtccagcgaatagttgatgttcgacccaccttggttcttgttgagaaaacagtgtctcggattgcccaggacatgttattggaacatggcattactttggtcattaatgtaaagtcagtgagtgtttttaattttctattgtattttaaaaaaatcacagcttttaaatttatatgcacgtatggtaggtatacaagaatctttttttctttgctgttctatttgtattgaaagtgggacagaagatatggcacctatttcattaagattcagaatttcagactggaactggaagaaagtataattgattatgtaatacttattggtgtctacaagaccaaaagtgaaatggacattagttaatcactaggtatttgttgatccttcatttttgttggagaattgctcaagcagaagacgttctctcccttgaaaagtttaccctgtgcagaacaggggccaattctaggaagttgtttttctccccacattatcttgtggtaatccatgatgcttaagtgcatttttataatgttattaatatgagtaaggtttcagtgggcatatattgattactaagcaactaagtgtagaagaatccagaataaaaaaattcatcatatatttattaaaaaggaaatttgtgaatgttattttttaatttttccattacttttccagattaaatttacaaagatttttattcttctctagttttgtaatacaatgtttttctgttttgtagcaagttttggaacgaatcagtcgaatgacccaaggtgatttagtgatgtcaatggaccagctgcttacgaaaccacacctgggcacttgtcacaaattttatatgcagatatttcagttgcctaatggtgagtgatctttagcatagattgacctgaggaaaagagttaacttatcactactacaatgtttacttactaggaaatacctattaatagtgggtaagtgaggataggcaactgtgatcttattcatgtaagagtttttcttttacagatattaagagagtgaattaattctatttgtacttctggattgccacctccaccatcttaactttctgtttgtttttgtttgtagaacaaaccaagacactgatgttttttgaaggttgtccacagcacctaggctgtacaatcaagctaagaggaggctctgattatgagctggctcgagttaaggagatcctaatatttatgatctgtgttgcttatcattctcaactagaaatatcctttctcatggatgaatttgctatgcctcccacattaatgcaaaacccttcattccattccctgattgagggacgagggcatgagggggctgtccaagagcagtacggtggaggttccatcccctgggatcctgacatccctcctgagtctctgccctgtgatgatagcagtttgctggaattgaggattgtgtttgagaagggtgagcaggaaaataaaaatcttccgcaggctgttgcctctgtgaagcatcaagaacatagcacaacagcttgcccggcgggtctcccttgtgctttctttgcacctgtaccggaatcattgttgccactccctgtggatgaccaacaagatgctttaggcagcgagcagccagagactttgcagcaaacagttgtgctgcaggatcccaaaagccagataagagcctttagagaccctctacaggatgacactggattatatgttactgaggaagtcacctcctctgaagataaacgaaagacttattctttggcctttaagcaggaattaaaagatgtgatcctctgtatctccccagtaatcacattccgagaaccctttcttttaactgaaaaggggatgagatgctctacccgagattattttgcagagcaggtttactggtctcctctcctcaataaagaattcaaagaaatggagaacaggaggaagaaacagctgctcagggatctctctggacttcagggcatgaatggaagtattcaggccaagtctattcaagtcttaccctcacatgagctagtgagcactagaattgctgagcatctgggcgatagccagagcttgggtagaatgctggccgattatcgagccagaggaggaagaattcagcccaaaaattcagacccttttgctcattcaaaggatgcatcaagtacttcaagtggccaatcaggaagcaaaaatgagggtgatgaagagagagggcttattctgagtgatgctgtgtggtcaacaaaggtgagccagaccactttctgatgctcctgtgcatttaggatgtgttgaatgactcctcaaaggcaggctaaaaaaatcagtttggcagactgcatgcagatttgtgaaatatttcttctcctatgctatttttgtttttgtcttgagtctgctggcttatgtgttactttaatatgtatgaagcagaattgtaatgaaactgggtttcagggttttaaggtgagcttataataagctaggaagatccctgaagtaaggggaaatgtggagctgcttaaaatttctgacaacatgacttggatgtggtatgataatcctggatccttgtgtcagggagtgagagatggaagcttattgtaggaaatagatgtgtttttttaaaaaagttagcagcaaatatatttgttttctcaaaaaaagttagctctttttttaacataaaacattttaccttgttatttaactatgacagtgatctttttctttttaggcataaattgagagcaaattaaggaaggtttatagatccattattgataatggtagcaataaaactaattaacaaatccattgctagatgaggaaatgcaattgtttaataaaacgagaagatgtccaatttcgccagtagtcaaggaagtgctaaataatgtatgttcaatttcactagtagtcaaggaagtgctaaataatagtggtgctaaataattttttatttgtcagattgacaaaaaggaaagtcgtaaaagacccatggttaatgatggcatggaaaaacacctttcataaattttatgtaagtttatacaaatgtaggtatatgtacatttgtataaatttatagaaagtgataggaaagtctccattaatttacacatacatgtatatacctttgacccaggactccaactttttataatctatcctgcagaaataattaagtatgcttatataagggtgcttgcattttttaagaatatatgtacaaagatgtttattgaaacagtttaaggatgagcaggatctcaagtcatttaatgacttggataaagcccatgatatatttgaagtgttaaaaagcaaattatgttttaatatgtgttgcattattccatatttataaaaacaaaaccccctgtttgtacttaggaagtgatgtgtgtatgtgggacaggtatgtaaactagcaaaaagaatagatattttaaaaccacagagtcaaactccgcagcttaggaggtggagtagaatcaggaggagcagttttgtgtattttatgaacttcttcatatgcttctatactgtttcaattttaacagagagctggttttacctttgtaatgatcaaaaacagccataagcatagataaataaacactttagaatttagatttctgggtgtctcagattgatgttatatcccatcagttcagactgttaaatatgggtatatttaattaatttttttccaaaataatttagtgcagttgtgtaaaataaagagtaagttaagaaaacaagctatctagacattgttttgtatttttaagtttaattgaaactttttgggttgtcttttctcaaaaaccaactgcccttccttatatttagttttctgtagttttaaatcttgaccagagcccccacagtgataattggaggctttacaaatagagtggagtgtgtttggtgcattggggttgaatgcggttgcagtcacttgctcatccattcattccttctatttcaggtggactgtctgaatcccattaatcaccagagactttgtgtgctcttcagcagctcttctgcccagtccagcaatgctcctagtgcctgtgtcagtccttggtaggattcttttccccctacactattccacataagaacagaattccagcaattaaaaaaaaacaaaaacaaaaaaacagtcattaggacctgtatttaggtacacagcacactgctggtcatatatgaattgatagaacttttaaacttgtgaagtaaatatgaaaatacatgtttgttcatgagtagaagtaaatgagagtatattaagagtgaaaaaagtgtgagggatagaagaggaatttgtattggttgactttcccttatctgaaatgcttgggaccagaagtgttttggatttaatattttttcaaatttggaaatacttgcatatacttaatgagttatcttggggatgggacccaagtctaaacatgaaattcatttgtgtttcatatacaccttgtacacgtagcctgaagttaattttatataagatttgtaataattttatgtataaaacaaagtttgtatacattgaaccaccagaaagcaaaggtttcagttgtggaatttccctcttgtggtaccatatcaggactcaaaagtttcagattttggagcattttggattttggattttcagattagggatgcccaacctgtacttaaaaatgtgagaaagttgacacacaaaaagcttgtttataagcattttgatgatagcatgtacaatttaggcaggttttccttcaggcataatttttataaaagcagtcaaccttttgctttattaatgggttttagtctaaaacttaagtagattgatagaaactgaaatacattttatgttttaaattatttttttcaaattattgtcacaaaaacctcacacagacatacatagccttacattacatgttcacctttagttttaaaagtactatggaacataacttcttgtttgggggcactgtctccatgccagaacatattctgtgttttaattttggataactgaaacacttatttccagcagtgagctcatgggttcttctagtagtatagcttcagcaggaagacttgggagtggcagaacccatgggaattatctctgggagtttatatataggtcaaggcttgaataagatgtggatttcaacagttatatagccaaactgtcttgtgagcatttataggttagataaaaagttagaaactggcactttagcaagaactgctgttttccatgctcagtaaaggcatggacaagagagtaggagaccagagtcccaccttgtgctgacagggcttaagcctttgtaaaggaaacctttacctcttcttaactcagatcagttttactaaattaatattatagtttatgcatcctcattagtttcataagacactagtggattgggagtgaaaaagatgctttagtgtggcaagtctaaacttgttttattaaaacctgtttcataacaacaaagaacattacctcctttcatacttcattgtaagtaccatacatttaataggtaatcataatttgtggtctcatgaaatgtctctggggcatggtaattcttatgtttctaagaggtggtctcttctttaggattgtaacaatggaattttatggaaagaatgatcttacattaggaatatttttagagagatactgtttcaggtaagaacatatttcttccttctgttccattacacatttttgatgctcaaaatttcaagctaaaacgtaaacatgagtcggatgttaagtgactgttacatgatcctcactttcatagtgcatatcagagtatatcattcaaagaagtaataatctatttcatatttaaactgagatctcttatccagaacatccctcatatctctgcccatctaattaccctaccatcaacatttttcggatacctgtttttttacagtgattcaaatgagaggtaaaatgttggaatcatctagacctcagtttaagtcttagcatcatcactcgccacttatatgacaaagcactttactagtcccctttatagggaataacaatccctagaattatcagaaggtaaaaggaaatgttttaaaattgtttggcaaatagaaggtgcttaacaaatggaaactatcaccgtgctgtagctgaagacgatgttcagctgcaggctcagagcagcatgagtaccttgtgcagattgttcactagtcatgctgcactttgacagtggttctgatttctgtttcatgcttaatgtgagaagggctcttttgtttgtcaaaggccttcttatcagtgtccaagcatgttctgtgatacccccatggtacatcatattcggcgctttgttcatggccaaggctgtgtgcagataatcctgaaggagttggattctccagtacctggatatcagcatacaattcttacatattcctggtgtagaatctgcaaacaggtaaatagaccctattactatattttgtttgccatttatttctttatttgtatgttttttttttttcctgaggaggtttcctatatggtactggattcgttatttgttatagagctctatttgtcattgttattttaccttccagtgctgtatgttcaatctagtattcgttttttgaagatcccatcttaggaatgtatcagtcattaaataaaatacaagtgccctatacagtaaagtatcttgaggttgtatcatgacatatctcaattttgtttattcattcaacaaacatttagcaccatttatgtgccagatactgtgctaggccctaggaatgaaaaaaagaataaggaatgacttctgttctctaggggaggagacagacttgtaaagcaataactgcagtatattaggatatttgtataataaattcgagcctatgtgagcatttctattcacatacagatgagaccatgttgaaattgatctctattggtttttatgcatgttcttgagaaaaggacatgtgttttgtttcttctttgtgcctttctttacttttctaactctggggcaaaaatttggtgccaatattgtgttttttttaaatatatgatttttatccagactgcttgcaatctggataaaatattccaaatttaacttactaccttaattaggttctttctttcttttttgagacagagtcttgctctatcgcccggtctggagtgcagtcgtgtgatctctgctcactgcaacctctgtctcctgagttcaagctgttctctgcctcagcctctggagtagctgggactacaggcatgcgccactatgcccagctaatttttgtatttttaatagagacaaggtttcactacgttagccaggctggtctcaaactcctgacctcgggtgatcgcccaccttggcctcccaaagtgttgggattacaggcctgagccacagcgcctggccttaattgggttatttcaaagtgtgcttttggaaggcacatatgggaattatatataaaataatagcacgacctacattaatcagtggtagagaagggcaaaatcaaagttgcacctgcagcagcaaacatacagacataaaattatttctagcaattctcagtcttagtttcataattgttcttaccgactttagttcctattagttagctggtaatagtttgttgtagaatatgttaactgggtagactgaccatggtactaaatgaataacttaaatttgatctttatgacttacatgctttgctatatgcactttattttgaaacaaaaacttctgcttttttatgctagaaaggaaaaggtaagttttgttacactaaaatgaaaaacttccagtgtcaaggaaaaatacaacatatatgactaaacataaatatatataatatcaaatttctatgagccatgataatagaaaaatgggcagaggacatgacaatgcaatttaatgcaaatggctactaaacaaataaataaatgttattcattagtagtcaggtaagcataaatcagtaaaatctctttaaaattttttctttaggaggttacatttcaaaaatttcaattctgattcctaggattgggtaagaaagtgggggaaattgatattcttaaatattgttggagactgtatacactggtgcacttgttttggaagataatgtggcaaaaattatcaaagatcatataaatgtttatgccctttgacttaccaattcccacctttattaatttatcccacgggaataattgtggatatttgtaaatatgtatatacaaggatgttcatcagaacatcatctcagtgtggaaaaacttgtaatgtcttaaaggcacatcagtaaggattggttcaatacatgtccagatactctgaagccatttgaaaagttgtgcaagaatattttatgtgggaaaatattcacgatatattgctaagtaaaagattatgaaactataggagtttttatttacaaatatactggctgtgtgtgtgtccatgtatgtatgtatatatgtatatgtgtgtgtgtgtatatatatatataaatacatcaaaaataaacatatactagtatattgatgttgttggtggatttattggtaatgctgctctcctccaccacccccaagtccacccacctttgaactttctcattcttttcccagttttctgcagtcagtatatattacgtttgtattagaaaagaaaatgttggccgggtgtggtggctcacacctgtaatcccagcactttgggaggctgaggcaggtggatcacgaggtcagaagatcgagaccaccctggctaacatcgtgaaaccctgtctctactaaaaatacaaaaaattagccgggcgtggtggcgcgtgcctgtagtcccagctactcaggaggctgaggcaggagaatggcctgaacctgagaggcggagcttgcagtgagctgagatcgagccactgcactccagcctgggtgacagagcgagactccatctcaaaaaaaaaaaagaaaatgttattttttgaaaaagagattctcaatatgtgattaggtaaactatttttgctgaattccaggtaacaccagttgttgctctttccaatgagtcctggtctatgtcatttgcaaaataccttgaacttaggttttatgggcaccagtatactcgcagagccaacgctgagccctgtggtcactccatccatcatgattatcaccagtatttctcctataaccagatggtggcgtctttcaggtaagaaatcctaggaatcttgtgcatgatctcagatctcataatcccttaagaatttttttaacattttcaccattactagattgaattaatttgtgggattccccatttatacccttataaatgcagaggaccagactgtattaagaactttcagtattattttatttactctttaccctacaaaatagttcttattatcctcacttacagagcttacgatcattcacttacagagctctcaagtggtggagtaatgatttgaaccttggtcgatctactaccagcctcactctttttttaaaaaaagttttaatatagaaattttagaaaaatatgagacaggatctcactgttacccaggctggtcttgaagtgtgctcaagtgattctcctgcctcggcctttcaaagtgtggggattagaagtgtgagccatcatgtcgggccctggcctaactcttaaccattgttttggcatctgatggcatctgaaagtctgtcagaatttttgccaagatattaaccatcagaaggataatcacataattctgatagagctaaaagtctgtgttcaaagaattgatagttgccattgtggtattaaataatttgagacagccttcaggttctgccttcaaaatatatcaagagtctgagcactttttaccaactctaccactaccactttgggctgagcctctgttatctttcacctggattattgtagtagccttagcagtctccctttggccttgtccctttagcctcttctcaacacagcagtcagagtggttctgttaaggggcaatcagctaaggccactcctctgctctagacactccaaagggtgtctgtttcacctggagtgaaagccaaagtccatttactggcctttaatctacctgcactggctgcccaggacctctctgctcttacctcctactgctctctcttcactcatcctgctgctgccccactggcctcctgactgggcctcaggtatgtcaggtaggttgctgcctgggagccttgtacttgttcttccctctctgaattgctcattccccagatatctgcttgttgcttccttgtgtcttacaagtctttgctcacatttcacctgccctgacactcccccatcctagcactctgcttctcttgctatatttttctccatagtactgaacatctgatatgtatttgttttgtttgttacctgaatcttgtttttcctaaattatgagaacaggaattgttagtgttatatcctcagcacctaaaacagtgtctggcacataatagaccctcaggggatatttgctaattagagcttatttccttttttaagcaagagtttttaaccgtgcatgtagggccttgcgggggagttgggttgtagcatttattagtcgatataaaccctcaggtggaaaggaaaccaaagactcatgaagaatcttcaggaggtggaaagtaatgaatacctacaattaggtgtaggtattttagtttccttattaatagggatggtttataacaatcgtttttggttttacagcaggtaaggataacttcaagggataaatgatggtactacagcataatgaaagataaaacaaaatccctccagaattggggaggatatttattgtaacagctaaaaattatttttgtgattgttttaaaatacgattttatagaaacttttcctcttatgcctcatagaatatagttgaacatcatgatacagctctattcaagatattaagctatttttcattttctattggtccttgtattttcttctcagttattctcccattcggcttcttgaagtatgtgttccactccccaaaatattcattaagcgtcaggccccattaaaagtgtcccttcttcaggatctgaaggacttctttcaaaagtaagttcagtttcttttatcatctttttttgtttatttacagctattttaaagtatcagatttattcattttattttctttgaaaatgtaaattttctaattgtatgctatgcatgcttggtgtacaaaaagaaaattgtagacgctatggaaagataataaaaaagaagccaaactcttttagacataacaacttaaaattttgaagtacatccttttaggtcttatttactctgcaaaactgtatgtaattcttaattaggtaaaacataatttcaattacattttgcatgtagcttttttttctatttatgtgagatagaaaatttgattcaccctttctaaagtgtttaatgctctcgctattgttagagtttcacaggtatatgttgccattgatgaaagacttgcatctttgaaaactgatacatttagtaaaacaagagaggaaaaaatggaagatatttttgcacagaaagaggtaatttatttctttggtagaaaattcaaaagttaattattgattaaaaattattgattaaaaatcaaaaattaataatagcagtttcttttggggtatgtctgtatagtgtctgtctcctgaagttgtttctgttccttgtagatggaagaaggtgagttcaagaactggattgagaagatgcaagcaaggctcatgtcttcctctgtagatacccctcagcaactgcagtcggtctttgagtcactcattgccaagaaacaaagtctctgtgaagtgctgcaagcttggaataacaggtgtgattttgcagtctggttttctttaatattattgccacatttgttctaatgtgatatcttaaaacttaaaaatttttaattatggatgtttcattttgtgctcaagttaaagagccttttgtttcctgtcactctcctttcctctttatattttaaaatatatgtggtatattttaggtagcttgtgaagatgtaaaaaccaaagaggagaacagcctttgatgctgtttctaatataatttgaaacaatgttgttcagatgtgtgtgctgttgaattgtttttaactgaattagtatgccataagtcactattggtttttggccagcaatttcaaatatattgtatattgatatgtttatcttatgcctttatttaaacatttattttttagaagtatttttctttataataatttgctattttttttttacaactatagtgagatcaacaaatgctcctggttaattagatatgacaaaaacttactcttatttatatataactttaaagactatggtaatccaaataggaaaactttgtgatttcaaagttgtaaagagaaaatttataaacagcactcaaggggctagaaacaaaccatatatatatatattttttgcttttggccacaggttgcaggaccttttccaacaggaaaagggtagaaagagaccttcagttcctccaagtcctggaagactgagacaaggggaagaaagcaaggtatgaaatgtagtcttgtctttggtccttaatcaatagaaatatagttagtttcatttataatacttagcagggatagtttaatatgtactttagtaatgatatccagtagggttttccattttgaatttgtgaggtttcaaggaactatgacctggattacttttatgttataacctgaaataaaatgatcacttataatttatttatttgaaatgtttataaaagctacagtttgctgcatcactgagggtagcatcatggtgacagcaagtgctacagaggaaagtcagtggatgctctgagtataatagggtgctcatttattttatactccgcagttttggttgtttttaaaatctccgtgcttaaataagaggtttttcttaaggtatttgaactgcgtaagttctttttcaactgtgttagttttctcttgctcagtgacagattatcccaaacatagtggccaaaaccacaaatttatagatagatatatctatatagatatttatagacagatacatctatatagatatatatagatataatacttaggattatcccttacacctaataatcttgtatagatctatatctatctatagatctatctagatatatatcaatctatctagatatatctatctgtctatcgatctatctatctagatagatagatatatctatacctagatgcttaattccaggaagcgagctactatttagaatgcaattctcaaagtgtggttcaaggactcctgatagtccaagaccttttcagggactctgcaaaaatttttttttttgagagatggagtctcactctgtcgtgcatgctggagtgcagtggtgcgatcgcggctcactgcaacctctgccttccgagttcaagcaattctccttcctcagcctctcgagtagctgggattacaggcgtgtgccaccacacccaggtaattttttgtatttttagtagagatggggtttctccatgttggccagactgatcttaaactcctgacctcaggtgatccatccaccttggcctcccgaagttttgggattacaggcgtaagccaccacacctggctcatttttattttttttttcgattgcatatctgtatgaagctagattttcttcatatacttcaatcataatggtgtatctcaacagattacatgcagaagccaaatatgaatctagctattttctatttagctagacattaaagatatttttcaaaatgtaaagcagtgctactctttcaccaattttttttttaaattttagaaaatcggtatgttacgtgtttaaatgttacttatattaatgggtttattatttataaatgagttcatgcacatttaataactttctctgttttaatttctaataaggtaaaaatttctaaggtaaacaaaagctaattaaaaatattaagggggtttttagtaattttttaacggtataaatgggtcctgagaccaaaaagtttaagattcactggattaaaaaattttttgaatgtgattttcatgttttccataagaacaaagtatttacttctctacagataagtgcgatggatgcatctccacggaatatttctccaggacttcagaatggagaaaaaggttggttcttgagtctggtgatcacttgccgtaggatttaaagaaatttcttattgtataagttgttttaaactgtttgagcagtttttccgatgctgttgttcatagacttcttttccttcctgtagagcacttgtaatcctatcagattaagattatcatatagtgtattgcaataagataaatcaggttatagaaatacatgaaatataaagaaggctgctattaggggaagaaaaaacagaaaaaagtaaattgaattttttttgcttattacaaagtcaatccccaggtttcattgcaaaaatcttataaagtttaatttacatttttaatgtcactaaaatattgtctagacctaagatgtctttcacagtatttcattgaataacttggtgtcatcatagaaactttgtggggaaatttacatagactgtagaaccaggagctcaacttacaattcttggcatttttctattataattgctttttctctcccagaataagcatgccgtctttctcagagtgtcctaatttcattctctattaaactaaaaactaatatttaggtaggcattgtggtataatggatagcgtgttggataggagtcagtcttggttgacagttgcggtactgctaccaggatttgattgtagtcaagtcatatataatctttctggatttttgtgtccatttctatgacatgagctatatgacctagatgatttttaaaaagtaaaaattctaccttttaggaagtaggcatatgaatgagcatttaaaaaatattaggtagacatggacttaatgtatgtaaatgactcatttaagattgtgtttttctttagaggatcgcttcttaacaactttgtccagccagagctccaccagttctactcatctccaattgcctacgccacctgaagtcatgtctgaacagtcagtgggagggccccctgagctagatacagccagcagttccgaaggtagaggttaagtcacttttaccatatttcattgtatctaagactgaaagatatatcattgatttacatgaattacagccatttctctaaggacagatatttgcttcataataacagattcatgccttgctttctgtttaggtggtaactacttaaattatagtataaactttgaaattaactttaaaattgctatttaactcgggtgtaatagccacagtgcttcagagatgtttaaaaaaaacaagtgtcccttggagtccatgctgtgtatgcttcactgttgtttcctgttttatcccatagtggtcttttcccttgatcagaaattttaaactgttattggtatagtatacatatgtccatattccgaaaggaaaagaaccaaagtcagcagtttatacttcttattctctgtgaatattaatatataagtagactatttttcattttagatgtgtttgatgggcatttgctgggatccacagacagccaagtgaaggaaaagtcaaccatgaaagccatctttgcaaatttgcttccaggaaatagctataatcctattccatttcctttgtaagtattattaaaaccagtggctcttagcaggggggttatttttccttagttgctcgcttcatatttaaatatatttatctgatgcatgatttttcaatcctaaattttatttcattttagtaaaagagatgtcttttgatacaatgtaatttgtagaatatataatcattatgatatagtagatgagtggataaagcactagccagtgttacttataaatgtttgaattacttgttctggaaacacttgtttgtttatcttttttcaaagatatgtggctatatccactgatttaataattataaaaacatattgctggttatatatgaggttggtagttgatttgctgggacatggcattgtcctagcactgatgatgaactacagtgacttgtacaagttttcatgtgatcttttttttatggggtgcactcaaatttggaaagaatataggctggctgtgaaagtttatttttcttccactggttgacccatgtggctatcaaatgctgcccttgttatggaacaagtatatccaaaaagtggacatttttgttaataaggccttttattttattaactgacttacatatttattcaatcagtatttaaacttttatttagtgcctgtttaagtgtcctgtgatgtagaataagatttgtaaggaactgtcatcactcttaggagctcataatctttgggaagatacagttagggctcacagtacagtatgtgatatgttacatgacagtaaaatataaaaggtcccttttttctctactttagacctgattatatcagtttcttgattgggcttggatccctcttatgttatggtctttgttcatgctctcacatttatttttatttttttaatttttattatttttgagacagagtctccctctgtcacccaggctggaatgtagtggtgtgatcttggctcattgcaacctctgcctcctagattcaagtgattctcctgcctcagcttcccaagtaactgggactgcaggtgtgcgccaccacacctggctaattttcgtatttttagtagagacgaggtttcaccatattggccaggctgctctcgaactcctgacctcaggtgatctgcccacctctgtctcccaaagtactgagctactggacccggccctttaaacttttttttttaatatagtttttttctttttgtttgtttctcttctctctaaaccactttttgtgaaacacagtcttttttaatcattcagtcctgttctttctaagaagaatttactagaccttcccccttacctcctgctccagtcagagtaggtttctattatatgctcttatgattcctgtcttggtccgttcatgctgctctgatgaattaccatagactgagttgcttgtagacagcagaaatttaattcttacagtcctggaggctagaagtctgagatgagggtgccagcatggttgggttctggcaagggtcctcttttgggttgtagattgctgttgtctctttgtattctcatgtggtggaaggaggctgagagagctgtgtccagtcttttttatgaggacactaatcccattcaagagtattccactctcatgacctaaattaccccctaaaggccctacctccttataccataacattgggggttaggattttaacatatgaattttgtgggagctgggagggtaaacattcattctgtagcaattccctcatagcacttaccacagagattatatttatttatttattggattatctgtttctttacccccactacactataaaccctatggtgccaattttttttttcgccattgtattgttaacatcttacacatgtaagatccttaaaaatagtttttcttcatattgtatccttggcatgtttggtgttcagtaagtagcacctaaattaaaaatgactgtgcctaacagtgcctaggtgagttagggtcggagtcaggggagcaaaatttaagttgaaatggagctacactgggtttttttgtgtgataatagtaaagtggttgtgttgcattgtttattattgaactagtagtatgtcttctccacaatttctagaagactttttctctctagcttgtaatggaacaaaatattttagacttctgctgttcattttaaattctttttgagattagtgtggccagagtcttggcagtatggaaaggtttgtttggagtgactattgcagaactctctgatttgaaacctaattaattctgataattttctaattgccttcaaaaactttctcttgaaatctttttctttcctaatgattcaggttatcatatctgcatacattttggctttcttaattatattcttaactctagagtacttagttaacttatgtattttaaaaatttgcatagtgatccagataaacactacttaatgtatgaacatgaacgagtgcccattgcagtctgcgagaaggaacccagctccatcattgcttttgctctcaggtattattcatgggactttgacttatgatgatatctatgtattttatgtatgtctattaatcccttatttttggaatctttcacataaagcaaaatgattaaatataagcaaaaatatatgttctttagtcgtgatagcttgttcttttttttttttttttttgagacagaatgtcactctgttgcccaggctggagtgcagtggtgcaatctcggctcactgcaacctctgcctcctgggttcaagcagttctcctgcctcagcctcccgagtagctgggactacagacacacaccaccatgcccagctaatttttgtatttttagtagagatggggtttcaccataatggttgggctggtcttgaactcctgacctcaggtgatccacccacctcggcctcccaaagtgctggaattacaggcgtgagccaccatgcctggcagcttgttctttttgtgcataaaaatcttctctgaacccaaataatgaagtataggatgattcctaagtaagctcctaataactaaattgacttggcaagttatttactgagccttgaatcctctatccattctgttaaaatatgttttcccttgtatcccttctctcagtttacttgttgacatagtacttggagcatcctgccttgtacaatctgaatatcttttttgtaccattcactcattttctactaccctacatacgaggtaaaatttttctacttagaaaacagcagtcacctcttacccattgaggatatgttctaagaacccctgtggatgtccgaacctacagatagtactgaaccctataaatgctatgttttttcctgtacatacatacctatgataaagttaaatttataaattagcctagccacagtaagagatgaacaacaacaataagataggacagttttataataatatactacaacaaaagttatgtgaatgtgctgtctctctttctaaatatgttaatgtttttggactgcagttgactgtgggtaattgaaaccatggaaagtgaaactatggatgaggagggactactgcaatgctgttcttatgccttattccgtagacatttctgactttttagtttatacagttatcatatatcttaaagctatatccactgtaaaaatatatagtatgtctaaaagggcaaagcttttaaacttttttttttttttttgagacagagtctttctcagttgcccaggctggagtgcagtggtgtgatctcggctcactgcaagctccgcctccgggtttcacgccgttctcctgcctcagcctcccaagtagcccgccaccatgcccggctaattttttttgtattttaagtagagacaggatttcaccatgttggccgggatggtctccatctcctgacctcgtgatccacccgcctcggcctcccaaagtgccgggcttacaggcgtgagccaccgcacccagccttaaatcttaagaacatcttggtggtagctgaacttagtttaaatagaatctaagatgtttaaagcaattttttcccactggaaggcattacagtttttaatttttcttgttagatggtggttccagggagtccatttattcaacaaatatttgaatgtttacaaaatgtgatatactagatattggatgtacaggagtaaataagatagatttacatgcctctgcctttatgcagtttagagtttactgtgtagataatgaggaatttttttcctttataagattccatatgataacatttttagcctttgcctttggcctttgtgaacataatataagcttctcactttatttaatagtcataatatattaataggtaagaaaggtaagagagtcaaccaaatcaaaagtattttttgtactattaggaagtaaggattctagctgagatcatcttttttcaatgtcttagaaaaacaatacatcctaacatgcctctcttgttgcatgacatttatctaaagcagggttcctaaactgctaacctaagagtcaggttttttttttttctttttggttaatagaacattaaacatttttttccccttaaagtttagtatgttaagtagaaaagcactctcaagttctttttttctctattgtactcaacactgaggcttcaaacttttacctgttcaatccctgaagtcatttgatctaaatattagtaagtaggcttttaaaatctttgttgagggtattgatactggttgatgagtcagtgggtcatattttgtattaccttctatgaagacattgaccttcctcatgaagcttctacttgattgcttttgtcagttaaccaaaattatttaattatggaaacgtataatgattgtgcttctataatgcaaaacttactctaaagcttatgatttacttttaaagaagaagttaatgtttaacgtttttcaacctatttctgccctaagttgtaaagaataccgaaatgccttagaggaattgtctaaagcgactcagtggaacagtgccgaagaagggcttccaacaaataggtgattcatgattgagtagaaactattttaatttagttatgttatcattttctatatgcttgtacttcagttttattacagcatttatcatcataaaaatttctaggattcagatagtggcactcatatttcagccaataattgagacaacaaagctcaaatatagcaaaagaacttaaacagtccctcaggatgcagttttatgataattgcaatagcagttaaatgtatattgcagatttaaggacatttttgaaaaaaatgaaaaggtgtttttatttaaaaaataataacttgtaaaattggtaagctcctgcttgacttggcaattcaaagtatctgtgaattgtttataataaataaaggtcctaatacttcaaaaaccaaacccaaaccagttattttggcaagttcagtattttagacttttagacagaaattgtcacatgctcattattgtgattcttactgaaatagagtttccatctctactaacattatagttttatctgaattgtcattgtgataactagtgtgagagaaaggaattgatattttctgtagttgtttgatttatgggtgctgatatagtgttaatggacttgggaaacataatgtattcctccatacatttttatagttacagcatttcactttaagtccagtagatatttcagacttttttttctgtgttctagcctgtgtttatgtaaattatgaatcagacttttgatattgtaggtgggcttaggtaggaagtaaatcagtgtacagcgtaattagtgtagagtacttactattttctgtttgacttgtataaaactaaatttttctttttttttcttttcattttagtacttcagatagcagaccaaagagtagcagccctatcagattacctgaaatgagtggaggacagacaaatcgtacaacagaaacagaaccacaaccaagtaagattacacatggaggaatatttataattagatttacctataattatggtttgaaaaagaaaatacataaaaacaaataagtactatctggcaatgatttactacttatgatctctgaaatacaaatttagatatttttgtatgccctctttatacgtgttttgcttttcttcgttgattcttgtctcaagtgtaagttcaaggtattgaattagaaacttttagaagctctttggtctatgcaaaaaggagaatctgcttcctagaagagctttcagtttaaaatatacaacctaagatgcaagatacgtttttttcctttctagttttacagtattactggcactatgaaaataaattttcatacattccctcctttaatccccctagtaaccacataagaattatttgacatatccttatttttcaagaaggaacttaaaatttagatttagtaacttgttcaagatttgacagtgctggagctgggatttgaatttgtgtttctctgactttgtcaggaacacattcagctacaagtaacacaaaattcaactgtaaggatttaaacaaatgacagcttatttttctaaaataaccaaacctggaggttggcagttgctggcatttgtttagctgctcagcattgttataacattaggggaatagtttctctatgattcctttacgttttcctcatggttaaaagtgtttcatttttgtttgcatttgaggtagaaacaagggtgttgcaagctgccgctgttctctttcattaggaaagcagaggctcctcctttagtcagtctcattggtcagaactgaggctcatggccactcctgactgaaagagacagagaaagtagggagcagcattgtcccggttgggttatacaaatcatgatcctttcctggtgctgtacacatagttacacaccccaccccgctcccctcttcccctaagttggcattctgttggtggggaaccaggtaggagtggatattaaagaggcaatactagtgtctgttacccaagtaagcacatttatgattgacattttctgtgagttttgtaaatggtaaatttgcaagaattagaatcatattaatagtatgatgaatgaagctcaaaactaggagtaaaagtatagagcaagctttagtaaattcttggctgtttgatggccttttttattttttgaaagatgctaaaaaggaaatcttatattttcctgtgttaaactgtaggaacacaaatgacacaaagatgttacctttaactgaattcttgtagcttgtgaaattgttttgtgttcatttagttaactatattgcaatactgtaagaactcagccctaatataatcttgttgcttaagctgtgactgcctttgaaagatatttttgaaaggctttctagataattccctatatttttatattaatctagctgagtgttaaccttctatctttttttatgtttttattaataggggtttttgttaaaaagggtgcttgattacgcaaaaacaacatgtgaaacaataagtaacaacatgaaggacaatgtgcaaacaaaagtatttgcattactcagattgattagcttcatatagtggttacaactaacaaagaaatgagctgaaaatttttatttttcatctgtagtgacctgtacttaaaattaatatgttacttcttatttttagccaaaaaggcttctggaatgttgtccttcttcagagggacagcagggaaaagccccgatctctcttcccagaagagagagaccttacgtggagcagatagtgcttactaccaggttgggcagacgggcaaggaggggaccgagaatcaaggcgttgagcctcaaggtgtgttaaagaagagtaaatgtgcttattctatgctttgatttatttattttagttgcatatatttcttatagccttaaatagataattcttagtgctgaaactggggcattccctctgttctcacttgtcagtgtggtagagtggaaaaagcatggcactgggagagtccaactactttcagatttcactgccatcgtgtactgtgtggtagaatcagaaatctttagtttccataacgtgtaaagtggcaaaaacaatgtcttccaaggttgatgtgacattccacatgtaaaaacatttgtcttaatatgtaactcatattatatactcaaatgttttcattccactactccctcaaatgttttctttcttttcaagtccctgtaaagttgcagctatttatgtcccctttcattttgacatttaaaaattatgtctaatgctgggtgagatggcccatgtctataatcccatcactttgggaggctgagacggcaggatcacttgaagccagcagtttgagaccagcctgggcaataaaatgagacctctgtctctaccaaaaaaaaaaaagtgtgtgtgtgtgtgtgtgtgtgtgtgtgtgtgtgtgtatctacctcattgtgtttgggtatgggtttagatcaggaataggcagactctgatgacctgtttttgtaaattaacgtttattgggaaacagacccattcatttacttatcatctggttgctttcactttatattggcaagttgagtagttggaactttttcagagtgaaatatttgctctgtctctatatagaaatagtttggtgacctctgggttagtccgtgaattagtagttttaaaaaatagcaactgctgggcccagtatggtaatcccagcactttgggaggctgaggcgggcggatcacttgagactaggagttggagatgagcctggccgacatagagaaaccccatctctactgaaaataaaaaaattagccgggcctggtggcgcatgcctgtaatctcaactacttgggaggctgaggcacgagaactgcatgaacccaggaggtataggttgcagtgagccgagattgcgccattgcactcctgcctgggtgacagcaagactctatctcaaacaaacaaaaaaaccaactgctgtaataaattttgactagtaattaagatatctgtttagtattctataggctgcttgctattgggctgtagctgtttataatgggatccttggaaggcatagatgcttcaaaaattattttgaaatgtgaaacttaggggactatttgttcttctagatattaagatgtattttaaatctctaataattaaacagaaagtagtaggcttgcaaagcaagtcagtcacatataaaaaaatagctctgacatagattctagtatattttatgtataaaaatatgttttaagtatattaatatataattgtattatatgttaattatgtatgttatataattatatatgttataggctaataaagttatatattatgtaactagtatataattttatgttttaaaattatgttttataaaataatattttatgcatattttatatagcttagtacatgataaagcattgcaaactattggggaaacattattctacacacatggggaaaatagtcataataaagaagccatggaagttcttggtataagaactgagttactatattgccactgtaggaatcattcagttgtaagaaaaagtattcaacctaaattctctatttttctaatttaaaaactgaaattccaaaaaacatacatttttattgtgatcatttagacagtttaattgtatatcttatgcttgcttgatagtgacttgagtaggatatttttacttttaatttgctaagtttttttgttacatcaaggaaaaaggaaaggacaaaatgaactgataattactcaaaaccttggctttactaatgatattaaaccttttagatgaagtagatggaggagatacacaaaagaagcaactcataaatcctcatgtggaacttcgtaagtatgagatattatatcattggtttggggagagggactacagttgagccatctctattatttgagaattctagcttcatactaaataaatgtcccagttcttgaaatgaatattaagtctttatgctgacatgaggtatatttaatcaacagtttataaaatataaatttaatttataaagaaatttaatcaaaagtataatttatttgtaaattatgctcttatttctaatagttaaacatttacttattatgtaatctagagccacagaattttgacagttgcatttagaattgtgtatatgtgtgccagaaacgtattatccagaataatatgtacagtacattttaagatatttattaagaattactataaaagtacaatgatgttaatatggtaggtaacaagtgtaaaattagaactgagtcttttttttatgtgatttttatttttttctctttttttggaaagtatgtaaggatggtcatacgatgttctttaattccccctgaagatcagtttaacaaaattgtgataaatagggatttggatctaacatgcaaaatgccatcatgaagaaacttaagtggttgcatttggccagggtagggagtgagggagcggaggaggaaagatattttctgagtcataaagctttttaaaaaaacttctgttgtttatagaattttcagatgctaatgccaagttttactgtcggctctactatgcgggagagtttcataagatgcgtgaagtgattctggacagcagtgaagaagatttcattcgttccctctcccactcatcaccctggcaggcccggggaggcaaatcaggagctgccttctatgcaactgagggtgagcctttctcatcgtttattgattggttcagtagtcttcatctctttacctcagaaaatgcagcaggaaggatattgcttaataagaactttcataactatgacttactagttttaacagaggtgtttataaagttttatttagtagttttaaaaagtagatcttctagaactgaacttagatgtgttattgacaaaagtagagggtttttgactatataaatccacaggatcccttcagttaattgagaattgtgttctgatcaaaattttcactgtgacattagaaaattccccttaaaatcataaaacaacctaatcatttaggatctttggaatgatgtgttatagattacagtatatattgtgattgagtaaacacataaaatttctgccttttagatgatagatttattttgaagcaaatgcctcgtctggaagtccagtccttcctcgactttgcaccacattacttcaattatattacaaatgctgttcaacaaaaggtagaaatctaaaaccatggtctgtaatcaaagtttggtggcttttttcacatcctgaatctttctgttagtctattcttgtgttgctataaagaaatacctgaggctgggtaatttgtaaagaaaagaggtttatttggctcatagttctgcaggctgtgtatacaaagcatagtgctggcggcgtctgtttctggtgagggcctcaggaagcttacagtcatggtggaaggcaaggggagccagcatgtcacatggtgagagacggagcaagagagcggggttgggggtgggggaaagtgctgcattcttttaactaaccagatcttgcgtgaactcagagcaagaacttattactgcaaggacggcaccaagccatgtatgagggttctgctcccatgacccaaatacctctcaccaggccgcacctccaacattgcagataacatttcaacatgagatttgcaggggcaaacatccaaactgtaacagtcctaatggcagttttctctgtggtaagatatatgcaggacacaaattgatctctgttatgctattgaggtattataggattcagagatcttaaactatatatttcctgcgtttgttagtgaggaaccaagtgatcttgagcaagttgcctttaattagtttatataaaaaatcacttaagacattaagactggatgaaattaggtgcacattcagtagttggtaattttgctgagactgaaatttgaaacgtgtattgagaaacaaggggatgcttcctttagctagaaagagaggcagccaatggcccaggtttgcccacttgtcattgagcagccaccagtgccccccaccccatcccaactgccatcttagaatcatgaaagagttacatagttttgggttttttttttgaagttatcatggtttattacgtagtattgggggaaattacatgtcataatgaagtttttgaatttaatcaactgaaggattattccttgggagaatgttaaggatctaatatttttattggcattgctgctgtactcacttctgagattcaagagtttgagtttttttcaaagtcctttggtcatattaatagaaaattatgttcagttattacatggataatccttatattggtattaaattataaataaccctttttgataagaatttattttgaccttctcttgattagaggcccacggcgttggccaaaattcttggagtttacagaattggttataagaactctcagaacaacactgagaagaagttagatctccttgtcatggaaaatcttttctacgggagaaagatggcacaggtaagggtattggactatgtgaagcatttagctactggaaccttttgtacctttggttcttaaatatagtgaatcagagagtaacatcttattttatagtaaatatttaactttggttactaggccatatttaacttttgacttgctcagccttggagaaagaatgaagaggaattattcttgtggagtggtcttaaaatacagcatatccaatattctagaatcacagaattggtagaataagaaggcagatctacttcccgcatcttttttgaagattaagaagctgaggcatacagaaattaagtacttcattaaaatcactcagttattcctagactcaggttagtctagattctaaaacagtactgttcaaagagctagtccttgaactgtttccagtctatgataagataaggaacgtgtgctagaatgtaattcagttcactgctttcttcattgagaacaccttctatgagaaaaatgtcagctgaactaaacaatatacttaataatgtaattgatttatattctggcactagttccctaatgttgtagtgagccaggagcaactgaccagcattggtctgtagaatatactacaggttgtactggtcctggtactcaagtcccctgtcctttgagtgcattgcttttcctaccacccaaatattcctgtgttatgctcttctacattgctgtaagtaattgtccagagtttggtatcaaaactatgtttcttaagactatttgcatctctctctttttttttttaacactgttttgtgatccaatgatgtatttctggagttacttgttaatgtatcttgaagttgattaaattggccagtgtatttcggaaacgtttcagtaattgagtacctaggtgttaattttaaatgatagaattatgttactgtgtattttatgtactgtacaatgatatcaccatgcgtttaaaaacttaaaaattatgattcaattttcagttaaactttggagtgtgtgtaatggtagtgactataaggagtctttctgccttttttatttacttacattaactaatcatttgtggcaacctgtacataaatgacaaagcattttctttttactaggtttttgatttgaagggctctcttaggaatcggaatgtaaaaactgacactggaaaagagagttgtgatgtggtcctgctagatgaaaatctcctaaagatggttcgagacaaccctctatatattcgttctcattccaaagctgtgctgagaacctcgatccatagtgactcccatttcctttctagccacctcattatagattattctttgctggttgggcgagatgatactagcaatgagctagtagttggaattataggtaagtcaatgagtaccctgcttatatttcatgattgaagtcagagtcaaattttaaagattctattgaacctggtctaataatagcttattgaatattttcacaaactttcatttgaaatttcaaaaataagttttttattctagtctcttctactcagtttcctcattttcattatctctaccaaaaatatgtgaattaaaaaagtcacttataaatgaggacttttgtgattgttgtgaggattaaacaaattagtgtggagaaaccctgagaacagcacttggcacattgttagcactcaataaaagttaactgtcatagaagtagtagtaatagtcattgctgttgtcatttgattatatttattaacatagctttattgctaatttcactccttctaccccccagattatattcgaacatttacatgggacaaaaagcttgagatggttgtgaaatcaacaggaattttaggtggacaaggtgagaatttttgttttgtttaaattgttcacatgtatttcttatcagttaaaattttaaaagtggatactgattcttttttttagttgtaaaaaataagtggttatcattgatttcattgtcatttctaaagtgaccaaacaataatgagaattatataggtggggaaatattgggtgtaaatatgtgttaaaatgtcagaggttctcaaaaagattttttaaaaagccagatgttctgaataagagattgtctttaattaacacttgagacttccaagttctctaagcactgttagcctcatttgtgctcttagacatcgctcatctctaggcaggagctagtctttttaaacagaaactcgtaccaggtaggactttgtcccatggcatgtggcacaacagataacctttcctgcccactcagactgcttctgcatgtgtatctttatggcagtttatatgaaagaacatggtcagttgacttcagttgcccaatcaattatataacagctttttcccccttttctctttcataatctcaggtaaaatgccaacagtggtgtctccggagttgtacaggactaggttttgtgaggcaatggacaagtatttcctaatggtaccagaccactggacaggcttgggtctgaattgctgaaatcaagcacatattttgaaatggactgtgaaggaaaaggggacaggaacaaaggaccaaaaataagctacatgttttatttcttcatcgtgttcaccactgtatgccaaggcttttcagttctgtggctgtttagactgtccgtaatggaatggtaaaactccatgaatttgcactttggtttttgatacctgtggagctgtctgtaggttgggaagtggcatgaaaattttcttaagctaaaatacagacatgtttcaaagggctaaagttggagatgagtagatagggtgaaaaatgggttaaatttgctagcttaattgttttaagaagaaaacagtgtctcataaattgactatcctggcatcacatttaacatgttatctacttagaaagcatttgtagagctgctgaatttgttttgtgtttttctgtaataatttaatgttacttattatcagaatttctgaaacctttacaaaaattctgatttattccattaatggccagttaaacacgtgggcatttattgttttattgaggaatttgacttaaactgggaatcctgtcatgttgtttatctttccagcttgcctgtttttgagtatgtttgatgtttttaaaattttgtcttctctgtggatgacaggaggctacagcaattaactttaagcctccttttagagatatttttaaagcttgtttaaaatttttgtgcaattcatatattaaattgcacttacttgcatacgctcatattctagggttttttctctatttttagggtatcatagtaaatcattagtaaatgagtctgtagttactaaaccctaatggaataattattaatgaaagatttttgaaatataaaaaataaattaggcccaatccaagaaattgagtgagaaggaaacacttgttttattcacagaggtaaagtgtcttttcaatataaccagcaatttaggtggcatctataaaataaaaaatttctactgtggacatccccttttccaactttctacataatggctagttctgactactaagaaatgttaagaaataggccaagtgcggtggctcacgcctataatcctatcactttgggaggcctaggcaggcagatcacctgaggtcaggagttcaagaccagcctggccaacatgaggaaaccccatctctactaaaaagacaaaaaattagctgggcatggtggcatatacctgtaaccccaactacttgggtggctgaggcaggagaattgtttgaacctgggaggcggaggctgcagtgagctgggattacaccattgcattccagcctgggcaacagagcaagactgtctcaaaaaacaaaacgaaacaaaaaaagaaagttattcttagtaaggaacttcttgtttaatagcatttttgtttattttaaaaagtgatcagaagtagtaaactatctttgaggaaatactgtaaccccagaatatttcctcttgacttctttttgtaacaaggataatttagggatttataaagttgtaaggatttcactgttttcggactgcctataataatagcacattaaccttcacataataagaaatctggacaagttcagttacacagtatgatgaatacttgaattaggaacattgtggaaaatttgctttagagaatcaaggcagtagtttggtatttggtgcttattaaaaatgtggtttgttttgaactggaagcaagttgaccaaggacttatgactaatgtgatgctaagttccacttggccccttttaaaaacgtgtatgtgccttttgaagatacacaaaacactgaggattttagttttgaaatcaaagactattaaaggagctgtacagaggtaaaaaaataaatgtggaacattattaacttattttgtgtctaggaacaatggattttgtatctgatttaaaatgccaacactgttttgtctctgttcattttttctgtgaggatacttaaggttattattcctgtctgtttcctgtactcccctagtcatgagcacttgaagtacaaggtgtctccccctaggtgcaattaggttgtttctttgtttttagtttcaattctatgtgcatagcaggaatgctccacaggaatggcttctgacaataatctgtcctgttgattttgttttccttgcccatgacttgaacaactgtgttttaaagtactgtagtctagtaggtaactttgtggcaaaaattttcaatataatacattctgaaacaatagttgctgccttgcaaaggtaatctctcattttaaaattggacagtattaatgaaggggaaatatacaatttatttctattgagtggtagaactatatgtctggtcccttgctgctcttgtttaggccactatcatagatatatttcaaatattgtactactcagtgttaagtattgaatgactgtttccctttccttcaaggcctagagtatattctgaaaatttaggaatgaggaagaaatcttaatacttccttccttaacatacaacatgagtcccgagaataattgatagtagcaagagaaaactatgtcagtaacatgttgctttgtataaaaatcttatttataaatgtgaagctttttgatgccatcaaaacttattaaaaaataggatttacttttttctaattctgacctaagaaaaataatgagaacaagctgttgcaagctcttttgtagtctattgaatattttatagatattcaaaatttcctacaaactataattttttccatgatttagcagtgagtgattttctagctttggctcttattaggtattgtaaatagtagggttatatcgatatcagcttttgtgatggcattgtggtcatcagcttcatgacattttacccatttgcagtgatcctgtgtaaaactgccaaggaaagtaattacctgtaggagtttgctgagcttgaagagtgaaaactgttgtgaatgagcctgatcataaaacggaccaggccattcattattcctcaagtgttaatatactgacttatgcagtattcaaaccatctagtgcaatgtttttgtttttgttttttttttggtaacacaggtgcagtgtattatagaaaaaataaaaactacaatcattagcagttttaatactgctgtgtcagttttgtaaaaaatgtacattatgtcttttgacatgttgaattttaaactagggaaatgacattgtaaatcatagtagcctcttttaatttaatatgaaaaatgccactatattgaaagtacttaatgtattgtatatatttctctactttggttctagctattttatatgattgacatgttatttaaaagataactgccttgaacttttggagacttgtactgtaaataaagaaatcttaacaataaactcagaatctacttactccacttagttttgaatctgcttgaagaaattgacaacaaaaggaaatggagtttgtcctgcaagcctagtctttgtgttactggtggagggtgtccaggttcttggcgttttgaacaaagaattggacaaaacacaaaaagcaaggaaagaatgaagcaacaaaagcagagatttattgaaaacgaaagtacattccacagagtaggagcccaggagcactggggcacaacagctcccgttacagaactttctggggtttaaataccctctagggatttaccattggttagttggtgtacaccctgtgtaaattaattagtggcctgcaatcagtgtgattggttgcagaaagcaacccatcaggctgaagtgaagttacaaaggttaaaccctatgcaagtgtcgttgtggaaaacaaccagtcagaggccgaagttacaaaggttacaccctatgcaagcatctcattggttgtggaaagcaaccaatcagaggctgaagtgaagttacaaagttacactcctatgccaatgtctggttgcagaaagcagccaatcagagatgctttcaattttccgtctgccatgcagaaaagggaagagggtgcttgcaaacggagtattccagtccttttgttacttagatgtagaaagttggggttttccttttgatttagttctagaaagtcagtgtgaatcggccttaggttccctgcctccagactattctgcctcacctggcctagcagtgtcgctttctgttcattctgttcattctcttcctggttgtttaaccactggagaatccgtaacttaaggtaaacccacagttcatttcagccaatactgggcaagcagcattccccttctttcttctgatagggcactaaaggaagtcctgccctcttccatttgcgattggctgaatttgtgggtgggcagggctctccgctgccagtgcctccgtgctccgagggaccacaggtccactacgtgaggggaggcttccctcccacaggaactcacacctgtgggcaggaagccgggaggcccgccccctgcctgggttcctgcaggggaaggcgagggtggctggatagagcgtaccctgagaggtggttgggcaggaggctgggctggtggccagctgcgcagggagaccgggaggtggcagatctgcggggcgcccagtcacttcctccacgttctcgtgctgggcgggaggagcggatggggcttgggaggcagcctgctctccagtccctatccacccacaggttttttgggtcggagaggaattatctgataaaattcctgggttaatatttttaaaaacggagagtttttaaaaatgatttttttccctcgaaaatgacctttttatgcttcgaagcagtttgtcaaccagcatagtgctttttcttttctcttctttttctacgataaatgaaagcatttcttcaagaaaaaggcacaggttccttgaacaggtaagaagagcgccccacttgttctcccgccttctgcgtgagcagcccctacggggtctcctactggtccctaggaactttcgccacacccggagcggcggcctccccgagcccctcggggccccttccctggcagctgggatgagggcagcgtgctcctcgcaggttacttggtcctgaggtcctccctcaggcgctgggcatgcctggggcgcagaaccgcagctctgggccagacccggctgagactgtgcttgtgcggcgagtggaggccgctcccggggccctgccgccctctcgctcgctcggggctctggagtctggcgggccgcaagccgcgctgccggctctacctggctttgtccatccagccacccaccaagcgccagggtaaaacagcacccggaagcagtcgtcgcctgctgccaccaggctgccttctgtggtgaaggtgggggccagggttgcaaggccctcctcttgttggcatcagccaggcacgcagtgctgtcg

SEQ ID NO: 9 - RNA, 2130 nt, Homo sapiens
caaccatgtaagcagcttcgcttcctgccgcaaccgtccgcggcctgaggagcccaccgccgctctcgggggccgacttccgggggctgagccgttgaagcggaggctggggcggggggcagccggcgcggccggggcaggaggcgcagactcatgaaatggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaagtttgattcatccagattcctcaaatactcctctttcaacaagacttgtatctgtgcaagaggatgctgggaaatctcctgctcgaaatagatcagccagcattactaacctgtcactggatagatctggttctcctatggtaccttcatatgagacatctgtcagtccccaggctaaccgaacatatgttaggacagagaccactgaggatgaacgcaaaattcttctggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaagggcacaagctatagcaattggacaagcaatggttgatggacgttggctggattgtgttagtcatcacgaccagcttttcagagatgagtatgcgctgtatagaccactgcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctaattctgccagtcctagcaagcgcacatcagtcagcagtttccagtccacagtggacagtgactcagccgcttctatcagcctgaacgtggagctggacaacgtgaacttccatatcaagaagccctccaagtacccacatgtgccccctcaccctgctgaccaaaaaggtaggaggtagtcaccatctggaatccaaacacaggctggacagctgggccataggatctcatgccagcctgtccttctggcttcctctgtcccacgtggctgagggtatttggtgtgggttttgttctaccctttctcatttctcccacacctcctccccataccttttttggttgattccttttatttcttgttttctccctcagcaccaccatgcccagtgtaaagcttgtagtctttaaaaggaactacatctgactgctcttcccataatgtgcaacttccaaggtggcatttctttgcatttataacatggatgttctgctctattcttttctctttcttcatttaacattttaaagcctatttgcctttgccgttacccagtaacataatatttcactaaacccattaagatgcttgaactagattgggggaaaatcattcacacattccaaaatatctccataataattcataatgcttattatgtatattctatatttattaaaacaatttcttctctaatgtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - RNA, 2421 nt, Homo sapiens
caaccatgtaagcagcttcgcttcctgccgcaaccgtccgcggcctgaggagcccaccgccgctctcgggggccgacttccgggggctgagccgttgaagcggaggctggggcggggggcagccggcgcggccggggcaggaggcgcagactcatgaaatggccacagatgataagacgtccccaacactggactctgctaatgatttgcctcgatctcctactagtccttctcatctcacacactttaaacctttgactcctgatcaagatgagcccccttttaaatcagcttatagttcttttgtaaatctctttcgttttaacaaagagagagcagaaggaggccagggagaacagcagcctttgagtggaagttggaccagccctcagctcccttcgaggacacagtctgttaggtcacccacaccttataaaaagcagcttaatgaggaactccagcggcgctcttcagcattagacacaagaaggaaagcagaacctacctttggaggtcatgaccctcgtacagctgttcagcttcgaagcctcagcacagtattaaaacgcctcaaggaaatcatggaggggaaaagccaggatagtgacctgaaacaatactggatgccagatagccaatgtaaagagtgctatgactgtagtgagaaatttacaacctttaggcgcagacaccattgccgactatgtgggcagattttctgcagtcgttgctgtaatcaagaaatccctggaaaatttatgggctatacaggagacctccgagcttgcacatattgtagaaaaatagccttaagttatgctcattccacagacagtaattctattggggaagacttgaatgctctttcagattctgcttgctctgtgtctgtgcttgatccaagtgaaccccgaacacctgttgggagtaggaaagccagccgtaacatatttttagaggatgatttggcctggcaaagtttgattcatccagattcctcaaatactcctctttcaacaagacttgtatctgtgcaagaggatgctgggaaatctcctgctcgaaatagatcagccagcattactaacctgtcactggatagatctggttctcctatggtaccttcatatgagacatctgtcagtccccaggctaaccgaacatatgttaggacagagaccactgaggatgaacgcaaaattcttctggacagtgtgcagttaaaagacctgtggaaaaaaatctgccatcacagcagtggaatggagtttcaggatcaccgctactggttgagaacgcatcccaactgcattgtaggaaaggaattagtcaactggctaatccgaaatgggcatattgccacaagggcacaagctatagcaattggacaagcaatggttgatggacgttggctggattgtgttagtcatcacgaccagcttttcagagatgagtatgcgctgtatagaccactgcagagtacagaattttctgagacgccttctcccgacagtgactcagtgaactccgtggaaggacactctgagccatcctggtttaaagacataaagtttgatgacagtgacacagaacagatagctgaagaaggtgacgataatttggctaattctgccagtcctagcaagcgcacatcagtcagcagtttccagtccacagtggacagtgactcagccgcttctatcagcctgaacgtggagctggacaacgtgaacttccatatcaagaagccctccaagtacccacatgtgccccctcaccctgctgaccaaaaaggtaggaggtagtcaccatctggaatccaaacacaggctggacagctgggccataggatctcatgccagcctgtccttctggcttcctctgtcccacgtggctgagggtatttggtgtgggttttgttctaccctttctcatttctcccacacctcctccccataccttttttggttgattccttttatttcttgttttctccctcagcaccaccatgcccagtgtaaagcttgtagtctttaaaaggaactacatctgactgctcttcccataatgtgcaacttccaaggtggcatttctttgcatttataacatggatgttctgctctattcttttctctttcttcatttaacattttaaagcctatttgcctttgccgttacccagtaacataatatttcactaaacccattaagatgcttgaactagattgggggaaaatcattcacacattccaaaatatctccataataattcataatgcttattatgtatattctatatttattaaaacaatttcttctctaatgtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - protein, 2098 aa, Homo sapiens
MATDDKTSPTLDSANDLPRSPTSPSHLTHFKPLTPDQDEPPFKSAYSSFVNLFRFNKERAEGGQGEQQPLSGSWTSPQLPSRTQSVRSPTPYKKQLNEELQRRSSALDTRRKAEPTFGGHDPRTAVQLRSLSTVLKRLKEIMEGKSQDSDLKQYWMPDSQCKECYDCSEKFTTFRRRHHCRLCGQIFCSRCCNQEIPGKFMGYTGDLRACTYCRKIALSYAHSTDSNSIGEDLNALSDSACSVSVLDPSEPRTPVGSRKASRNIFLEDDLAWQSLIHPDSSNTPLSTRLVSVQEDAGKSPARNRSASITNLSLDRSGSPMVPSYETSVSPQANRTYVRTETTEDERKILLDSVQLKDLWKKICHHSSGMEFQDHRYWLRTHPNCIVGKELVNWLIRNGHIATRAQAIAIGQAMVDGRWLDCVSHHDQLFRDEYALYRPLQSTEFSETPSPDSDSVNSVEGHSEPSWFKDIKFDDSDTEQIAEEGDDNLANSASPSKRTSVSSFQSTVDSDSAASISLNVELDNVNFHIKKPSKYPHVPPHPADQKEYLISDTGGQQLSISDAFIKESLFNRRVEEKSKELPFTPLGWHHNNLELLREENGEKQAMERLLSANHNHMMALLQQLLHSDSLSSSWRDIIVSLVCQVVQTVRPDVKNQDDDMDIRQFVHIKKIPGGKKFDSVVVNGFVCTKNIAHKKMSSCIKNPKILLLKCSIEYLYREETKFTCIDPIVLQEREFLKNYVQRIVDVRPTLVLVEKTVSRIAQDMLLEHGITLVINVKSQVLERISRMTQGDLVMSMDQLLTKPHLGTCHKFYMQIFQLPNEQTKTLMFFEGCPQHLGCTIKLRGGSDYELARVKEILIFMICVAYHSQLEISFLMDEFAMPPTLMQNPSFHSLIEGRGHEGAVQEQYGGGSIPWDPDIPPESLPCDDSSLLELRIVFEKGEQENKNLPQAVASVKHQEHSTTACPAGLPCAFFAPVPESLLPLPVDDQQDALGSEQPETLQQTVVLQDPKSQIRAFRDPLQDDTGLYVTEEVTSSEDKRKTYSLAFKQELKDVILCISPVITFREPFLLTEKGMRCSTRDYFAEQVYWSPLLNKEFKEMENRRKKQLLRDLSGLQGMNGSIQAKSIQVLPSHELVSTRIAEHLGDSQSLGRMLADYRARGGRIQPKNSDPFAHSKDASSTSSGQSGSKNEGDEERGLILSDAVWSTKVDCLNPINHQRLCVLFSSSSAQSSNAPSACVSPWIVTMEFYGKNDLTLGIFLERYCFRPSYQCPSMFCDTPMVHHIRRFVHGQGCVQIILKELDSPVPGYQHTILTYSWCRICKQVTPVVALSNESWSMSFAKYLELRFYGHQYTRRANAEPCGHSIHHDYHQYFSYNQMVASFSYSPIRLLEVCVPLPKIFIKRQAPLKVSLLQDLKDFFQKVSQVYVAIDERLASLKTDTFSKTREEKMEDIFAQKEMEEGEFKNWIEKMQARLMSSSVDTPQQLQSVFESLIAKKQSLCEVLQAWNNRLQDLFQQEKGRKRPSVPPSPGRLRQGEESKISAMDASPRNISPGLQNGEKEDRFLTTLSSQSSTSSTHLQLPTPPEVMSEQSVGGPPELDTASSSEDVFDGHLLGSTDSQVKEKSTMKAIFANLLPGNSYNPIPFPFDPDKHYLMYEHERVPIAVCEKEPSSIIAFALSCKEYRNALEELSKATQWNSAEEGLPTNSTSDSRPKSSSPIRLPEMSGGQTNRTTETEPQPTKKASGMLSFFRGTAGKSPDLSSQKRETLRGADSAYYQVGQTGKEGTENQGVEPQDEVDGGDTQKKQLINPHVELQFSDANAKFYCRLYYAGEFHKMREVILDSSEEDFIRSLSHSSPWQARGGKSGAAFYATEDDRFILKQMPRLEVQSFLDFAPHYFNYITNAVQQKRPTALAKILGVYRIGYKNSQNNTEKKLDLLVMENLFYGRKMAQVFDLKGSLRNRNVKTDTGKESCDVVLLDENLLKMVRDNPLYIRSHSKAVLRTSIHSDSHFLSSHLIIDYSLLVGRDDTSNELVVGIIDYIRTFTWDKKLEMVVKSTGILGGQGKMPTVVSPELYRTRFCEAMDKYFLMVPDHWTGLGLNC

SEQ ID NO: 12 - protein, 451 aa, Homo sapiens
MATDDKTSPTLDSANDLPRSPTSPSHLTHFKPLTPDQDEPPFKSAYSSFVNLFRFNKERAEGGQGEQQPLSGSWTSPQLPSRTQSVRSPTPYKKQLNEELQRRSSALGDLRACTYCRKIALSYAHSTDSNSIGEDLNALSDSACSVSVLDPSEPRTPVGSRKASRNIFLEDDLAWQSLIHPDSSNTPLSTRLVSVQEDAGKSPARNRSASITNLSLDRSGSPMVPSYETSVSPQANRTYVRTETTEDERKILLDSVQLKDLWKKICHHSSGMEFQDHRYWLRTHPNCIVGKELVNWLIRNGHIATRAQAIAIGQAMVDGRWLDCVSHHDQLFRDEYALYRPLQSTEFSETPSPDSDSVNSVEGHSEPSWFKDIKFDDSDTEQIAEEGDDNLANSASPSKRTSVSSFQSTVDSDSAASISLNVELDNVNFHIKKPSKYPHVPPHPADQKGRR

SEQ ID NO: 13 - protein, 548 aa, Homo sapiens
MATDDKTSPTLDSANDLPRSPTSPSHLTHFKPLTPDQDEPPFKSAYSSFVNLFRFNKERAEGGQGEQQPLSGSWTSPQLPSRTQSVRSPTPYKKQLNEELQRRSSALDTRRKAEPTFGGHDPRTAVQLRSLSTVLKRLKEIMEGKSQDSDLKQYWMPDSQCKECYDCSEKFTTFRRRHHCRLCGQIFCSRCCNQEIPGKFMGYTGDLRACTYCRKIALSYAHSTDSNSIGEDLNALSDSACSVSVLDPSEPRTPVGSRKASRNIFLEDDLAWQSLIHPDSSNTPLSTRLVSVQEDAGKSPARNRSASITNLSLDRSGSPMVPSYETSVSPQANRTYVRTETTEDERKILLDSVQLKDLWKKICHHSSGMEFQDHRYWLRTHPNCIVGKELVNWLIRNGHIATRAQAIAIGQAMVDGRWLDCVSHHDQLFRDEYALYRPLQSTEFSETPSPDSDSVNSVEGHSEPSWFKDIKFDDSDTEQIAEEGDDNLANSASPSKRTSVSSFQSTVDSDSAASISLNVELDNVNFHIKKPSKYPHVPPHPADQKGRR